SEQUENCE LISTING (142 sequences)

SEQ ID NO: 1 - protein, 5 aa, Artificial Sequence
  Linker EP2_2
GSGGS

SEQ ID NO: 2 - protein, 4 aa, Artificial Sequence
  Linker of E7
SKRG

SEQ ID NO: 3 - protein, 4 aa, Artificial Sequence
  Linker of C9
WRLG

SEQ ID NO: 4 - protein, 4 aa, Artificial Sequence
  Linker of B5
QTGW

SEQ ID NO: 5 - protein, 4 aa, Artificial Sequence
  Linker of E3
GGWG

SEQ ID NO: 6 - protein, 4 aa, Artificial Sequence
  Linker of Clone #1 (F8)
GLRG

SEQ ID NO: 7 - protein, 5 aa, Artificial Sequence
  Linker of Clone #2 (A11)
PFGSS

SEQ ID NO: 8 - protein, 5 aa, Artificial Sequence
  Linker of Clone #4 (D12)
PLGDH

SEQ ID NO: 9 - protein, 4 aa, Artificial Sequence
  Linker of Clone #5 (H1)
GRGG

SEQ ID NO: 10 - protein, 5 aa, Artificial Sequence
  Linker of Clone #6 (E4)
AGIDH

SEQ ID NO: 11 - protein, 4 aa, Artificial Sequence
  Linker of Clone #8 (C1)
SPKG

SEQ ID NO: 12 - protein, 5 aa, Artificial Sequence
  Linker of Clone #10 (C6)
YSGQW

SEQ ID NO: 13 - protein, 4 aa, Artificial Sequence
  Linker of Clone #13 (H10)
GDWG

SEQ ID NO: 14 - protein, 61 aa, Artificial Sequence
  DENV sequence on Figure 3, residues 60-120, Dengue1
CIEAKISNTTTDSRCPTQGEATLVEEQDANFVCRRTFVDRGWGNGCGLFGKGSLITCAKFT

SEQ ID NO: 15 - protein, 61 aa, Artificial Sequence
  ZIKA sequence on Figure 3, residues 60-120
CYEASISDMASDSRCPTQGEAYLDKQSDTQYVCYRTLVDRGWGNGCGLFGKGSLVTCAKFA

SEQ ID NO: 16 - protein, 90 aa, Artificial Sequence
  Amino acid sequence on Fig. 13A
SYSMCTGKFKVVKEIAETQHGTIVIRVQYEGDGSPCKIPFEIMDLEKRHVLGRLITVNPIVTEKDSPVNIEAEPPFGDSYIIIGVEPGQL

SEQ ID NO: 17 - protein, 89 aa, Artificial Sequence
  Amino acid sequence on Fig. 13B
MGMSYVMCTGSFKLEKEVAETQHGTVLVQVKYEGTDAPCKIPFSTQDEKGVTQNGRLITANPIVTDKEKPVNIETEPPFGESYIIVGAG

SEQ ID NO: 18 - protein, 90 aa, Artificial Sequence
  Amino acid sequence on Fig. 13C
MGMSYSMCTGKFKIVKEIAETQHGTIVIRVQYEGDGSPCKIPFEIMDLEKRHVLGRLITVNPIVTEKDSPVNIEAEPPFGDSYIIIGVEP

SEQ ID NO: 19 - protein, 89 aa, Artificial Sequence
  Amino acid sequence on Fig. 13D
KGMSYAMCLNTFVLKKEVSETQHGTILIKVEYKGEDAPCKIPFSTEDGQGKAHNGRLITANPVVTKKEEPVNIEAEPPFGESNIVIGIG

SEQ ID NO: 20 - protein, 88 aa, Artificial Sequence
  Amino acid sequence on Fig. 13E
MGMSYTMCSGKFSIDKEMAETQHGTTVVKVKYEGAGAPCKVPIEIRDVNKEKVVGRIISSTPFAENTNSVTNIELEPPFGDSYIVIGV

SEQ ID NO: 21 - protein, 90 aa, Artificial Sequence
  Amino acid sequence on Fig. 14A
TLKGISYVMCTGSFKLEKEVAETQHGTVLVQVKYEGTDAPCKIPFSSQDEKGVTQNGRLVTANPIVTDKEKPVNIEAEPPFGESYIVVGA

SEQ ID NO: 22 - protein, 90 aa, Artificial Sequence
  Amino acid sequence on Fig. 14B
KGMSYSMCTGKFKVVKEIAETQHGTIVIRVQYEGDGSPCKIPFEIMDLEKRHVLGRLITVNPIVTEKDSPVNIEAEPPFGDSYIIIGVEP

SEQ ID NO: 23 - protein, 89 aa, Artificial Sequence; identical to SEQ ID NO: 19
  Amino acid sequence on Fig. 14C

SEQ ID NO: 24 - protein, 85 aa, Artificial Sequence
  Amino acid sequence on Fig. 14D
KGMSYTMCSGKFSIDKEMAETQHGTTVVKVKYEGAGAPCKVPIEIRDVNKEKVVGRIISSTPLAENTNSVTNIELEPPFGDSYIV

SEQ ID NO: 25 - protein, 57 aa, Artificial Sequence
  Dengue1, residues 64-120
KISNTTTDSRCPTQGEATLVEEQDANFVCRRTFVDRGWGNGCGLFGKGSLITCAKFK

SEQ ID NO: 26 - protein, 57 aa, Artificial Sequence
  Dengue2, residues 64-120
KLTNTTTESRCPIQGEPSLNEEQDKRFVCKHSMVDRGWGNGCGLFGKGGIVTCAMFT

SEQ ID NO: 27 - protein, 57 aa, Artificial Sequence
  Dengue3, residues 64-120
KITNITTDSRCPTQGEAILPEEQDQNYVCKHTYVDRGWGNGCGLFGKGSLVTCAKFQ

SEQ ID NO: 28 - protein, 57 aa, Artificial Sequence
  Dengue4, residues 64-120
SISNITTATRCPTQGEPYLKEEQDQQYICRRDMVDRGWGNGCGLFGKGGVVTCAKFS

SEQ ID NO: 29 - protein, 62 aa, Artificial Sequence
  Dengue1, clone EP1.1, residues 64-120
FRKISNTTTDSRCPTQGEATLVEEQDANFVCRRTFVDRGWGNGCGLFGKGSLITCAKFKTSR

SEQ ID NO: 30 - protein, 61 aa, Artificial Sequence
  Dengue1, clone EP1.2, residues 64-120
ALHKISNTTTDSRCPTQGEATLVEEQDANFVCRRTFVDRGWGNGCGLFGKGSLITCAKFKT

SEQ ID NO: 31 - protein, 59 aa, Artificial Sequence
  Dengue1, clone EP1.3, residues 64-120
SKISNTTTDSRCPTQGEATLVEEQDANFVCRRTFVDRGWGNGCGLFGKGSLITCAKFKH

SEQ ID NO: 32 - protein, 61 aa, Artificial Sequence
  Dengue1, clone EP1.4, residues 64-120
IKRKISNTTTDSRCPTQGEATLVEEQDANFVCRRTFVDRGWGNGCGLFGKGSLITCAKFKP

SEQ ID NO: 33 - protein, 60 aa, Artificial Sequence
  Dengue1, clone EP1.5, residues 64-120
RKISNTTTDSRCPTQGEATLVEEQDANFVCRRTFVDRGWGNGCGLFGKGSLITCAKFKST

SEQ ID NO: 34 - protein, 62 aa, Artificial Sequence
  Dengue1, clone EP1.6, residues 64-120
HLLKISNTTTDSRCPTQGEATLVEEQDANFVCRRTFVDRGWGNGCGLFGKGSLITCAKFKTT

SEQ ID NO: 35 - protein, 60 aa, Artificial Sequence
  Dengue1, clone EP1.7, residues 64-120
NAPKISNTTTDSRCPTQGEATLVEEQDANFVCRRTFVDRGWGNGCGLFGKGSLITCAKFK

SEQ ID NO: 36 - protein, 61 aa, Artificial Sequence
  Dengue1, clone EP1.8, residues 64-120
GKISNTTTDSRCPTQGEATLVEEQDANFVCRRTFVDRGWGNGCGLFGKGSLITCAKFKTRT

SEQ ID NO: 37 - protein, 60 aa, Artificial Sequence
  Dengue1, clone EP1.9, residues 64-120
SQIKISNTTTDSRCPTQGEATLVEEQDANFVCRRTFVDRGWGNGCGLFGKGSLITCAKFK

SEQ ID NO: 38 - protein, 62 aa, Artificial Sequence
  Dengue1, clone EP1.10, residues 64-120
CALKISNTTTDSRCPTQGEATLVEEQDANFVCRRTFVDRGWGNGCGLFGKGSLITCAKFKHT

SEQ ID NO: 39 - protein, 70 aa, Artificial Sequence
  Dengue1, residues 301-370
MCTGSFKLEKEVAETQHGTVLVQVKYEGTDAPCKIPFSSQDEKGVTQNGRLITANPIVTDKEKPVNIEAE

SEQ ID NO: 40 - protein, 70 aa, Artificial Sequence
  Dengue2, residues 301-370
MCTGKFKIVKEIAETQHGTIVIRVQYEGDGSPCKIPFEIMDLEKRHVLGRLITVNPIVTEKDSPVNIEAE

SEQ ID NO: 41 - protein, 70 aa, Artificial Sequence
  Dengue3, residues 301-370
MCLNTFVLKKEVSETQHGTILIKVEYKGEDAPCKIPFSTEDGQGKAHNGRLITANPVVTKKEEPVNIEAE

SEQ ID NO: 42 - protein, 70 aa, Artificial Sequence
  Dengue4, residues 301-370
MCSGKFSIDKEMAETQHGTTVVKVKYEGAGAPCKVPIEIRDVNKEKVVGRIISSTPLAENTNSVTNIELE

SEQ ID NO: 43 - protein, 20 aa, Artificial Sequence
  Dengue1, residues 336-355
PFSSQDEKGVTQNGRLITAN

SEQ ID NO: 44 - protein, 20 aa, Artificial Sequence
  Dengue2, residues 336-355
PFEIMDLEKRHVLGRLITVN

SEQ ID NO: 45 - protein, 20 aa, Artificial Sequence
  Dengue3, residues 336-355
PFSTEDGQGKAHNGRLITAN

SEQ ID NO: 46 - protein, 20 aa, Artificial Sequence
  Dengue4, residues 336-355
PIEIRDVNKEKVVGRIISST

SEQ ID NO: 47 - protein, 59 aa, Artificial Sequence
  Linker EP2_2
IWEMCTGSFKLEKEVAETQHGTVLVQVKYEGTDAPCKIGSGGSPIVTDKEKPVNIEAEK

SEQ ID NO: 48 - protein, 5 aa, Artificial Sequence
  Linker EP2 8
ANLVD

SEQ ID NO: 49 - protein, 4 aa, Artificial Sequence
  Linker EP2 11
RLNY

SEQ ID NO: 50 - protein, 4 aa, Artificial Sequence
  Linker EP2 13
EGLD

SEQ ID NO: 51 - protein, 4 aa, Artificial Sequence
  Linker EP2 20
CLVN

SEQ ID NO: 52 - protein, 4 aa, Artificial Sequence
  Linker EP2 21
ISVV

SEQ ID NO: 53 - DNA, 36 bp, Artificial Sequence
  PrimerI53_H1_F, Oligonucleotides of Table 3
gaaggagatatacatatgcctgctatgtgcaccggt

SEQ ID NO: 54 - DNA, 21 bp, Artificial Sequence
  21mer oligonucleotides of table 3 (#26)
aatgtgtggaattgtgagcgg

SEQ ID NO: 55 - protein, 4 aa, Artificial Sequence
  Linker EP2 40
RLNY

SEQ ID NO: 56 - protein, 56 aa, Artificial Sequence
  Linker of E7
NMCTGSFKLEKEVAETQHGTVLVQVKYEGTDAPCKISKRGPIVTDKEKPVNIEAET

SEQ ID NO: 57 - protein, 57 aa, Artificial Sequence
  Linker of C9
SRMCTGSFKLEKEVAETQHGTVLVQVKYEGTDAPCKIWRLGPIVTDKEKPVNIEAEY

SEQ ID NO: 58 - protein, 57 aa, Artificial Sequence
  Linker of B5
GRMCTGSFKLEKEVAETQHGTVLVQVKYEGTDAPCKIQTGWPIVTDKEKPVNIEAEL

SEQ ID NO: 59 - protein, 55 aa, Artificial Sequence
  Linker of E3
KMCTGSFKLEKEVAETQHGTVLVQVKYEGTDAPCKIGGWGPIVTDKEKPVNIEAE

SEQ ID NO: 60 - protein, 56 aa, Artificial Sequence
  Linker of F6
PMCTGSFKLEKEVAETQHGTVLVQVKYEGTDAPCKIRRLPIVTDKEKPVNIEAERS

SEQ ID NO: 61 - protein, 58 aa, Artificial Sequence
  Linker of H9
RGAMCTGSFKLEKEVAETQHGTVLVQVKYEGTDAPCKINNGPIVTDKEKPVNIEAEYL

SEQ ID NO: 62 - protein, 56 aa, Artificial Sequence
  Linker of B12
SMCTGSFKLEKEVAETQHGTVLVQVKYEGTDAPCKIGGGPIVTDKEKPVNIEAERL

SEQ ID NO: 63 - protein, 59 aa, Artificial Sequence
  Linker of Clone #1(F8)
TSRMCTGSFKLEKEVAETQHGTVLVQVKYEGTDAPCKIGLRGPIVTDKEKPVNIEAETL

SEQ ID NO: 64 - protein, 56 aa, Artificial Sequence
  Linker of Clone #2 (A11)
GMCTGSFKLEKEVAETQHGTVLVQVKYEGTDAPCKIPFGSSPIVTDKEKPVNIEAE

SEQ ID NO: 65 - protein, 57 aa, Artificial Sequence
  Linker of C7
RMCTGSFKLEKEVAETQHGTVLVQVKYEGTDAPCKIKGGPIVTDKEKPVNIEAENLA

SEQ ID NO: 66 - protein, 62 aa, Artificial Sequence
  Linker of Clone #4 (D12)
GFPMCTGSFKLEKEVAETQHGTVLVQVKYEGTDAPCKIGPLGDHPIVTDKEKPVNIEAERPV

SEQ ID NO: 67 - protein, 58 aa, Artificial Sequence
  Linker of Clone #5 (H1)
PAMCTGSFKLEKEVAETQHGTVLVQVKYEGTDAPCKIGRGGPIVTDKEKPVNIEAELL

SEQ ID NO: 68 - protein, 56 aa, Artificial Sequence
  Linker of Clone #6 (E4)
PMCTGSFKLEKEVAETQHGTVLVQVKYEGTDAPCKIAGIDHPIVTDKEKPVNIEAE

SEQ ID NO: 69 - DNA, 33 bp, Artificial Sequence
  PrimerI53_H1_R, Oligonucleotides of Table 3
tagctcctccatcttcatcagcagttccgcttc

SEQ ID NO: 70 - protein, 55 aa, Artificial Sequence
  Linker of Clone #8 (C1)
PMCTGSFKLEKEVAETQHGTVLVQVKYEGTDAPCKISPKGPIVTDKEKPVNIEAE

SEQ ID NO: 71 - protein, 56 aa, Artificial Sequence
  Linker of Clone #8 (C2)
HMCTGSFKLEKEVAETQHGTVLVQVKYEGTDAPCKIQGGPIVTDKEKPVNIEAETW

SEQ ID NO: 72 - protein, 61 aa, Artificial Sequence
  Linker of Clone #10 (C6)
TGLMCTGSFKLEKEVAETQHGTVLVQVKYEGTDAPCKIYSGQWPIVTDKEKPVNIEAETTQ

SEQ ID NO: 73 - protein, 57 aa, Artificial Sequence
  Linker of Clone #8 (D3)
PMCTGSFKLEKEVAETQHGTVLVQVKYEGTDAPCKIRFGPIVTDKEKPVNIEAEYMR

SEQ ID NO: 74 - protein, 56 aa, Artificial Sequence
  Linker of Clone #8 (E2)
VPMCTGSFKLEKEVAETQHGTVLVQVKYEGTDAPCKIEWNPIVTDKEKPVNIEAET

SEQ ID NO: 75 - protein, 57 aa, Artificial Sequence
  Linker of Clone #13 (H10)
RMCTGSFKLEKEVAETQHGTVLVQVKYEGTDAPCKIGDWGPIVTDKEKPVNIEAENI

SEQ ID NO: 76 - protein, 83 aa, Artificial Sequence
  FIGURE 15, Dengue1, residues 295-377
KGMSYVMCTGSFKLEKEVAETQHGTVLVQVKYEGTDAPCKIPFSSQDEKGVTQNGRLITANPIVTDKEKPVNIEAEPPFGESY

SEQ ID NO: 77 - protein, 83 aa, Artificial Sequence
  FIGURE 15, Dengue2, residues 295-377
MGMSYSMCTGKFKIVKEIAETQHGTIVIRVQYEGDGSPCKIPFEIMDLEKRHVLGRLITVNPIVTEKDSPVNIEAEPPFGDSY

SEQ ID NO: 78 - protein, 83 aa, Artificial Sequence
  FIGURE 15, Dengue3, residues 295-377
SGMSYAMCLNTFVLKKEVSETQHGTILIKVEYKGEDAPCKIPFSTEDGQGKAHNGRLITANPVVTKKEEPVNIEAEPPFGESN

SEQ ID NO: 79 - protein, 83 aa, Artificial Sequence
  FIGURE 15, Dengue 4, residues 295-377
SGMSYTMCSGKFSIDKEMAETQHGTTVVKVKYEGAGAPCKVPIEIRDVNKEKVVGRIISSTPLAENTNSVTNIELEPPFGDSY

SEQ ID NO: 80 - protein, 94 aa, Artificial Sequence
  FIGURE 5, Dengue1, residues 298-394
SYVMCTGSFKLEKEVAETQHGTVLVQIKYEGTDAPCKIPFSTQDEKGVTQNGRLITANPIVTDKEKPVNIEAEPPFGESYIVIGAGEKALKLSW

SEQ ID NO: 81 - protein, 94 aa, Artificial Sequence
  FIGURE 5, Dengue2, residues 298-394
SYSMCTGKFKVVKEIAETQHGTIVIRVQYEGDGSPCKIPFEIMDLEKRHVLGRLITVNPIVTEKDSPVNIEAEPPFGDSYIIIGVEPGQLKLSW

SEQ ID NO: 82 - protein, 94 aa, Artificial Sequence
  FIGURE 5, Dengue3, residues 298-394
SYAMCLNTFVLKKEVSETQHGTILIKVEYKGEDAPCKIPFSTEDGQGKAHNGRLITANPVVTKKEEPVNIEAEPPFGESNIVIGIGDKALKINW

SEQ ID NO: 83 - protein, 94 aa, Artificial Sequence
  FIGURE 5, Dengue 4, residues 298-394
SYTMCSGKFSIDKEMAETQHGTTVVKVKYEGAGAPCKVPIEIRDVNKEKVVGRVISATPLAENTNSVTNIELEPPFGDSYIVIGVGNSALTLHW

SEQ ID NO: 84 - protein, 50 aa, Artificial Sequence
  FIGURE 6B, Dengue1, residues M301-I335/P356-E370
MCTGSFKLEKEVAETQHGTVLVQIKYEGTDAPCKIPIVTDKEKPVNIEAE

SEQ ID NO: 85 - protein, 50 aa, Artificial Sequence
  FIGURE 6B, Dengue2, residues M301-I335/P356-E370
MCTGKFKVVKEIAETQHGTIVIRVQYEGDGSPCKIPIVTEKDSPVNIEAE

SEQ ID NO: 86 - protein, 50 aa, Artificial Sequence
  FIGURE 6B, Dengue3, residues M301-I335/P356-E370
MCLNTFVLKKEVSETQHGTILIKVEYKGEDAPCKIPVVTKKEEPVNIEAE

SEQ ID NO: 87 - protein, 50 aa, Artificial Sequence
  FIGURE 6B, Dengue4, residues M301-I335/P356-E370
MCSGKFSIDKEMAETQHGTTVVKVKYEGAGAPCKVPLAENTNSVTNIELE

SEQ ID NO: 88 - protein, 35 aa, Artificial Sequence
  RESIDUES 301-335 OF DENGUE SEROTYPE 1
MCTGSFKLEKEVAETQHGTVLVQVKYEGTDAPCKI

SEQ ID NO: 89 - protein, 35 aa, Artificial Sequence
  RESIDUES 301-335 OF DENGUE SEROTYPE 2
MCTGKFKIVKEIAETQHGTIVIRVQYEGDGSPCKI

SEQ ID NO: 90 - protein, 35 aa, Artificial Sequence
  RESIDUES 301-335 OF DENGUE SEROTYPE 3
MCLNTFVLKKEVSETQHGTILIKVEYKGEDAPCKI

SEQ ID NO: 91 - protein, 35 aa, Artificial Sequence
  RESIDUES 301-335 OF DENGUE SEROTYPE 4
MCSGKFSIDKEMAETQHGTTVVKVKYEGAGAPCKV

SEQ ID NO: 92 - protein, 15 aa, Artificial Sequence
  RESIDUES 356-370 OF DENGUE SEROTYPE 1
PIVTDKEKPVNIEAE

SEQ ID NO: 93 - protein, 15 aa, Artificial Sequence
  RESIDUES 356-370 OF DENGUE SEROTYPE 2
PIVTEKDSPVNIEAE

SEQ ID NO: 94 - protein, 15 aa, Artificial Sequence
  RESIDUES 356-370 OF DENGUE SEROTYPE 3
PVVTKKEEPVNIEAE

SEQ ID NO: 95 - protein, 15 aa, Artificial Sequence
  RESIDUES 356-370 OF DENGUE SEROTYPE 4
PLAENTNSVTNIELE

SEQ ID NO: 96 - protein, 542 aa, Dengue virus type 1
SMAMRCVGIGSRDFVEGLSGATWVDVVLEHGSCVTTMAKDKPTLDIELLKTEVTDPAVLRKLCIEAKISNTTTDSRCPTQGEATLVEEQDANFVCRRTFVDRGWGNGCGLFGKGSLITCAKFKCVTKLEGKIVQYENLKYSVIVTVHTGDQHQVGNESTEHGTTATITPQAPTTEIQLTDYGALTLDCSPRTGLDFNEMVLLTMKEKSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQIKYEGTDAPCKIPFSTQDEKGVTQNGRLITANPIVTDKEKPVNIEAEPPFGESYIVIGAGEKALKLSWFKKGSSIGKMFEATARGARRMAILGDTAWDFGSIGGVFTSVGKLVHQIFGTAYGVLFSGVSWTMKIGIGVLLTWLGLNSRSTSLSMTCIAVGLVTLYLGVMVQADSGCVINWKGRELKCGSGIFVTNEVHTWTEQYKFQADSPKRLSA

SEQ ID NO: 97 - protein, 542 aa, Dengue virus type 2
SMTMRCIGISNRDFVEGVSGGSWVDIVLEHGSCVTTMAKNKPTLDFELIKTEAKHPATLRKYCIEAKLTNTTTASRCPTQGEPSLNEEQDKRFVCKHSMVDRGWGNGCGLFGKGGIVTCAMFTCKKNMEGKIVQPENLEYTIVITPHSGEENAVGNDTGKHGKEIKVTPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLSWFKKGSSIGQMFETTMRGAKRMAILGDTAWDFGSLGGVFTSIGKALHQVFGAIYGAAFSGVSWTMKILIGVVITWIGMNSRSTSLSVSLVLVGIVTLYLGVMVQADSGCVVSWKNKELKCGSGIFITDNVHTWTEQYKFQPESPSKLAS

SEQ ID NO: 98 - protein, 540 aa, Dengue virus type 3
SMAMRCVGVGNRDFVEGLSGATWVDVVLEHGGCVTTMAKNKPTLDIELQKTEATQLATLRKLCIEGKITNITTDSRCPTQGEAILPEEQDQNYVCKHTYVDRGWGNGCGLFGKGSLVTCAKFQCLESIEGKVVQHENLKYTVTITVHTGDQHQVGNDTQGVTAEITPQASTVEAILPEYGTLGLECSPRTGLDFNEMILLTMKNKAWMVHRQWFFDLPLPWTSGATTETPTWNRKELLVTFKNAHAKKQEVVVLGSQEGAMHTALTGATEIQNSGGTSIFAGHLKCRLKMDKLELKGMSYAMCLNTFVLKKEVSETQHGTILIKVEYKGEDAPCKIPFSTEDGQGKAHNGRLITANPVVTKKEEPVNIEAEPPFGESNIVIGIGDKALKINWYKKGSSIGKMFEATARGARRMAILGDTAWDFGSVGGVLNSLGKMVHQIFGSAYTALFGGVSWIMKIGIGVLLTWIGLNSKNTSMSFSCIVIGIITLYLGTVVQADMGCVINWKGKELKCGSGIFVTNEVHTWTEQYKFQADSPKRLAT

SEQ ID NO: 99 - protein, 542 aa, Dengue virus type 4
SYGMRCVGVGNRDFVEGVSGGAWVDLVLEHGGCVTTMAQGKPTLDFELTKTTAKEVALLRTYCIEALISNITTATRCPTQGEPYLKEEQDQQYICRRDVVDRGWGNGCGLFGKGGVVTCAKFSCSGKITGNLVQIENLEYTVVVTVHNGDTHAVGNDTSNHGVTATITPRSPSVEVKLPDYGELTLDCEPRSGIDFNEMILMKMKKKTWLVHKQWFLDLPLPWTTGADTSEVHWNHKERMVTFKVPHAKRQDVTVLGSQEGAMHSALAGATEVDSGDGNHMFAGHLKCKVRMEKLRIKGMSYTMCSGKFSIDKEMAETQHGTTVVKVKYEGAGAPCKVPIEIRDVNKEKVVGRVISATPLAENTNSVTNIELEPPFGDSYIVIGVGNSALTLHWFRKGSSIGKMFESTYRGAKRMAILGETAWDFGSVGGLFTSLGKAVHQVFGSVYTTMFGGVSWMIRILIGFLVLWIGTNSRNTSMAMTCIAVGGITLFLGFTVQADMGCVVSWSGRELKCGSGIFVVDNVHTWTEQYKFQPESPARLAS

SEQ ID NO: 100 - protein, 504 aa, ZIKA virus
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSA

SEQ ID NO: 101 - protein, 493 aa, Yellow fever virus
AHCIGITDRDFIEGVHGGTWVSATLEQGKCVTVMAPDKPSLDISLQTVAIDGPAEARKVCYSAVLTHVKINDKCPSTGEAHLAEENDGDNACKRTYSDRGWGNGCGLFGKGSIVACAKFTCAKSMSLFEVDRTKIQYVIRAQLHVGAKQENWNTDIKTLKFDALSGSQEAEFTGYGKATLECQVQTAVDFGNSYIAEMEKDSWIVDRQWAQDLTLPWQSGSGGTWREMHHLVEFEPPHAATIRVLALGNQEGSLKTALTGAMRVTKDENDNNLYKLHGGHVSCRVKLSALTLKGTSYKMCTDKMSFVKNPTDTGHGTVVMQVKVPKGAPCKIPVIVADDLTAAVNKGILVTVNPIASTNDDEVLIEVNPPFGDSYIIVGTGDSRLTYQWHKEGSSIGKLFTQTMKGAERLAVMGDAAWDFSSAGGFFTSVGKGIHTVFGSAFQGLFGGLSWITKVIMGAVLIWVGINTRNMTMSMSMILVGVIMMFLSLGVGA

SEQ ID NO: 102 - protein, 501 aa, West Nile virus
FNCLGMSNRDFLEGVSGATWVDLVLEGDSCVTIMSKDKPTIDVKMMNMEAANLAEVRSYCYLATVSDLSTKAACPTMGEAHNDKRADPAFVCRQGVVDRGWGNGCGLFGKGSIDTCAKFACSTKATGRTILKENIKYEVAIFVHGPTTVESHGNYSTQIGATQAGRFSITPAAPSYTLKLGEYGEVTVDCEPRSGIDTNAYYVMTVGTKTFLVHREWFMDLNLPWSSAGSTVWRNRETLMEFEEPHATKQSVIALGSQEGALHQALAGAIPVEFSSNTVKLTSGHLKCRVKMEKLQLKGTTYGVCSKAFKFLGTPADTGHGTVVLELQYTGTDGPCKVPISSVASLNDLTPVGRLVTVNPFVSVATANAKVLIELEPPFGDSYIVVGRGEQQINHHWHKSGSSIGKAFTTTLKGAQRLAALGDTAWDFGSVGGVFTSVGKAVHQVFGGAFRSLFGGMSWITQGLLGALLLWMGINARDRSIALTFLAVGGVLLFLSVNVHA

SEQ ID NO: 103 - DNA, 210 bp, Artificial Sequence
  Oligonucleotides of Table 3, Native DIII-with loop
atgtgcaccggttctttcaaactggaaaaagaagttgcggaaacccagcacggtaccgttctggttcaggttaaatacgaaggtaccgacgcgccgtgcaaaatcccgttctctacccaggacgaaaaaggtgttacccagaacggtcgtctgatcaccgcgaacccgatcgttaccgacaaagaaaaaccggttaacatcgaagcggaa

SEQ ID NO: 104 - DNA, 150 bp, Artificial Sequence
  Oligonucleotides of Table 3, Loop-less DIII
atgtgcaccggttctttcaaactggaaaaagaagttgcggaaacccagcacggtaccgttctggttcaggttaaatacgaaggtaccgacgcgccgtgcaaaatcccgatcgttaccgacaaagaaaaaccggttaacatcgaagcggaa

SEQ ID NO: 105 - DNA, 153 bp, Artificial Sequence
  Oligonucleotides of Table 3, DIII- 1NNK
  misc_feature (106)..(107): n is a, c, g, or t
  misc_feature (108)..(108): k is g or t
atgtgcaccggttctttcaaactggaaaaagaagttgcggaaacccagcacggtaccgttctggttcaggttaaatacgaaggtaccgacgcgccgtgcaaaatcnnkccgatcgttaccgacaaagaaaaaccggttaacatcgaagcggaa

SEQ ID NO: 106 - DNA, 156 bp, Artificial Sequence
  Oligonucleotides of Table 3, DIII- 2NNK
  misc_feature (106)..(107): n is a, c, g, or t
  misc_feature (108)..(108): k is g or t
  misc_feature (109)..(110): n is a, c, g, or t
  misc_feature (111)..(111): k is g or t
atgtgcaccggttctttcaaactggaaaaagaagttgcggaaacccagcacggtaccgttctggttcaggttaaatacgaaggtaccgacgcgccgtgcaaaatcnnknnkccgatcgttaccgacaaagaaaaaccggttaacatcgaagcggaa

SEQ ID NO: 107 - DNA, 159 bp, Artificial Sequence
  Oligonucleotides of Table 3, DIII- 3NNK
  misc_feature (106)..(107): n is a, c, g, or t
  misc_feature (108)..(108): k is g or t
  misc_feature (109)..(110): n is a, c, g, or t
  misc_feature (111)..(111): k is g or t
  misc_feature (112)..(113): n is a, c, g, or t
  misc_feature (114)..(114): k is g or t
atgtgcaccggttctttcaaactggaaaaagaagttgcggaaacccagcacggtaccgttctggttcaggttaaatacgaaggtaccgacgcgccgtgcaaaatcnnknnknnkccgatcgttaccgacaaagaaaaaccggttaacatcgaagcggaa

SEQ ID NO: 108 - DNA, 162 bp, Artificial Sequence
  Oligonucleotides of Table 3, DIII- 4NNK
  misc_feature (106)..(107): n is a, c, g, or t
  misc_feature (108)..(108): k is g or t
  misc_feature (109)..(110): n is a, c, g, or t
  misc_feature (111)..(111): k is g or t
  misc_feature (112)..(113): n is a, c, g, or t
  misc_feature (114)..(114): k is g or t
  misc_feature (115)..(116): n is a, c, g, or t
  misc_feature (117)..(117): k is g or t
atgtgcaccggttctttcaaactggaaaaagaagttgcggaaacccagcacggtaccgttctggttcaggttaaatacgaaggtaccgacgcgccgtgcaaaatcnnknnknnknnkccgatcgttaccgacaaagaaaaaccggttaacatcgaagcggaa

SEQ ID NO: 109 - DNA, 165 bp, Artificial Sequence
  Oligonucleotides of Table 3, DIII- 5NNK
  misc_feature (106)..(107): n is a, c, g, or t
  misc_feature (108)..(108): k is g or t
  misc_feature (109)..(110): n is a, c, g, or t
  misc_feature (111)..(111): k is g or t
  misc_feature (112)..(113): n is a, c, g, or t
  misc_feature (114)..(114): k is g or t
  misc_feature (115)..(116): n is a, c, g, or t
  misc_feature (117)..(117): k is g or t
  misc_feature (118)..(119): n is a, c, g, or t
  misc_feature (120)..(120): k is g or t
atgtgcaccggttctttcaaactggaaaaagaagttgcggaaacccagcacggtaccgttctggttcaggttaaatacgaaggtaccgacgcgccgtgcaaaatcnnknnknnknnknnkccgatcgttaccgacaaagaaaaaccggttaacatcgaagcggaa

SEQ ID NO: 110 - DNA, 20 bp, Artificial Sequence
  Oligonucleotides of Table 3, DIII_complementary
ttccgcttcgatgttaaccg

SEQ ID NO: 111 - DNA, 38 bp, Artificial Sequence
  Oligonucleotides of Table 3, 0NNK_F
actccgctccagagctctggatgtgcaccggttctttc

SEQ ID NO: 112 - DNA, 41 bp, Artificial Sequence
  Oligonucleotides of Table 3, 1NNK_F
  misc_feature (21)..(22): n is a, c, g, or t
  misc_feature (23)..(23): k is g or t
actccgctccagagctctggnnkatgtgcaccggttctttc

SEQ ID NO: 113 - DNA, 44 bp, Artificial Sequence
  Oligonucleotides of Table 3, 2NNK_F
  misc_feature (21)..(22): n is a, c, g, or t
  misc_feature (23)..(23): k is g or t
  misc_feature (24)..(25): n is a, c, g, or t
  misc_feature (26)..(26): k is g or t
actccgctccagagctctggnnknnkatgtgcaccggttctttc

SEQ ID NO: 114 - DNA, 47 bp, Artificial Sequence
  Oligonucleotides of Table 3, 3NNK_F
  misc_feature (21)..(22): n is a, c, g, or t
  misc_feature (23)..(23): k is g or t
  misc_feature (24)..(25): n is a, c, g, or t
  misc_feature (26)..(26): k is g or t
  misc_feature (27)..(28): n is a, c, g, or t
  misc_feature (29)..(29): k is g or t
actccgctccagagctctggnnknnknnkatgtgcaccggttctttc

SEQ ID NO: 115 - DNA, 40 bp, Artificial Sequence
  Oligonucleotides of Table 3, 0NNK_R
acagtttcccagacgtctggttccgcttcgatgttaaccg

SEQ ID NO: 116 - DNA, 43 bp, Artificial Sequence
  Oligonucleotides of Table 3, 1NNK_R
  misc_feature (21)..(21): m is a or c
  misc_feature (22)..(23): n is a, c, g, or t
acagtttcccagacgtctggmnnttccgcttcgatgttaaccg

SEQ ID NO: 117 - DNA, 46 bp, Artificial Sequence
  Oligonucleotides of Table 3, 2NNK_R
  misc_feature (21)..(21): m is a or c
  misc_feature (22)..(23): n is a, c, g, or t
  misc_feature (24)..(24): m is a or c
  misc_feature (25)..(26): n is a, c, g, or t
acagtttcccagacgtctggmnnmnnttccgcttcgatgttaaccg

SEQ ID NO: 118 - DNA, 49 bp, Artificial Sequence
  Oligonucleotides of Table 3, 3NNK_R
  misc_feature (21)..(21): m is a or c
  misc_feature (22)..(23): n is a, c, g, or t
  misc_feature (24)..(24): m is a or c
  misc_feature (25)..(26): n is a, c, g, or t
  misc_feature (27)..(27): m is a or c
  misc_feature (28)..(29): n is a, c, g, or t
acagtttcccagacgtctggmnnmnnmnnttccgcttcgatgttaaccg

SEQ ID NO: 119 - DNA, 42 bp, Artificial Sequence
  Oligonucleotides of Table 3, MBP_A11_F
ttccagggcatttcacatatgggtatgtgcaccggttctttc

SEQ ID NO: 120 - DNA, 42 bp, Artificial Sequence
  Oligonucleotides of Table 3, MBP_A11_R
gttttatttgaagcttatttattccgcttcgatgttaaccgg

SEQ ID NO: 121 - DNA, 42 bp, Artificial Sequence
  Oligonucleotides of Table 3, MBP_C6_F
ttccagggcatttcacatatgactggtttgatgtgcaccggt

SEQ ID NO: 122 - DNA, 42 bp, Artificial Sequence
  Oligonucleotides of Table 3, MBP_C6_R
gttttatttgaagcttatttactgagtagtttccgcttcgat

SEQ ID NO: 123 - DNA, 42 bp, Artificial Sequence
  Oligonucleotides of Table 3, MBP_F8_F
ttccagggcatttcacatatgacgagtcgtatgtgcaccggt

SEQ ID NO: 124 - DNA, 42 bp, Artificial Sequence
  Oligonucleotides of Table 3, MBP_F8_R
gttttatttgaagcttatttaaagagtttccgcttcgatgtt

SEQ ID NO: 125 - DNA, 42 bp, Artificial Sequence
  Oligonucleotides of Table 3, MBP_H1_F
ttccagggcatttcacatatgcctgctatgtgcaccggttct

SEQ ID NO: 126 - DNA, 42 bp, Artificial Sequence
  Oligonucleotides of Table 3, MBP_H1_R
gttttatttgaagcttatttacagcagttccgcttcgatgtt

SEQ ID NO: 127 - DNA, 21 bp, Artificial Sequence
  Oligonucleotides of Table 3, 20mer
ccctcatagttagcgtaacga

SEQ ID NO: 128 - DNA, 39 bp, Artificial Sequence
  PrimerI53_A11_F, Oligonucleotides of Table 3
taagaaggagatatacatatgggtatgtgcaccggttct

SEQ ID NO: 129 - DNA, 39 bp, Artificial Sequence
  PrimerI53_A11_R, Oligonucleotides of Table 3
gaatagctcctccatcttcatttccgcttcgatgttaac

SEQ ID NO: 130 - DNA, 36 bp, Artificial Sequence
  PrimerI53_F8_F, Oligonucleotides of Table 3
gaaggagatatacatatgacgagtcgtatgtgcacc

SEQ ID NO: 131 - DNA, 39 bp, Artificial Sequence
  PrimerI53_F8_R, Oligonucleotides of Table 3
gaatagctcctccatcttcataagagtttccgcttcgat

SEQ ID NO: 132 - protein, 462 aa, Artificial Sequence
  MBP-A11, MBP fusion with A11 clone
HHHHHHDIGSSKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGDIENLYFQGISHMGMCTGSFKLEKEVAETQHGTVLVQVKYEGTDAPCKIPFGSSPIVTDKEKPVNIEAE

SEQ ID NO: 133 - protein, 467 aa, Artificial Sequence
  MBP-C6, MBP fusion with C6 clone
HHHHHHDIGSSKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGDIENLYFQGISHMTGLMCTGSFKLEKEVAETQHGTVLVQVKYEGTDAPCKIYSGQWPIVTDKEKPVNIEAETTQ

SEQ ID NO: 134 - protein, 465 aa, Artificial Sequence
  MBP-F8, MBP fusion with F8 clone
HHHHHHDIGSSKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGDIENLYFQGISHMTSRMCTGSFKLEKEVAETQHGTVLVQVKYEGTDAPCKIGLRGPIVTDKEKPVNIEAETL

SEQ ID NO: 135 - protein, 464 aa, Artificial Sequence
  MBP-H1, MBP fusion with H1 clone
HHHHHHDIGSSKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGDIENLYFQGISHMPAMCTGSFKLEKEVAETQHGTVLVQVKYEGTDAPCKIGRGGPIVTDKEKPVNIEAELL

SEQ ID NO: 136 - protein, 289 aa, Artificial Sequence
  GST-A11, GST fusion with A11 clone
HHHHHHSGSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSGMCTGSFKLEKEVAETQHGTVLVQVKYEGTDAPCKIPFGSSPIVTDKEKPVNIEAE

SEQ ID NO: 137 - protein, 294 aa, Artificial Sequence
  GST-C6, GST fusion with C6 clone
HHHHHHSGSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSTGLMCTGSFKLEKEVAETQHGTVLVQVKYEGTDAPCKIYSGQWPIVTDKEKPVNIEAETTQ

SEQ ID NO: 138 - protein, 292 aa, Artificial Sequence
  GST-F8, GST fusion with F8 clone
HHHHHHSGSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSTSRMCTGSFKLEKEVAETQHGTVLVQVKYEGTDAPCKIGLRGPIVTDKEKPVNIEAETL

SEQ ID NO: 139 - protein, 291 aa, Artificial Sequence
  GST-H1, GST fusion with H1 clone
HHHHHHSGSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPAMCTGSFKLEKEVAETQHGTVLVQVKYEGTDAPCKIGRGGPIVTDKEKPVNIEAELL

SEQ ID NO: 140 - protein, 271 aa, Artificial Sequence
  I53-50A-A11, I53 fusion with A11 clone
GMCTGSFKLEKEVAETQHGTVLVQVKYEGTDAPCKIPFGSSPIVTDKEKPVNIEAEMKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHDILKLFPGEVVGPQFVKAMKGPFPNVKFVPTGGVNLDNVAKWFKAGVLAVGVGKALVKGKPDEVREKAKKFVKKIRGATEGSLEHHHHHH

SEQ ID NO: 141 - protein, 274 aa, Artificial Sequence
  I53-50A-F8, I53 fusion with F8 clone
TSRMCTGSFKLEKEVAETQHGTVLVQVKYEGTDAPCKIGLRGPIVTDKEKPVNIEAETLMKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHDILKLFPGEVVGPQFVKAMKGPFPNVKFVPTGGVNLDNVAKWFKAGVLAVGVGKALVKGKPDEVREKAKKFVKKIRGATEGSLEHHHHHH

SEQ ID NO: 142 - protein, 273 aa, Artificial Sequence
  I53-50A-H1, I53 fusion with H1clone
PAMCTGSFKLEKEVAETQHGTVLVQVKYEGTDAPCKIGRGGPIVTDKEKPVNIEAELLMKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHDILKLFPGEVVGPQFVKAMKGPFPNVKFVPTGGVNLDNVAKWFKAGVLAVGVGKALVKGKPDEVREKAKKFVKKIRGATEGSLEHHHHHH

(19) United States
(12) Patent Application Publication
(10) Pub. No.: US 2025/0276052 A1
(43) Pub. Date: Sep. 4, 2025

(54) RECONSTITUTION AND USES OF FLAVIVlRUS EPITOPES

(71) Applicant: RAMOT AT TEL-AVIV UNIVERSITY LTD., Tel Aviv (IL)

(72) Inventors: Jonathan M. GERSHONI, Tel Aviv (IL); Chen PILLER, Tel Aviv (IL)

(21) Appl. No.: 18/565,623

(22) PCT Filed: May 31, 2022
(86) PCT No.: PCT/IL2022/050578

§ 371 Date: Nov. 30, 2023

Related U.S. Application Data
(60) Provisional application No. 63/202,193, filed on May 31, 2021.

(51) Int. Cl. A61K 39/12; A61P 31/14; C07K 14/005; C07K 16/10; C12N 7/00; G01N 33/569; G01N 33/68

(52) U.S. Cl. CPC A61K 39/12; A61P 31/14; C07K 14/005; C07K 16/1081; C12N 7/00; G01N 33/56983; G01N 33/6854; C12N 2770/24122; C12N 2770/24134; G01N 2333/185; G01N 2469/20

(57) ABSTRACT

The present disclosure provides reconstituted epitopes derived from viruses of the Flaviviridae family, as well as various polypeptides comprising the reconstituted epitopes disclosed herein. Specifically, the E protein of viruses of the Flaviviridae, and any domains thereof, particularly, the Dill domain, that comprise the reconstituted epitope of the present disclosure. The invention further provides vaccines, and compositions comprising the reconstituted epitopes, as well as therapeutic and prophylactic methods thereof.

### FIELD OF THE INVENTION

The present invention relates to polypeptides and nucleic acid vaccines for viruses of the Flaviviridae family. More specifically, the invention provides reconstituted neutralizing epitope of Flaviviruses e.g., Dengue viruses, Zika viruses, Yellow fever virus and West Nile virus, compositions, vaccines, prophylactic, therapeutic and diagnostic methods and uses thereof.

### BACKGROUND ART

References considered to be relevant as background to the presently disclosed subject matter are listed below:
[1] Villar, Luis, et al. “Efficacy of a tetravalent dengue vaccine in children in Latin America.” New England Journal of Medicine 372.2 (2015): 113-123.
[2] Guy, Bruno, Melanie Saville, and Jean Lang. “Development of Sanofi Pasteur tetravalent dengue vaccine.” Human vaccines 6.9 (2010): 696-705.
[3] Simmons, Cameron P. “A candidate dengue vaccine walks a tightrope.” (2015): 1263-1264.
[4] Kirkpatrick, Beth D., et al. “Robust and balanced immune responses to all 4 dengue virus serotypes following administration of a single dose of a live attenuated tetravalent dengue vaccine to healthy, flavivirus-naive adults.” The Journal of infectious diseases 212.5 (2015): 702-710.

[5] Osorio, Jorge E., et al. “Safety and immunogenicity of a recombinant live attenuated tetravalent dengue vaccine (DENVax) in flavivirus-naive healthy adults in Colombia: a randomised, placebo-controlled, phase 1 study.” The Lancet Infectious Diseases 14.9 (2014): 830-838.
[6] Sabchareon, Arunee, et al. “Protective efficacy of the recombinant, live-attenuated, CYD tetravalent dengue vaccine in Thai schoolchildren: a randomised, controlled phase 2b trial.” The Lancet 380.9853 (2012): 1559-1567.
[7] Aguiar, Maíra, Nico Stollenwerk, and Scott B. Halstead. “The impact of the newly licensed dengue vaccine in endemic countries.” PLOS neglected tropical diseases 10.12 (2016): e0005179.
[8] Halstead, Scott B., and Philip K. Russell. “Protective and immunological behavior of chimeric yellow fever dengue vaccine.” Vaccine 34.14 (2016): 1643-1647.
[9] Halstead, Scott B. “Safety issues from a Phase 3 clinical trial of a live-attenuated chimeric yellow fever tetravalent dengue vaccine.” Human vaccines & immunotherapeutics just-accepted (2018): 00-00.

Acknowledgement of the above references herein is not to be inferred as meaning that these are in any way relevant to the patentability of the presently disclosed subject matter.

### BACKGROUND OF THE INVENTION

Infectious diseases have enormous impacts on global health and economics. Tropical diseases can cause death and are a huge economic and social burden in low- and middle-income countries. The present application focuses on developing therapies and vaccines against Flaviviruses such as Dengue, Zika virus, Yellow fever or West Nile viruses.

Dengue virus (DENV) is endemic in 100 countries with an estimated 390 million annual infections and 3.6 billion people at risk. Infection by the virus is either asymptomatic or causes febrile illness of Dengue fever (DF) with different levels of severity, with lethal illness in 1% of cases. Due to DF's debilitating effects, outbreaks have a severe economic impact in affected countries. Because several viral serotypes can lead to the disease, it is critical for effective treatment that therapeutic agents target all viral variants. Current vaccines are only partially effective, and thus there is an unmet need for effective vaccines for use as next-generation primary vaccines or as boosting agents for existing vaccines. Thus far, Dengue vaccines have been based on the intact virus envelope which can elicit an excessive antibody response and potentially lead to Antibody-Dependent Enhancement (ADE). There is a need to limit the production of excessive antibodies and focusing the immune response to specific neutralizing targets, thereby providing a means to avoid ADE. Considering the complexity of DENV infection and the risk of induction of enhancing antibodies with sub-optimal vaccines, an attractive alternative approach is to use subunits of DENV as boosting agents or next-generation vaccines.

Several Dengue vaccine-candidates are being developed and tested clinically, based on tetranomial mixtures of attenuated or chimeric viruses [1-4]. Although, some are relatively effective against serotypes 1, 3 and 4, none protect against all 4 serotypes contained in the vaccine. Hence, serotype 2 seems to be especially challenging [5-6]. Other vaccine-candidates appeared to induce Dengue infection-enhancing antibodies, resulting in increased risk of developing hospitalized disease during a subsequent wild type DENV exposure [7-9].

Zika virus is a mosquito-borne Flavivirus which causes an infection known as Zika fever or Zika virus disease. Outbreaks of Zika virus disease have been recorded in Africa, the Americas, Asia and the Pacific. In 2015, Brazil reported a large outbreak of rash illness, soon identified as Zika virus infection, and in July 2015, found to be associated with Guillain-Barré syndrome as well as microcephaly. Outbreaks and evidence of transmission soon appeared throughout the Americas, Africa, and other regions of the world. To date, a total of 86 countries and territories have reported evidence of mosquito-transmitted Zika infection.

Yellow fever virus is a Flavivirus transmitted to people primarily through the bite of infected Aedes or Haemagogus species mosquitoes. Mosquitoes acquire the virus by feeding on infected primates (human or non-human) and then can transmit the virus to other primates (human or non-human). People infected with yellow fever virus are infectious to mosquitoes (referred to as being “viremic”) shortly before the onset of fever and up to 5 days after onset.

West Nile virus (WNV) is a Flavivirus primarily transmitted by mosquitoes, mostly species of Culex. The primary hosts of WNV are birds, so that the virus remains within a “bird-mosquito-bird” transmission cycle. Humans and horses both exhibit viral disease symptoms that rarely occur in other animals.

There is therefore need in the art to develop novel vaccine candidates-engineered epitope focused immunogens (EFIs) that specifically focus the immune response towards neutralizing epitopes of these viruses. To avoid developing immunogens that do not recapitulate native antigens, as is often the case with isolated proteins fragments. The EFIs should be designed such that they would reconstitute minimal requirements of targeted antigens and adopt a conformation similar to the natural conformation. The present application benefits human health and subsistence, as well as the economy, by providing novel treatments and diagnostics against viral diseases.

### SUMMARY OF THE INVENTION

A first aspect of the present disclosure relates to a polypeptide comprising an amino acid sequence of at least one reconstituted epitope of a viral envelope protein (E protein). In some embodiments, the viral envelop protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII. It should be noted that the reconstituted epitope comprises at least one linker and at least one fragment of the native envelope protein.

In yet another aspect thereof, the present disclosure provides a DIII domain of an E protein of a virus of the Flaviviridae family, comprising the native DIII domain of an E protein of a virus of the Flaviviridae family or any fragments thereof and at least one linker. More specifically, at least one of such linker/s replaces a loop in the DIII domain, or any part thereof or amino acid residue/s thereof. In some specific embodiments, (a), the virus of the Flaviviridae family is a Dengue virus. In such case the loop may comprises an amino acid sequence that may start at any one of the amino acid residues 336, 333, 334, 335, 337, 338 or 339, and end at any one of the amino acid residues 355, 352, 353, 354, 356, 357 or 358.

A further aspect of the present disclosure relates to an envelope protein (E protein) of a virus of the Flaviviridae family, comprising the native E protein of a virus of the Flaviviridae family or any fragments thereof and at least one linker. In some embodiments, at least one of the linker/s replaces a loop in the DIII domain of the envelope protein, or any part thereof or amino acid residue/s thereof.

A further aspect of the present disclosure relates to a multimeric and/or multivalent antigen displaying platform and/or a nanoparticle scaffold comprising at least one reconstituted epitope of a viral envelope protein, any polypeptide, domain or viral envelope protein comprising the reconstituted epitope, any domain or viral envelope protein comprising at least one linker that replaces at least one loop or any part thereof or amino acid residue/s thereof in the viral protein. More specifically, in some embodiments, the viral envelope protein, specifically, the native E protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII. It should be further noted that the reconstituted epitope of the multimeric and/or multivalent antigen displaying platform disclosed herein may comprise at least one linker and at least one fragment of the native envelope protein. In further aspect of the present disclosure relates to at least one nucleic acid sequence encoding at least one reconstituted epitope of a viral envelope protein, any polypeptide, domain or viral envelope protein comprising the reconstituted epitope, any domain or viral envelope protein comprising at least one linker that replaces at least one loop or any part thereof or amino acid residue/s thereof in the viral protein, any multimeric and/or multivalent antigen displaying platform thereof, and any combinations thereof, any fusion protein, conjugate, polyvalent dendrimer thereof. More specifically, the viral envelope protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII. Still further, the reconstituted epitope comprises at least one linker and at least one fragment of the native envelope protein.

A further aspect provided by the present disclosure relates to a composition comprising an effective amount of at least one of, at least one polypeptide comprising an amino acid sequence of at least one reconstituted epitope of a viral envelope protein, and/or any polypeptide, domain or viral envelope protein comprising the reconstituted epitope, and/or any domain or viral envelope protein comprising at least one linker that replaces at least one loop or any part thereof or amino acid residue/s thereof in the viral protein, and/or any multimeric and/or multivalent antigen displaying platform thereof, and any combinations thereof, any derivative, enantiomer, fusion protein, conjugate, polyvalent dendrimer thereof, and/or any nucleic acid sequence encoding the same or any matrix, nano- or micro-particle thereof. In some embodiments, the viral envelope protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII. Still further, the reconstituted epitope as disclosed herein comprises at least one linker and at least one fragment of the native envelope protein. In some embodiments, the composition of the present disclosure may optionally further comprise at least one pharmaceutically acceptable carrier/s, excipient/s, adjuvant/s, auxiliaries, and/or diluent/s. A further aspect of the present disclosure relates to an anti-viral vaccine comprising at least one polypeptide comprising an amino acid sequence of at least one reconstituted epitope of a viral envelope protein, and/or any polypeptide, domain or viral envelope protein comprising the reconstituted epitope, any domain or viral envelope protein comprising at least one linker that replaces at least one loop or any part thereof, or amino acid residue/s thereof in said viral protein, and/or any multimeric and/or multivalent antigen displaying platform thereof, and any combinations thereof, any derivative, enantiomer, fusion protein, conjugate, polyvalent dendrimer thereof, and any nucleic acid sequence encoding the same, or any matrix, nano- or micro-particle thereof. The viral envelope protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII. Still further, in some embodiments, the reconstituted epitope comprises at least one linker and at least one fragment of the native envelope protein, said vaccine optionally further comprises at least one pharmaceutically acceptable carrier/s, excipient/s, adjuvant/s, auxiliaries, and/or diluent/s.

A further aspect of the present disclosure relates to a method for treating, preventing, inhibiting, reducing, eliminating, protecting or delaying the onset of an infection or an infectious clinical condition caused by a virus in a subject in need thereof. In some embodiments, the method comprising the step of administering to the subject an effective amount of at least one polypeptide comprising an amino acid sequence of at least one reconstituted epitope of a viral envelope protein, and/or any polypeptide, domain or viral envelope protein comprising the reconstituted epitope, and/or any domain or viral envelope protein comprising at least one linker that replaces at least one loop or any part thereof or amino acid residue/s thereof in said viral protein. In some embodiments the viral envelope protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII. The reconstituted epitope comprises at least one linker and at least one fragment of the native envelope protein. Still further, in some alternative or additional embodiments the methods disclosed herein may involve the administration of any multimeric and/or multivalent antigen displaying platform of the polypeptides disclosed herein, any derivative, enantiomer, fusion protein, conjugate, polyvalent dendrimer thereof, any nucleic acid sequence encoding the same, and any combinations thereof, any compositions thereof and any vaccine thereof. A further aspect of the present disclosure provides least one of, at least one polypeptide as defined by the present disclosure, at least one domain or viral envelope protein comprising at least one linker, as defined by the present disclosure, at least one multimeric and/or multivalent antigen displaying platform as defined by the present disclosure, at least one nucleic acid sequence as defined by the present disclosure, at least one composition as defined by the present disclosure, and at least one vaccine as defined by the present disclosure, for use in a method for treating, preventing, inhibiting, reducing, eliminating, protecting or delaying the onset of an infection or an infectious clinical condition caused by a virus in a subject in need thereof.

A further aspect of the present disclosure relates to a method of inducing an immune response against a virus of the Flaviviridae family in a subject in need thereof. In some embodiments, the method comprises administering to the subject an immunogenic effective amount of at least one polypeptide comprising an amino acid sequence of at least one of, at least one viral envelope protein, any polypeptide, domain or viral envelope protein comprising said reconstituted epitope, any domain or viral envelope protein comprising at least one linker that replaces at least one loop or any part thereof or amino acid residue/s thereof in said viral protein, any multimeric and/or multivalent antigen displaying platform thereof, and any combinations thereof, any derivative, enantiomer, fusion protein, conjugate, polyvalent dendrimer thereof, and any nucleic acid sequence encoding the same or any matrix, nano- or micro-particle thereof, or any composition or vaccine thereof. Still further aspect provided herein relates to least one of, at least one polypeptide as defined by the present disclosure, at least one domain or viral envelope protein comprising at least one linker, as defined by the present disclosure, at least one multimeric and/or multivalent antigen displaying platform as defined by the present disclosure, at least one nucleic acid sequence as defined by the present disclosure, at least one composition as defined by the present disclosure, and at least one vaccine as defined by the present disclosure, for use in a method for use in a method of inducing an immune response against a virus in a subject in need thereof. A further aspect of the present disclosure relates to a method for the preparation of a functional reconstituted epitope of a viral envelope protein. In some embodiments, the viral envelope protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII. More specifically, the method comprises the step of: first (a), screening a conformer library of epitopes of the viral envelope protein with at least one binding molecule. More specifically, the library comprising plurality of combinatorial display platforms or any display vehicles, each expressing a reconstituted epitope comprising at least one linker and at least one fragment of the native envelope protein. The next step (b), involves identifying and producing reconstituted epitope peptides which bind at least one of said binding molecules. A further aspect of the present disclosure relates to a method for producing an anti-viral vaccine comprising at least one reconstituted epitope of a viral envelope protein. More specifically, the viral envelop protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII. In some embodiments, the reconstituted epitope comprises at least one linker and at least one fragment of the native envelope protein. More specifically, the method may comprise the steps of: First step (a), involves preparing a reconstituted functional epitope of an envelope protein of the virus by a method as defined by the preset disclosure. The second step (b), involves admixing at least one of the reconstituted functional epitope/s of an envelope protein of said virus or any derivative or enantiomer thereof, or any fusion protein, conjugate, or polyvalent dendrimer comprising the same with at least one adjuvant/s, carrier/s, excipient/s, auxiliaries, and/or diluent/s.

A further aspect of the present disclosure relates to a method for the preparation, affinity selection and/or isolation of neutralizing antibodies that neutralize a virus. In some embodiments, the method comprising the steps of: First (a), contacting a serum or lymphocytes of at least one donor with an effective amount of reconstituted epitope of a viral envelope protein, any polypeptide, domain or viral envelope protein comprising the reconstituted epitope, any domain or viral envelope protein comprising at least one linker that replaces at least one loop or any part thereof or amino acid residue/s thereof in the viral protein. The viral envelope protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII. Still further, the reconstituted epitope comprises at least one linker and at least one fragment of the native envelope protein, or with any multimeric and/or multivalent antigen displaying platform thereof, any derivative, enantiomer, fusion protein, conjugate, polyvalent dendrimer thereof, and any combinations thereof. The second step (b), involves recovering the antibodies or at least one lymphocyte bound to the reconstituted epitope.

In a further aspect thereof, the present disclosure provides a method for treating, inhibiting, reducing, eliminating, protecting or delaying the onset of a viral infection in a subject in need thereof. The method comprising the step of administering to the subject an effective amount of a therapeutic passive vaccine comprising neutralizing antibodies that neutralize the virus. A further aspect of the present disclosure relates to a diagnostic method for the detection of a viral infection of at least one virus of the Flaviviridae family, in a mammalian subject. The diagnostic method discussed herein may comprise the steps of: (a) contacting at least one biological sample of the subject with at least one of (i) at least one reconstituted epitope of a viral envelope protein, any polypeptide, domain or viral envelope protein comprising said reconstituted epitope, any domain or viral envelope protein comprising at least one linker that replaces at least one loop or any part thereof or amino acid residue/s thereof in said viral protein, associated directly or indirectly to a solid support and/or a detectable moiety; with (ii) antibodies specific for said reconstituted virus envelope protein associated directly or indirectly to a solid support and/or a detectable moiety; or with (iii) any virus envelope protein binding molecule associated directly or indirectly to a solid support and/or a detectable moiety; and (b), determining that the subject is infected with said virus of the Flaviviridae family, if said detectable moiety is detected in said sample. These and other aspects of the invention will become apparent by the hand of the following disclosure.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1B: Dengue Virus polyprotein

FIG. 1A. Dengue virus positive RNA (11 kb) codes for one continuous polyprotein containing 3 structural proteins (Capsid-C, membrane protein-prM and Envelope-E) followed by 7 Non-Structural (NS) proteins.

FIG. 1B. The cleavage and membrane topology of the proteins relative to the ER lumen and cytoplasm is shown. Numbers indicate the amino acid lengths for each protein. Arrows indicate cleavage sites.

FIG. 2A-2E: Detail of E protein of Dengue virus

FIG. 2A: In the mature virus, the E protein forms a dimer in a “head to tail” orientation. One E protein copy is depicted as backbone at the upper right side of the structure, the other monomer is depicted as a backbone on the lower left side of the dimer. Each E monomer contains three Domains (domain I (DI—red), domain II (DII—yellow) and domain III (DIII—blue).

FIG. 2B. Linear diagram representing the three domains.

FIG. 2C. Three functional features are emphasized: the beta-strand (b-s—green), the Fusion Loop (FL—violet) and the 150 Loop (150L—cyan). Note that FL and b-s of DII of one protein oppose the 150L of the other.

FIG. 2D. Details of one E protein monomer.

FIG. 2E. Schematic presentation of the first proposed epitope to be reconstituted. Note that residues 64-120 (SEQ ID NO: 14) produce a compact and highly structured feature, supported by extensive hydrogen bonding and further locked into position by two disulfide bonds (74-105 and 92-116). Combinatorial linkers are to be introduced just preceding residue 64 and following residue 120 (grey arrows) [Rasmol depiction of 4UT6 pdb].

FIG. 3A-3B: Homology between DENV and ZV

FIG. 3A. The proposed reconstituted epitope of Dengue and Zika viruses. E protein residues 64-120 is shown. Note that identical residues for Zika virus are indicated in green, conserved residue-exchanges are shown in cyan and non-homologous residues are yellow.

FIG. 3B. The sequences of residues 60-120 of DENV and ZV are given below and are denoted as SEQ ID NO: 14 and 15, respectively. [Rasmol depic>on of 4UT6 pdb].

FIG. 4A-4B: Construct of Epitope 1

FIG. 4A. Epitope with combinatorial linkers introduced just preceding residue 64 and following residue 120.

FIG. 4B. Residues 64-120 (SEQ ID NO: 25, 26, 27, 28 for serotypes 1, 2, 3 and 4, respectively) produce a compact and highly structured feature, supported by extensive hydrogen bonding and further locked into position by two disulfide bonds (74-105 and 92-116).

FIG. 5A-5D: Reconstitution of the partial epitope bound by mAbs 1A1D and 4E5A

The DIII domain of Dengue virus serotype 2 envelope complexed with the mAb 1A1D was solved and depicted in the PDBID: 2R69.

FIG. 5A. The intact Domain III (residues 298 to 394) is shown indicating the seven strands of the lateral ridge in backbone presentation (residues 298-394, SEQ ID NO: 80, 81, 82, 83, for serotypes 1, 2, 3 and 4, respectively). The contact residues with mAb 1A1D are shown: in strand A (cyan), in the loop following strand B (orange) and the loop connecting strands D and E (violet) and in strand G in yellow. The positions of cysteine residues 302 and 333 are given in green.

FIG. 5B. Shows a slightly rotated view of A, plus the extensive hydrogen bonding within the Domain III and the disulfide connecting residues 302 to 333.

FIG. 5C. The same as B but showing the Heavy chain (red) and Light chain (blue) of mAb 1A1D.

FIG. 5D. Schematic presentation of the reconstituted epitope; removal of the F and G strands at residue 370 further reduces the bulk of the proposed reconstituted epitope. Hence the final construct starts at Methionine 301 and ends at Glutamic 370. Note that the removal of residues 336 through 355 (SEQ ID NO: 43, 44, 45, 46, for serotypes 1, 2, 3 and 4, respectively) creates a gap of 6.85A which is bridged by a comprehensive combinatorial linker library (red L). Further note the grey arrows shown at residues 301 and 370 which indicate the positions of potential combinatorial linkers as well.

FIG. 6A-6G: Templates of the Epitope 2 library

FIG. 6A. Native complete sequence of DENV envelope protein from 301 to 370 (SEQ ID NO: 39, 40, 41, 42 for serotypes 1, 2, 3 and 4, respectively)-including the loop (336-355, SEQ ID NO: 43, 44, 45, 46, for serotypes 1, 2, 3 and 4, respectively).

FIG. 6B. Sequence of DENV envelope protein from 301 to 370 with the loop omitted and residue 335 linked directly to 356 (i.e., no internal linker), also denoted by SEQ ID NO: 84, 85, 86, 87, for serotypes 1, 2, 3 and 4, respectively.

FIG. 6C-6G. Templates containing 1-5 NNK codons bridging residue 335 to 356. It should be noted that residues 301-335, are as denoted by any one of SEQ ID NO: 88, 89, 90, 91, for serotypes 1, 2, 3 and 4, respectively, and residues 356-370, are denoted by any one of SEQ ID NO: 92, 93, 94, 95, for serotypes 1, 2, 3 and 4, respectively.

FIG. 7: Testing positive clones for cross-binding with 1A1D and 4E5A Figure shows dot blot of positive clones to check for cross-reactivity with the 4E5A and 1A1D antibodies.

FIG. 8: Binding of mAbs 1A1D and 4E5A to a collection of phage-displayed reconstituted DIII domain epitopes.

Comparison between 12 positive clones for their ability to be bound by 1A1D and 4E5A mAbs. Clones A11, C6, F8 and H1 (boxes) exhibited cross-reactive features, by being recognized by both 1A1D and 4E5A. In addition, clone C9 was used as positive control for 1A1D, clone H9 as positive control for 4E5A and fth1 as negative control.

FIG. 9A-9C: ELISA results of mAb 4E5A binding to cross-reactive conjugated constructs FIG. 9A. Maltose-binding protein (MBP) constructs A11 and H1 were strongly bond by 4E5A, while F8 was less detectable and C6 was not detectable at all.

FIG. 9B. Glutathione S-transferase (GST) construct H1 exhibited high binding, whereas constructs A11, C6 and F8 low binding.

FIG. 9C. I53-50A constructs (153) A11, F8 and H1 showed high binding to the 4E5A Ab and no binding to the negative control of 153 vector.

FIG. 10: Diagram of the affinity selection procedure

Dengue antigens are incubated with anti-DENV mAbs, then protein-G magnetic beads are added to the antigen-mAbs complexes. The unbound constructs are washed away and the bound antigens are further eluted and eventually transferred to nitrocellulose membranes for dot-blot evaluation. Next, the membranes are incubated with primary antibody, followed by a secondary antibody conjugated to HRP. By adding a substrate, a signal indicating binding is detected.

FIG. 11: Affinity pull down dot-blot test results

Phage-displayed constructs (A11, F8 and H1) selected by mAbs 4E5A or 1A1D. Constructs C9 and H9 are discriminatory controls for 1A1D and 4E5A, respectively. The Right panel is a control representing a membrane incubated with only secondary Ab.

FIG. 12A-12B. Affinity pull down dot-blot test results

FIG. 12A. MBP-conjugates selected by 4E5A or 1A1D.

FIG. 12B. GST-conjugates selected by 4E5A or 1A1D. E5 is a negative control. Right panel is a control representing a membrane incubated with only secondary Ab.

FIG. 13A-13F: Schematic presentations of the structures of Domain III of the envelope protein of Dengue, in complexes with the neutralizing mAbs employed herein.

Figures show schematic presentation of the Dengue DIII domain of various strains (Yellow and orange in FIGS. 13A-13F), complexed with the antibody chains (blue and green in FIGS. 13A-13F). The residues in bold (301 to 335, and 356-370) are included in the constructs of FIG. 6.

FIG. 13A. Fab 1A1D complexed with E-DIII of Dengue 2 (Thailand), PDB ID: 2R69. The amino acid sequence appearing on the figure is as denoted by SEQ ID NO: 16. The residues in blue are included in the constructs described in FIGS. 5 and 6.

FIG. 13B. Crystal structure of the Dengue virus serotype 1 envelope protein domain III in complex with the variable domains of Mab 4E11 (4E5A) (Western Pacific), PDB ID: 3UZQ. The amino acid sequence appearing on the figure is as denoted by SEQ ID NO: 17.

FIG. 13C. Crystal structure of the Dengue virus serotype 2 envelope protein domain III in complex with the variable domains of Mab 4E11 (4E5A) (Jamaica), PDB ID: 3UZV. The amino acid sequence appearing on the figure is as denoted by SEQ ID NO: 18.

FIG. 13D. Crystal structure of the Dengue virus serotype 3 envelope protein domain III in complex with the variable domains of Mab 4E11 (4E5A) (Philippines), PDB ID: 3UZE. The amino acid sequence appearing on the figure is as denoted by SEQ ID NO: 19.

FIG. 13E. Crystal structure of the Dengue virus serotype 4 envelope protein domain III in complex with the variable domains of Mab 4E11 (4E5A) (Burma), PDB ID: 3UYP. The amino acid sequence appearing on the figure is as denoted by SEQ ID NO: 20.

FIG. 13F. Cryo EM structure of Dengue complexed with CRD of DC-SIGN (CD209), PDB ID: 2B6B.

FIG. 14A-14D: Schematic presentation of the structure of different serotypes of the Dengue virus

Figures show schematic presentation of the Dengue DIII domain of various serotypes The residues in bold (301 to 335, and 356-370) are included in the constructs described in FIG. 6.

FIG. 14A. Three-dimensional structure of the E glycoprotein of Dengue virus serotype 1, PDB ID: 3J05. The amino acid sequence appearing on the figure is as denoted by SEQ ID NO: 21.

FIG. 14B. The structure of E glycoprotein of Dengue virus serotype 2, PDB ID: 1TG8. The amino acid sequence appearing on the figure is as denoted by SEQ ID NO: 22.

FIG. 14C. Crystal structure of the E glycoprotein of Dengue serotype 3, PDB ID: 1UZG. The amino acid sequence appearing on the figure is as denoted by SEQ ID NO: 23.

FIG. 14D. Crystal structure of the E glycoprotein ectodomain from Dengue virus serotype 4 (strain 814669, Dominica), PDB ID: 3UAJ. The amino acid sequence appearing on the figure is as denoted by SEQ ID NO: 24.

FIG. 15A-15B: Dengue serotypes

FIG. 15A. The figure shows Dengue serotype phylogenetic tree.

FIG. 15B. Shows the alignment and homology of the DIII construct starting at residue Met 301 and ending at Glu 370. The sequences show a few residues before and after residues 301 and 370. Note, that residues 336 through 355 are deleted in the reconstituted constructs and bridged with combinatorial linkers. Residues 295-377 for the various serotypes of DENV as shown in the figure are denoted by SEQ ID NO: 76, 77, 78, 79, for serotypes 1, 2, 3 and 4, respectively.

FIG. 16A-16D: Use of reconstituted Domain III epitopes as immunogens

FIG. 16A. The figure shows the design of the in vivo experiment. The reconstituted epitopes (A11, F8 and H1) were expressed as MBP fusions and used to immunize mice. Three experimental groups of C57BL/6 mice (5 mice per group) immunized with A11, F8 or H1 MBP fusions respectively, were compared to controls immunized with either MBP alone or fused to full length construct (containing the native sequence 336-355 and no linker-FL) or the loopless construct (direct binding of 335 to 356 without a linker-LL). Pre-immune (baseline) sera were also collected from naive mice (BL). The animals were immunized and boosted with immunogens mixed with alum adjuvant. Three weeks after the initial immunization a boost was given and followed by a second boost three weeks later (a total of three injections). Animals were sacrificed and sera were collected three weeks after the second boost.

FIG. 16B. The figure shows a histogram presenting sera from F8 and H1 mice cross reacted with the phage displayed F8 and H1 immunogens. The reaction of the A11 derived sera was insignificant and similar to the baseline of pre-immune mice.

FIG. 16C. The figure demonstrates binding to 153 displayed epitopes by sera derived from mice immunized with the A11, F8 and H1 MBP-constructs.

FIG. 16D. The figure shows dot blot demonstrating binding of VLPs representing Dengue virus serotypes 1 through 4 by sera derived from the mice immunized with the MBP constructs for A11, F8 and H1 and the controls as described in FIG. 16A.

FIG. 17: The envelope protein structure of Zika virus

Zika is a Flavivirus whose envelope protein is comprised of 3 domains as it is illustrated in the Figure. DI (red), DII (orange) and DIII- to be reconstituted (blue and yellow). DIII showed separately and enlarged at the right bottom of the envelope. Note, the intact DIII contains residues 302-405 of SEQ ID NO: 100. The reconstituted DIII contains residues 307-380 of SEQ ID NO: 100. The yellow strands are flanking segments that have been removed from the reconstituted construct. The structures shown are depictions based on PDB ID: 5JHM. The reconstituted epitope includes residues 307-340 and 362-380 of the Zika virus envelope protein of SEQ ID NO: 100, with a linker that bridges between residues 340-362 (1-10 NNK).

FIG. 18: The envelope protein structure of Yellow fever virus

Yellow fever virus is a Flavivirus whose envelope protein is comprised of 3 domains as it is illustrated in the Figure. DI (red), DII (orange) and DIII- to be reconstituted (blue and yellow). DIII showed separately and enlarged at the right bottom of the envelope. Note, the intact DIII contains residues 292-392, of SEQ ID NO: 101. The reconstituted DIII contains residues 299-369, of SEQ ID NO: 101. The yellow strands are flanking segments that have been removed from the reconstituted construct. The structures shown are depictions based on PDB ID: 6IW4. The reconstituted epitope includes residues 299-332 and 354-369 of the Yellow fever virus envelope protein of SEQ ID NO: 101, with a linker that bridges between residues 332-354 (1-10 NNK).

FIG. 19: The envelope protein structure of West Nile virus

West Nile virus is a Flavivirus whose envelope protein is comprised of 3 domains as it is illustrated in the Figure. DI (red), DII (orange) and DIII- to be reconstituted (blue and yellow). DIII showed separately and enlarged at the right bottom of the envelope. Note, the intact DIII contains residues 297-400, of SEQ ID NO: 102. The reconstituted DIII contains residues 304-377, of SEQ ID NO: 102. The yellow strands are flanking segments that have been removed from the reconstituted construct. The structures shown are depictions based on PDB ID: 2HG0. The reconstituted epitope includes residues 304-338 and 360-377 of the West Nile virus envelope protein of SEQ ID NO: 102, with a linker that bridges between residues 338-360 (1-10 NNK).

FIG. 20: The DIII domain of Dengue virus E protein and various derived peptides

The figure schematically illustrates some of the various peptides derived from residues 301 to 370, and/or adjacent flanking residues, of the E protein of the Dengue virus as denoted by SEQ ID NO: 96, 97, 98, 99 (serotypes 1, 2, 3, 4 respectively), generating peptides of various lengths. Replacement of the loop region in theses peptides with at least one linker results in the various reconstituted epitopes.

### DETAILED DESCRIPTION OF THE INVENTION

In the light of the complexity of Flavivirus infections, and specifically, in cases of DENV infection where the risk of induction of enhancing antibodies with sub-optimal vaccines is prevalent, alternative approaches for next-generation vaccines that avoid antibody dependent enhancement (ADE) are required. This can be achieved by focusing the immune response to specific neutralizing targets, thereby providing a means to avoid ADE. Moreover, there is need to avoid developing immunogens that do not recapitulate native antigens, as is often the case with isolated proteins fragments. Epitope-focused immunogens (EFIs) should be designed such that they would reconstitute minimal requirements of targeted antigens and adopt a conformation similar to the natural conformation.

Thus, in a first aspect of the present disclosure relates to a polypeptide comprising an amino acid sequence of at least one reconstituted epitope of a viral envelope protein. In some embodiments, the viral envelop protein is composed of three domains domain I, domain II and domain III (DI, DII and DIII, respectively), and is presented on the viral coat of an enveloped virus as a dimer, oriented head to tail, with the DII domain of one subunit juxtaposed across, specifically placed side by side, from the DIII domain of the opposing subunit and the DI domain bridging DII and DIII. It should be noted that the reconstituted epitope comprises at least one linker and at least one fragment of the native, or the naturally occurring envelope protein. As will be discussed in more detail herein after, at least one of the linkers of the reconstituted epitopes is a non-natural or non-native linker that is not derived from the corresponding amino acid sequence of the naturally occurring, original sequence. Specifically, the linker comprises at least one amino acid residue or more, that differs from the amino acid sequence of the original natural sequence of the specific domain. In some embodiments, the at least one reconstituted epitope of the polypeptide of the present disclosure is of a viral envelop protein of an RNA virus. In yet some further embodiments, the envelop protein is of an RNA positive strand virus. In yet some further non-limiting embodiments, at least one fragment of the native envelope protein comprises at least two cysteine residues forming a stabilizing disulfide bridge. In some embodiments, the reconstituted epitope of the polypeptide of the present disclosure is of an enveloped virus of the Flaviviridae family.

Still further, in some embodiments the reconstituted epitope of the polypeptide of the present disclosure is of an enveloped virus of virus of the Flaviviridae family, specifically, a virus of the Flavivirus genus.

More specifically, the present disclosure provides polypeptides, reconstituted epitopes, specifically, neutralizing epitopes, vaccines and methods (as described herein after) that are specifically derived from and applicable for any virus of any genus of the Flaviviridae. The Flaviviridae, as used herein, is a family of enveloped positive-strand RNA viruses which mainly infect mammals and birds. There are 89 species in the family divided among four genera that include the genus Flavivirus (includes Dengue virus, Japanese encephalitis, Kyasanur Forest disease, Powassan virus, West Nile virus, Yellow fever virus, and Zika virus), the genus Hepacivirus (includes Hepacivirus C (hepatitis C virus) and Hepacivirus B (GB virus B)), the genus Pegivirus (includes Pegivirus A (GB virus A), Pegivirus C (GB virus C), and Pegivirus B (GB virus D)), and Genus Pestivirus (includes Pestivirus A (bovine viral diarrhea virus 1) and Pestivirus C (classical swine fever virus, previously hog cholera virus)). Viruses in this genus infect nonhuman mammals. Still further, in some embodiments, the polypeptides of the present disclosure, and specifically, the reconstituted epitopes, vaccines and uses thereof as discussed herein, are applicable and derived from any virus of the Flavivirus genus. More specifically, Flavivirus is a genus of positive-strand RNA viruses in the family Flaviviridae. The genus includes for example the West Nile virus, Dengue virus, tick-borne encephalitis virus, yellow fever virus, Zika virus and several other viruses which may cause encephalitis, as well as insect-specific Flaviviruses (ISFs) such as cell fusing agent virus (CFAV), Palm Creek virus (PCV), and Parramatta River virus (PaRV). While dual-host Flaviviruses can infect vertebrates as well as arthropods, insect-specific Flaviviruses are restricted to their competent arthropods.

Flaviviruses share several common aspects: common size (40-65 nm), symmetry (enveloped, icosahedral nucleocapsid), nucleic acid (positive-sense, single-stranded RNA around 10,000-11,000 bases), and appearance in the electron microscope.

Most of these viruses are primarily transmitted by the bite from an infected arthropod (mosquito or tick), and hence are classified as arboviruses. Human infections with most of these arboviruses are incidental, as humans are unable to replicate the virus to high enough titers to re-infect the arthropods needed to continue the virus lifecycle-humans are then a dead-end host. The exceptions to this are the Yellow fever, Dengue, and Zika viruses. These three viruses still require mosquito vectors, but are well-enough adapted to humans as to not necessarily depend upon animal hosts (although they still maintain animal transmission routes). Other virus transmission routes for arboviruses include handling infected animal carcasses, blood transfusion, sex, child birth and consumption of non-pasteurized milk products.

Flaviviruses have positive-sense, single-stranded RNA genomes which are non-segmented and around 10-11 kb in length. In general, the genome encodes three structural proteins (Capsid, prM, and Envelope) and seven non-structural proteins (NS1, NS2A, NS2B, NS3, NS4A, NS4B, NS5). The genomic RNA is modified at the 5′ end of positive-strand genomic RNA with a cap-1 structure (me7-GpppA-me2). A G protein-coupled receptor kinase 2 (also known as ADRBK1) appears to be important in entry and replication for several Flaviviridae. The envelope protein, E protein, a structural protein of Flavivirus, plays an important role in host cell viral infections. It is composed of three separate structural envelope domains I, II, and III (EDI, EDII, and EDIII). EDI is a structurally central domain of the envelope protein which stabilizes the overall orientation of the protein, and the glycosylation sites in EDI are related to virus production, pH sensitivity, and neuroinvasiveness. EDII plays an important role in membrane fusion because of the immunodominance of the fusion loop epitope and the envelope dimer epitope. Additionally, EDIII is the major target of neutralization antibodies.

The DIII domain, also referred to herein as EDIII is globular and is connected by a flexible structure to the opposite side of the EDI domain and is located at the C-terminus of the E protein. EDIII contains approximately 100 amino acids. EDIII is anchored at the C terminus to the two “stem”-helices and two transmembrane helices and is stabilized by disulfide bridges. EDIII has a β-barrel shape formed by six anti-parallel β-strands (β1, β2, β3, β4, β5 and β6). The β-strands are closed to the N-terminal residues and fold into an immunoglobulin-like conservative and relatively independent domain which is thought to interact with cellular receptors. EDIII vertically stretches out of the smooth particle surface to form apophysises, which include the type and subtype epitopes that induce specific neutralizing antibodies. EDIII also contains important linear antigenic epitopes that directly interact with potent neutralizing antibodies. These epitopes are the main target cell receptor-binding sites that assist viral entry into host cells; the target cell surface receptors include heparan sulfates, ribosomal protein SA, carbohydrate receptors, and low-density lipoprotein receptor-related protein 1 (LRP1). The neutralizing epitope region is particularly conserved across viruses. For instance, the known neutralizing epitopes in EDIII contain the residues 306, 307, 308, 330, 332, 366, 391 of WNV; 306, 331, 333, 337, 360, 373-399, and 387 in JEV; and residues 307, 333-351, and 383-389 in DENV.

In more specific embodiments of the polypeptide disclosed herein, the reconstituted epitope is of viral envelope protein of at least one of: Dengue virus, Zika virus, Yellow fever virus, West Nile virus, Tick-borne encephalitis virus, Japanese encephalitis virus and Tembusu virus, and any serotype/s, variant/s or mutant/s thereof. In more particular embodiments, the polypeptide of the present disclosure comprises at least one reconstituted epitope of at least one Dengue virus, and/or any serotype or subtype thereof, and/or any variant/s or mutant/s thereof. Thus, according to some embodiments, the present disclosure is particularly applicable for Dengue virus. The Dengue virus (also indicated herein as DENV, DNV or Dengue) is the virus causing dengue fever. It is a mosquito-borne, single positive-stranded RNA virus of the family Flaviviridae; genus Flavivirus. Four serotypes of the virus have been found, all of which can cause the full spectrum of disease. Dengue virus associated diseases have increased dramatically within the last 20 years, becoming one of the worst mosquito-borne human pathogens with which tropical countries have to deal. Current estimates indicate that as many as 390 million infections occur each year, and many dengue infections are increasingly understood to be asymptomatic or subclinical. The DENV genome is about 11000 bases of positive-sense, single stranded RNA (ssRNA) that codes for three structural proteins (capsid protein C, membrane protein M, envelope protein E and seven nonstructural proteins (NS1, NS2a, NS2b, NS3, NS4a, NS4b, NS5). The DENV E (envelope) protein, found as a dimer on the surface of the mature viral particle, is important in the initial attachment of this particle to the host cell. Each E protein monomer comprises three ectodomains, EDI to EDIII, and a transmembrane segment. EDII includes the dimerization interface, two glycosylation sites, and the peptide of fusion with the cellular membrane. EDIII is a continuous polypeptide segment; its fold is compact and immunoglobulin-like. Dengue virus is transmitted by species of the mosquito genus Aedes. Several molecules that interact with the viral E protein (ICAM3-grabbing nonintegrin, CD209, Rab 5, GRP 78, and the mannose receptor) have been shown to be important factors mediating attachment and viral entry. The membrane form of ribosomal protein SA may also be involved in the attachment. In some embodiments, the E protein of DENV comprises the amino acid sequence as denoted by any one of SEQ ID NO: 96, 97, 98, 99, or any variants and mutants thereof. As indicated herein, it should be understood that in some embodiment, the present disclosure relates to any Dengue virus or any serotype and any subtypes thereof. More specifically, there is a strain variation within each Dengue serotype, dividing them into distinct genetic subtypes. The first genetic evidence for differences between Dengue viruses of the same serotype came from RNA fingerprinting studies (Repik, Patricia M., et al., (1983), The American journal of tropical medicine and hygiene 32:3: 577-589; Vezza et al., (1980) The American journal of tropical medicine and hygiene 29:4 643-652). As determined using phylogenetic analyses, within each serotype, there are multiple genetically distinct genotypes, which are more closely related to each other than they are to the other serotypes (Weaver and Vasilakis, (2009), Infection, genetics and evolution 9.4:523-540). Initial genetic characterizations of DENV in all serotypes were defined by geographic variants by T1 RNase fingerprinting (Repik et al., 1983). Later, nucleic acid sequencing confirmed the homology of the 4 serotypes as well as their conserved genetic organization and allowed for the more precise and broad classification of DENV into genetically distinct groups or genotypes within each serotype (Rico-Hesse, (1990) Virology 174.2:479-493). Rico-Hesse defined DENV “genotypes” as clusters of DENV with sequence divergence not greater than 6% within the chosen genome region.

Dengue virus classification into subtypes is useful for studying the global distribution and movement of Dengue serotypes, which contributes to the identification of viral factors that influence disease severity and risk factors associated with the transmission of particular strains. Still further the present disclosure encompasses in some particular and non-limiting embodiments thereof, any of the following DENV serotypes and subtypes: In some embodiments, the Dengue virus is of serotype 1. In more specific embodiments, this serotype encompasses the subtypes isolated from Vietnam, Brazil and Angola. In yet some further embodiments, the subtypes are denoted by gene accession number BBG62286.1, AKQ00038.1 and AGW21594.1, respectively. In some embodiments, the Dengue virus is of serotype 2. In more specific embodiments, this serotype encompasses the subtypes isolated from Thailand, Ecuador and Kenya. In yet some further embodiments, the subtypes are denoted by gene accession number BBG31502.1 AUN35139.1 and AXY40350.1, respectively. In some embodiments, the Dengue virus is of serotype 3. In more specific embodiments, this serotype encompasses the subtypes isolated from China, Colombia and Gabon. In yet some further embodiments, the subtypes are denoted by gene accession number AHL17465.1, AXG22237.1 and BBD74779.1, respectively. In some embodiments, the Dengue virus is of serotype 4. In more specific embodiments, this serotype encompasses the subtypes isolated from Thailand, Brazil and New Caledonia. In yet some further embodiments, the subtypes are denoted by gene accession number BBG31514.1, AKQ00029.1 and AFY10037.1, respectively.

Still further in some embodiments, the reconstituted epitope of the polypeptide of the present disclosure is of at least one envelope protein that comprise an amino acid sequence as denoted by any one of SEQ ID NO: 96, 97, 98 and 99, and any variants, mutants and homologs thereof. For example, any variant, homolog, or ortholog that display between about 50%, 55%, 60%, 65%, 70%, 75%, 80%, 85%, 90%, 95%, 97%, 98%, 99%, 99.99% or 100% homology, identity or similarity to the entire sequence of the envelope protein, or to the entire sequence of any of the E protein domains DIII, DII, DI (as defined by the present disclosure), of the Dengue virus discussed herein, and/or of any serotype/s, variant/s or mutant/s thereof.

In more specific embodiments, the reconstituted epitope of the polypeptide of the present disclosure comprises at least in part, at least one amino acid sequence of the DIII domain of the native envelope protein (E protein) of the Dengue virus, or at least one amino acid sequence derived from the DIII domain, and any fragments thereof. More specifically, “Fragment” with respect to polypeptide sequences (e.g., the E protein or DIII, DII, DI), means polypeptides that comprise at least 10%, at least 20%, at least 30%, at least 40%, at least 50%, at least 60%, at least 70%, at least 80%, at least 90% or at least 95% of the complete segment of the native E envelope protein. In some embodiments, fragments of the E envelope protein may comprise at least 5, at least 10, at least 15, at least 20 amino acids or more, at least 30 amino acids or more, at least 40 amino acids or more, at least 50 amino acids or more, at least 60 amino acids or more, at least 70 amino acids or more, at least 80 amino acids or more, at least 90 amino acids or more and at least 100 amino acids or more of said native protein.

It should be understood that the term “native” as indicated herein and throughout the present disclosure, refers to the naturally occurring amino acid sequence of the specific E protein or any fragments thereof, having the original an unmodified sequence that appear in nature. This term however encompasses any natural variants, serotype/s, and mutants that naturally occur. The reconstituted epitopes of the disclosed polypeptides, although comprise sequences that derived from naturally occurring sequences, cannot be considered as naturally occurring polypeptides, and differ from the natural counterparts, at least structurally, in the linker sequence and position.

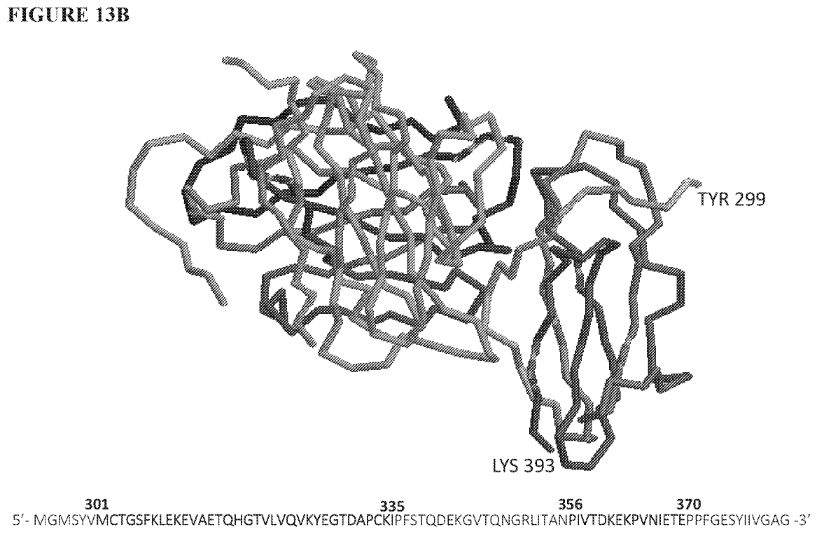

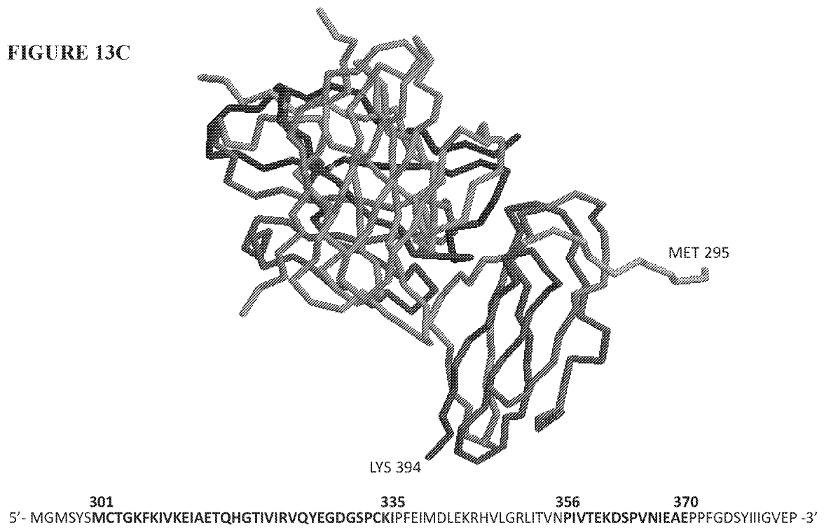

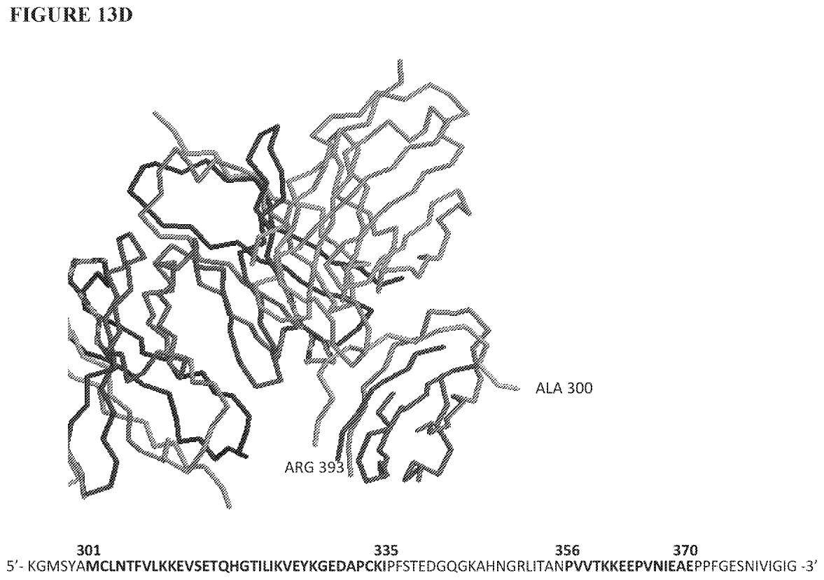

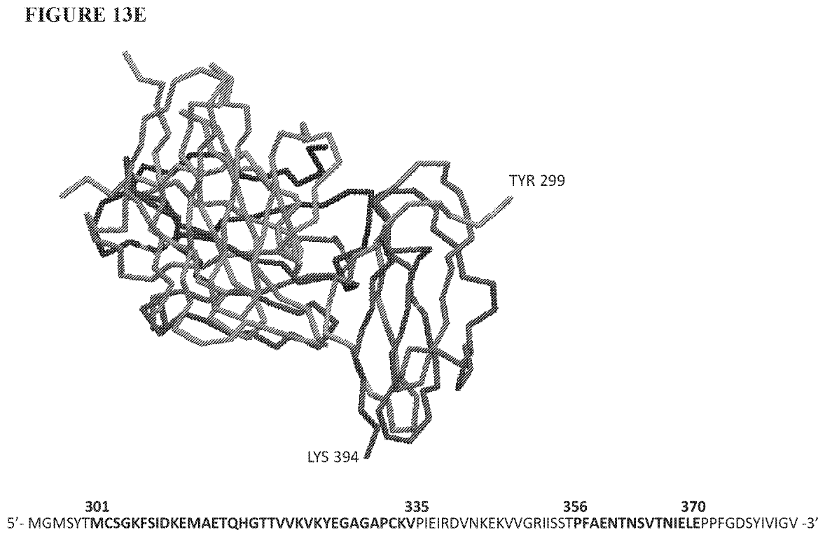

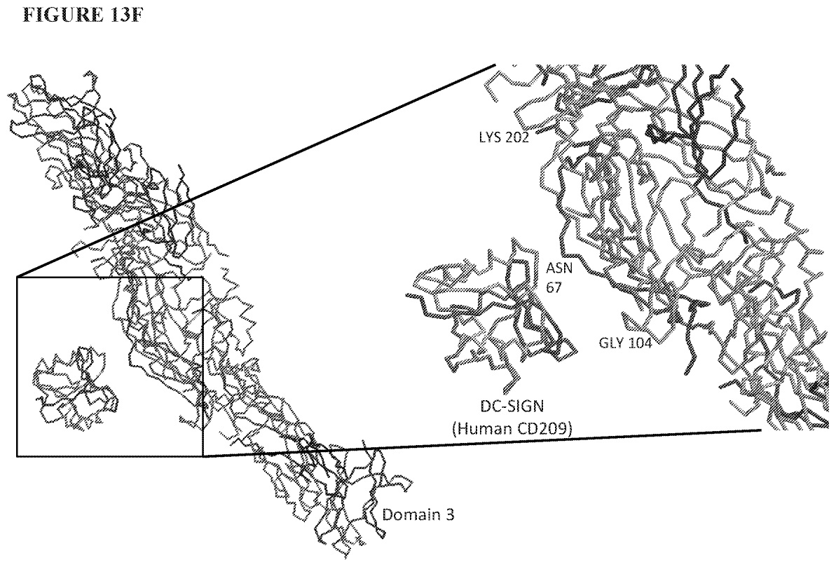

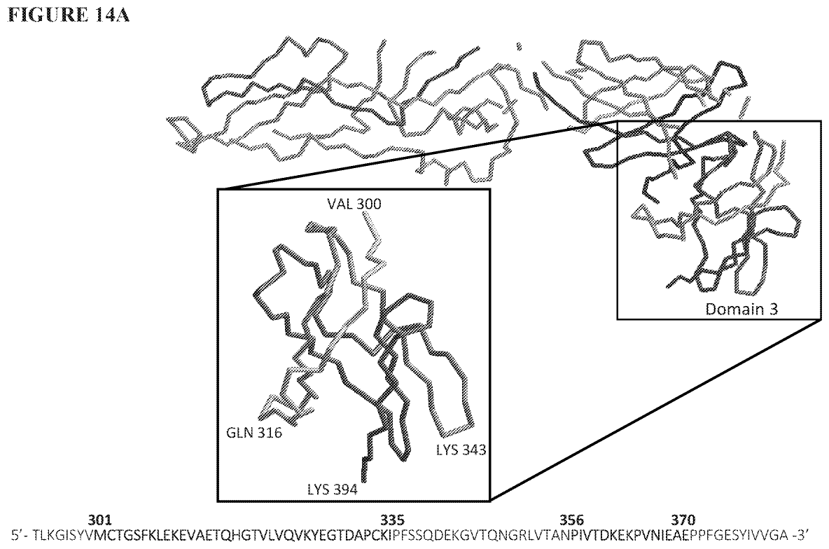

It should be noted that in some embodiments, at least part of the reconstituted peptide of the present disclosure comprises or is composed of amino acid sequence of the DIII domain, or sequence derived, at least partially, from the DIII domain or from any fragments or parts thereof, as discussed herein after. In yet some further embodiments, the DIII domain comprises residues S298 to K394, of the native E envelope protein of Dengue serotype 2. In yet some further embodiments, the DIII domain comprises residues Y299 to K393, of the native E protein of Dengue serotype 1. In some further embodiments, the DIII domain comprises residues M295 to K394, of the native E protein of Dengue serotype 2. Still further, the DIII domain comprises residues A300 to K394, of the native E protein of Dengue serotype 3. In some further embodiments, the DIII domain comprises residues Y299 to K394, of the native E protein of Dengue serotype 4. The specific regions of the DIII domain for each of the Dengue serotypes are also disclosed in FIGS. 13A-13D. Still further, in some embodiments, at least one fragment of the native or naturally occurring Dengue E protein, used for, and comprised within the reconstituted epitope of the polypeptide of the present disclosure, may comprises at least one of the following options: in one option (a), at least one amino acid sequence starting at any one of the amino acid residues 301, 296, 297, 298, 299, 300, 302, 303, 304, 305 or 306, and ending at any one of the amino acid residues 370, 365, 366, 367, 368, 369, 371, 372, 373, 374, 375, 376, 377, 378, 379, 380, 381, 382, 383, 384, 385, 386, 387, 388, 389, 390, 391, 392, 393, 39, 395, 396, 397 or 398. In another option (b), at least one amino acid sequence starting at any one of the amino acid residues 301, 296, 297, 298, 299, 300, 302, 303, 304, 305 and 306 and ending at any one of the amino acid residues 335, 329, 330, 331, 332, 333, 334, 336, 337, 338, 339 or 340. In yet some other option (c), at least one amino acid sequence starting at any one of the amino acid residues 356, 351, 352, 353, 354, 355, 357, 358, 359, 360 or 361, and ending at any one of the amino acid residues 370, 365, 366, 367, 368, 369, 371, 372, 373, 374, 375, 376, 377, 378, 379, 380, 381, 382, 383, 384, 385, 386, 387, 388, 389, 390, 391, 392, 393, 39, 395, 396, 397 or 398. FIG. 20 schematically illustrates several possible combinations of start and end residues derived from the DIII domain of the E protein of Dengue virus, that may be used in the preparation of the reconstituted epitopes of the preset disclosure as disclosed herein. In some embodiments, the reconstituted epitope/s of the disclosed polypeptides may comprise amino acid sequences as defined in (a), in (b), in (c), or in any combinations thereof. In some specific embodiments, the reconstituted epitope may comprise any of the amino acid sequences defined in (a) and at least one linker. In yet some further embodiments, the reconstituted epitope may comprise any of the amino acid sequences defined in (b), any of the sequences as defined in (c) and at least one linker, optionally at least one linker that links between both amino acid sequences. More specifically, in some embodiments, the native DIII domain of the Dengue virus comprises the amino acid sequence starting at residue M301 and ending at residue E370 of the E protein of Dengue virus as denoted by any one of SEQ ID Nos: 96, 97, 98, 99 (for serotypes 1, 2, 3, 4, respectively), or any homologs and variants thereof. In some specific and non-limiting embodiments, the M301 to E370 is denoted by SEQ ID NOs: 39, 40, 41, 42 (for serotypes 1, 2, 3, 4, respectively), and any variants thereof, specifically, as discussed herein. In some embodiments, the native DIII domain comprises the amino acid sequence starting at residue M301 and ending at residue P371 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue M301 and ending at residue P372 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue M301 and ending at residue F373 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M301 and ending at residue G374 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue M301 and ending at residue E375 of the E protein of Dengue virus as denoted by SEQ ID Nos: 96, 98, (for serotypes 1, 3, respectively), or D375 of the E protein of Dengue virus as denoted by SEQ ID NOs: 97, 99 for serotypes 2, 4, respectively), or any homologs and variants thereof. In some embodiments, the native DIII domain comprises the amino acid sequence starting at residue M301 and ending at residue S376 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue M301 and ending at residue Y377 of the E protein of Dengue virus as denoted by SEQ ID NOs: 96, 97, 99 (for serotypes 1, 2, 4, respectively), and N377, for of the E protein of Dengue virus SEQ ID NO: 98 (for serotype 3), or any homologs and variants thereof. In some embodiments, the native DIII domain comprises the amino acid sequence starting at residue M301 and ending at residue I378 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M301 and ending at residue V379 of the E protein of Dengue virus as denoted by SEQ ID NOs: 96, 98, 99 (for serotypes 1, 3, 4, respectively), and I377, for of the E protein of Dengue virus SEQ ID NO: 97 (for serotype 2), or any homologs and variants thereof. In some embodiments, the native DIII domain comprises the amino acid sequence starting at residue M301 and ending at residue I380 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M301 and ending at residue G381 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M301 and ending at residue A382 of the E protein of Dengue virus as denoted by SEQ ID NOs: 96 (serotype 1), V382 as denoted by SEQ ID NO: 97, and 99 (for serotypes 2, 4, respectively), or I382, of SEQ ID NO: 98 (serotype 3), or any homologs and variants thereof. In some embodiments, the native DIII domain comprises the amino acid sequence starting at residue M301 and ending at residue G383 of the E protein of Dengue virus as denoted by SEQ ID NOs: 96, 98, 99 (for serotypes 13, 4, respectively), or E383, as denoted by SEQ ID NO: 97 (serotype 2), or any homologs and variants thereof. In some embodiments, the native DIII domain comprises the amino acid sequence starting at residue M301 and ending at residue E384 of the E protein of Dengue virus as denoted by SEQ ID NO: 96 (serotype 1), or P384 of SEQ ID NO: 97 (serotype 2), D384 of SEQ ID NO: 98 (serotype 3), or N384 of SEQ ID NO: 99 (for serotype 4), or any homologs and variants thereof. In some embodiments, the native DIII domain comprises the amino acid sequence starting at residue M301 and ending at residue K385 of the E protein of Dengue virus as denoted by SEQ ID NOs: 96, 98, (for serotypes 1, 3, respectively), or G385, as denoted by SEQ ID NO: 97 (serotype 2), or S385, of SEQ ID NO: 99 (serotype 4), or any homologs and variants thereof. In some embodiments, the native DIII domain comprises the amino acid sequence starting at residue M301 and ending at residue A386 of the E protein of Dengue virus as denoted by SEQ ID NOs: 96, 98, 99 (for serotypes 1, 3, 4, respectively), or Q386, of SEQ ID NO: 97 (serotype 2), or any homologs and variants thereof. In some embodiments, the native DIII domain comprises the amino acid sequence starting at residue M301 and ending at residue L387 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M301 and ending at residue K388 of the E protein of Dengue virus as denoted by SEQ ID NOs: 96, 97, 98, (for serotypes 1, 2, 3, respectively), or T388, of SEQ ID NO: 99 (serotype 4), or any homologs and variants thereof. In some embodiments, the native DIII domain comprises the amino acid sequence starting at residue M301 and ending at residue L389 of the E protein of Dengue virus as denoted by SEQ ID NOs: 96, 97, 99 (for serotypes 1, 2, 4, respectively), or I389, of SEQ ID NO: 98 (serotype 3), or any homologs and variants thereof. In some embodiments, the native DIII domain comprises the amino acid sequence starting at residue M301 and ending at residue S390 of the E protein of Dengue virus as denoted by SEQ ID NOs: 96, 97, (for serotypes 1, 2, respectively), or N390, of SEQ ID NO: 98 (serotype 3), or H390, of SEQ ID NO: 99 (serotype 4), or any homologs and variants thereof. In some embodiments, the native DIII domain comprises the amino acid sequence starting at residue M301 and ending at residue W391 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M301 and ending at residue F392 of the E protein of Dengue virus as denoted by SEQ ID NOs: 96, 97, 99 (for serotypes 1, 2, 4, respectively), or Y392, of SEQ ID NO: 98 (serotype 3), or H390, of SEQ ID NO: 99 (serotype 4), or any homologs and variants thereof. In some embodiments, the native DIII domain comprises the amino acid sequence starting at residue M301 and ending at residue K393 of the E protein of Dengue virus as denoted by SEQ ID NOs: 96, 97, 98 (for serotypes 1, 2, 3, respectively), or R393, of SEQ ID NO: 99 (serotype 4), or any homologs and variants thereof. In some embodiments, the native DIII domain comprises the amino acid sequence starting at residue M301 and ending at residue K394 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M301 and ending at residue G395 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M301 and ending at residue S396 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M301 and ending at residue S397 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M301 and ending at residue I398 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M301 and ending at residue N365 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M301 and ending at residue I366 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M301 and ending at residue I367 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue M301 and ending at residue E368 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue M301 and ending at residue A369 of the E protein of Dengue virus as denoted by SEQ ID Nos: 96, 97, 98 (for serotypes 1, 2, 3, respectively) and at L369 of the E protein of Dengue virus as denoted by SEQ ID NO: 99 (for serotype 4), or any homologs and variants thereof. More specifically, in some embodiments, the native DIII domain of the Dengue virus comprises the amino acid sequence starting at residue G296 and ending at residue E370 of the E protein of Dengue virus as denoted by any one of SEQ ID Nos: 96, 97, 98, 99 (for serotypes 1, 2, 3, 4, respectively), or any homologs and variants thereof. In some embodiments, the native DIII domain comprises the amino acid sequence starting at residue G296 and ending at residue P371 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue G296 and ending at residue P372 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue G296 and ending at residue F373 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G296 and ending at residue G374 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue G296 and ending at residue E375 of the E protein of Dengue virus as denoted by SEQ ID Nos: 96, 98, (for serotypes 1, 3, respectively), or D375 of the E protein of Dengue virus as denoted by SEQ ID NOs: 97, 99 for serotypes 2, 4, respectively), or any homologs and variants thereof. In some embodiments, the native DIII domain comprises the amino acid sequence starting at residue G296 and ending at residue S376 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue G296 and ending at residue Y377 of the E protein of Dengue virus as denoted by SEQ ID NOs: 96, 97, 99 (for serotypes 1, 2, 4, respectively), and N377, for of the E protein of Dengue virus SEQ ID NO: 98 (for serotype 3), or any homologs and variants thereof. In some embodiments, the native DIII domain comprises the amino acid sequence starting at residue G296 and ending at residue I378 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G296 and ending at residue V379 of the E protein of Dengue virus as denoted by SEQ ID NOs: 96, 98, 99 (for serotypes 1, 3, 4, respectively), and I377, for of the E protein of Dengue virus SEQ ID NO: 97 (for serotype 2), or any homologs and variants thereof. In some embodiments, the native DIII domain comprises the amino acid sequence starting at residue G296 and ending at residue I380 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G296 and ending at residue G381 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G296 and ending at residue A382 of the E protein of Dengue virus as denoted by SEQ ID NOs: 96 (serotype 1), V382 as denoted by SEQ ID NO: 97, and 99 (for serotypes 2, 4, respectively), or I382, of SEQ ID NO: 98 (serotype 3), or any homologs and variants thereof. In some embodiments, the native DIII domain comprises the amino acid sequence starting at residue G296 and ending at residue G383 of the E protein of Dengue virus as denoted by SEQ ID NOS: 96, 98, 99 (for serotypes 13, 4, respectively), or E383, as denoted by SEQ ID NO: 97 (serotype 2), or any homologs and variants thereof. In some embodiments, the native DIII domain comprises the amino acid sequence starting at residue G296 and ending at residue E384 of the E protein of Dengue virus as denoted by SEQ ID NO: 96 (serotype 1), or P384 of SEQ ID NO: 97 (serotype 2), D384 of SEQ ID NO: 98 (serotype 3), or N384 of SEQ ID NO: 99 (for serotype 4), or any homologs and variants thereof. In some embodiments, the native DIII domain comprises the amino acid sequence starting at residue G296 and ending at residue K385 of the E protein of Dengue virus as denoted by SEQ ID NOs: 96, 98, (for serotypes 1, 3, respectively), or G385, as denoted by SEQ ID NO: 97 (serotype 2), or S385, of SEQ ID NO: 99 (serotype 4), or any homologs and variants thereof. In some embodiments, the native DIII domain comprises the amino acid sequence starting at residue G296 and ending at residue A386 of the E protein of Dengue virus as denoted by SEQ ID NOs: 96, 98, 99 (for serotypes 1, 3, 4, respectively), or Q386, of SEQ ID NO: 97 (serotype 2), or any homologs and variants thereof. In some embodiments, the native DIII domain comprises the amino acid sequence starting at residue G296 and ending at residue L387 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G296 and ending at residue K388 of the E protein of Dengue virus as denoted by SEQ ID NOs: 96, 97, 98, (for serotypes 1, 2, 3, respectively), or T388, of SEQ ID NO: 99 (serotype 4), or any homologs and variants thereof. In some embodiments, the native DIII domain comprises the amino acid sequence starting at residue G296 and ending at residue L389 of the E protein of Dengue virus as denoted by SEQ ID NOs: 96, 97, 99 (for serotypes 1, 2, 4, respectively), or I389, of SEQ ID NO: 98 (serotype 3), or any homologs and variants thereof. In some embodiments, the native DIII domain comprises the amino acid sequence starting at residue G296 and ending at residue S390 of the E protein of Dengue virus as denoted by SEQ ID NOs: 96, 97, (for serotypes 1, 2, respectively), or N390, of SEQ ID NO: 98 (serotype 3), or H390, of SEQ ID NO: 99 (serotype 4), or any homologs and variants thereof. In some embodiments, the native DIII domain comprises the amino acid sequence starting at residue G296 and ending at residue W391 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G296 and ending at residue F392 of the E protein of Dengue virus as denoted by SEQ ID NOs: 96, 97, 99 (for serotypes 1, 2, 4, respectively), or Y392, of SEQ ID NO: 98 (serotype 3), or H390, of SEQ ID NO: 99 (serotype 4), or any homologs and variants thereof. In some embodiments, the native DIII domain comprises the amino acid sequence starting at residue G296 and ending at residue K393 of the E protein of Dengue virus as denoted by SEQ ID NOs: 96, 97, 98 (for serotypes 1, 2, 3, respectively), or R393, of SEQ ID NO: 99 (serotype 4), or any homologs and variants thereof. In some embodiments, the native DIII domain comprises the amino acid sequence starting at residue G296 and ending at residue K394 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G296 and ending at residue G395 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G296 and ending at residue S396 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G296 and ending at residue S397 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G296 and ending at residue I398 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G296 and ending at residue N365 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G296 and ending at residue I366 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G296 and ending at residue I367 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue G296 and ending at residue E368 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue G296 and ending at residue A369 of the E protein of Dengue virus as denoted by SEQ ID Nos: 96, 97, 98 (for serotypes 1, 2, 3, respectively) and at L369 of the E protein of Dengue virus as denoted by SEQ ID NO: 99 (for serotype 4), or any homologs and variants thereof. More specifically, in some embodiments, the native DIII domain of the Dengue virus comprises the amino acid sequence starting at residue M297 and ending at residue E370 of the E protein of Dengue virus as denoted by any one of SEQ ID Nos: 96, 97, 98, 99 (for serotypes 1, 2, 3, 4, respectively), or any homologs and variants thereof. In some embodiments, the native DIII domain comprises the amino acid sequence starting at residue M297 and ending at residue P371 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue M297 and ending at residue P372 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue M297 and ending at residue F373 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M297 and ending at residue G374 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue M297 and ending at residue E375 of the E protein of Dengue virus as denoted by SEQ ID Nos: 96, 98, (for serotypes 1, 3, respectively), or D375 of the E protein of Dengue virus as denoted by SEQ ID NOs: 97, 99 for serotypes 2, 4, respectively), or any homologs and variants thereof. In some embodiments, the native DIII domain comprises the amino acid sequence starting at residue M297 and ending at residue S376 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue M297 and ending at residue Y377 of the E protein of Dengue virus as denoted by SEQ ID NOs: 96, 97, 99 (for serotypes 1, 2, 4, respectively), and N377, for of the E protein of Dengue virus SEQ ID NO: 98 (for serotype 3), or any homologs and variants thereof. In some embodiments, the native DIII domain comprises the amino acid sequence starting at residue M297 and ending at residue I378 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M297 and ending at residue V379 of the E protein of Dengue virus as denoted by SEQ ID NOs: 96, 98, 99 (for serotypes 1, 3, 4, respectively), and I377, for of the E protein of Dengue virus SEQ ID NO: 97 (for serotype 2), or any homologs and variants thereof. In some embodiments, the native DIII domain comprises the amino acid sequence starting at residue M297 and ending at residue I380 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M297 and ending at residue G381 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M297 and ending at residue A382 of the E protein of Dengue virus as denoted by SEQ ID NOs: 96 (serotype 1), V382 as denoted by SEQ ID NO: 97, and 99 (for serotypes 2, 4, respectively), or I382, of SEQ ID NO: 98 (serotype 3), or any homologs and variants thereof. In some embodiments, the native DIII domain comprises the amino acid sequence starting at residue M297 and ending at residue G383 of the E protein of Dengue virus as denoted by SEQ ID NOs: 96, 98, 99 (for serotypes 13, 4, respectively), or E383, as denoted by SEQ ID NO: 97 (serotype 2), or any homologs and variants thereof. In some embodiments, the native DIII domain comprises the amino acid sequence starting at residue M297 and ending at residue E384 of the E protein of Dengue virus as denoted by SEQ ID NO: 96 (serotype 1), or P384 of SEQ ID NO: 97 (serotype 2), D384 of SEQ ID NO: 98 (serotype 3), or N384 of SEQ ID NO: 99 (for serotype 4), or any homologs and variants thereof. In some embodiments, the native DIII domain comprises the amino acid sequence starting at residue M297 and ending at residue K385 of the E protein of Dengue virus as denoted by SEQ ID NOs: 96, 98, (for serotypes 1, 3, respectively), or G385, as denoted by SEQ ID NO: 97 (serotype 2), or S385, of SEQ ID NO: 99 (serotype 4), or any homologs and variants thereof. In some embodiments, the native DIII domain comprises the amino acid sequence starting at residue M297 and ending at residue A386 of the E protein of Dengue virus as denoted by SEQ ID NOs: 96, 98, 99 (for serotypes 1, 3, 4, respectively), or Q386, of SEQ ID NO: 97 (serotype 2), or any homologs and variants thereof. In some embodiments, the native DIII domain comprises the amino acid sequence starting at residue M297 and ending at residue L387 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M297 and ending at residue K388 of the E protein of Dengue virus as denoted by SEQ ID NOs: 96, 97, 98, (for serotypes 1, 2, 3, respectively), or T388, of SEQ ID NO: 99 (serotype 4), or any homologs and variants thereof. In some embodiments, the native DIII domain comprises the amino acid sequence starting at residue M297 and ending at residue L389 of the E protein of Dengue virus as denoted by SEQ ID NOs: 96, 97, 99 (for serotypes 1, 2, 4, respectively), or I389, of SEQ ID NO: 98 (serotype 3), or any homologs and variants thereof. In some embodiments, the native DIII domain comprises the amino acid sequence starting at residue M297 and ending at residue S390 of the E protein of Dengue virus as denoted by SEQ ID NOs: 96, 97, (for serotypes 1, 2, respectively), or N390, of SEQ ID NO: 98 (serotype 3), or H390, of SEQ ID NO: 99 (serotype 4), or any homologs and variants thereof. In some embodiments, the native DIII domain comprises the amino acid sequence starting at residue M297 and ending at residue W391 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M297 and ending at residue F392 of the E protein of Dengue virus as denoted by SEQ ID NOs: 96, 97, 99 (for serotypes 1, 2, 4, respectively), or Y392, of SEQ ID NO: 98 (serotype 3), or H390, of SEQ ID NO: 99 (serotype 4), or any homologs and variants thereof. In some embodiments, the native DIII domain comprises the amino acid sequence starting at residue M297 and ending at residue K393 of the E protein of Dengue virus as denoted by SEQ ID NOs: 96, 97, 98 (for serotypes 1, 2, 3, respectively), or R393, of SEQ ID NO: 99 (serotype 4), or any homologs and variants thereof. In some embodiments, the native DIII domain comprises the amino acid sequence starting at residue M297 and ending at residue K394 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M297 and ending at residue G395 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M297 and ending at residue S396 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M297 and ending at residue S397 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M297 and ending at residue I398 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M297 and ending at residue N365 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M297 and ending at residue I366 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue M297 and ending at residue I367 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue M297 and ending at residue E368 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue M297 and ending at residue and ending at residue A369 of the E protein of Dengue virus as denoted by SEQ ID Nos: 96, 97, 98 (for serotypes 1, 2, 3, respectively) and at L369 of the E protein of Dengue virus as denoted by SEQ ID NO: 99 (for serotype 4), or any homologs and variants thereof. More specifically, in some embodiments, the native DIII domain of the Dengue virus comprises the amino acid sequence starting at residue S298 and ending at residue E370 of the E protein of Dengue virus as denoted by any one of SEQ ID Nos: 96, 97, 98, 99 (for serotypes 1, 2, 3, 4, respectively), or any homologs and variants thereof. In some embodiments, the native DIII domain comprises the amino acid sequence starting at residue S298 and ending at residue P371 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue S298 and ending at residue P372 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue S298 and ending at residue F373 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S298 and ending at residue G374 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue S298 and ending at residue E375 of the E protein of Dengue virus as denoted by SEQ ID Nos: 96, 98, (for serotypes 1, 3, respectively), or D375 of the E protein of Dengue virus as denoted by SEQ ID NOs: 97, 99 for serotypes 2, 4, respectively), or any homologs and variants thereof. In some embodiments, the native DIII domain comprises the amino acid sequence starting at residue S298 and ending at residue S376 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue S298 and ending at residue Y377 of the E protein of Dengue virus as denoted by SEQ ID NOs: 96, 97, 99 (for serotypes 1, 2, 4, respectively), and N377, for of the E protein of Dengue virus SEQ ID NO: 98 (for serotype 3), or any homologs and variants thereof. In some embodiments, the native DIII domain comprises the amino acid sequence starting at residue S298 and ending at residue I378 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S298 and ending at residue V379 of the E protein of Dengue virus as denoted by SEQ ID NOs: 96, 98, 99 (for serotypes 1, 3, 4, respectively), and I377, for of the E protein of Dengue virus SEQ ID NO: 97 (for serotype 2), or any homologs and variants thereof. In some embodiments, the native DIII domain comprises the amino acid sequence starting at residue S298 and ending at residue I380 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S298 and ending at residue G381 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S298 and ending at residue A382 of the E protein of Dengue virus as denoted by SEQ ID NOs: 96 (serotype 1), V382 as denoted by SEQ ID NO: 97, and 99 (for serotypes 2, 4, respectively), or I382, of SEQ ID NO: 98 (serotype 3), or any homologs and variants thereof. In some embodiments, the native DIII domain comprises the amino acid sequence starting at residue S298 and ending at residue G383 of the E protein of Dengue virus as denoted by SEQ ID NOs: 96, 98, 99 (for serotypes 13, 4, respectively), or E383, as denoted by SEQ ID NO: 97 (serotype 2), or any homologs and variants thereof. In some embodiments, the native DIII domain comprises the amino acid sequence starting at residue S298 and ending at residue E384 of the E protein of Dengue virus as denoted by SEQ ID NO: 96 (serotype 1), or P384 of SEQ ID NO: 97 (serotype 2), D384 of SEQ ID NO: 98 (serotype 3), or N384 of SEQ ID NO: 99 (for serotype 4), or any homologs and variants thereof. In some embodiments, the native DIII domain comprises the amino acid sequence starting at residue S298 and ending at residue K385 of the E protein of Dengue virus as denoted by SEQ ID NOs: 96, 98, (for serotypes 1, 3, respectively), or G385, as denoted by SEQ ID NO: 97 (serotype 2), or S385, of SEQ ID NO: 99 (serotype 4), or any homologs and variants thereof. In some embodiments, the native DIII domain comprises the amino acid sequence starting at residue S298 and ending at residue A386 of the E protein of Dengue virus as denoted by SEQ ID NOs: 96, 98, 99 (for serotypes 1, 3, 4, respectively), or Q386, of SEQ ID NO: 97 (serotype 2), or any homologs and variants thereof. In some embodiments, the native DIII domain comprises the amino acid sequence starting at residue S298 and ending at residue L387 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S298 and ending at residue K388 of the E protein of Dengue virus as denoted by SEQ ID NOs: 96, 97, 98, (for serotypes 1, 2, 3, respectively), or T388, of SEQ ID NO: 99 (serotype 4), or any homologs and variants thereof. In some embodiments, the native DIII domain comprises the amino acid sequence starting at residue S298 and ending at residue L389 of the E protein of Dengue virus as denoted by SEQ ID NOs: 96, 97, 99 (for serotypes 1, 2, 4, respectively), or I389, of SEQ ID NO: 98 (serotype 3), or any homologs and variants thereof. In some embodiments, the native DIII domain comprises the amino acid sequence starting at residue S298 and ending at residue S390 of the E protein of Dengue virus as denoted by SEQ ID NOs: 96, 97, (for serotypes 1, 2, respectively), or N390, of SEQ ID NO: 98 (serotype 3), or H390, of SEQ ID NO: 99 (serotype 4), or any homologs and variants thereof. In some embodiments, the native DIII domain comprises the amino acid sequence starting at residue S298 and ending at residue W391 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S298 and ending at residue F392 of the E protein of Dengue virus as denoted by SEQ ID NOs: 96, 97, 99 (for serotypes 1, 2, 4, respectively), or Y392, of SEQ ID NO: 98 (serotype 3), or H390, of SEQ ID NO: 99 (serotype 4), or any homologs and variants thereof. In some embodiments, the native DIII domain comprises the amino acid sequence starting at residue S298 and ending at residue K393 of the E protein of Dengue virus as denoted by SEQ ID NOs: 96, 97, 98 (for serotypes 1, 2, 3, respectively), or R393, of SEQ ID NO: 99 (serotype 4), or any homologs and variants thereof. In some embodiments, the native DIII domain comprises the amino acid sequence starting at residue S298 and ending at residue K394 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S298 and ending at residue G395 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S298 and ending at residue S396 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S298 and ending at residue S397 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S298 and ending at residue I398 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S298 and ending at residue N365 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S298 and ending at residue I366 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S298 and ending at residue I367 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue S298 and ending at residue E368 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue S298 and ending at residue and ending at residue A369 of the E protein of Dengue virus as denoted by SEQ ID Nos: 96, 97, 98 (for serotypes 1, 2, 3, respectively) and at L369 of the E protein of Dengue virus as denoted by SEQ ID NO: 99 (for serotype 4), or any homologs and variants thereof. More specifically, in some embodiments, the native DIII domain of the Dengue virus comprises the amino acid sequence starting at residue Y299 and ending at residue E370 of the E protein of Dengue virus as denoted by any one of SEQ ID Nos: 96, 97, 98, 99 (for serotypes 1, 2, 3, 4, respectively), or any homologs and variants thereof. In some embodiments, the native DIII domain comprises the amino acid sequence starting at residue Y299 and ending at residue P371 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue Y299 and ending at residue P372 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue Y299 and ending at residue F373 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue Y299 and ending at residue G374 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue Y299 and ending at residue E375 of the E protein of Dengue virus as denoted by SEQ ID Nos: 96, 98, (for serotypes 1, 3, respectively), or D375 of the E protein of Dengue virus as denoted by SEQ ID NOs: 97, 99 for serotypes 2, 4, respectively), or any homologs and variants thereof. In some embodiments, the native DIII domain comprises the amino acid sequence starting at residue Y299 and ending at residue S376 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue Y299 and ending at residue Y377 of the E protein of Dengue virus as denoted by SEQ ID NOs: 96, 97, 99 (for serotypes 1, 2, 4, respectively), and N377, for of the E protein of Dengue virus SEQ ID NO: 98 (for serotype 3), or any homologs and variants thereof. In some embodiments, the native DIII domain comprises the amino acid sequence starting at residue Y299 and ending at residue I378 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue Y299 and ending at residue V379 of the E protein of Dengue virus as denoted by SEQ ID NOs: 96, 98, 99 (for serotypes 1, 3, 4, respectively), and 1377, for of the E protein of Dengue virus SEQ ID NO: 97 (for serotype 2), or any homologs and variants thereof. In some embodiments, the native DIII domain comprises the amino acid sequence starting at residue Y299 and ending at residue I380 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue Y299 and ending at residue G381 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue Y299 and ending at residue A382 of the E protein of Dengue virus as denoted by SEQ ID NOs: 96 (serotype 1), V382 as denoted by SEQ ID NO: 97, and 99 (for serotypes 2, 4, respectively), or I382, of SEQ ID NO: 98 (serotype 3), or any homologs and variants thereof. In some embodiments, the native DIII domain comprises the amino acid sequence starting at residue Y299 and ending at residue G383 of the E protein of Dengue virus as denoted by SEQ ID NOs: 96, 98, 99 (for serotypes 13, 4, respectively), or E383, as denoted by SEQ ID NO: 97 (serotype 2), or any homologs and variants thereof. In some embodiments, the native DIII domain comprises the amino acid sequence starting at residue Y299 and ending at residue E384 of the E protein of Dengue virus as denoted by SEQ ID NO: 96 (serotype 1), or P384 of SEQ ID NO: 97 (serotype 2), D384 of SEQ ID NO: 98 (serotype 3), or N384 of SEQ ID NO: 99 (for serotype 4), or any homologs and variants thereof. In some embodiments, the native DIII domain comprises the amino acid sequence starting at residue Y299 and ending at residue K385 of the E protein of Dengue virus as denoted by SEQ ID NOs: 96, 98, (for serotypes 1, 3, respectively), or G385, as denoted by SEQ ID NO: 97 (serotype 2), or S385, of SEQ ID NO: 99 (serotype 4), or any homologs and variants thereof. In some embodiments, the native DIII domain comprises the amino acid sequence starting at residue Y299 and ending at residue A386 of the E protein of Dengue virus as denoted by SEQ ID NOs: 96, 98, 99 (for serotypes 1, 3, 4, respectively), or Q386, of SEQ ID NO: 97 (serotype 2), or any homologs and variants thereof. In some embodiments, the native DIII domain comprises the amino acid sequence starting at residue Y299 and ending at residue L387 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue Y299 and ending at residue K388 of the E protein of Dengue virus as denoted by SEQ ID NOs: 96, 97, 98, (for serotypes 1, 2, 3, respectively), or T388, of SEQ ID NO: 99 (serotype 4), or any homologs and variants thereof. In some embodiments, the native DIII domain comprises the amino acid sequence starting at residue Y299 and ending at residue L389 of the E protein of Dengue virus as denoted by SEQ ID NOs: 96, 97, 99 (for serotypes 1, 2, 4, respectively), or I389, of SEQ ID NO: 98 (serotype 3), or any homologs and variants thereof. In some embodiments, the native DIII domain comprises the amino acid sequence starting at residue Y299 and ending at residue S390 of the E protein of Dengue virus as denoted by SEQ ID NOs: 96, 97, (for serotypes 1, 2, respectively), or N390, of SEQ ID NO: 98 (serotype 3), or H390, of SEQ ID NO: 99 (serotype 4), or any homologs and variants thereof. In some embodiments, the native DIII domain comprises the amino acid sequence starting at residue Y299 and ending at residue W391 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue Y299 and ending at residue F392 of the E protein of Dengue virus as denoted by SEQ ID NOs: 96, 97, 99 (for serotypes 1, 2, 4, respectively), or Y392, of SEQ ID NO: 98 (serotype 3), or H390, of SEQ ID NO: 99 (serotype 4), or any homologs and variants thereof. In some embodiments, the native DIII domain comprises the amino acid sequence starting at residue Y299 and ending at residue K393 of the E protein of Dengue virus as denoted by SEQ ID NOs: 96, 97, 98 (for serotypes 1, 2, 3, respectively), or R393, of SEQ ID NO: 99 (serotype 4), or any homologs and variants thereof. In some embodiments, the native DIII domain comprises the amino acid sequence starting at residue Y299 and ending at residue K394 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue Y299 and ending at residue G395 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue Y299 and ending at residue S396 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue Y299 and ending at residue S397 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue Y299 and ending at residue I398 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue Y299 and ending at residue N365 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue Y299 and ending at residue I366 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue Y299 and ending at residue I367 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue Y299 and ending at residue E368 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue Y299 and ending at residue and ending at residue A369 of the E protein of Dengue virus as denoted by SEQ ID Nos: 96, 97, 98 (for serotypes 1, 2, 3, respectively) and at L369 of the E protein of Dengue virus as denoted by SEQ ID NO: 99 (for serotype 4), or any homologs and variants thereof. More specifically, in some embodiments, the native DIII domain of the Dengue virus comprises the amino acid sequence starting at residue 300 (V300, S330, A300 or T300, of serotypes 1, 2, 3, 4, respectively) and ending at residue E370 of the E protein of Dengue virus as denoted by any one of SEQ ID Nos: 96, 97, 98, 99 (for serotypes 1, 2, 3, 4, respectively), or any homologs and variants thereof. In some embodiments, the native DIII domain comprises the amino acid sequence starting at residue 300 (V300, S330, A300 or T300, of serotypes 1, 2, 3, 4, respectively) and ending at residue P371 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue 300 (V300, S330, A300 or T300, of serotypes 1, 2, 3, 4, respectively) and ending at residue P372 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue 300 (V300, S330, A300 or T300, of serotypes 1, 2, 3, 4, respectively) and ending at residue F373 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue 300 (V300, S330, A300 or T300, of serotypes 1, 2, 3, 4, respectively) and ending at residue G374 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue 300 (V300, S330, A300 or T300, of serotypes 1, 2, 3, 4, respectively) and ending at residue E375 of the E protein of Dengue virus as denoted by SEQ ID Nos: 96, 98, (for serotypes 1, 3, respectively), or D375 of the E protein of Dengue virus as denoted by SEQ ID NOs: 97, 99 for serotypes 2, 4, respectively), or any homologs and variants thereof. In some embodiments, the native DIII domain comprises the amino acid sequence starting at residue 300 (V300, S330, A300 or T300, of serotypes 1, 2, 3, 4, respectively) and ending at residue S376 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue 300 (V300, S330, A300 or T300, of serotypes 1, 2, 3, 4, respectively) and ending at residue Y377 of the E protein of Dengue virus as denoted by SEQ ID NOs: 96, 97, 99 (for serotypes 1, 2, 4, respectively), and N377, for of the E protein of Dengue virus SEQ ID NO: 98 (for serotype 3), or any homologs and variants thereof. In some embodiments, the native DIII domain comprises the amino acid sequence starting at residue 300 (V300, S330, A300 or T300, of serotypes 1, 2, 3, 4, respectively) and ending at residue I378 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue 300 (V300, S330, A300 or T300, of serotypes 1, 2, 3, 4, respectively) and ending at residue V379 of the E protein of Dengue virus as denoted by SEQ ID NOs: 96, 98, 99 (for serotypes 1, 3, 4, respectively), and I377, for of the E protein of Dengue virus SEQ ID NO: 97 (for serotype 2), or any homologs and variants thereof. In some embodiments, the native DIII domain comprises the amino acid sequence starting at residue 300 (V300, S330, A300 or T300, of serotypes 1, 2, 3, 4, respectively) and ending at residue I380 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue 300 (V300, S330, A300 or T300, of serotypes 1, 2, 3, 4, respectively) and ending at residue G381 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue 300 (V300, S330, A300 or T300, of serotypes 1, 2, 3, 4, respectively) and ending at residue A382 of the E protein of Dengue virus as denoted by SEQ ID NOs: 96 (serotype 1), V382 as denoted by SEQ ID NO: 97, and 99 (for serotypes 2, 4, respectively), or I382, of SEQ ID NO: 98 (serotype 3), or any homologs and variants thereof. In some embodiments, the native DIII domain comprises the amino acid sequence starting at residue 300 (V300, S330, A300 or T300, of serotypes 1, 2, 3, 4, respectively) and ending at residue G383 of the E protein of Dengue virus as denoted by SEQ ID NOs: 96, 98, 99 (for serotypes 13, 4, respectively), or E383, as denoted by SEQ ID NO: 97 (serotype 2), or any homologs and variants thereof. In some embodiments, the native DIII domain comprises the amino acid sequence starting at residue 300 (V300, S330, A300 or T300, of serotypes 1, 2, 3, 4, respectively) and ending at residue E384 of the E protein of Dengue virus as denoted by SEQ ID NO: 96 (serotype 1), or P384 of SEQ ID NO: 97 (serotype 2), D384 of SEQ ID NO: 98 (serotype 3), or N384 of SEQ ID NO: 99 (for serotype 4), or any homologs and variants thereof. In some embodiments, the native DIII domain comprises the amino acid sequence starting at residue 300 (V300, S330, A300 or T300, of serotypes 1, 2, 3, 4, respectively) and ending at residue K385 of the E protein of Dengue virus as denoted by SEQ ID NOs: 96, 98, (for serotypes 1, 3, respectively), or G385, as denoted by SEQ ID NO: 97 (serotype 2), or S385, of SEQ ID NO: 99 (serotype 4), or any homologs and variants thereof. In some embodiments, the native DIII domain comprises the amino acid sequence starting at residue 300 (V300, S330, A300 or T300, of serotypes 1, 2, 3, 4, respectively) and ending at residue A386 of the E protein of Dengue virus as denoted by SEQ ID NOs: 96, 98, 99 (for serotypes 1, 3, 4, respectively), or Q386, of SEQ ID NO: 97 (serotype 2), or any homologs and variants thereof. In some embodiments, the native DIII domain comprises the amino acid sequence starting at residue 300 (V300, S330, A300 or T300, of serotypes 1, 2, 3, 4, respectively) and ending at residue L387 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue 300 (V300, S330, A300 or T300, of serotypes 1, 2, 3, 4, respectively) and ending at residue K388 of the E protein of Dengue virus as denoted by SEQ ID NOs: 96, 97, 98, (for serotypes 1, 2, 3, respectively), or T388, of SEQ ID NO: 99 (serotype 4), or any homologs and variants thereof. In some embodiments, the native DIII domain comprises the amino acid sequence starting at residue 300 (V300, S330, A300 or T300, of serotypes 1, 2, 3, 4, respectively) and ending at residue L389 of the E protein of Dengue virus as denoted by SEQ ID NOs: 96, 97, 99 (for serotypes 1, 2, 4, respectively), or I389, of SEQ ID NO: 98 (serotype 3), or any homologs and variants thereof. In some embodiments, the native DIII domain comprises the amino acid sequence starting at residue 300 (V300, S330, A300 or T300, of serotypes 1, 2, 3, 4, respectively) and ending at residue S390 of the E protein of Dengue virus as denoted by SEQ ID NOs: 96, 97, (for serotypes 1, 2, respectively), or N390, of SEQ ID NO: 98 (serotype 3), or H390, of SEQ ID NO: 99 (serotype 4), or any homologs and variants thereof. In some embodiments, the native DIII domain comprises the amino acid sequence starting at residue 300 (V300, S330, A300 or T300, of serotypes 1, 2, 3, 4, respectively) and ending at residue W391 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue 300 (V300, S330, A300 or T300, of serotypes 1, 2, 3, 4, respectively) and ending at residue F392 of the E protein of Dengue virus as denoted by SEQ ID NOs: 96, 97, 99 (for serotypes 1, 2, 4, respectively), or Y392, of SEQ ID NO: 98 (serotype 3), or H390, of SEQ ID NO: 99 (serotype 4), or any homologs and variants thereof. In some embodiments, the native DIII domain comprises the amino acid sequence starting at residue 300 (V300, S330, A300 or T300, of serotypes 1, 2, 3, 4, respectively) and ending at residue K393 of the E protein of Dengue virus as denoted by SEQ ID NOs: 96, 97, 98 (for serotypes 1, 2, 3, respectively), or R393, of SEQ ID NO: 99 (serotype 4), or any homologs and variants thereof. In some embodiments, the native DIII domain comprises the amino acid sequence starting at residue 300 (V300, S330, A300 or T300, of serotypes 1, 2, 3, 4, respectively) and ending at residue K394 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue 300 (V300, S330, A300 or T300, of serotypes 1, 2, 3, 4, respectively) and ending at residue G395 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue 300 (V300, S330, A300 or T300, of serotypes 1, 2, 3, 4, respectively) and ending at residue S396 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue 300 (V300, S330, A300 or T300, of serotypes 1, 2, 3, 4, respectively) and ending at residue S397 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue 300 (V300, S330, A300 or T300, of serotypes 1, 2, 3, 4, respectively) and ending at residue I398 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue 300 (V300, S330, A300 or T300, of serotypes 1, 2, 3, 4, respectively) and ending at residue N365 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue 300 (V300, S330, A300 or T300, of serotypes 1, 2, 3, 4, respectively) and ending at residue I366 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue 300 (V300, S330, A300 or T300, of serotypes 1, 2, 3, 4, respectively) and ending at residue I367 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue 300 (V300, S330, A300 or T300, of serotypes 1, 2, 3, 4, respectively) and ending at residue E368 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue 300 (V300, S330, A300 or T300, of serotypes 1, 2, 3, 4, respectively) and ending at residue and ending at residue A369 of the E protein of Dengue virus as denoted by SEQ ID Nos: 96, 97, 98 (for serotypes 1, 2, 3, respectively) and at L369 of the E protein of Dengue virus as denoted by SEQ ID NO: 99 (for serotype 4), or any homologs and variants thereof. More specifically, in some embodiments, the native DIII domain of the Dengue virus comprises the amino acid sequence starting at residue C302 and ending at residue E370 of the E protein of Dengue virus as denoted by any one of SEQ ID Nos: 96, 97, 98, 99 (for serotypes 1, 2, 3, 4, respectively), or any homologs and variants thereof. In some embodiments, the native DIII domain comprises the amino acid sequence starting at residue C302 and ending at residue P371 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue C302 and ending at residue P372 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue C302 and ending at residue F373 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue C302 and ending at residue G374 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue C302 and ending at residue E375 of the E protein of Dengue virus as denoted by SEQ ID Nos: 96, 98, (for serotypes 1, 3, respectively), or D375 of the E protein of Dengue virus as denoted by SEQ ID NOs: 97, 99 for serotypes 2, 4, respectively), or any homologs and variants thereof. In some embodiments, the native DIII domain comprises the amino acid sequence starting at residue C302 and ending at residue S376 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue C302 and ending at residue Y377 of the E protein of Dengue virus as denoted by SEQ ID NOs: 96, 97, 99 (for serotypes 1, 2, 4, respectively), and N377, for of the E protein of Dengue virus SEQ ID NO: 98 (for serotype 3), or any homologs and variants thereof. In some embodiments, the native DIII domain comprises the amino acid sequence starting at residue C302 and ending at residue 1378 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue C302 and ending at residue V379 of the E protein of Dengue virus as denoted by SEQ ID NOs: 96, 98, 99 (for serotypes 1, 3, 4, respectively), and I377, for of the E protein of Dengue virus SEQ ID NO: 97 (for serotype 2), or any homologs and variants thereof.

In some embodiments, the native DIII domain comprises the amino acid sequence starting at residue C302 and ending at residue I380 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue C302 and ending at residue G381 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue C302 and ending at residue A382 of the E protein of Dengue virus as denoted by SEQ ID NOs: 96 (serotype 1), V382 as denoted by SEQ ID NO: 97, and 99 (for serotypes 2, 4, respectively), or I382, of SEQ ID NO: 98 (serotype 3), or any homologs and variants thereof. In some embodiments, the native DIII domain comprises the amino acid sequence starting at residue C302 and ending at residue G383 of the E protein of Dengue virus as denoted by SEQ ID NOs: 96, 98, 99 (for serotypes 13, 4, respectively), or E383, as denoted by SEQ ID NO: 97 (serotype 2), or any homologs and variants thereof. In some embodiments, the native DIII domain comprises the amino acid sequence starting at residue C302 and ending at residue E384 of the E protein of Dengue virus as denoted by SEQ ID NO: 96 (serotype 1), or P384 of SEQ ID NO: 97 (serotype 2), D384 of SEQ ID NO: 98 (serotype 3), or N384 of SEQ ID NO: 99 (for serotype 4), or any homologs and variants thereof. In some embodiments, the native DIII domain comprises the amino acid sequence starting at residue C302 and ending at residue K385 of the E protein of Dengue virus as denoted by SEQ ID NOs: 96, 98, (for serotypes 1, 3, respectively), or G385, as denoted by SEQ ID NO: 97 (serotype 2), or S385, of SEQ ID NO: 99 (serotype 4), or any homologs and variants thereof. In some embodiments, the native DIII domain comprises the amino acid sequence starting at residue C302 and ending at residue A386 of the E protein of Dengue virus as denoted by SEQ ID NOs: 96, 98, 99 (for serotypes 1, 3, 4, respectively), or Q386, of SEQ ID NO: 97 (serotype 2), or any homologs and variants thereof. In some embodiments, the native DIII domain comprises the amino acid sequence starting at residue C302 and ending at residue L387 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue C302 and ending at residue K388 of the E protein of Dengue virus as denoted by SEQ ID NOs: 96, 97, 98, (for serotypes 1, 2, 3, respectively), or T388, of SEQ ID NO: 99 (serotype 4), or any homologs and variants thereof. In some embodiments, the native DIII domain comprises the amino acid sequence starting at residue C302 and ending at residue L389 of the E protein of Dengue virus as denoted by SEQ ID NOs: 96, 97, 99 (for serotypes 1, 2, 4, respectively), or I389, of SEQ ID NO: 98 (serotype 3), or any homologs and variants thereof. In some embodiments, the native DIII domain comprises the amino acid sequence starting at residue C302 and ending at residue S390 of the E protein of Dengue virus as denoted by SEQ ID NOs: 96, 97, (for serotypes 1, 2, respectively), or N390, of SEQ ID NO: 98 (serotype 3), or H390, of SEQ ID NO: 99 (serotype 4), or any homologs and variants thereof. In some embodiments, the native DIII domain comprises the amino acid sequence starting at residue C302 and ending at residue W391 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue C302 and ending at residue F392 of the E protein of Dengue virus as denoted by SEQ ID NOs: 96, 97, 99 (for serotypes 1, 2, 4, respectively), or Y392, of SEQ ID NO: 98 (serotype 3), or H390, of SEQ ID NO: 99 (serotype 4), or any homologs and variants thereof. In some embodiments, the native DIII domain comprises the amino acid sequence starting at residue C302 and ending at residue K393 of the E protein of Dengue virus as denoted by SEQ ID NOs: 96, 97, 98 (for serotypes 1, 2, 3, respectively), or R393, of SEQ ID NO: 99 (serotype 4), or any homologs and variants thereof. In some embodiments, the native DIII domain comprises the amino acid sequence starting at residue C302 and ending at residue K394 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue C302 and ending at residue G395 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue C302 and ending at residue S396 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue C302 and ending at residue S397 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue C302 and ending at residue I398 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue C302 and ending at residue N365 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue C302 and ending at residue I366 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue C302 and ending at residue I367 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue C302 and ending at residue E368 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue C302 and ending at residue and ending at residue A369 of the E protein of Dengue virus as denoted by SEQ ID Nos: 96, 97, 98 (for serotypes 1, 2, 3, respectively) and at L369 of the E protein of Dengue virus as denoted by SEQ ID NO: 99 (for serotype 4), or any homologs and variants thereof. More specifically, in some embodiments, the native DIII domain of the Dengue virus comprises the amino acid sequence starting at residue T303 (or L303, or S303, for serotypes 3 and 4) and ending at residue E370 of the E protein of Dengue virus as denoted by any one of SEQ ID Nos: 96, 97, 98, 99 (for serotypes 1, 2, 3, 4, respectively), or any homologs and variants thereof. In some embodiments, the native DIII domain comprises the amino acid sequence starting at residue T303 (or L303, or S303, for serotypes 3 and 4) and ending at residue P371 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue T303 (or L303, or S303, for serotypes 3 and 4) and ending at residue P372 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue T303 (or L303, or S303, for serotypes 3 and 4) and ending at residue F373 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue T303 (or L303, or S303, for serotypes 3 and 4) and ending at residue G374 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue T303 (or L303, or S303, for serotypes 3 and 4) and ending at residue E375 of the E protein of Dengue virus as denoted by SEQ ID Nos: 96, 98, (for serotypes 1, 3, respectively), or D375 of the E protein of Dengue virus as denoted by SEQ ID NOs: 97, 99 for serotypes 2, 4, respectively), or any homologs and variants thereof. In some embodiments, the native DIII domain comprises the amino acid sequence starting at residue T303 (or L303, or S303, for serotypes 3 and 4) and ending at residue S376 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue T303 (or L303, or S303, for serotypes 3 and 4) and ending at residue Y377 of the E protein of Dengue virus as denoted by SEQ ID NOs: 96, 97, 99 (for serotypes 1, 2, 4, respectively), and N377, for of the E protein of Dengue virus SEQ ID NO: 98 (for serotype 3), or any homologs and variants thereof. In some embodiments, the native DIII domain comprises the amino acid sequence starting at residue T303 (or L303, or S303, for serotypes 3 and 4) and ending at residue I378 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue T303 (or L303, or S303, for serotypes 3 and 4) and ending at residue V379 of the E protein of Dengue virus as denoted by SEQ ID NOs: 96, 98, 99 (for serotypes 1, 3, 4, respectively), and 1377, for of the E protein of Dengue virus SEQ ID NO: 97 (for serotype 2), or any homologs and variants thereof. In some embodiments, the native DIII domain comprises the amino acid sequence starting at residue T303 (or L303, or S303, for serotypes 3 and 4) and ending at residue I380 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue T303 (or L303, or S303, for serotypes 3 and 4) and ending at residue G381 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue T303 (or L303, or S303, for serotypes 3 and 4) and ending at residue A382 of the E protein of Dengue virus as denoted by SEQ ID NOs: 96 (serotype 1), V382 as denoted by SEQ ID NO: 97, and 99 (for serotypes 2, 4, respectively), or I382, of SEQ ID NO: 98 (serotype 3), or any homologs and variants thereof. In some embodiments, the native DIII domain comprises the amino acid sequence starting at residue T303 (or L303, or S303, for serotypes 3 and 4) and ending at residue G383 of the E protein of Dengue virus as denoted by SEQ ID NOs: 96, 98, 99 (for serotypes 13, 4, respectively), or E383, as denoted by SEQ ID NO: 97 (serotype 2), or any homologs and variants thereof. In some embodiments, the native DIII domain comprises the amino acid sequence starting at residue T303 (or L303, or S303, for serotypes 3 and 4) and ending at residue E384 of the E protein of Dengue virus as denoted by SEQ ID NO: 96 (serotype 1), or P384 of SEQ ID NO: 97 (serotype 2), D384 of SEQ ID NO: 98 (serotype 3), or N384 of SEQ ID NO: 99 (for serotype 4), or any homologs and variants thereof. In some embodiments, the native DIII domain comprises the amino acid sequence starting at residue T303 (or L303, or S303, for serotypes 3 and 4) and ending at residue K385 of the E protein of Dengue virus as denoted by SEQ ID NOs: 96, 98, (for serotypes 1, 3, respectively), or G385, as denoted by SEQ ID NO: 97 (serotype 2), or S385, of SEQ ID NO: 99 (serotype 4), or any homologs and variants thereof.

In some embodiments, the native DIII domain comprises the amino acid sequence starting at residue T303 (or L303, or S303, for serotypes 3 and 4) and ending at residue A386 of the E protein of Dengue virus as denoted by SEQ ID NOs: 96, 98, 99 (for serotypes 1, 3, 4, respectively), or Q386, of SEQ ID NO: 97 (serotype 2), or any homologs and variants thereof. In some embodiments, the native DIII domain comprises the amino acid sequence starting at residue T303 (or L303, or S303, for serotypes 3 and 4) and ending at residue L387 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue T303 (or L303, or S303, for serotypes 3 and 4) and ending at residue K388 of the E protein of Dengue virus as denoted by SEQ ID NOs: 96, 97, 98, (for serotypes 1, 2, 3, respectively), or T388, of SEQ ID NO: 99 (serotype 4), or any homologs and variants thereof. In some embodiments, the native DIII domain comprises the amino acid sequence starting at residue T303 (or L303, or S303, for serotypes 3 and 4) and ending at residue L389 of the E protein of Dengue virus as denoted by SEQ ID NOs: 96, 97, 99 (for serotypes 1, 2, 4, respectively), or I389, of SEQ ID NO: 98 (serotype 3), or any homologs and variants thereof. In some embodiments, the native DIII domain comprises the amino acid sequence starting at residue T303 (or L303, or S303, for serotypes 3 and 4) and ending at residue S390 of the E protein of Dengue virus as denoted by SEQ ID NOs: 96, 97, (for serotypes 1, 2, respectively), or N390, of SEQ ID NO: 98 (serotype 3), or H390, of SEQ ID NO: 99 (serotype 4), or any homologs and variants thereof. In some embodiments, the native DIII domain comprises the amino acid sequence starting at residue T303 (or L303, or S303, for serotypes 3 and 4) and ending at residue W391 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue T303 (or L303, or S303, for serotypes 3 and 4) and ending at residue F392 of the E protein of Dengue virus as denoted by SEQ ID NOs: 96, 97, 99 (for serotypes 1, 2, 4, respectively), or Y392, of SEQ ID NO: 98 (serotype 3), or H390, of SEQ ID NO: 99 (serotype 4), or any homologs and variants thereof. In some embodiments, the native DIII domain comprises the amino acid sequence starting at residue T303 (or L303, or S303, for serotypes 3 and 4) and ending at residue K393 of the E protein of Dengue virus as denoted by SEQ ID NOs: 96, 97, 98 (for serotypes 1, 2, 3, respectively), or R393, of SEQ ID NO: 99 (serotype 4), or any homologs and variants thereof. In some embodiments, the native DIII domain comprises the amino acid sequence starting at residue T303 (or L303, or S303, for serotypes 3 and 4) and ending at residue K394 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue T303 (or L303, or S303, for serotypes 3 and 4) and ending at residue G395 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue T303 (or L303, or S303, for serotypes 3 and 4) and ending at residue S396 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue T303 (or L303, or S303, for serotypes 3 and 4) and ending at residue S397 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue T303 (or L303, or S303, for serotypes 3 and 4) and ending at residue I398 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue T303 (or L303, or S303, for serotypes 3 and 4) and ending at residue N365 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue T303 (or L303, or S303, for serotypes 3 and 4) and ending at residue I366 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue T303 (or L303, or S303, for serotypes 3 and 4) and ending at residue I367 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue T303 (or L303, or S303, for serotypes 3 and 4) and ending at residue E368 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue T303 (or L303, or S303, for serotypes 3 and 4) and ending at residue and ending at residue A369 of the E protein of Dengue virus as denoted by SEQ ID Nos: 96, 97, 98 (for serotypes 1, 2, 3, respectively) and at L369 of the E protein of Dengue virus as denoted by SEQ ID NO: 99 (for serotype 4), or any homologs and variants thereof. More specifically, in some embodiments, the native DIII domain of the Dengue virus comprises the amino acid sequence starting at residue G304 (or N304 for serotype 3) and ending at residue E370 of the E protein of Dengue virus as denoted by any one of SEQ ID Nos: 96, 97, 98, 99 (for serotypes 1, 2, 3, 4, respectively), or any homologs and variants thereof. In some embodiments, the native DIII domain comprises the amino acid sequence starting at residue G304 (or N304 for serotype 3) and ending at residue P371 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue G304 (or N304 for serotype 3) and ending at residue P372 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue G304 (or N304 for serotype 3) and ending at residue F373 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G304 (or N304 for serotype 3) and ending at residue G374 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue G304 (or N304 for serotype 3) and ending at residue E375 of the E protein of Dengue virus as denoted by SEQ ID Nos: 96, 98, (for serotypes 1, 3, respectively), or D375 of the E protein of Dengue virus as denoted by SEQ ID NOs: 97, 99 for serotypes 2, 4, respectively), or any homologs and variants thereof. In some embodiments, the native DIII domain comprises the amino acid sequence starting at residue G304 (or N304 for serotype 3) and ending at residue S376 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue G304 (or N304 for serotype 3) and ending at residue Y377 of the E protein of Dengue virus as denoted by SEQ ID NOs: 96, 97, 99 (for serotypes 1, 2, 4, respectively), and N377, for of the E protein of Dengue virus SEQ ID NO: 98 (for serotype 3), or any homologs and variants thereof. In some embodiments, the native DIII domain comprises the amino acid sequence starting at residue G304 (or N304 for serotype 3) and ending at residue I378 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G304 (or N304 for serotype 3) and ending at residue V379 of the E protein of Dengue virus as denoted by SEQ ID NOs: 96, 98, 99 (for serotypes 1, 3, 4, respectively), and I377, for of the E protein of Dengue virus SEQ ID NO: 97 (for serotype 2), or any homologs and variants thereof. In some embodiments, the native DIII domain comprises the amino acid sequence starting at residue G304 (or N304 for serotype 3) and ending at residue I380 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G304 (or N304 for serotype 3) and ending at residue G381 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G304 (or N304 for serotype 3) and ending at residue A382 of the E protein of Dengue virus as denoted by SEQ ID NOs: 96 (serotype 1), V382 as denoted by SEQ ID NO: 97, and 99 (for serotypes 2, 4, respectively), or I382, of SEQ ID NO: 98 (serotype 3), or any homologs and variants thereof. In some embodiments, the native DIII domain comprises the amino acid sequence starting at residue G304 (or N304 for serotype 3) and ending at residue G383 of the E protein of Dengue virus as denoted by SEQ ID NOs: 96, 98, 99 (for serotypes 13, 4, respectively), or E383, as denoted by SEQ ID NO: 97 (serotype 2), or any homologs and variants thereof. In some embodiments, the native DIII domain comprises the amino acid sequence starting at residue G304 (or N304 for serotype 3) and ending at residue E384 of the E protein of Dengue virus as denoted by SEQ ID NO: 96 (serotype 1), or P384 of SEQ ID NO: 97 (serotype 2), D384 of SEQ ID NO: 98 (serotype 3), or N384 of SEQ ID NO: 99 (for serotype 4), or any homologs and variants thereof. In some embodiments, the native DIII domain comprises the amino acid sequence starting at residue G304 (or N304 for serotype 3) and ending at residue K385 of the E protein of Dengue virus as denoted by SEQ ID NOs: 96, 98, (for serotypes 1, 3, respectively), or G385, as denoted by SEQ ID NO: 97 (serotype 2), or S385, of SEQ ID NO: 99 (serotype 4), or any homologs and variants thereof. In some embodiments, the native DIII domain comprises the amino acid sequence starting at residue G304 (or N304 for serotype 3) and ending at residue A386 of the E protein of Dengue virus as denoted by SEQ ID NOs: 96, 98, 99 (for serotypes 1, 3, 4, respectively), or Q386, of SEQ ID NO: 97 (serotype 2), or any homologs and variants thereof. In some embodiments, the native DIII domain comprises the amino acid sequence starting at residue G304 (or N304 for serotype 3) and ending at residue L387 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G304 (or N304 for serotype 3) and ending at residue K388 of the E protein of Dengue virus as denoted by SEQ ID NOs: 96, 97, 98, (for serotypes 1, 2, 3, respectively), or T388, of SEQ ID NO: 99 (serotype 4), or any homologs and variants thereof. In some embodiments, the native DIII domain comprises the amino acid sequence starting at residue G304 (or N304 for serotype 3) and ending at residue L389 of the E protein of Dengue virus as denoted by SEQ ID NOs: 96, 97, 99 (for serotypes 1, 2, 4, respectively), or I389, of SEQ ID NO: 98 (serotype 3), or any homologs and variants thereof. In some embodiments, the native DIII domain comprises the amino acid sequence starting at residue G304 (or N304 for serotype 3) and ending at residue S390 of the E protein of Dengue virus as denoted by SEQ ID NOs: 96, 97, (for serotypes 1, 2, respectively), or N390, of SEQ ID NO: 98 (serotype 3), or H390, of SEQ ID NO: 99 (serotype 4), or any homologs and variants thereof. In some embodiments, the native DIII domain comprises the amino acid sequence starting at residue G304 (or N304 for serotype 3) and ending at residue W391 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G304 (or N304 for serotype 3) and ending at residue F392 of the E protein of Dengue virus as denoted by SEQ ID NOs: 96, 97, 99 (for serotypes 1, 2, 4, respectively), or Y392, of SEQ ID NO: 98 (serotype 3), or H390, of SEQ ID NO: 99 (serotype 4), or any homologs and variants thereof. In some embodiments, the native DIII domain comprises the amino acid sequence starting at residue G304 (or N304 for serotype 3) and ending at residue K393 of the E protein of Dengue virus as denoted by SEQ ID NOs: 96, 97, 98 (for serotypes 1, 2, 3, respectively), or R393, of SEQ ID NO: 99 (serotype 4), or any homologs and variants thereof. In some embodiments, the native DIII domain comprises the amino acid sequence starting at residue G304 (or N304 for serotype 3) and ending at residue K394 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G304 (or N304 for serotype 3) and ending at residue G395 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G304 (or N304 for serotype 3) and ending at residue S396 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G304 (or N304 for serotype 3) and ending at residue S397 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G304 (or N304 for serotype 3) and ending at residue I398 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G304 (or N304 for serotype 3) and ending at residue N365 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G304 (or N304 for serotype 3) and ending at residue I366 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue G304 (or N304 for serotype 3) and ending at residue I367 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue G304 (or N304 for serotype 3) and ending at residue E368 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue G304 (or N304 for serotype 3) and ending at residue and ending at residue A369 of the E protein of Dengue virus as denoted by SEQ ID Nos: 96, 97, 98 (for serotypes 1, 2, 3, respectively) and at L369 of the E protein of Dengue virus as denoted by SEQ ID NO: 99 (for serotype 4), or any homologs and variants thereof. More specifically, in some embodiments, the native DIII domain of the Dengue virus comprises the amino acid sequence starting at residue S305 (or K305 for serotypes 2 and 4, and T305 for serotype 3) and ending at residue E370 of the E protein of Dengue virus as denoted by any one of SEQ ID Nos: 96, 97, 98, 99 (for serotypes 1, 2, 3, 4, respectively), or any homologs and variants thereof. In some embodiments, the native DIII domain comprises the amino acid sequence starting at residue S305 (or K305 for serotypes 2 and 4, and T305 for serotype 3) and ending at residue P371 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue S305 (or K305 for serotypes 2 and 4, and T305 for serotype 3) and ending at residue P372 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue S305 (or K305 for serotypes 2 and 4, and T305 for serotype 3) and ending at residue F373 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S305 (or K305 for serotypes 2 and 4, and T305 for serotype 3) and ending at residue G374 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue S305 (or K305 for serotypes 2 and 4, and T305 for serotype 3) and ending at residue E375 of the E protein of Dengue virus as denoted by SEQ ID Nos: 96, 98, (for serotypes 1, 3, respectively), or D375 of the E protein of Dengue virus as denoted by SEQ ID NOs: 97, 99 for serotypes 2, 4, respectively), or any homologs and variants thereof. In some embodiments, the native DIII domain comprises the amino acid sequence starting at residue S305 (or K305 for serotypes 2 and 4, and T305 for serotype 3) and ending at residue S376 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue S305 (or K305 for serotypes 2 and 4, and T305 for serotype 3) and ending at residue Y377 of the E protein of Dengue virus as denoted by SEQ ID NOs: 96, 97, 99 (for serotypes 1, 2, 4, respectively), and N377, for of the E protein of Dengue virus SEQ ID NO: 98 (for serotype 3), or any homologs and variants thereof. In some embodiments, the native DIII domain comprises the amino acid sequence starting at residue S305 (or K305 for serotypes 2 and 4, and T305 for serotype 3) and ending at residue 1378 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S305 (or K305 for serotypes 2 and 4, and T305 for serotype 3) and ending at residue V379 of the E protein of Dengue virus as denoted by SEQ ID NOs: 96, 98, 99 (for serotypes 1, 3, 4, respectively), and 1377, for of the E protein of Dengue virus SEQ ID NO: 97 (for serotype 2), or any homologs and variants thereof. In some embodiments, the native DIII domain comprises the amino acid sequence starting at residue S305 (or K305 for serotypes 2 and 4, and T305 for serotype 3) and ending at residue I380 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S305 (or K305 for serotypes 2 and 4, and T305 for serotype 3) and ending at residue G381 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S305 (or K305 for serotypes 2 and 4, and T305 for serotype 3) and ending at residue A382 of the E protein of Dengue virus as denoted by SEQ ID NOs: 96 (serotype 1), V382 as denoted by SEQ ID NO: 97, and 99 (for serotypes 2, 4, respectively), or I382, of SEQ ID NO: 98 (serotype 3), or any homologs and variants thereof. In some embodiments, the native DIII domain comprises the amino acid sequence starting at residue S305 (or K305 for serotypes 2 and 4, and T305 for serotype 3) and ending at residue G383 of the E protein of Dengue virus as denoted by SEQ ID NOs: 96, 98, 99 (for serotypes 13, 4, respectively), or E383, as denoted by SEQ ID NO: 97 (serotype 2), or any homologs and variants thereof. In some embodiments, the native DIII domain comprises the amino acid sequence starting at residue S305 (or K305 for serotypes 2 and 4, and T305 for serotype 3) and ending at residue E384 of the E protein of Dengue virus as denoted by SEQ ID NO: 96 (serotype 1), or P384 of SEQ ID NO: 97 (serotype 2), D384 of SEQ ID NO: 98 (serotype 3), or N384 of SEQ ID NO: 99 (for serotype 4), or any homologs and variants thereof. In some embodiments, the native DIII domain comprises the amino acid sequence starting at residue S305 (or K305 for serotypes 2 and 4, and T305 for serotype 3) and ending at residue K385 of the E protein of Dengue virus as denoted by SEQ ID NOs: 96, 98, (for serotypes 1, 3, respectively), or G385, as denoted by SEQ ID NO: 97 (serotype 2), or S385, of SEQ ID NO: 99 (serotype 4), or any homologs and variants thereof. In some embodiments, the native DIII domain comprises the amino acid sequence starting at residue S305 (or K305 for serotypes 2 and 4, and T305 for serotype 3) and ending at residue A386 of the E protein of Dengue virus as denoted by SEQ ID NOs: 96, 98, 99 (for serotypes 1, 3, 4, respectively), or Q386, of SEQ ID NO: 97 (serotype 2), or any homologs and variants thereof. In some embodiments, the native DIII domain comprises the amino acid sequence starting at residue S305 (or K305 for serotypes 2 and 4, and T305 for serotype 3) and ending at residue L387 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S305 (or K305 for serotypes 2 and 4, and T305 for serotype 3) and ending at residue K388 of the E protein of Dengue virus as denoted by SEQ ID NOs: 96, 97, 98, (for serotypes 1, 2, 3, respectively), or T388, of SEQ ID NO: 99 (serotype 4), or any homologs and variants thereof. In some embodiments, the native DIII domain comprises the amino acid sequence starting at residue S305 (or K305 for serotypes 2 and 4, and T305 for serotype 3) and ending at residue L389 of the E protein of Dengue virus as denoted by SEQ ID NOs: 96, 97, 99 (for serotypes 1, 2, 4, respectively), or I389, of SEQ ID NO: 98 (serotype 3), or any homologs and variants thereof. In some embodiments, the native DIII domain comprises the amino acid sequence starting at residue S305 (or K305 for serotypes 2 and 4, and T305 for serotype 3) and ending at residue S390 of the E protein of Dengue virus as denoted by SEQ ID NOs: 96, 97, (for serotypes 1, 2, respectively), or N390, of SEQ ID NO: 98 (serotype 3), or H390, of SEQ ID NO: 99 (serotype 4), or any homologs and variants thereof. In some embodiments, the native DIII domain comprises the amino acid sequence starting at residue S305 (or K305 for serotypes 2 and 4, and T305 for serotype 3) and ending at residue W391 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S305 (or K305 for serotypes 2 and 4, and T305 for serotype 3) and ending at residue F392 of the E protein of Dengue virus as denoted by SEQ ID NOs: 96, 97, 99 (for serotypes 1, 2, 4, respectively), or Y392, of SEQ ID NO: 98 (serotype 3), or H390, of SEQ ID NO: 99 (serotype 4), or any homologs and variants thereof. In some embodiments, the native DIII domain comprises the amino acid sequence starting at residue S305 (or K305 for serotypes 2 and 4, and T305 for serotype 3) and ending at residue K393 of the E protein of Dengue virus as denoted by SEQ ID NOs: 96, 97, 98 (for serotypes 1, 2, 3, respectively), or R393, of SEQ ID NO: 99 (serotype 4), or any homologs and variants thereof. In some embodiments, the native DIII domain comprises the amino acid sequence starting at residue S305 (or K305 for serotypes 2 and 4, and T305 for serotype 3) and ending at residue K394 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S305 (or K305 for serotypes 2 and 4, and T305 for serotype 3) and ending at residue G395 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S305 (or K305 for serotypes 2 and 4, and T305 for serotype 3) and ending at residue S396 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S305 (or K305 for serotypes 2 and 4, and T305 for serotype 3) and ending at residue S397 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S305 (or K305 for serotypes 2 and 4, and T305 for serotype 3) and ending at residue I398 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S305 (or K305 for serotypes 2 and 4, and T305 for serotype 3) and ending at residue N365 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S305 (or K305 for serotypes 2 and 4, and T305 for serotype 3) and ending at residue I366 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue S305 (or K305 for serotypes 2 and 4, and T305 for serotype 3) and ending at residue I367 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue S305 (or K305 for serotypes 2 and 4, and T305 for serotype 3) and ending at residue E368 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue S305 (or K305 for serotypes 2 and 4, and T305 for serotype 3) and ending at residue and ending at residue A369 of the E protein of Dengue virus as denoted by SEQ ID Nos: 96, 97, 98 (for serotypes 1, 2, 3, respectively) and at L369 of the E protein of Dengue virus as denoted by SEQ ID NO: 99 (for serotype 4), or any homologs and variants thereof. More specifically, in some embodiments, the native DIII domain of the Dengue virus comprises the amino acid sequence starting at residue F306 and ending at residue E370 of the E protein of Dengue virus as denoted by any one of SEQ ID Nos: 96, 97, 98, 99 (for serotypes 1, 2, 3, 4, respectively), or any homologs and variants thereof. In some embodiments, the native DIII domain comprises the amino acid sequence starting at residue F306 and ending at residue P371 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue F306 and ending at residue P372 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue F306 and ending at residue F373 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue F306 and ending at residue G374 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue F306 and ending at residue E375 of the E protein of Dengue virus as denoted by SEQ ID Nos: 96, 98, (for serotypes 1, 3, respectively), or D375 of the E protein of Dengue virus as denoted by SEQ ID NOs: 97, 99 for serotypes 2, 4, respectively), or any homologs and variants thereof. In some embodiments, the native DIII domain comprises the amino acid sequence starting at residue F306 and ending at residue S376 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue F306 and ending at residue Y377 of the E protein of Dengue virus as denoted by SEQ ID NOs: 96, 97, 99 (for serotypes 1, 2, 4, respectively), and N377, for of the E protein of Dengue virus SEQ ID NO: 98 (for serotype 3), or any homologs and variants thereof. In some embodiments, the native DIII domain comprises the amino acid sequence starting at residue F306 and ending at residue 1378 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue F306 and ending at residue V379 of the E protein of Dengue virus as denoted by SEQ ID NOs: 96, 98, 99 (for serotypes 1, 3, 4, respectively), and 1377, for of the E protein of Dengue virus SEQ ID NO: 97 (for serotype 2), or any homologs and variants thereof. In some embodiments, the native DIII domain comprises the amino acid sequence starting at residue F306 and ending at residue I380 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue F306 and ending at residue G381 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue F306 and ending at residue A382 of the E protein of Dengue virus as denoted by SEQ ID NOs: 96 (serotype 1), V382 as denoted by SEQ ID NO: 97, and 99 (for serotypes 2, 4, respectively), or I382, of SEQ ID NO: 98 (serotype 3), or any homologs and variants thereof. In some embodiments, the native DIII domain comprises the amino acid sequence starting at residue F306 and ending at residue G383 of the E protein of Dengue virus as denoted by SEQ ID NOs: 96, 98, 99 (for serotypes 13, 4, respectively), or E383, as denoted by SEQ ID NO: 97 (serotype 2), or any homologs and variants thereof. In some embodiments, the native DIII domain comprises the amino acid sequence starting at residue F306 and ending at residue E384 of the E protein of Dengue virus as denoted by SEQ ID NO: 96 (serotype 1), or P384 of SEQ ID NO: 97 (serotype 2), D384 of SEQ ID NO: 98 (serotype 3), or N384 of SEQ ID NO: 99 (for serotype 4), or any homologs and variants thereof. In some embodiments, the native DIII domain comprises the amino acid sequence starting at residue F306 and ending at residue K385 of the E protein of Dengue virus as denoted by SEQ ID NOs: 96, 98, (for serotypes 1, 3, respectively), or G385, as denoted by SEQ ID NO: 97 (serotype 2), or S385, of SEQ ID NO: 99 (serotype 4), or any homologs and variants thereof. In some embodiments, the native DIII domain comprises the amino acid sequence starting at residue F306 and ending at residue A386 of the E protein of Dengue virus as denoted by SEQ ID NOs: 96, 98, 99 (for serotypes 1, 3, 4, respectively), or Q386, of SEQ ID NO: 97 (serotype 2), or any homologs and variants thereof. In some embodiments, the native DIII domain comprises the amino acid sequence starting at residue F306 and ending at residue L387 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue F306 and ending at residue K388 of the E protein of Dengue virus as denoted by SEQ ID NOs: 96, 97, 98, (for serotypes 1, 2, 3, respectively), or T388, of SEQ ID NO: 99 (serotype 4), or any homologs and variants thereof. In some embodiments, the native DIII domain comprises the amino acid sequence starting at residue F306 and ending at residue L389 of the E protein of Dengue virus as denoted by SEQ ID NOs: 96, 97, 99 (for serotypes 1, 2, 4, respectively), or I389, of SEQ ID NO: 98 (serotype 3), or any homologs and variants thereof. In some embodiments, the native DIII domain comprises the amino acid sequence starting at residue F306 and ending at residue S390 of the E protein of Dengue virus as denoted by SEQ ID NOs: 96, 97, (for serotypes 1, 2, respectively), or N390, of SEQ ID NO: 98 (serotype 3), or H390, of SEQ ID NO: 99 (serotype 4), or any homologs and variants thereof. In some embodiments, the native DIII domain comprises the amino acid sequence starting at residue F306 and ending at residue W391 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue F306 and ending at residue F392 of the E protein of Dengue virus as denoted by SEQ ID NOs: 96, 97, 99 (for serotypes 1, 2, 4, respectively), or Y392, of SEQ ID NO: 98 (serotype 3), or H390, of SEQ ID NO: 99 (serotype 4), or any homologs and variants thereof. In some embodiments, the native DIII domain comprises the amino acid sequence starting at residue F306 and ending at residue K393 of the E protein of Dengue virus as denoted by SEQ ID NOs: 96, 97, 98 (for serotypes 1, 2, 3, respectively), or R393, of SEQ ID NO: 99 (serotype 4), or any homologs and variants thereof. In some embodiments, the native DIII domain comprises the amino acid sequence starting at residue F306 and ending at residue K394 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue F306 and ending at residue G395 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue F306 and ending at residue S396 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue F306 and ending at residue S397 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue F306 and ending at residue I398 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue F306 and ending at residue N365 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue F306 and ending at residue I366 of the E protein of Dengue virus as denoted by SEQ ID NOs: 96, 97, 98, 99 (for serotypes 1, 2, 3, 4, respectively), or any homologs and variants thereof. In some embodiments, the native DIII domain comprises the amino acid sequence starting at residue F306 and ending at residue I367 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue F306 and ending at residue E368 of the E protein of Dengue virus as denoted by SEQ ID Nos: 96, 97, 98, 99 (for serotypes 1, 2, 3, 4, respectively), or any homologs and variants thereof. In some embodiments, the native DIII domain comprises the amino acid sequence starting at residue F306 and ending at residue and ending at residue A369 of the E protein of Dengue virus as denoted by SEQ ID Nos: 96, 97, 98 (for serotypes 1, 2, 3, respectively) and at L369 of the E protein of Dengue virus as denoted by SEQ ID NO: 99 (for serotype 4), or any homologs and variants thereof. In some specific embodiments, the reconstituted epitope of the polypeptide of the present disclosure comprises an amino acid sequence of the native DIII domain of the E protein starting at any one of the amino acid residues 301, 296, 297, 298, 299, 300, 302, 303, 304, 305 or 306 and ending at any one of the amino acid residues 370, 365, 366, 367, 368, 369, 371, 372, 373, 374, 375, 376, 377, 378, 379, 380, 381, 382, 383, 384, 385, 386, 387, 388, 389, 390, 391, 392, 393, 39, 395, 396, 397 or 398. In some embodiments, the DIII domain comprises residues 301 to 370, of the Dengue virus E protein. In more specific embodiments, the native DIII domain comprises a loop comprising an amino acid sequence starting at any one of the amino acid residues 336, 333, 334, 335, 337, 338 or 339, and ending at any one of the amino acid residues 355, 352, 353, 354, 356, 357 or 358. In some embodiments, the loop comprises residues 336-355. In some embodiments, the bridging linker may bridge the amino acid residue I335 with P356. In some embodiments, the bridging linker may bridge the amino acid residue I335 with I351L (or I351). In some embodiments, the bridging linker may bridge the amino acid residue I335 with I352. In some embodiments, the bridging linker may bridge the amino acid residue P356 with T353 (or S353). In some embodiments, the bridging linker may bridge the amino acid residue I335 with A354 (or V354, or S354). In some alternative embodiments, the bridging linker may bridge the amino acid residue I335 with N355 (or T355). In some further embodiments, the bridging linker may bridge the amino acid residue I335 with I357 (or V357, or I357). In some embodiments, the bridging linker may bridge the amino acid residue I335 with V358 (or A358). In some embodiments, the bridging linker may bridge the amino acid residue I335 with T359 (or E359). In some embodiments, the bridging linker may bridge the amino acid residue P332 with P356. In some embodiments, the bridging linker may bridge the amino acid residue P332 with I351L (or I351). In some embodiments, the bridging linker may bridge the amino acid residue P332 with I352. In some embodiments, the bridging linker may bridge the amino acid residue P356 with T353 (or S353). In some embodiments, the bridging linker may bridge the amino acid residue P332 with A354 (or V354, or S354). In some alternative embodiments, the bridging linker may bridge the amino acid residue P332 with N355 (or T355). In some further embodiments, the bridging linker may bridge the amino acid residue P332 with I357 (or V357, or I357). In some embodiments, the bridging linker may bridge the amino acid residue P332 with V358 (or A358). In some embodiments, the bridging linker may bridge the amino acid residue P332 with T359 (or E359). In some embodiments, the bridging linker may bridge the amino acid residue C333 with P356. In some embodiments, the bridging linker may bridge the amino acid residue C333 with I351L (or I351). In some embodiments, the bridging linker may bridge the amino acid residue C333 with I352. In some embodiments, the bridging linker may bridge the amino acid residue P356 with T353 (or S353). In some embodiments, the bridging linker may bridge the amino acid residue C333 with A354 (or V354, or S354). In some alternative embodiments, the bridging linker may bridge the amino acid residue C333 with N355 (or T355). In some further embodiments, the bridging linker may bridge the amino acid residue C333 with I357 (or V357, or I357). In some embodiments, the bridging linker may bridge the amino acid residue C333 with V358 (or A358). In some embodiments, the bridging linker may bridge the amino acid residue C333 with T359 (or E359). In some embodiments, the bridging linker may bridge the amino acid residue K334 with P356. In some embodiments, the bridging linker may bridge the amino acid residue K334 with I351L (or I351). In some embodiments, the bridging linker may bridge the amino acid residue K334 with I352. In some embodiments, the bridging linker may bridge the amino acid residue P356 with T353 (or S353). In some embodiments, the bridging linker may bridge the amino acid residue K334 with A354 (or V354, or S354). In some alternative embodiments, the bridging linker may bridge the amino acid residue K334 with N355 (or T355). In some further embodiments, the bridging linker may bridge the amino acid residue K334 with I357 (or V357, or I357). In some embodiments, the bridging linker may bridge the amino acid residue K334 with V358 (or A358). In some embodiments, the bridging linker may bridge the amino acid residue K334 with T359 (or E359). In some embodiments, the bridging linker may bridge the amino acid residue P336 with P356. In some embodiments, the bridging linker may bridge the amino acid residue P336 with I351L (or I351). In some embodiments, the bridging linker may bridge the amino acid residue P336 with I352. In some embodiments, the bridging linker may bridge the amino acid residue P356 with T353 (or S353). In some embodiments, the bridging linker may bridge the amino acid residue P336 with A354 (or V354, or S354). In some alternative embodiments, the bridging linker may bridge the amino acid residue P336 with N355 (or T355). In some further embodiments, the bridging linker may bridge the amino acid residue P336 with I357 (or V357, or I357). In some embodiments, the bridging linker may bridge the amino acid residue P336 with V358 (or A358). In some embodiments, the bridging linker may bridge the amino acid residue P336 with T359 (or E359). In some embodiments, the bridging linker may bridge the amino acid residue F337 (or 1337) with P356. In some embodiments, the bridging linker may bridge the amino acid residue F337 (or I337) with I351L (or I351). In some embodiments, the bridging linker may bridge the amino acid residue F337 (or I337) with I352. In some embodiments, the bridging linker may bridge the amino acid residue F337 with T353 (or S353). In some embodiments, the bridging linker may bridge the amino acid residue F337 (or I337) with A354 (or V354, or S354). In some alternative embodiments, the bridging linker may bridge the amino acid residue F337 (or I337) with N355 (or T355). In some further embodiments, the bridging linker may bridge the amino acid residue F337 (or I337) with I357 (or V357, or I357). In some embodiments, the bridging linker may bridge the amino acid residue F337 (or I337) with V358 (or A358). In some embodiments, the bridging linker may bridge the amino acid residue F337 (or I337) with T359 (or E359). In some embodiments, the bridging linker may bridge the amino acid residue S338 (or E338) with P356. In some embodiments, the bridging linker may bridge the amino acid residue S338 (or E338) with I351L (or I351). In some embodiments, the bridging linker may bridge the amino acid residue S338 (or E338) with I352. In some embodiments, the bridging linker may bridge the amino acid residue P356 with T353 (or S353). In some embodiments, the bridging linker may bridge the amino acid residue S338 (or E338) with A354 (or V354, or S354). In some alternative embodiments, the bridging linker may bridge the amino acid residue S338 (or E338) with N355 (or T355). In some further embodiments, the bridging linker may bridge the amino acid residue S338 (or E338) with 1357 (or V357, or I357). In some embodiments, the bridging linker may bridge the amino acid residue S338 (or E338) with V358 (or A358). In some embodiments, the bridging linker may bridge the amino acid residue S338 (or E338) with T359 (or E359). In some embodiments, the bridging linker may bridge the amino acid residue S339 (or I339, or T339) with P356. In some embodiments, the bridging linker may bridge the amino acid residue S339 (or I339, or T339) with I351L (or I351). In some embodiments, the bridging linker may bridge the amino acid residue S339 (or I339, or T339) with I352. In some embodiments, the bridging linker may bridge the amino acid residue P356 with T353 (or S353). In some embodiments, the bridging linker may bridge the amino acid residue S339 (or I339, or T339) with A354 (or V354, or S354). In some alternative embodiments, the bridging linker may bridge the amino acid residue S339 (or I339, or T339) with N355 (or T355). In some further embodiments, the bridging linker may bridge the amino acid residue S339 (or I339, or T339) with I357 (or V357, or I357). In some embodiments, the bridging linker may bridge the amino acid residue S339 (or I339, or T339) with V358 (or A358). In some embodiments, the bridging linker may bridge the amino acid residue S339 (or I339, or T339) with T359 (or E359). In some embodiments, the bridging linker may bridge the amino acid residue Q340 (or M340, or M340, or R340) with P356. In some embodiments, the bridging linker may bridge the amino acid residue Q340 (or M340, or M340, or R340) with I351L (or I351). In some embodiments, the bridging linker may bridge the amino acid residue Q340 (or M340, or M340, or R340) with I352. In some embodiments, the bridging linker may bridge the amino acid residue P356 with T353 (or S353). In some embodiments, the bridging linker may bridge the amino acid residue Q340 (or M340, or M340, or R340) with A354 (or V354, or S354). In some alternative embodiments, the bridging linker may bridge the amino acid residue Q340 (or M340, or M340, or R340) with N355 (or T355). In some further embodiments, the bridging linker may bridge the amino acid residue Q340 (or M340, or M340, or R340) with I357 (or V357, or 1357). In some embodiments, the bridging linker may bridge the amino acid residue Q340 (or M340, or M340, or R340) with V358 (or A358). In some embodiments, the bridging linker may bridge the amino acid residue Q340 (or M340, or M340, or R340) with T359 (or E359). Still further, the at least one of said linker/s of the reconstituted epitope of the polypeptide of the present disclosure replaces this loop or any part thereof or amino acid residue/s thereof and any DIII domain fragment or amino acid residue/s thereof. In some embodiments, the reconstituted epitopes of the invention comprise one or more amino acid sequence/s of the native viral envelop protein (E protein), that may be in some embodiments derived from at least one of domains DI, DII and DIII of such envelope protein, any partial sequences or amino acid residue/s thereof, and at least one linker. In some embodiments, the linker/s of the reconstituted epitope may replace at least one amino acid residues of the native E protein (from any one of DIII, DII, DI, domains), that is not directly involved or participate in neutralizing antibodies (nAb/s) binding. More specifically, residues not directly involved in binding or contact, of naturalizing antibodies or alternatively or additionally, of the cognate receptor, include residues that may not serve necessarily as “contact residues”, or “immunogenic residues” but impact nAb/s binding, for example by conferring or maintaining certain conformation required for such binding. These specific residues may be replaced, substituted, excluded or removed in or from the functional reconstituted neutralizing epitope polypeptides of the invention, or alternatively, in or from the entire E protein domain (for example, at least one of domains DI, DII, DIII), in or from the entire envelope protein, and/or in or from the entire virus (e.g., Dengue virus). In yet some further alternative embodiments, residues that may function as “contact residues” for the nAbs, may be replaced by at least one linker/s. In yet some further embodiments, at least one residue involved directly or indirectly in nAb/s binding, may be replaced by said linker/s. Nevertheless, at least one residue not involved in the nAbs interactions, may be replaced by the linker. In certain embodiments, sequences or residues derived from the native E protein or specific domain (e.g., DIII), that are not essential for binding, may be replaced by at least one linker in any of the disclosed polypeptides. In yet some further alternative embodiments, the reconstituted epitope polypeptides of the invention may comprise at least 1, 2, 3, 4, 5, 6, 7, 8, 9, 10, 11, 1, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or more residues that directly or indirectly participate in nAb/s binding. According to some embodiments, these sequences are retained in the reconstituted epitopes and are not replaced by the at least one linker. As noted above, the reconstituted epitope, specifically, reconstituted neutralizing epitope of the present disclosure comprise at least one fragment or amino acid residue or sequence of the native or natural epitope of the envelope protein, and at least one linker. In some embodiments, the linker is a non-native linker, synthetic linker or exogenous linker. In yet some further embodiments, the linker is not a natural part of the native epitope on the envelope protein or of any variants and mutants thereof. Specific embodiments for the reconstituted epitopes, specifically, reconstituted neutralizing epitope provided by the invention are described in more detail herein after. Native protein, e.g., the vital envelope protein as used herein refers to a protein in its properly folded and/or assembled form, which is operative and functional. The native state of a protein may possess all four levels of bio-molecular structure, with the secondary through quaternary structure being formed from weak interactions along the covalently-bonded backbone. In still further embodiments, this term relates to the neutralizing epitope of the natural E protein as appropriately expressed and presented in the natural viral envelop or capsid, and thereby targeted and recognized by neutralizing antibodies. Therefore, in some embodiments, the linker used must differ from the replaced native sequence, that originally and naturally resides within the E protein, in at least one amino acid residue, and specifically, two, three, four, five, six, seven or more residues. In yet some further embodiments, the linker used to replace the native sequences (e.g., the loop or any fragments thereof), differs from the native replaced sequence, such that the reconstituted epitope that comprise the at least one linker cannot be considered as a natural product.

Nevertheless, it should be understood that in some embodiments, the reconstituted epitopes of the invention may comprise at least one or more, specifically, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 45, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, or more neutralizing epitope amino acid residues, specifically, residues that derived from, and are identical to the residues in the native protein, that are directly or indirectly involved in nAb/s binding. In this connection, it should be noted that in certain embodiments, amino acid sequences or amino acid residues that are not directly or indirectly involved in interaction with various neutralizing antibodies, may be also replaced, removed, excluded or substituted by at least one linker. As indicated above, the reconstituted epitopes of the invention comprise at least one linker that replaces in some embodiments, a loop structure of the native domain, specifically, the DIII domain, or any part thereof, or amino acid residue/s thereof. As such, in some embodiments the reconstituted epitope of the invention lacks at least part of the native loop. In further embodiments, the reconstituted epitopes of the invention may comprise more than one linker, for example, 2, 3, 5, 6, 7, 8, 9, 10 or more linkers, that replace at least part of the loop of the DIII domain, or a sequence that comprise at least part of the loop. It should be further appreciated that in some particular embodiments, in addition to linker/s that replace the loop, the reconstituted epitope of the invention may further comprise at least one linker that replace/s at least one amino acid residue/s located in other segments of the native epitope in the E protein. In yet some further embodiments, the reconstituted epitope polypeptide of the invention may comprise at least one linker that replaces at least one amino acid residue of the native epitope, or any fragments thereof not directly involved in nAb/s binding. Alternatively, the linker/s may replace at least one amino acid residue of the epitope directly or indirectly involved and participate in nAb/s binding. Still further, the reconstitute epitope polypeptides of the invention may comprise between about 10 to 100 amino acid residues, specifically, between about 20 to 75 amino acid residues. Specifically, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 45, 46, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 96, 97, 98, 99, 100 or more amino acid residues. In more specific embodiments, the polypeptide of the invention may comprise reconstituted epitope comprising at least one linker that replace/s the loop in the DIII domain or any part thereof, or at least one amino acid residue thereof. In yet further embodiments, the reconstituted epitope of the invention may further comprise additional linker/s that may replace or may be added to further residues of other epitope of DIII domain segments, for example, residues that are located out of the loop. In yet some further particular embodiments, the reconstituted epitope of the polypeptide of the present disclosure comprises at least one linker and at least two fragments of the native E protein. More specifically, these at least two fragments comprise: As one fragment, optionally, a first fragment (a), the amino acid sequence of any one of: (i) residues M301 to I335 of the envelope protein; (ii) residues M301 to I335 of the envelope protein with at least one or two flanking amino acid residue/s; or (iii) any variant, mutant, parts or fragments of the amino acid sequence of residues M301 to I335 of the envelope protein, for example, as defined in (i) and (ii). As another fragment, optionally, a second fragment (b), the amino acid sequence of any one of: (i) residues P356 to E370 of the envelope protein; (ii) residues P356 to E370 of the envelope protein with at least one or two flanking amino acid residue/s; or (iii) any variant, mutant, parts or fragments of the amino acid sequence of residues P356 to E370 of the envelope protein, for example, as defined in (i) and (ii). In yet some further embodiments, the at least one linker of the reconstituted epitope of the polypeptide of the present disclosure may be at least one of: (a), a bridging linker that bridges residue 335 with residue 356 of the of the envelope protein; (b), a linker attached to the N′ terminus of the at least one fragment; and (c), a linker attached to the C′ terminus of said at least one fragment.

In some embodiments, the present disclosure provides any of the reconstituted epitopes, specifically, neutralizing epitopes described herein and in the Examples. The invention further encompasses any polypeptide comprising the reconstituted epitopes, for example, DIII domain polypeptide comprising the reconstituted epitope/s, replacing the corresponding amino acids in the native DIII domain, or in the E protein polypeptide that comprises the reconstituted epitope/s of the present disclosure, replacing the corresponding amino acids of the native E protein. Still further, the present disclosure further encompasses any of the polypeptides of the invention, specifically any one of the More specifically, the DIII domain polypeptide and/or the E protein that comprise at least of the linkers disclosed herein in the examples, and specifically, in any one of Tables 4-7. As indicated herein, each of the polypeptides provide by the present disclosure comprise at least one of the linkers disclosed herein. In some embodiments, the linker replaces fragments of the native DIII domain, for example, the loop structure, and therefore may be located between two fragments of the native DIII sequences. However, it should be appreciated that the polypeptides disclosed herein may comprise more than one linker, specifically, additional linkers that are located in the N′ and/or the C′ termini of the DIII sequences.

In some particular embodiments, the reconstituted epitope of the present disclosure, or any polypeptide thereof (e.g., DIII, E protein), may comprise at least one of the following linkers, TSR (Thr, Ser, Arg), GLRG (Gly, Leu, Ar, Gly), as also denoted by SEQ ID NO: 6, and/or TL (Thr, Leu). In some embodiments, the TSR linker may be an N′ terminal linker. In some embodiments, such linker may replace the corresponding native residues in the DIII, or E protein. In some further embodiments, the GLRG, may be an internal linker. Still further, in some embodiments, such an internal linker may replace the loop sequence (e.g., residues 336-355) of the native DIII domain of the reconstituted epitope of the present disclosure. In yet some further embodiments, the TL linker may be a C′ terminal linker. In some embodiments, this linker replaces the native corresponding residues in the native DIII domain. In some embodiments, the polypeptide of the invention is a reconstitute epitope that comprises the TSR, GLRG (SEQ ID NO: 6) and TL linkers. Such reconstituted epitope comprises residues 301-335 and residues 356-370 of the DNV DIII domain, bridged by the internal linker of SEQ ID NO:6, and flanked by the N′ and C′ linkers TSR and TL, respectively. Such reconstituted epitope comprises according to some embodiments the amino acid sequence:

5′-TSR₃₀₁MCTGSFKLEKEVAETQHGTVLVQVKYEGTDAPCKI₃₃₅GLRG₃₅₆PIVTDKEKPVNIE AE₃₇₀TL-3′, as also denoted by SEQ ID NO: 63. In some embodiments, the reconstitute epitope is also designated clone F8.

In some particular embodiments, the reconstituted epitope of the present disclosure, or any polypeptide thereof (e.g., DIII, E protein), may comprise at least one of the following linkers, G (Gly), PFGSS (Pro, Phe, Gly, Ser, Ser), as also denoted by SEQ ID NO: 7. In some embodiments, the G linker may be an N′ terminal linker. In some embodiments, such linker may replace the corresponding native residues in the DIII, or in the E protein. In some further embodiments, the PFGSS, may be an internal linker. Still further, in some embodiments, such an internal linker may replace the loop sequence (e.g., residues 336-355) of the native DIII domain of the reconstituted epitope of the present disclosure. In some embodiments, the polypeptide of the invention is a reconstitute epitope that comprises the G and PFGSS (SEQ ID NO: 7) linkers.

Such reconstituted epitope comprises residues 301-335 and residues 356-370 of the DNV DIII domain, bridged by the internal linker of SEQ ID NO:7, and flanked by the N′ linker G. Such reconstituted epitope comprises according to some embodiments the amino acid sequence: 5′-G₃₀₁MCTGSFKLEKEVAETQHGTVLVQVKYEGTDAPCKI₃₃₅PFGSS₃₅₆PIVTDKEKPVNIEA E₃₇₀-3′, as also denoted by SEQ ID NO: 64. In some embodiments, the reconstitute epitope is also designated clone A11.

In some particular embodiments, the reconstituted epitope of the present disclosure, or any polypeptide thereof (e.g., DIII, E protein), may comprise at least one of the following linkers, PA (Pro, Ala), GRGG (Gly, Ar, Gly, Gly), as also denoted by SEQ ID NO: 9, and/or LL (Leu, Leu). In some embodiments, the PA linker may be an N′ terminal linker. In some embodiments, such linker may replace the corresponding native residues in the DIII, or in the E protein. In some further embodiments, the GRGG, may be an internal linker. Still further, in some embodiments, such an internal linker may replace the loop sequence (e.g., residues 336-355) of the native DIII domain of the reconstituted epitope of the present disclosure. In yet some further embodiments, the TL linker may be a C′ terminal linker. In some embodiments, this linker replaces the native corresponding residues in the native DIII domain. In some embodiments, the polypeptide of the invention is a reconstitute epitope that comprises the PA, GRGG (SEQ ID NO: 9) and LL linkers. Such reconstituted epitope comprises residues 301-335 and residues 356-370 of the DNV DIII domain, bridged by the internal linker of SEQ ID NO:9, and flanked by the N′ and C′ linkers PA and LL, respectively. Such reconstituted epitope comprises according to some embodiments the amino acid sequence:

5′-PA₃₀₁MCTGSFKLEKEVAETQHGTVLVQVKYEGTDAPCKI₃₃₅GRGG₃₅₆PIVTDKEKPVNIE AE₃₇₀LL-3′, as also denoted by SEQ ID NO: 67. In some embodiments, the reconstitute epitope is also designated clone H1.

In some particular embodiments, the reconstituted epitope of the present disclosure, or any polypeptide thereof (e.g., DIII, E protein), may comprise at least one of the following linkers, TGL (Thr, Gly, Leu), YSGQW (Tyr, Ser, Gly, Gln, Trp), as also denoted by SEQ ID NO: 12, and/or TTQ (Thr, Thr, Gln). In some embodiments, the TGL linker may be an N′ terminal linker. In some embodiments, such linker may replace the corresponding native residues in the DIII, or in the E protein. In some further embodiments, the GRGG sequence may be an internal linker. Still further, in some embodiments, such an internal linker may replace the loop sequence (e.g., residues 336-355) of the native DIII domain of the reconstituted epitope of the present disclosure. In yet some further embodiments, the TTQ linker may be a C′ terminal linker. In some embodiments, this linker replaces the native corresponding residues in the native DIII domain. In some embodiments, the polypeptide of the invention is a reconstitute epitope that comprises the TGL, YSGQW (SEQ ID NO: 12) and TTQ linkers. Such reconstituted epitope comprises residues 301-335 and residues 356-370 of the DNV DIII domain, bridged by the internal linker of SEQ ID NO:12, and flanked by the N′ and C′ linkers TGL and TTQ, respectively. Such reconstituted epitope comprises according to some embodiments the amino acid sequence:

5′-TGL₃₀₁MCTGSFKLEKEVAETQHGTVLVQVKYEGTDAPCKI₃₃₅YSGQW₃₅₆PIVTDKEKPV NIEAE₃₇₀TTQ-3′, as also denoted by SEQ ID NO: 72. In some embodiments, the reconstitute epitope is also designated clone C6.

Additional reconstituted epitopes provided by the present disclosure are presented by Tables 6 and 7.

More specifically, in some embodiments, the reconstituted epitope may comprise the amino acid sequence:

5′-N₃₀₁MCTGSFKLEKEVAETQHGTVLVQVKYEGTDAPCKI₃₃₅SKRG₃₅₆PIVTDKEKPVNIEAE ₃₇₀T-3′, as also denoted by SEQ ID NO: 56. In some embodiments, this reconstituted epitope comprises an internal linker of SEQ ID NO: 2, that replaces the loop of residues 336-355 of the DNV DIII domain, and additional N′ and C′ terminal linkers of N, and T, respectively.

In yet some further embodiments, the reconstituted epitope may comprise the amino acid sequence:

5′-SR₃₀₁MCTGSFKLEKEVAETQHGTVLVQVKYEGTDAPCKI₃₃₅WRLG₃₅₆PIVTDKEKPVNIE AE₃₇₀Y-3′, as also denoted by SEQ ID NO: 57. In some embodiments, this reconstituted epitope comprises an internal linker of SEQ ID NO: 3, that replaces the loop of residues 336-355 of the DNV DIII domain, and additional N′ and C′ terminal linkers of SR, and Y, respectively.

In yet some further embodiments, the reconstituted epitope may comprise the amino acid sequence: 5′-GR₃₀₁MCTGSFKLEKEVAETQHGTVLVQVKYEGTDAPCKI₃₃₅, QTGW₃₅₆PIVTDKEKPVNIE AE₃₇₀L-3′, as also denoted by SEQ ID NO: 58. In some embodiments, this reconstituted epitope comprises an internal linker of SEQ ID NO: 4, that replaces the loop of residues 336-355 of the DNV DIII domain, and additional N′ and C′ terminal linkers of GR, and L, respectively.

In yet some further embodiments, the reconstituted epitope may comprise the amino acid sequence: 5′-K₃₀₁MCTGSFKLEKEVAETQHGTVLVQVKYEGTDAPCKI₃₃₅GGWG₃₅₆PIVTDKEKPVNIEA E₃₇₀-3′, as also denoted by SEQ ID NO: 59. In some embodiments, this reconstituted epitope comprises an internal linker of SEQ ID NO: 5, that replaces the loop of residues 336-355 of the DNV DIII domain, and additional N′ terminal linker K.

In yet some further embodiments, the reconstituted epitope may comprise the amino acid sequence:

5′-P₃₀₁MCTGSFKLEKEVAETQHGTVLVQVKYEGTDAPCKI₃₃₅RRL₃₅₆PIVTDKEKPVNIEAE_{37 0}RS-3′, as also denoted by SEQ ID NO: 60. In some embodiments, this reconstituted epitope comprises an internal linker of RRL, that replaces the loop of residues 336-355 of the DNV DIII domain, and additional N′ and C′ terminal linkers of P, and RS, respectively.

In yet some further embodiments, the reconstituted epitope may comprise the amino acid sequence:

5′-RGA₃₀₁MCTGSFKLEKEVAETQHGTVLVQVKYEGTDAPCKI₃₃₅, NNG₃₅₆PIVTDKEKPVNIE AE₃₇₀YL-3′, as also denoted by SEQ ID NO: 61. In some embodiments, this reconstituted epitope comprises an internal linker of NNG, that replaces the loop of residues 336-355 of the DNV DIII domain, and additional N′ and C′ terminal linkers of RGA, and YL, respectively.

In yet some further embodiments, the reconstituted epitope may comprise the amino acid sequence:

5′-S₃₀₁MCTGSFKLEKEVAETQHGTVLVQVKYEGTDAPCKI₃₃₅GGG₃₅₆PIVTDKEKPVNIEAE_{37 0}RL-3′, as also denoted by SEQ ID NO: 62. In some embodiments, this reconstituted epitope comprises an internal linker of GGG, that replaces the loop of residues 336-355 of the DNV DIII domain, and additional N′ and C′ terminal linkers of S, and RL, respectively.

In yet some further embodiments, the reconstituted epitope may comprise the amino acid sequence: 5′-R₃₀₁MCTGSFKLEKEVAETQHGTVLVQVKYEGTDAPCKI₃₃₅KGG₃₅₆PIVTDKEKPVNIEAE_{3 70}NLA-3′, as also denoted by SEQ ID NO: 65. In some embodiments, this reconstituted epitope comprises an internal linker of KGG, that replaces the loop of residues 336-355 of the DNV DIII domain, and additional N′ and C′ terminal linkers of R, and NLA, respectively.

In yet some further embodiments, the reconstituted epitope may comprise the amino acid sequence:

5′-GFP₃₀₁MCTGSFKLEKEVAETQHGTVLVQVKYEGTDAPCKI₃₃₅GPLGDH₃₅₆PIVTDKEKPV NIEAE₃₇₀RPV-3′, as also denoted by SEQ ID NO: 66. In some embodiments, this reconstituted epitope comprises an internal linker of SEQ ID NO: 8, that replaces the loop of residues 336-355 of the DNV DIII domain, and additional N′ and C′ terminal linkers of GFP, and RPV, respectively. In yet some further embodiments, the reconstituted epitope may comprise the amino acid sequence:
5′-P₃₀₁MCTGSFKLEKEVAETQHGTVLVQVKYEGTDAPCKI₃₃₅AGIDH₃₅₆PIVTDKEKPVNIE AE₃₇₀-3′, as also denoted by SEQ ID NO: 68. In some embodiments, this reconstituted epitope comprises an internal linker of EQ ID NO: 10, that replaces the loop of residues 336-355 of the DNV DIII domain, and additional N′ linker of P.

In yet some further embodiments, the reconstituted epitope may comprise the amino acid sequence: 5′-P₃₀₁MCTGSFKLEKEVAETQHGTVLVQVKYEGTDAPCKI₃₃₅SPKG₃₅₆PIVTDKEKPVNIEAE ₃₇₀-3′, as also denoted by SEQ ID NO: 70. In some embodiments, this reconstituted epitope comprises an internal linker of SEQ ID NO: 11, that replaces the loop of residues 336-355 of the DNV DIII domain, and additional N′ linker of P.

In yet some further embodiments, the reconstituted epitope may comprise the amino acid sequence:

5′-H₃₀₁MCTGSFKLEKEVAETQHGTVLVQVKYEGTDAPCKI₃₃₅QGG₃₅₆PIVTDKEKPVNIEAE_{3 70}TW-3′, as also denoted by SEQ ID NO: 71. In some embodiments, this reconstituted epitope comprises an internal linker of QGG, that replaces the loop of residues 336-355 of the DNV DIII domain, and additional N′ and C′ terminal linkers of H, and TW, respectively.

In yet some further embodiments, the reconstituted epitope may comprise the amino acid sequence:

5′-P₃₀₁MCTGSFKLEKEVAETQHGTVLVQVKYEGTDAPCKI₃₃₅RFG₃₅₆PIVTDKEKPVNIEAE_{37 0}YMR-3′, as also denoted by SEQ ID NO: 73. In some embodiments, this reconstituted epitope comprises an internal linker of RFG, that replaces the loop of residues 336-355 of the DNV DIII domain, and additional N′ and C′ terminal linkers of P, and YMR, respectively.

In yet some further embodiments, the reconstituted epitope may comprise the amino acid sequence:

5′-VP₃₀₁MCTGSFKLEKEVAETQHGTVLVQVKYEGTDAPCKI₃₃₅EWN₃₅₆PIVTDKEKPVNIEA E₃₇₀T-3′, as also denoted by SEQ ID NO: 74. In some embodiments, this reconstituted epitope comprises an internal linker of EWN, that replaces the loop of residues 336-355 of the DNV DIII domain, and additional N′ and C′ terminal linkers of VP, and T, respectively.

In yet some further embodiments, the reconstituted epitope may comprise the amino acid sequence:

5′-R₃₀₁MCTGSFKLEKEVAETQHGTVLVQVKYEGTDAPCKI₃₃₅GDWG₃₅₆PIVTDKEKPVNIEA E₃₇₀NI-3′, as also denoted by SEQ ID NO: 75. In some embodiments, this reconstituted epitope comprises an internal linker of SEQ ID NO; 13, that replaces the loop of residues 336-35 of the DNV DIII domain, and additional N′ and C′ terminal linkers of R, and NI, respectively.

Additional reconstituted epitopes derived from the DIII domain of DNV, are disclosed by Table 5, and include the N′, C′ termini linkers as discussed therein, as well as any one of the internal linkers: EAG, RF, ANLVD (SEQ ID NO: 48), RLNY (SEQ ID NO: 49), TKV, EGLD (SEQ ID NO: 50), GGR, CLVN (SEQ ID NO: 51), ISV (SEQ ID O: 52), V, R, GSGGS (SEQ ID NO: 1), A, ELV, EAG, RF, TKV, RNLY (SEQ ID NO: 55).

Still further, additional reconstituted epitope encompassed by the present disclosure include any of the polypeptides that comprise the amino acid sequence of SEQ ID NO: 29, 30, 31, 32, 33, 34, 35, 36, 37 and 38, and based on sequences derived from the DII domain of the DNV, comprising the linkers as disclosed in Table 4, specifically, the N′ and the C′ terminal linkers of: FR and TSR, ALH and T, S and H, IKR and P, R and ST, HLL and TT, NAP with no C′ terminal linker, G and TRT, SQI with no C′ terminal linker, CAL and HT, respectively. In yet some further embodiments, the present disclosure further provides any polypeptide, specifically, any reconstituted epitope derived from the DIII domain, or any DIII domain or E protein that comprise at least one of any of the disclosed linkers, specifically, at least one of the following internal linkers: SEQ ID NO: 1 SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 49, SEQ ID NO: 50, SEQ ID NO: 51, SEQ ID NO: 52, SEQ ID NO: 55, QGG, RFG, EWN, RRL, NNG, EAG, RF, TKV, GGR, ISV, V, R, A, ELV, EAG, RF, TKV, GGG and KGG, that replace the loop of residues 336-35 of the DNV DIII domain. In some alternative or additional embodiments, the present disclosure provides at least one polypeptide comprising at least one reconstituted epitope of a Zika virus. Thus, in some further embodiments, the present disclosure is particularly applicable for Zika virus. The Zika virus (ZIKV) has become one of the major threats to public health systems worldwide. Like its relatives, ZIKV is transmitted to humans through the bite of infected Aedes mosquitoes. ZIKV can also be transmitted from an infected pregnant woman to her fetus during gestation leading to severe birth defects as congenital microcephaly. Other forms of transmission have also been described, including sexual and blood-borne. ZIKV is a positive single-stranded RNA virus with a 10.7 kb genome translated into a single polyprotein of about 3,000 amino acids. During the viral replication, the polyprotein is cleaved to produce three structural proteins involved in the viral particle assembly, namely the glycoprotein E (protein E), the capsid protein C (protein C), and the protein prM. Whereas seven non-structural proteins are responsible for the viral replication, assembly and evasion from the host defense: NS1, NS2A, NS2B, NS3, NS4A, NS4B and NS5. In some embodiments, the E protein of ZIKV comprises the amino acid sequence as denoted by SEQ ID NO: 100, or any variants and mutants thereof. Several studies have shown that the ZIKV surface shares similar structure and composition to other Flaviviruses like DENV and WNV.

ZIKV protein E dimer structures (PDB ID: 5LBV and 5JHM) show that each protein E is composed by three domains: domain I (DI), domain II (DII) and domain III (DIII). DI is a non-continuous β-shaped domain, which is responsible for linking DII to DIII. It acts on fundamental conformation changes in protein E during Flavivirus infection. DII is a non-continuous finger-like domain. Many DII residues participate on the hydrogen and electrostatic interaction net stabilizing the protein E dimers. The fusion peptide is also located in the DII and has a conserved amino-acid sequence that interacts with host cell endosomal membrane during the virus-host cell membrane fusion process. The C-terminal immunoglobulin-like DIII has high homology to DENV. The interaction between DIII and some glycosaminoglycans is associated with the primary interaction between the viruses and host cells. In yet some further embodiments, the reconstituted epitope of the polypeptide of the present disclosure is of a Zika virus envelope protein. In more specific embodiments, such E protein may comprise an amino acid sequence as denoted by SEQ ID NO: 100, and any variants, mutants and homologs thereof. For example, any variant, homolog, or ortholog that display between about 50%, 55%, 60%, 65%, 70%, 75%, 80%, 85%, 90%, 95%, 97%, 98%, 99%, 99.99% or 100% homology to the Zika virus discussed herein, and/or any serotype/s, variant/s or mutant/s thereof. Still further, in some embodiments, the reconstituted epitope of the polypeptide of the present disclosure may comprise at least in part, at least one amino acid sequence of the DIII domain of the native E protein of said Zika virus, and any fragments thereof. In some embodiments, the DIII domain of the Zika virus E protein comprises residues 302 to 405 of the amino acid sequence as denoted by SEQ ID NO: 100, and any variants, mutants and homologs thereof, as also shown in FIG. 17.

In more specific embodiments, the reconstituted epitope of the polypeptide of the present disclosure may comprise at least one fragment of the native Zika virus E protein comprised within the reconstitute epitope. In more particular embodiments, the reconstituted epitopes may comprise at least one of the following Zika virus E protein sequences: In some embodiments (a), at least one amino acid sequence starting at any one of the amino acid residues 307, 302, 303, 304, 305, 306, 308, 309, 310 or 311, and ending at any one of the amino acid residues 380, 375, 376, 377, 378, 379, 381, 382, 383, 384 or 385. In some embodiments (b), at least one amino acid sequence starting at any one of the amino acid residues 307, 302, 303, 304, 305, 306, 308, 309, 310 or 311, and ending at least one of the amino acid residues 340, 335, 336, 337, 338, 339, 341, 342, 343, 344 or 345. In yet some further embodiments (c), at least one amino acid sequence starting at any one of the amino acid residues 362, 357, 358, 359, 360 or 361 and ending at least one of the amino acid residues 380, 375, 376, 377, 378, 379, 381, 382, 383, 384 or 385. In some embodiments, the reconstituted epitope/s of the disclosed polypeptides may comprise amino acid sequences as defined in (a), in (b), in (c), or in any combinations thereof. In some specific embodiments, the reconstituted epitope may comprise any of the amino acid sequences defined in (a) and at least one linker. In yet some further embodiments, the reconstituted epitope may comprise any of the amino acid sequences defined in (b), any of the sequences as defined in (c) and at least one linker, optionally at least one linker that links between both amino acid sequences. In some embodiments, the reconstitute epitope comprises an amino acid sequence of the native DIII domain of said Zika virus E protein starting at any one of the amino acid residues 307, 302, 303, 304, 305, 306, 308, 309, 310 or 311, and ending at any one of the amino acid residues 380, 375, 376, 377, 378, 379, 381, 382, 383, 384 or 385, wherein said native DIII domain comprises a loop comprising an amino acid sequence starting at any one of the amino acid residues 341, 336, 337, 338, 339, 340, 342, 343, 344, 345 or 346 and ending at any one of the amino acid residues 361, 356, 357, 358, 359, 360, 362, 363, 364, 365 or 366, and wherein at least one of said linker/s replaces said loop or any part thereof or amino acid residue/s thereof and any DIII domain fragment or amino acid residue/s thereof. In some embodiments, the reconstituted epitope comprised within the polypeptide of the present disclosure may comprise at least one linker and at least two fragments of the native E protein. More specifically, these at least two fragments may comprise in some embodiments, as one fragment (a), the amino acid sequence of any one of: (i) residues L307 to K340 of the envelope protein; (ii) residues L307 to K340 of the envelope protein with at least one or two flanking amino acid residue/s; or (iii) any variant, mutant, parts or fragments of the amino acid sequence of residues L307 to K340 of the envelope protein, specifically, as defined for (i) and (ii). The second fragment (b), may comprise the amino acid sequence of any one of: (i) residues N362 to P380 of the envelope protein; (ii) residues N362 to P380 of the envelope protein with at least one or two flanking amino acid residue/s; or (iii) any variant, mutant, parts or fragments of the amino acid sequence of residues N362 to P380 of the envelope protein. In yet some further embodiments, at least one linker of the reconstituted epitope of the polypeptide of the present disclosure may be at least one of: (a), a bridging linker that bridges residue 340 with residue 362 of the of the envelope protein; (b), a linker attached to the N′ terminus of said at least one fragment; and (c), a linker attached to the C′ terminus of said at least one fragment. In some embodiments, the at least one linker may replace residues 341 to 361 of the DIII domain of the E protein of Zika virus.

In yet some further embodiments, the reconstituted epitope of the polypeptide of the present disclosure is of a Yellow Fever virus. Thus, in some further embodiments, the present disclosure is particularly applicable for Yellow Fever virus. Yellow Fever Virus (YFV) is endemic in sub-Saharan Africa and tropical South America. YF disease ranges from asymptomatic to severe jaundice and hemorrhagic fever. As no antiviral therapies exist, the primary disease control strategy is vaccination with the live attenuated vaccine, strain 17D. Despite the availability of a safe and effective vaccine, YFV still causes large, periodic outbreaks. During infection, the E protein binds to cell receptors (that are currently not known for YFV) and the virus is internalized by receptor-mediated endocytosis. The virus is then trafficked through the cytoplasm inside endosomes. The YFV E protein is 493 amino acids in length with the 400 N-terminal amino acids containing the ectodomain (EDI, EDII, and EDIII). EDI and EDII are discontinuous in sequence, while EDIII is continuous. EDI contains a nine-stranded b-barrel and is a linker between EDII and EDIII. It connects to EDII by four flexible linkers and EDIII by one. These linkages are the hinges that allow for conformational change to occur during the replication cycle. EDII, an elongated finger domain, contains the fusion loop that interacts with target cell membranes during attachment and fusion. EDIII is an immunoglobulin-like domain that is thought to be involved in receptor binding. Amino acid substitutions in EDIII are associated with Flavivirus pathogenicity. The C-terminal 100 amino acids contain the stem-anchor region that connects the two transmembrane domains that anchor the E protein in the viral membrane and is necessary for the rearrangement of E on the surface of the virion through its interactions with prM. In some embodiments, the E protein of YFV comprises the amino acid sequence as denoted by SEQ ID NO: 101, or any variants and mutants thereof. Still further in some embodiments, the reconstituted epitope of the polypeptide of the present disclosure if of at least one envelope protein of Yellow fever virus that comprises an amino acid sequence as denoted by SEQ ID NO: 101, and/or any variants, mutants and homologs thereof. For example, any variant, homolog, or ortholog that display between about 50%, 55%, 60%, 65%, 70%, 75%, 80%, 85%, 90%, 95%, 97%, 98%, 99%, 99.99% or 100% homology to the Yellow fever virus discussed herein, and/or any serotype/s, variant/s or mutant/s thereof. Still further, in some embodiments, the reconstituted epitope of the polypeptide of the present disclosure, comprises at least in part, at least one amino acid sequence of the DIII domain of the native E protein of said Yellow Fever virus, and any fragments thereof.

In some embodiments, the DIII domain of the Yellow Fever virus (YFV) E protein used for the reconstituted epitope of the polypeptide of the present disclosure comprises residues 292 to 392 of the amino acid sequence as denoted by SEQ ID NO: 101, and any variants, mutants and homologs thereof. The DIII domain of the E protein of YFV, is also shown in FIG. 18.

In certain embodiments, at least one fragment of the native E protein of the YFV, specifically of the DIII domain of the E protein, is comprised within the reconstituted epitope of the polypeptides of the preset disclosure. In some embodiments, the at least one fragment may comprise at least one of the following options: In some embodiments (a), at least one amino acid sequence starting at any one of the amino acid residues 299, 294, 295, 296, 297, 298, 300, 301, 302, 303 or 304, and ending at any one of the amino acid residues 369, 364, 365, 366, 367, 368, 370, 371, 372, 373 or 374. In some alternative or additional embodiments, at least one fragment may comprise (b), at least one amino acid sequence starting at any one of the amino acid residues 299, 294, 295, 296, 297, 298, 300, 301, 302, 303 or 304, and ending at any one of the amino acid residues 332, 327, 328, 329, 330 or 331. This fragment is also referred to herein as fragment A. Still further alternative or additional embodiments, at least one of the fragments may be (c), at least one amino acid sequence starting at any one of the amino acid residues 354, 349, 350, 351, 352, 353, 355, 356, 357, 358 or 359, and ending at any one of the amino acid residues 369, 364, 365, 366, 367, 368, 370, 371, 372, 373 or 374. In some embodiments, the reconstituted epitope/s of the disclosed polypeptides may comprise amino acid sequences as defined in (a), in (b), in (c), or in any combinations thereof. In some specific embodiments, the reconstituted epitope may comprise any of the amino acid sequences defined in (a) and at least one linker. In yet some further embodiments, the reconstituted epitope may comprise any of the amino acid sequences defined in (b), any of the sequences as defined in (c) and at least one linker, optionally at least one linker that links between both amino acid sequences. In some embodiments, the epitope comprises an amino acid sequence of the native DIII domain of the Yellow Fever virus E protein starting at any one of the amino acid residues 299, 294, 295, 296, 297, 298, 300, 301, 302, 303 or 304, and ending at any one of the amino acid residues 369, 364, 365, 366, 367, 368, 370, 371, 372, 373 or 374. More specifically, the native DIII domain comprises a loop comprising an amino acid sequence starting at any one of the amino acid residues 333, 328, 329, 330, 331, 332, 334, 335, 336, 337, or 338 and ending at any one of the amino acid residues 353, 348, 349, 350, 351, 352, 354, 355, 356, 357, or 358. In some embodiments, at least one of the linker/s of the reconstituted epitope of the polypeptide of the present disclosure replaces this loop or any part thereof, or amino acid residue/s thereof and any DIII domain fragment or amino acid residue/s thereof. In yet some further specific embodiments, the reconstituted epitope of the polypeptide of the present disclosure comprises at least one linker and at least two fragments of the native E protein. More specifically, such at least two fragments comprise: as one fragment, optionally, a first fragment (a), the amino acid sequence of any one of: (i) residues 1299 to I332 of the envelope protein; (ii) residues 1299 to I332 of the envelope protein with at least one or two flanking amino acid residue/s; or (iii) any variant, mutant, parts or fragments of the amino acid sequence of residues 1299 to I332 of the envelope protein. The other fragment, optionally the second fragment (b), may comprise the amino acid sequence of any one of: (i) residues P354 to P369 of the envelope protein; (ii) residues P354 to P369 of the envelope protein with at least one or two flanking amino acid residue/s; or (iii) any variant, mutant, parts or fragments of the amino acid sequence of residues P354 to P369 of the envelope protein. In yet some further embodiments, the at least one linker of the reconstituted epitope of the polypeptide of the present disclosure, may be at least one of: (a), a bridging linker that bridges residue 332 with residue 354 of the of the Yellow Fever virus envelope protein; (b), a linker attached to the N′ terminus of said at least one fragment; and (c), a linker attached to the C′ terminus of said at least one fragment. In some embodiments, the at least one linker may replace residues 333 to 353 of the DIII domain of the E protein of YFV.

In yet some further embodiments, the reconstituted epitope of the polypeptide of the present disclosure is of a West Nile virus (WNV), and/or any serotype/s, variant/s or mutant/s thereof. Thus, in some further embodiments, the present disclosure is particularly applicable for West Nile virus. West Nile virus (WNV), a Flavivirus of the Flaviviridae family, is maintained in nature in an enzootic transmission cycle between avian hosts and ornithophilic mosquito vectors, although the virus occasionally infects other vertebrates. WNV causes sporadic disease outbreaks in horses and humans, which may result in febrile illness, meningitis, encephalitis and flaccid paralysis. West Nile virus (WNV) is a small enveloped virus about 50 nm in diameter. The genomic RNA is enclosed within a nucleocapsid formed by the capsid (C) protein that constitutes the core of the virion and is enveloped by a lipid bilayer derived from the host cell. Mature virions display a smooth outer surface with no projections or spikes. This outer shell is constituted by 180 copies of the small membrane (M) protein and an equal number of copies of the E glycoprotein disposed as 90 anti-parallel homodimers arranged in three distinct symmetry environments, thus resulting in a particle of icosahedral symmetry. The WNV genome is constituted by a single-stranded RNA molecule of positive polarity of about 11 000 nucleotides in length encodes a polyprotein. The polyprotein is proteolytically processed by viral and cellular proteases rendering ten major viral proteins: three structural (C, prM and E) and seven non-structural, NS (NS1, NS2A, NS2B, NS3, NS4A, NS4B, and NS5). The envelope (E) is a transmembrane protein anchored to the lipid envelope by a C-terminal α-helical hairpin. It is the most immunogenic protein of the virus and the target for most neutralizing antibodies. The protein is glycosylated on position 154 on most WNV strains. Glycosylation is important for efficient transmission in mosquito and birds and may be related to neuroinvasiveness. The structure of the E glycoprotein presents the typical folding of the Flavivirus E glycoproteins and is organized in three domains: DI, DII, that contains a hydrophobic peptide responsible for virus fusion termed fusion loop, and DIII, an immunoglobulin-like domain. DII mediates the homodimerization of the protein on the surface of the virion. DIII is involved in receptor binding and contains multiple epitopes that are recognized by neutralizing antibodies. Upon acid exposure, the E glycoprotein undergoes conformational rearrangements and changes from dimers to trimers, exposing the fusion loop to enable viral fusion of the virion with cellular endosomal target membranes.

Still further, in some embodiments, the envelope protein of the WNV comprises an amino acid sequence as denoted by SEQ ID NO:102, and any mutants, variants and homologs thereof. The DIII domain of the E protein of WNV, is also shown in FIG. 19.

In some embodiments, the reconstituted epitope of the polypeptide of the present disclosure may comprise at least in part, at least one amino acid sequence of the DIII domain of the native E protein of said West Nile virus, and any fragments thereof.

In some embodiments, the DIII domain comprises residues 297 to 400, of the native E protein of West Nile virus, of the amino acid sequence as denoted by SEQ ID NO: 102, and any variants, mutants and homologs thereof. For example, any variant, homolog, or ortholog that display between about 50%, 55%, 60%, 65%, 70%, 75%, 80%, 85%, 90%, 95%, 97%, 98%, 99%, 99.99% or 100% homology to the West Nile virus discussed herein.

In certain embodiments, at least one fragment of the native E protein of the WNV, specifically of the DIII domain of the E protein, is comprised within the reconstituted epitope of the polypeptides of the preset disclosure. In more specific embodiments, at least one of these fragments may be at least one of the following fragments or any combinations thereof. In some embodiments, such fragment may be (a), at least one amino acid sequence starting at any one of residues 304, 299, 300, 301, 302, 303, 305, 306, 307, 308 or 309 and ending at any one of the amino acid residues 377, 372, 373, 374, 375, 376, 378, 379, 380, 381 or 382. Still further, in some alternative or additional embodiments, at least one of the fragments may be (b), at least one amino acid sequence starting at any one of residues 304, 299, 300, 301, 302, 303, 305, 306, 307, 308 or 309 and ending at any one of the amino acid residues 338, 333, 334, 335, 336, 337, 339, 340, 341, 342, or 343. In some embodiments, this fragment is also referred to herein as fragment A. Still further, the at least one fragment may be (c), at least one amino acid sequence starting at any one of residues 360, 35, 356, 357, 358, 359, 361, 362, 363, 364 or 365 and ending at any one of the amino acid residues 377, 372, 373, 374, 375, 376, 378, 379, 380, 381 or 382. In some embodiments, the reconstituted epitope/s of the disclosed polypeptides may comprise amino acid sequences as defined in (a), in (b), in (c), or in any combinations thereof. In some specific embodiments, the reconstituted epitope may comprise any of the amino acid sequences defined in (a) and at least one linker. In yet some further embodiments, the reconstituted epitope may comprise any of the amino acid sequences defined in (b), any of the sequences as defined in (c) and at least one linker, optionally at least one linker that links between both amino acid sequences. In some embodiments, the epitope comprises an amino acid sequence of the native DIII domain of said West Nile virus E protein starting at any one of residues 304, 299, 300, 301, 302, 303, 305, 306, 307, 308 or 309 and ending at any one of the amino acid residues 377, 372, 373, 374, 375, 376, 378, 379, 380, 381 or 382. In some embodiments, the native DIII domain comprises a loop comprising an amino acid sequence starting at any one of the amino acid residues 339, 334, 335, 336, 337, 338, 340, 341, 342, 343, or 344 and ending at any one of the amino acid residues 359, 354, 355, 356, 357, 358, 360, 361, 362, 363 or 364. Still further, at least one of the linker/s of the reconstituted epitope of the polypeptide of the present disclosure, replaces the loop or any part thereof or amino acid residue/s thereof and any DIII domain fragment or amino acid residue/s thereof. Thus, in some specific embodiments, the reconstituted epitope of the polypeptide provided by the present disclosure may comprise at least one linker and at least two fragments of the native E protein. In more specific embodiments, the at least two fragments comprise: as one fragment, optionally, a first fragment (a), the amino acid sequence of any one of: (i) residues V304 to V338 of the envelope protein; (ii) residues V304 to V338 of the envelope protein with at least one or two flanking amino acid residue/s; or (iii) any variant, mutant, parts or fragments of the amino acid sequence of residues V304 to V338 of the envelope protein. The reconstituted epitope comprises as another fragment, optionally, as a second fragment (b), the amino acid sequence of any one of: (i) residues P360 to P377 of the envelope protein; (ii) residues P360 to P377 of the envelope protein with at least one or two flanking amino acid residue/s; or (iii) any variant, mutant, parts or fragments of the amino acid sequence of residues P360 to P377 of the amino acid sequence as denoted by SEQ ID NO: 102 of the envelope protein, and any variants, mutants and homologs thereof. In yet some further embodiments, the at least one linker of the reconstituted epitope of the polypeptide of the present disclosure is at least one of: (a), a bridging linker that bridges residue 338 with residue 360 of the of the envelope protein; (b), a linker attached to the N′ terminus of said at least one fragment; and (c), a linker attached to the C′ terminus of said at least one fragment. In some embodiments, the at least one linker may replace residues 338 to 360 of the DIII domain of the E protein of WNV.

Still further, in some embodiments the present disclosure is particularly applicable for Tick-borne encephalitis virus. Tick-borne encephalitis (TBE) virus is a member of the genus Flavivirus (family Flaviviridae). This small isometric virus is composed of a structurally ill-defined nucleocapsid containing the positive-stranded RNA genome and a lipid envelope carrying 180 copies of glycoprotein E envelope and the small membrane-associated protein M. The structure of E revealed a specific icosahedral arrangement of E at the viral surface (26-30). The M protein is located beneath the E protein dimer. The external part of E (sE), lacking the hydrophobic C-terminal double membrane-spanning anchor and the membrane-proximal region (called the stem), is composed of three distinct structural domains (DI, DII, and DIII). Because of its essential function in receptor binding and entry, E is the major target of virus neutralizing antibodies, and their induction correlates with protection against flavivirus-induced disease, including TBE. Studies using monoclonal antibodies (MAbs) demonstrated that binding to each of the three domains of E can lead to virus neutralization, and highly potent antibodies were shown to be directed at a surface-exposed epitope within DIII, the so-called DIII lateral ridge (DIII-lr) epitope. As indicated herein, the polypeptide of the present disclosure, and specifically, the reconstituted epitopes disclosed herein, comprise at least one linker. The term “linker” in the context of the invention concerns an amino acid sequence of from about 1 to about 10 or more amino acid residues positioned within and/or flanking the reconstituted epitope of the invention. The linker may be positioned in the central region of the reconstituted epitope of the invention and/or in at least one of its termini, namely at the C-terminus and/or at the N-terminus thereof. The linker is covalently linked or joined to the amino acid residues in its vicinity. For example, a linker in accordance with the invention may be of 1, 2, 3, 4, 5, 6, 7, 8, 9 or 10 or more amino acid residues long. In yet some further embodiments, the linker/s used by the invention may be a combinatorial linker screened from a combinatorial library comprising some or all possible linkers composed of 1, 2, 3, 4, 5, 6, 7, 8, 9, 10 or more amino acid residues that are tested and screened for functionality, i.e., to produce a functional epitope that is able to functionally be bound by a receptor and/or neutralizing antibody. The term linker in accordance with the present invention encompasses any amino acid residue, as dictated by the encoding NNK nucleic acid motif. In some embodiments the linker according to the present invention encompasses 1-10, 1-9, 1-8, 1-7, 1-6, 1-5, 1-4, 1-3 or 1-2 or 1 amino acid residue/s. In other embodiments the linker encompasses 1, 2, 3, 4, 5, 6, 7, 8, 9, 10 or more amino acid residues, and thus, in certain embodiments the linkers may be referred to as NNK₁, NNK₂, NNK₃, NNK₄, NNK₅, NNK₆ NNK₇, NNK₈, NNK₉ and NNK₁₀. In some specific embodiments, useful linkers may include NNK₁, NNK₂, NNK₃, NNK₄, NNK₅, NNK₆, and NNK₇. As known in the art, the term “NNK” refers to a nucleic acid triad encoding an amino acid residue, where “N” denotes any nucleotide (namely a natural or a non-natural nucleotide, e.g. nucleotides based on the DNA nucleobases cytosine (C), guanine (G), adenine (A) and thymine (T)) and where “K” denotes a nucleotide based on guanine (G) or thymine (T). However, the NNN codon is also possible. The original use of NNK is to reduce the possibility of abortive termination. The UAG codon which is possible for NNK is overcome when expressing the library in a bacterial strain that contains a suppression mutation reading UAG for the incorporation of a glutamine residue (such as the SupE mutation). As detailed below, the linkers as herein defined are based on nucleotide triads of the type “NNK”. The linker, when present, may have a length of n repeats which may be the same or different one from the other. In particular the linker may include one NNK (denoted as “NNK₁”), two NNK (denoted as “NNK₂”), “NNK₃”, “NNK₄” when three or four NNKs are present, respectively, etc. Specifically, the index n may have a value of between 0 to 10. In some embodiments, the index n may have a value of between 3 to 7.

As noted above, the reconstituted epitope polypeptide of the invention comprises at least one linker. It should be appreciated that any linkers or any combination of linkers may be used for the polypeptide of the invention. In certain and non-limiting embodiments, an amino acid linker may be used. In some specific embodiments, at least one of the at least one linker of the reconstituted epitope of the polypeptide of the present disclosure may be an amino acid linker. In certain embodiments, the linker may comprise 1 to 10 amino acid residues. Specific embodiments for linkers found to be useful in the reconstituted epitopes disclosed herein may be any one of the linkers of SEQ ID NO: 1 SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 49, SEQ ID NO: 50, SEQ ID NO: 51, SEQ ID NO: 52, SEQ ID NO: 55, QGG, RFG, EWN, RRL, NNG, EAG, RF, TKV, GGR, ISV, V, R, A, ELV, EAG, RF, TKV, GGG and KGG, or any derivatives thereof.

In some embodiments, the at least one polypeptide provided by the preset disclosure, may be at least one DIII domain of a native E protein of a virus of the Flaviviridae family, or any fragment of such domain. According to such embodiments, the DIII that serves as the polypeptide of the present disclosure comprises any of the reconstituted epitopes disclosed herein. In yet some further embodiments, the reconstituted epitope disclosed by the invention replaces the corresponding amino acid residues within the DIII domain. Still further, the reconstituted epitopes of the DIII domain polypeptide disclosed herein comprise at least one amino acid sequence derived from the DIII domain and at least one linker. In more specific embodiments, at least one of the linkers is a linker that replaces the loop of the DIII domain. In some particular embodiments, the DIII domain of the preset disclosure is derived from Dengue virus. Still further, in some embodiments, the DIII polypeptide may comprise any of the reconstituted epitopes disclosed herein. Non limiting embodiments for such reconstituted epitopes are any one of the epitopes comprising the amino acid sequence as denoted by any one of: SEQ ID NO: 63, 64, 67, 72, 56-62, 65, 66, 68, 70, 71 and 73 to 75. In some specific embodiments, the polypeptide of the present disclosure comprises a DIII domain of a Dengue virus, that comprises the reconstituted epitope of SEQ ID NO: 67 (clone H1). In yet some further embodiments, the polypeptide of the present disclosure comprises a DIII domain of a Dengue virus, that comprises the reconstituted epitope of SEQ ID NO: 63 (clone F8). Still further, in some embodiments, the polypeptide of the present disclosure comprises a DIII domain of a Dengue virus, that comprises the reconstituted epitope of SEQ ID NO: 64 (clone A11). Still further, in some embodiments, the polypeptide of the present disclosure comprises a DIII domain of a Dengue virus, that comprises the reconstituted epitope of SEQ ID NO: 72 (clone C6). Still further, it should be appreciated that according to some embodiments, the invention further encompasses as the polypeptide, at least one DII domain that comprise DII domain derived reconstituted epitopes, for example, any one of the epitopes of SEQ ID NOs: 29 to 38. In yet some further embodiments, the at least one polypeptide provided by the present disclosure may be at least one envelope protein (E protein) of a virus of the Flaviviridae family. Thus, according to some embodiments, the disclosed polypeptide may be the E protein of any Flavivirus, specifically, any one of Dengue virus, Zika virus, WNV or YFV, that comprise any of the reconstituted epitopes disclosed by the present invention. Still further, in some embodiments, the present disclosure provides as the disclosed polypeptide, an E protein of Dengue virus, that comprises any of the reconstituted epitopes of the present disclosure. Non limiting embodiments for such reconstituted epitopes are any one of the epitopes comprising the amino acid sequence as denoted by any one of: SEQ ID NO: 63, 64, 67, 72, 56-62, 65, 66, 68, 70, 71 and 73 to 75. In some specific embodiments, the polypeptide of the present disclosure comprises an E protein of a Dengue virus, that comprises the reconstituted epitope of SEQ ID NO: 67 (clone H1). In yet some further embodiments, the polypeptide of the present disclosure comprises an E protein of a Dengue virus, that comprises the reconstituted epitope of SEQ ID NO: 63 (clone F8). Still further, in some embodiments, the polypeptide of the present disclosure comprises an E protein of a Dengue virus, that comprises the reconstituted epitope of SEQ ID NO: 64 (clone A11). Still further, in some embodiments, the polypeptide of the present disclosure comprises an E protein of a Dengue virus, that comprises the reconstituted epitope of SEQ ID NO: 72 (clone C6).

Thus, the disclosed polypeptides may be according to some embodiments, either the reconstituted epitopes as disclosed herein, or DIII domain of an E protein, or an E protein of a Flavivirus, specifically, any one of Dengue virus, Zika virus, WNV or YFV, hat comprise the reconstituted epitopes disclosed herein that replace the corresponding amino acid sequences in said DIII domain or E protein. It should be however noted that the invention further encompasses any of these proteins, either the entire E protein, or the DIII thereof, that comprise the specific at least one linker that replaces the corresponding sequences in the DIII domain or E protein.

Thus, in yet another aspect thereof, the present disclosure provides a DIII domain of an E protein of a virus of the Flaviviridae family, comprising the native DIII domain of an E protein of a virus of the Flaviviridae family or any fragments thereof and at least one linker. It should be noted that in some embodiments, the linker comprises amino acid sequence that differ from the original native sequence, such that the resulting DIII domain provided herein, structurally differs from the natural DIII domain, and as such, cannot be considered as a product of nature. Therefore, in some embodiments, the DIII domain provided herein may be also considered as an engineered or modified DIII domain. More specifically, at least one of such linker/s replaces a loop in the DIII domain, or any part thereof or amino acid residue/s thereof. In some specific embodiments of the disclosed DIII domain (a), the virus of the Flaviviridae family is a Dengue virus. Thus, according to such embodiments, the disclosed DIII domain is not the native Dengue DIII domain. In such case, the loop may comprise an amino acid sequence that starts at any one of the amino acid residues 336, 333, 334, 335, 337, 338 or 339, and end at any one of the amino acid residues 355, 352, 353, 354, 356, 357 or 358. In some embodiments, the loop comprises residues 336 to 355, and is replaced by at least one linker as disclosed in the present disclosure. In some particular and non-limiting embodiments, suitable linkers may include, but are not limited to any of the linkers disclosed by the present disclosure, specifically, any of the linkers disclosed in any one of Tables 4, 5, 6 and 7. In more specific embodiments, suitable linkers that may replace the loop of the disclosed DIII domain may be any one of the linkers that comprise an amino acid sequence as denoted by any one of SEQ ID NO: 1 SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 49, SEQ ID NO: 50, SEQ ID NO: 51, SEQ ID NO: 52, SEQ ID NO: 55, QGG, RFG, EWN, RRL, NNG, EAG, RF, TKV, GGR, ISV, V, R, A, ELV, EAG, RF, TKV, GGG and KGG, or any variants or derivatives thereof. It should be noted that the disclosed linkers are internal linkers that may be used to replace the loop of the DIII or any parts thereof, however, these linkers may be used as N′ and/or C′ terminal linkers, or any of the N′ and/or C′ terminal linkers disclosed in Tables 4-7 may be used for the DIII domain disclosed herein. In yet some further additional or alternative embodiments (b), the virus of the Flaviviridae family is a Zika virus. According to such embodiments, the loop replaced by the at least one linker, may comprise an amino acid sequence starting at any one of the amino acid residues 341, 336, 337, 338, 339, 340, 342, 343, 344, 345 or 346 and ending at any one of the amino acid residues 361, 356, 357, 358, 359, 360, 362, 363, 364, 365 or 366. Thus, the invention provides DIII domain of Zika virus that comprise at least one linker. Specifically, at least one inker that replaces the loop of the DIII domain of an E protein of the Zika virus, as discussed herein. In yet some further alternative or additional embodiments (c), the virus of the Flaviviridae family, may be a Yellow Fever virus. In such case, the domain III provided by the present disclosure may comprise a loop comprising an amino acid sequence starting at any one of the amino acid residues 333, 328, 329, 330, 331, 332, 334, 335, 336, 337, or 338 and ending at any one of the amino acid residues 353, 348, 349, 350, 351, 352, 354, 355, 356, 357, or 358. Thus, the invention provides DIII domain of YFV that comprise at least one linker. Specifically, at least one inker that replaces the loop of the DIII domain of an E protein of the YFV, as discussed herein. In yet some further alternative or additional embodiments (d), the virus of the Flaviviridae family is a West Nile virus. In such case, the loop replaced by the linkers provided by the present disclosure may comprise an amino acid sequence starting at any one of the amino acid residues 339, 334, 335, 336, 337, 338, 340, 341, 342, 343, or 344 and ending at any one of the amino acid residues 359, 354, 355, 356, 357, 358, 360, 361, 362, 363 or 364. Thus, the invention provides DIII domain of WNV that comprise at least one linker. Specifically, at least one inker that replaces the loop of the DIII domain of an E protein of the WNV, as discussed herein. In some embodiments, the at least one of linker used herein is a bridging linker. In yet some optional embodiments, the DIII domain provided by the present aspect may further comprises at least one linker flanking the N′ and/or C′ termini thereof. A further aspect of the present disclosure relates to an envelope protein (E protein) of a virus of the Flaviviridae family, comprising the native E protein of a virus of the Flaviviridae family or any fragments thereof and at least one linker. It should be noted that in some embodiments, the linker comprises amino acid sequence that differ from the original native sequence, such that the resulting E protein provided herein, structurally differs from the natural E protein, and as such, cannot be considered as a product of nature. Therefore, in some embodiments, the E protein provided herein may be also considered as an engineered or modified E protein. In some embodiments, at least one of the linker/s replaces a loop in the DIII domain of the envelope protein, or any part thereof or amino acid residue/s thereof. Still further, the envelope protein of the present disclosure may be further characterized by at least one of the following features: In some embodiments (a), the virus of the Flaviviridae family may be a Dengue virus. Thus, the present disclosure provides a modified E protein of a Dengue virus, that differs structurally from the native E protein. It should be noted that the naïve E protein of the Dengue virus comprises an amino acid sequence as denoted by any one of SEQ ID NO; 96, 97, 98, 99 (of serotypes 1, 2, 3, and 4, respectively). According to such embodiments, the loop of the envelope protein disclosed herein may comprise an amino acid sequence starting at any one of the amino acid residues 336, 333, 334, 335, 337, 338 or 339, and ending at any one of the amino acid residues 355, 352, 353, 354, 356, 357 or 358. Thus, in some embodiments, the modified Dengue virus E protein may comprise a sequence derived from any one of SEQ ID NO: 96, 97, 98, 99, with at least one additional linker that in some embodiments, is an internal linker that replaces the discussed loop. In some embodiments, the loop comprises residues 336 to 355, and is replaced by at least one linker as disclosed in the present disclosure. In some particular and non-limiting embodiments, suitable linkers may include, but are not limited to any of the linkers disclosed by the present disclosure, specifically, any of the linkers disclosed in any one of Tables 4, 5, 6 and 7. In more specific embodiments, suitable linkers that may replace the loop of the disclosed E protein (specifically, in the DIII domain thereof), may be any one of the linkers that comprise an amino acid sequence as denoted by any one of SEQ ID NO: 1 SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 49, SEQ ID NO: 50, SEQ ID NO: 51, SEQ ID NO: 52, SEQ ID NO: 55, QGG, RFG, EWN, RRL, NNG, EAG, RF, TKV, GGR, ISV, V, R, A, ELV, EAG, RF, TKV, GGG and KGG, or any variants or derivatives thereof. It should be noted that the disclosed linkers are internal linkers that may be used to replace the loop of the DIII domain of the disclosed E protein, or any parts thereof, however, these linkers may be alternatively, or additionally used as N′ and/or C′ terminal linkers, or any of the N′ and/or C′ terminal linkers disclosed in Tables 4-7 may be used for the DIII domain of the E protein disclosed herein.

In yet some further alternative or additional embodiments (b), the virus of the Flaviviridae family may be a Zika virus. Thus, the present disclosure provides a modified E protein of a Zika virus, that differs structurally from the native E protein. It should be noted that the naïve E protein of the Zika virus comprises an amino acid sequence as denoted by SEQ ID NO: 100. In such case, the loop may comprise an amino acid sequence starting at any one of the amino acid residues 341, 336, 337, 338, 339, 340, 342, 343, 344, 345 or 346 and ending at any one of the amino acid residues 361, 356, 357, 358, 359, 360, 362, 363, 364, 365 or 366. Thus, in some embodiments, the modified Zika virus E protein may comprise a sequence derived from SEQ ID NO: 100, with at least one additional linker that in some embodiments, is an internal linker that replaces the discussed loop. Still further, in some additional or alternative embodiments (c), the virus of the Flaviviridae family is a Yellow Fever virus. Thus, the present disclosure provides a modified E protein of a Yellow Fever virus, that differs structurally from the native E protein. It should be noted that the naïve E protein of the Yellow Fever virus comprises an amino acid sequence as denoted by SEQ ID NO: 101. In such case, the loop of the envelope protein of the present disclosure may comprise an amino acid sequence starting at any one of the amino acid residues 333, 328, 329, 330, 331, 332, 334, 335, 336, 337, or 338 and ending at any one of the amino acid residues 353, 348, 349, 350, 351, 352, 354, 355, 356, 357, or 358. Thus, in some embodiments, the modified Yellow Fever virus E protein may comprise a sequence derived from SEQ ID NO: 101, with at least one additional linker that in some embodiments, is an internal linker that replaces the discussed loop. In some further embodiments, (d), the virus of the Flaviviridae family is a West Nile virus. Thus, the present disclosure provides a modified E protein of a West Nile virus, that differs structurally from the native E protein. It should be noted that the naïve E protein of the Zika virus comprises an amino acid sequence as denoted by SEQ ID NO: 102. Accordingly, the loop of the envelope protein of the present disclosure may comprise an amino acid sequence starting at any one of the amino acid residues 339, 334, 335, 336, 337, 338, 340, 341, 342, 343, or 344 and ending at any one of the amino acid residues 359, 354, 355, 356, 357, 358, 360, 361, 362, 363 or 364. Thus, in some embodiments, the modified West Nile virus E protein may comprise a sequence derived from SEQ ID NO: 102, with at least one additional linker that in some embodiments, is an internal linker that replaces the discussed loop. In et some further embodiments, at least one of the linker/s of the envelope protein of the present disclosure is a bridging linker. In yet some further optional embodiments, any of the E protein/s of the present disclosure may further comprise at least one linker flanking the N′ and/or C′ termini of the DIII domain or the disclosed E protein. It should be noted that the present disclosure further encompasses any multimeric or multivalent displaying platform, and/or a composition and/or any vaccine that comprise any of the DIII domains, and/or E proteins of the present disclosure.

The invention further encompasses any attenuated or killed Flavivirus, specifically, any Dengue, Zika virus, Yellow fever and West Nile fever viruses that comprise any of the linkers disclosed herein, specifically, linkers that replace at least one loop in the E protein domains DIII, DII, DI as defined herein, or any variant or mutant thereof, and any mixture, combination, composition or vaccine thereof. It should be appreciated that the present disclosure further provides any fusion proteins and/or conjugates that comprise any of the polypeptides of the present disclosure, specifically, of any of the reconstituted epitopes disclosed herein, and/or any of the DIII domain polypeptides and/or of an of the E proteins disclosed herein. Still further, in some embodiments, the present disclosure provides fusion proteins of any of the disclosed polypeptides, for example, with any one of GST, MBP, 153-A50. In some specific embodiments, the present disclosure provides fusion proteins of any of the reconstituted epitopes of any one of SEQ ID NO: 63, 64, 67, 72, 56-62, 65, 66, 68, 70, 71, 73-75. Still further, in some embodiments, the present disclosure provides a fusion protein of the reconstituted epitope of SEQ ID NO: 67 (clone H1), with GST. In some embodiments, such fusion protein may comprise the amino acid sequence as denoted by SEQ ID NO: 139. Still further, in some embodiments, the present disclosure provides a fusion protein of the reconstituted epitope of SEQ ID NO: 63 (clone F8), with GST. In some embodiments, such fusion protein may comprise the amino acid sequence as denoted by SEQ ID NO: 138. In yet some further embodiments, the present disclosure provides a fusion protein of the reconstituted epitope of SEQ ID NO: 64 (clone A11), with GST. In some embodiments, such fusion protein may comprise the amino acid sequence as denoted by SEQ ID NO: 136. In some further embodiments, the present disclosure provides a fusion protein of the reconstituted epitope of SEQ ID NO: 72 (clone C6), with GST. In some embodiments, such fusion protein may comprise the amino acid sequence as denoted by SEQ ID NO: 137. Still further, in some embodiments, the present disclosure provides a fusion protein of the reconstituted epitope of SEQ ID NO: 67 (clone H1), with MBP. In some embodiments, such fusion protein may comprise the amino acid sequence as denoted by SEQ ID NO: 135. Still further, in some embodiments, the present disclosure provides a fusion protein of the reconstituted epitope of SEQ ID NO: 63 (clone F8), with MBP. In some embodiments, such fusion protein may comprise the amino acid sequence as denoted by SEQ ID NO: 134. In yet some further embodiments, the present disclosure provides a fusion protein of the reconstituted epitope of SEQ ID NO: 64 (clone A11), with MBP. In some embodiments, such fusion protein may comprise the amino acid sequence as denoted by SEQ ID NO: 132. In some further embodiments, the present disclosure provides a fusion protein of the reconstituted epitope of SEQ ID NO: 72 (clone C6), with MBP. In some embodiments, such fusion protein may comprise the amino acid sequence as denoted by SEQ ID NO: 133.

Still further, in some embodiments, the present disclosure provides a fusion protein of the reconstituted epitope of SEQ ID NO: 67 (clone H1), with 153-A50. In some embodiments, such fusion protein may comprise the amino acid sequence as denoted by SEQ ID NO: 142. Still further, in some embodiments, the present disclosure provides a fusion protein of the reconstituted epitope of SEQ ID NO: 63 (clone F8), with 153-A50. In some embodiments, such fusion protein may comprise the amino acid sequence as denoted by SEQ ID NO: 141. In yet some further embodiments, the present disclosure provides a fusion protein of the reconstituted epitope of SEQ ID NO: 64 (clone A11), with 153-A50. In some embodiments, such fusion protein may comprise the amino acid sequence as denoted by SEQ ID NO: 140. In some further embodiments, the present disclosure provides a fusion protein of the reconstituted epitope of SEQ ID NO: 72 (clone C6), with 153-A50. A further aspect of the present disclosure relates to a multimeric and/or multivalent antigen displaying platform, and/or nanoparticle scaffold, comprising at least one reconstituted epitope of a viral envelope protein, any polypeptide, domain or viral envelope protein comprising the reconstituted epitope, any domain or viral envelope protein comprising at least one linker that replaces at least one loop or any part thereof or amino acid residue/s thereof in the viral protein. More specifically, in some embodiments, the viral envelope protein, specifically of any of the Flaviviridae, specifically, any of the Flavivirus genus, specifically, the native E protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII. It should be further noted that the reconstituted epitope of the multimeric and/or multivalent antigen displaying platform disclosed herein may comprise at least one linker and at least one fragment of the native envelope protein as disclosed herein. In certain embodiments, the reconstituted epitope comprised within the multimeric and/or multivalent antigen displaying platform of the present disclosure is any of the polypeptide/s as defined by the present disclosure herein before. Still further, the domain or viral envelope protein comprising at least one linker of the multimeric and/or multivalent antigen displaying platform is as defined by the present invention in connection with previous aspects. In some embodiments, the multimeric and/or multivalent antigen displaying platform comprises a self-assembling nanostructure.

In yet some further embodiments, the reconstituted epitope of the invention may be presented as a multimeric/multivalent antigen, using the bacteriophage Protein 3 scaffold as shown by the examples, or alternatively, the bacteriophage Protein 8 scaffold, or Proteins 7 or 9 scaffold. In some embodiments, the reconstituted epitope of the invention is selected from a conformer library displayed on a bacteriophage. In some specific embodiments, such conformer library is displayed on filamentous bacteriophages based on the fth1 phage vector previously described by the inventor (Enshell D et al., Nucleic Acids Res. 2001 May 15; 29 (10): e50). The combinatorial linker library is expressed on Protein 3 which exists in five copies. Thus, in accordance with some embodiments of the invention, the reconstituted epitope of the invention, is expressed and displayed on Protein 3 scaffold. In some embodiments, the reconstituted epitope of the invention displayed on Protein 3 scaffold, may be referred to herein as a multimeric version of the reconstituted epitope, containing a pentamer of the reconstituted epitope on five copies of the P3 protein. Similarly, any of the domain DIIIs and/or E proteins of the invention that comprise at least one exogeneous linker may be displayed using the P3 protein scaffold. In yet, some further embodiments, the reconstituted epitope of the invention is expressed and displayed on Protein 8 scaffold. In some embodiments, the reconstituted epitope of the invention displayed on Protein 8 scaffold, may be referred to herein as a polyvalent version of the reconstituted epitope, as the number or recombinant Protein 8 molecules in any chimeric phage could be greater than 10 and possibly hundreds of copies per phage. Still further, in some embodiments, any of the reconstituted epitopes, DIII domains or E proteins disclosed herein in connection with other aspects of the present disclosure, may be presented by any of the nanoparticle scaffolds disclosed herein. As will be discussed in more detail in connection with other aspects of the invention, should be appreciated that any of the reconstituted epitopes, domain DIII and/or E proteins and any multimeric/multivalent antigen displaying platform thereof, may be connected directly or indirectly to at least one tag, detectable moiety, affinity moiety or solid support. It should be understood however, that any display vehicle can be used in the multimeric/multivalent antigen displaying platform of the invention, for example, bacteriophage (e.g., M13, fd, fl, T4 and T7), yeast, ribosome, peptide, or any other display systems, or any combinations thereof. Still further, when bacteriophage display systems are used, any phage protein can be used as a scaffold. In yet some further embodiments, the reconstituted epitope polypeptides or any DIII, DII, DI domains and E proteins of the invention, or any variants thereof or any derivative, enantiomer, fusion protein or conjugate thereof, may be presented in the vaccines of the invention as a polyvalent antigen by incorporation thereof in a polyvalent dendrimer. This embodiment is based on the knowledge in the art that a multiple antigen peptide carrying a multiplicity of epitopes induces superior immune responses compared to responses following immunization with corresponding equal amounts of monovalent epitopes. Thus, in some embodiments, the present invention is intended to broadly encompass antigenic products carrying multiple copies of the reconstituted epitopes polypeptides of the present invention an in a multiple antigen peptide system. The present dendritic polymers are antigenic products in which the reconstituted epitope or any polypeptides of the present disclosure (DIII, E protein), are covalently bound to the branches that radiate from a core molecule. These dendritic polymers are characterized by higher concentrations of functional groups per unit of molecular volume than ordinary polymers. Generally, they are based upon two or more identical branches originating from a core molecule having at least two functional groups. The polymers are often referred to as dendritic polymers because their structure may be symbolized as a tree with a core trunk and several branches. Unlike a tree, however, the branches in dendritic polymers are substantially identical. The dendrite system has been termed the “multiple antigen peptide system” (MAPS), which is the commonly used name for a combination antigen/antigen carrier that is composed of two or more, usually identical, antigenic molecules, specifically, the reconstituted epitope polypeptides of the invention covalently attached to a dendritic core which is composed of principal units which are at least bifunctional/difunctional. Each bifunctional unit in a branch provides a base for added growth. The dendritic core of a multiple antigen peptide system may be composed of lysine molecules. For example, a lysine is attached via peptide bonds through each of its amino groups to two additional lysine residues. This second-generation molecule has four free amino groups each of which can be covalently linked to an additional lysine to form a third-generation molecule with eight free amino groups. A peptide may be attached to each of these free groups to form an octavalent multiple peptide antigen (MAP). The process can be repeated to form fourth or even higher generations of molecules. With each generation, the number of free amino groups increases geometrically and can be represented by 2ⁿ, where n is the number of the generation. Alternatively, the second-generation molecule having four free amino groups can be used to form a tetravalent MAP with four peptides covalently linked to the core. Many other molecules, including, e.g., the amino acids Asp and Glu, both of which have two carboxyl groups and one amino group to produce poly Asp or poly Glu with 2n free carboxyl groups, can be used to form the dendritic core of MAPS. The term “dendritic polymer” is sometimes used herein to define a product of the invention. The term includes carrier molecules which are sufficiently large to be regarded as polymers as well as those which may contain as few as three monomers. The chemistry for synthesizing dendritic polymers is known and available. With amino acids, the chemistry for blocking functional groups which should not react and then removing the blocking groups when it is desired that the functional groups should react has been described in detail in numerous patents and scientific publications. The dendritic polymers and the entire MAP can be produced on a resin and then removed from the polymer. Ammonia or ethylenediamine may be utilized as the core molecule. In this procedure, the core molecule is reacted with an acrylate ester and the ester groups removed by hydrolysis. The resulting first-generation molecules contain three free carboxyl groups in the case of ammonia and four free carboxyl groups when ethylenediamine is employed. The dendritic polymer may be further extended with ethylenediamine followed by another acrylic ester monomer and repeats the sequence until the desired molecular weight was attained. It is readily apparent to one skilled in the art, that each branch of the dendritic polymer can be lengthened by any of a number of selected procedures. For example, each branch can be extended by multiple reactions with Lys molecules. Some important features of the dendritic polymer as an immunogenic carrier are that the precise structure is known, there are no “antigenic” contaminants or those that irritate tissue or provoke other undesirable reactions. The precise concentration of the reconstituted epitope polypeptides of the invention is known; and is symmetrically distributed on the carrier; and the carrier can be utilized as a base for more than one reconstituted epitope polypeptides or any DIII, DII, DI domains and E proteins disclosed herein, so that multivalent immunogens or vaccines can be produced. When the MAPS is to be employed to produce a vaccine or immunogenic composition, it is preferred that the core molecule of the dendrimer be a naturally occurring amino acid such as Lys so that it can be properly metabolized. However, non-natural amino acids residues may be also employed. The amino acids used in building the core molecule can be in either the D or L-form. As indicated above, the various aspects of the present disclosure disclosed herein above provide various polypeptides (e.g., reconstituted epitopes, DIII domains, E proteins, fusion proteins thereof, and multimeric forms thereof), and as such the present disclosure relates to polypeptides, specifically, isolated polypeptides or any proteineous material. The present disclosure provides multimeric and/or multivalent antigen displaying platform. It should be however understood that the present disclosure further provides any nanoparticle scaffold comprising any of the disclosed reconstituted epitopes. As noted above, multivalent antigen presentation, in which antigens are presented to the immune system in a repetitive array, has been demonstrated to increase the potency of humoral immune responses. This has been attributed to increased cross-linking of antigen-specific B cell receptors at the cell surface and modulation of immunogen trafficking to and within lymph nodes. An ongoing challenge has been to develop multimerization scaffolds capable of presenting complex oligomeric or engineered antigens, as these can be difficult to stably incorporate into non-protein-based nanomaterials (e.g., liposomes, polymers, transition metals and their oxides). Epitope accessibility, proper folding of the antigen, and stability are also important considerations in any strategy for antigen presentation. Several reports have utilized non-viral, naturally occurring protein scaffolds, such as self-assembling ferritin, lumazine synthase, or encapsulin nanoparticles, to present a variety of complex oligomeric or engineered antigens. More recently, computationally designed one- and two-component protein nanoparticles [King et al., Nature. (2014) Jun. 5; 510 (7503): 103-108; Bale et al., Science 353:389-393 (2016)] have been used to present complex oligomeric antigens, conferring additional advantages such as high stability, robust assembly, ease of production and purification, and increased potency upon immunization. As shown in the Examples the inventors demonstrated the successful use of the I53-50 nanoparticle platform. More specifically, I53-50 is a computationally designed two-component protein complex comprising 20 trimeric “A” components and 12 pentameric “B” components for a total of 120-subunit icosahedral protein nanostructures with molecular weights (1.8 to 2.8 megadaltons) and dimensions (24 to 40 nanometers in diameter) comparable to those of small viral capsids. Thus, in some embodiments, the present disclosure provides nanoparticle scaffold comprising any of the reconstituted epitopes disclosed herein.

An ‘isolated polypeptide’ is a polypeptide that is essentially free from contaminating cellular components, such as carbohydrate, lipid, or other proteinaceous impurities associated with the polypeptide in nature. Typically, a preparation of isolated polypeptide contains the polypeptide in a highly purified form, i.e., at least about 80% pure, at least about 90% pure, at least about 95% pure, greater than 95% pure, or greater than 99% pure. One way to show that a particular protein preparation contains an isolated polypeptide is by the appearance of a single band following sodium dodecyl sulfate (SDS)-polyacrylamide gel electrophoresis of the protein preparation and Coomassie Brilliant Blue staining of the gel. However, the term “isolated” does not exclude the presence of the same polypeptide in alternative physical forms, such as dimers or alternatively glycosylated or derivatized forms. By definition, isolated peptides are also non-naturally occurring, synthetic peptides. Methods for isolating or synthesizing peptides of interest with known amino acid sequences are well known in the art. The polypeptides of the invention are therefore considered as proteinaceous material. A “proteinaceous material” is any protein, or fragment thereof, or complex containing one or more proteins formed by any means, such as covalent peptide bonds, disulfide bonds, chemical crosslinks, etc., or non-covalent associations, such as hydrogen bonding, van der Waal's contacts, electrostatic salt bridges, etc. An ‘amino acid/s’ or an ‘amino acid residue/s’ can be a natural or non-natural amino acid residue/s linked by peptide bonds or bonds different from peptide bonds. The amino acid residues can be in D-configuration or L-configuration (referred to herein as D- or L-enantiomers). An amino acid residue comprises an amino terminal part (NH2) and a carboxy terminal part (COOH) separated by a central part (R group) comprising a carbon atom, or a chain of carbon atoms, at least one of which comprises at least one side chain or functional group. NH2 refers to the amino group present at the amino terminal end of an amino acid or peptide, and COOH refers to the carboxy group present at the carboxy terminal end of an amino acid or peptide. The generic term amino acid comprises both natural and non-natural amino acids. Natural amino acids of standard nomenclature are listed in 37 C.F.R. 1.822(b)(2). Examples of non-natural amino acids are also listed in 37 C.F.R. 1.822(b)(4), other non-natural amino acid residues include, but are not limited to, modified amino acid residues, L-amino acid residues, and stereoisomers of D-amino acid residues. Naturally occurring amino acids may be further modified, e.g., hydroxyproline, γ-carboxyglutamate, and O-phosphoserine. Further, amino acids may be amino acid analogs or amino acid mimetics. Amino acid analogs refer to compounds that have the same fundamental chemical structure as naturally occurring amino acids, but modified R groups or modified peptide backbones, e.g., homoserine, norleucine, methionine sulfoxide, methionine methyl sulfonium. Amino acid mimetics refers to chemical compounds that have a structure that is different from the general chemical structure of an amino acid, but that function in a manner similar. Amino acids may be referred to herein by either their commonly known three letter symbols or by the one-letter symbols recommended by the IUPAC-IUB Biochemical Nomenclature Commission. Further, the reconstituted epitope polypeptides of the invention may comprise ‘equivalent amino acid residues’. This term refers to an amino acid residue capable of replacing another amino acid residue in a polypeptide without substantially altering the structure and/or functionality of the polypeptide. Equivalent amino acids thus have similar properties such as bulkiness of the side-chain, side chain polarity (polar or non-polar), hydrophobicity (hydrophobic or hydrophilic), pH (acidic, neutral or basic) and side chain organization of carbon molecules (aromatic/aliphatic). As such, equivalent amino acid residues can be regarded as conservative amino acid substitutions. In the context of the present invention, within the meaning of the term ‘equivalent amino acid substitution’ as applied herein, is meant that in certain embodiments one amino acid may be substituted for another within the groups of amino acids indicated herein below: (i) Amino acids having polar side chains (Asp, Glu, Lys, Arg, His, Asn, Gln, Ser, Thr, Tyr, and Cys); (ii) Amino acids having non-polar side chains (Gly, Ala, Val, Leu, Ile, Phe, Trp, Pro, and Met); (iii) Amino acids having aliphatic side chains (Gly, Ala Val, Leu, Ile); (iv) Amino acids having cyclic side chains (Phe, Tyr, Trp, His, Pro); (v) Amino acids having aromatic side chains (Phe, Tyr, Trp); (vi) Amino acids having acidic side chains (Asp, Glu); (vii) Amino acids having basic side chains (Lys, Arg, His); (viii) Amino acids having amide side chains (Asn, Gln); (ix) Amino acids having hydroxy side chains (Ser, Thr); (x) Amino acids having sulphur-containing side chains (Cys, Met); (xi) Neutral, weakly hydrophobic amino acids (Pro, Ala, Gly, Ser, Thr); (xii) Hydrophilic, acidic amino acids (Gln, Asn, Glu, Asp), and (xiii) Hydrophobic amino acids (Leu, Ile, Val).

Still further, the reconstituted epitope polypeptide of the invention may have secondary modifications, such as phosphorylation, acetylation, glycosylation, sulfhydryl bond formation, cleavage and the likes, as long as said modifications retain the functional properties of the original protein. In some specific embodiments, the functional properties of the neutralizing epitopes of the present disclosure, or any of the polypeptides disclosed herein, are specifically, the ability to interact with the neutralizing antibodies or any other binding molecule, thereby disrupting, inhibiting, reducing and/or eliminating the viral penetration to the target cell, in at least about 5%-99.9999%, about 10%-90%, about 15%-85%, about 20%-80%, about 25%-75%, about 30%-70%, about 35%-65%, about 40%-60% or about 45%-55%, and more specifically, by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 99.99%, 99.999%, 99.9999% or about 100%. In some embodiments, the reconstituted epitope of the invention, for example, any epitope based on the DIII domain that does not always bind the receptor, serves as an effective neutralizing epitope towards which the most potent neutralizing antibodies bind. In yet some alternative or additional embodiments, the functional properties of the neutralizing epitopes of the present disclosure may be the ability to interact with the viral receptor thereby disrupting, inhibiting, reducing and/or eliminating the viral penetration to the target cell.

Secondary modifications are often referred to in terms of relative position to certain amino acid residues. For example, a certain sequence positioned carboxyl-terminal to a reference sequence within a polypeptide is located proximal to the carboxyl terminus of the reference sequence but is not necessarily at the carboxyl terminus of the complete polypeptide. The invention further encompasses any derivatives, enantiomers, analogues, variants or homologues of any of the reconstituted epitope polypeptides disclosed herein. The term “derivative” is used to define amino acid sequences (polypeptide), with any insertions, deletions, substitutions and modifications to the amino acid sequences (polypeptide) that do not alter the activity of the original polypeptides. By the term “derivative” it is also referred to homologues, variants and analogues thereof, as well as covalent modifications of a polypeptides made according to the present invention. Thus, any variant or derivative as disclosed below, must retain at least one of the functional properties of the reconstituted epitopes, or any of the polypeptides disclosed by the present disclosure.

It should be noted that the reconstituted epitope polypeptides according to the invention can be produced either synthetically, or by recombinant DNA technology. Methods for producing polypeptides peptides are well known in the art. In some embodiments, derivatives include, but are not limited to, polypeptides that differ in one or more amino acids in their overall sequence from the polypeptides defined herein, polypeptides that have deletions, substitutions, inversions or additions. In some embodiments, derivatives refer to polypeptides, which differ from the polypeptides specifically defined in the present invention by insertions of amino acid residues. It should be appreciated that by the terms “insertions” or “deletions”, as used herein it is meant any addition or deletion, respectively, of amino acid residues to the polypeptides used by the invention, of between 1 to 50 amino acid residues, between 20 to 1 amino acid residues, and specifically, between 1 to 10 amino acid residues. More particularly, insertions or deletions may be of any one of 1, 2, 3, 4, 5, 6, 7, 8, 9, or 10 amino acids. It should be noted that the insertions or deletions encompassed by the invention may occur in any position of the modified peptide, as well as in any of the N′ or C′ termini thereof. It should be appreciated that in cases the deletion/s or insertion/s are in the N or C-terminus of the peptide, such derivatives may be also referred to as fragments. The reconstituted epitope polypeptide of the invention of the invention may all be positively charged, negatively charged or neutral. In addition, they may be in the form of a dimer, a multimer or in a constrained conformation, which can be attained by internal bridges, short-range cyclization, extension or other chemical modifications. The polypeptides of the invention can be coupled (conjugated) through any of their residues to another peptide or agent. For example, the polypeptides of the invention can be coupled through their N-terminus to a lauryl-cysteine (LC) residue and/or through their C-terminus to a cysteine (C) residue.

Further, the reconstituted epitope polypeptide of the invention, or any of the polypeptides disclosed by the present disclosure (DIII domain, E protein, that comprise the reconstituted epitopes, and/or at least one linker), may be extended at the N-terminus and/or C-terminus thereof with various identical or different amino acid residues. As an example for such extension, the peptide may be extended at the N-terminus and/or C-terminus thereof with identical or different amino acid residue/s, which may be naturally occurring or synthetic amino acid residue/s. An additional example for such an extension may be provided by peptides extended both at the N-terminus and/or C-terminus thereof with a cysteine residue. Naturally, such an extension may lead to a constrained conformation due to Cys-Cys cyclization resulting from the formation of a disulfide bond. Another example may be the incorporation of an N-terminal lysyl-palmitoyl tail, the lysine serving as linker and the palmitic acid as a hydrophobic anchor. In addition, the peptides may be extended by aromatic amino acid residue/s, which may be naturally occurring or synthetic amino acid residue/s, for example, aromatic amino acid residue such as tryptophan, tyrosine or phenyl alanine. The peptides may be extended at the N-terminus and/or C-terminus thereof with various identical or different organic moieties, which are not naturally occurring or synthetic amino acids. As an example for such extension, the reconstituted epitope polypeptide may be extended at the N-terminus and/or C-terminus thereof with an N-acetyl group. For every single peptide sequence defined by the invention and disclosed herein, this invention includes the corresponding retro-inverse sequence wherein the direction of the peptide chain has been inverted and wherein all or part of the amino acids belong to the D-series. It should be understood that the present invention includes embodiments wherein one or more of the L-amino acids is replaced with its D isomer. In yet some further embodiments, the reconstituted epitope polypeptide of the invention may comprise at least one amino acid residue in the D-form. It should be noted that every amino acid (except glycine) can occur in two isomeric forms, because of the possibility of forming two different enantiomers (stereoisomers) around the central carbon atom. By convention, these are called L- and D-forms, analogous to left-handed and right-handed configurations. It should be appreciated that in some embodiments, the enantiomer or any derivatives of the reconstituted epitope of the invention may exhibit at least one of enhanced activity, and superiority, specifically, in at least one of the properties discussed above, particularly, neutralization properties. In more specific embodiments, such derivatives and enantiomers may exhibit increased affinity to any binding molecule, for example, antibodies (either neutralizing or not), that may be polyclonal convalescent sera, monoclonal antibodies, (mAbs) or the viral receptor, enhanced stability, and increased resistance to proteolytic degradation. The invention also encompasses any homologues of the polypeptides specifically defined by their amino acid sequence according to the invention. The term “homologues” is used to define amino acid sequences (polypeptide) which maintain a minimal homology to the amino acid sequences defined by the invention, e.g. preferably have at least about 65%, more preferably at least about 70%, at least about 75%, even more preferably at least about 80%, at least about 85%, most preferably at least about 90%, at least about 95% overall sequence homology with the entire amino acid sequence of any of the polypeptide as structurally defined above, e.g. of a specified sequence, more specifically, an amino acid sequence of the polypeptides as denoted by any one of SEQ ID NOs: 1-102, and any derivatives, enantiomers and fusion proteins thereof. In yet some further specific embodiments, derivatives, homologs and variants as discussed herein may specifically apply to any of the reconstituted epitopes of the present disclosure, specifically, any of the reconstituted epitopes derived from the DIII domain of the E protein of the Dengue virus. Specifically, any of the reconstituted epitopes that comprise the amino acid sequence as denoted by any one of SEQ ID NOs: 63, 64, 67, 72, 56-62, 65, 66, 68, 70, 71, 73-75, as well as those derived from the DII domain, as disclosed in any one of SEQ ID NO: 29-38. More specifically, “Homology” with respect to a native polypeptide and its functional derivative is defined herein as the percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Neither N- nor C-terminal extensions nor insertions or deletions shall be construed as reducing identity or homology. Methods and computer programs for the alignment are well known in the art.

In some embodiments, the present invention also encompasses polypeptides which are variants of, or analogues to, the polypeptides specifically defined in the invention by their amino acid sequence. With respect to amino acid sequences, one of skill will recognize that individual substitutions, deletions or additions to peptide, polypeptide, or protein sequence thereby altering, adding or deleting a single amino acid or a small percentage of amino acids in the encoded sequence is a “conservatively modified variant”, where the alteration results in the substitution of an amino acid with a chemically similar amino acid. Conservative substitution tables providing functionally similar amino acids are well known in the art and disclosed herein before. Such conservatively modified variants are in addition to and do not exclude polymorphic variants, interspecies homologues, and alleles and analogous peptides of the invention. More specifically, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. As noted above, the peptides of the invention may be modified by omitting their N-terminal sequence. It should be appreciated that the invention further encompasses the omission of about 1, 2, 3, 4, 5, 6, 7, 8 and more amino acid residues from both, the N′ and/or the C′ termini of the peptides of the invention. Certain commonly encountered amino acids which also provide useful substitutions include, but are not limited to, β-alanine (β-Ala) and other omega-amino acids such as 3-aminopropionic acid, 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine or sarcosine (MeGIy); ornithine (Orn); citrulline (Cit); t-butylalanine (t-BuA); t-butylglycine (t-BuG); N-methylisoleucine (Melle); phenylglycine (Phg); cyclohexylalanine (Cha); norleucine (NIe); naphthylalanine (NaI); 4-chlorophenylalanine (Phe(4-C1)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); β-2-thienylalanine (Thi); methionine sulfoxide (MSO); homoarginine (hArg); N-acetyl lysine (AcLys); 2,4-diaminobutyric acid (Dbu); 2,4-diaminobutyric acid (Dab); p-aminophenylalanine (Phe (pNH.sub.2)); N-methyl valine (MeVal); homocysteine (hCys), homophenylalanine (hPhe) and homoserine (hSer); hydroxyproline (Hyp), homoproline (hPro), N-methylated amino acids (e.g., N-substituted glycine). Covalent Modifications of Amino Acids and the Peptide Covalent modifications of the peptide are included and may be introduced by reacting targeted amino acid residues of the peptide with an organic derivatizing agent that is capable of reacting with selected side chains or terminal residues. Cysteinyl residues most commonly are reacted with α-haloacetates (and corresponding amines) to give carboxymethyl or carboxyamidomethyl derivatives. Cysteinyl residues also are derivatized by reaction with bromotrifluoroacetone, α-bromo-β-(5-imidozoyl) propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole. Histidyl residues are derivatized by reaction with diethylprocarbonate (pH 5.5-7.0) which agent is relatively specific for the histidyl side chain. Bromophenacyl bromide also is useful; the reaction is preferably performed in 0.1 M sodium cacodylate at pH 6.0. Lysinyl and amino terminal residues are reacted with succinic or other carboxylic acid anhydrides. Derivatization with these agents reverses the charge of the lysinyl residues. Other suitable reagents for derivatizing α-amino-containing residues include imidoesters such as methylpicolinimidate; pyridoxal phosphate; pyridoxal; chloroborohydride; trinitrobenzenesulfonic acid; O-methylisourea; 2,4 pentanedione; and transaminase-catalyzed reaction with glyoxylate. Arginyl residues are modified by reaction with one or several conventional reagents, including phenylglyoxal, 2,3-butanedione, 1,2-cyclohexanedione, and ninhydrin. Such derivatization requires that the reaction be performed in alkaline conditions because of the high pKa of the guanidine functional group. Furthermore, these reagents may react with the groups of lysine as well as the arginine s-amino group. Modification of tyrosyl residues has permits introduction of spectral labels into a peptide. This is accomplished by reaction with aromatic diazonium compounds or tetranitromethane. Most commonly, N-acetylimidizol and tetranitromethane are used to create O-acetyl tyrosyl species and 3-nitro derivatives, respectively. Carboxyl side groups (aspartyl or glutamyl) are selectively modified by reaction with carbodiimides (R′—N—C—N—R′) such as 1-cyclohexyl-3-(2-morpholinyl-(4-ethyl) carbodiimide or 1-ethyl-3-(4-azonia-4,4-dimethylpentyl) carbodiimide. Aspartyl and glutamyl residues are converted to asparaginyl and glutaminyl residues by reaction with ammonium ions. Conversely, glutaminyl and asparaginyl residues may be deamidated to the corresponding glutamyl and aspartyl residues. Deamidation can be performed under mildly acidic conditions. Either form of these residues falls within the scope of this invention. Derivatization with bifunctional agents is useful for cross-linking the peptide to a water-insoluble support matrix or other macromolecular carrier. Commonly used cross-linking agents include 1, 1-bis(diazoacetyl)-2-phenylethane, glutaraldehyde, N-hydroxysuccinimide esters, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio] propioimidate yield photoactivatable intermediates that are capable of forming crosslinks in the presence of light. Other chemical modifications include hydroxylation of proline and lysine, phosphorylation of the hydroxyl groups of seryl or threonyl residues, methylation of the α-amino groups of lysine, arginine, and histidine side chains (Creighton, supra), acetylation of the N-terminal amine, and, in some instances, amidation of the C-terminal carboxyl. Such chemically modified and derivatized moieties may improve the peptide's solubility, absorption, biological half-life, and the like. These changes may eliminate or attenuate undesirable side effects of the proteins in vivo. It should be appreciated that the invention further encompasses any of the peptides of the invention referred herein, any surrogates thereof, any salt, base, ester or amide thereof, any enantiomer, stereoisomer or disterioisomer thereof, or any combination or mixture thereof. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts.

As noted above, the invention further encompasses any fusion protein comprising the reconstituted epitope polypeptides of the invention as described herein, or any DIII, DII, DI domains and E proteins of the invention, or any variants thereof or any derivative, enantiomer or conjugate thereof. More specifically, additional peptide sequences can be added to the polypeptides of the invention thereby forming fusion proteins, which act to promote stability, purification, and/or detection. For example, a reporter peptide portion (e.g., green fluorescent protein (GFP), β-galactosidase, His Tag, a detectable domain thereof, or any other immunogenic determinants) can be used. Purification-facilitating peptide sequences include those derived or obtained from maltose binding protein (MBP), glutathione-S-transferase (GST), I53-A50 platform, or thioredoxin (TRX). Still further, in some embodiments, a protease cleavage site may be introduced between the construct and the fusion scaffold. Such a cleavage site could be for example the tetrapeptide Ile Glu Gly Arg (IEGR) which is recognized by the enzyme coagulation factor Xa which cleaves carboxy terminal to the arginine residue. Specific examples for fusion proteins provided by the present disclosure are disclosed in the Example, and in connection with other aspects of the invention herein before. In further aspect of the present disclosure relates to at least one nucleic acid sequence encoding at least one reconstituted epitope of a viral envelope protein, any polypeptide, domain (e.g., DIII, DII, or DI), or viral envelope protein (E protein) comprising the reconstituted epitope, any domain or viral envelope protein comprising at least one linker that replaces at least one loop or any part thereof or amino acid residue/s thereof in the viral protein, any multimeric and/or multivalent antigen displaying platform thereof, and any combinations thereof, any fusion protein, conjugate, polyvalent dendrimer thereof. More specifically, the viral envelope protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII. Still further, the reconstituted epitope comprises at least one linker and at least one fragment of the native envelope protein. In some embodiments, the nucleic acid sequence of the present disclosure may encode any of the polypeptide/s that comprise at least one reconstituted epitope as defined by the present disclosure. In yet some further embodiments, the nucleic acid sequence of the present disclosure may encode any of the multimeric and/or multivalent antigen displaying platform as defined by the present disclosure. Still further embodiments provide nucleic acid sequences that encode any domain or viral envelope protein comprising at least one linker as defined by any one of the embodiments of the present disclosure. In some embodiments, the nucleic acid sequences disclosed herein may encode any of the reconstituted epitopes disclosed by the invention and any E proteins, or DIII domains comprising the disclosed reconstituted epitopes or E proteins, or DIII domains that comprise at least one of the linkers disclosed by the present disclosure. In ye some further specific and non-limiting embodiments, the present disclosure provides nucleic acid sequences that encode any of the reconstituted epitopes of SEQ ID NO: 63, 64, 67, 72, 56-62, 65, 66, 68, 70, 71, 73-75. Specific embodiments relate to any nucleic acid sequences encoding any one of the reconstituted epitopes of SEQ ID NO: 63, 64, 67, 72, or of any fusion proteins thereof, as denoted by SEQ ID NOs: 132 to 142.

As used herein, the term ‘polynucleotide’ or a ‘nucleic acid sequence’ refers to a polymer of nucleic acids, such as deoxyribonucleic acid (DNA) or ribonucleic acid (RNA), any combination or hybrids thereof, or any combinations or conjugates thereof with peptides. As used herein, ‘nucleic acid’ (also or nucleic acid molecule or nucleotide) refers to any DNA or RNA polynucleotides, oligonucleotides, fragments generated by the polymerase chain reaction (PCR) and fragments generated by any of ligation, scission, endonuclease action, and exonuclease action, either single- or double-stranded. Nucleic acid molecules can be composed of monomers that are naturally-occurring nucleotides (such as DNA and RNA), or analogs of naturally-occurring nucleotides (e.g., alpha-enantiomeric forms of naturally-occurring nucleotides), or modified nucleotides or any combination thereof. Herein this term also encompasses a cDNA, i.e. complementary or copy DNA produced from an RNA template by the action of reverse transcriptase (RNA-dependent DNA polymerase). Exemplary nucleic acids or polynucleotides of the invention include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, a-LNA having an a-L-ribo configuration (a diastereomer of LNA), 2′-amino-LNA having a 2′-amino functionalization, and 2′-amino-a-LNA having a 2′-amino functionalization), ethylene nucleic acids (ENA), cyclohexenyl nucleic acids (CeNA) or hybrids or combinations thereof. In other embodiments, the polynucleotides of the present invention which have portions or regions which differ in size and/or chemical modification pattern, chemical modification position, chemical modification percent or chemical modification population and combinations of the foregoing are known as “chimeric polynucleotides.” A “chimera” according to the present invention is an entity having two or more incongruous or heterogeneous parts or regions. As used herein a “part” or “region” of a polynucleotide is defined as any portion of the polynucleotide which is less than the entire length of the polynucleotide. In yet another embodiment, the polynucleotides of the present invention that are circular are known as “circular polynucleotides” or “circP.” As used herein, “circular polynucleotides” or “circP” means a single stranded circular polynucleotide which acts substantially like, and has the properties of, an RNA. The term “circular” is also meant to encompass any secondary or tertiary configuration of the circP. In some embodiments, the polynucleotide includes from about 30 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000).

In this connection an ‘isolated polynucleotide’ is a nucleic acid molecule that is separated from the genome of an organism. For example, a DNA molecule that encodes any of the reconstituted epitope polypeptides of the invention or any derivative, variant, fragment or fusion protein thereof that has been separated from the genomic DNA of a cell is an isolated DNA molecule. Another example of an isolated nucleic acid molecule is a chemically-synthesized nucleic acid molecule that is not integrated in the genome of an organism. A nucleic acid molecule that has been isolated from a particular species is smaller than the complete DNA molecule of a chromosome from that species. In one embodiment, the polynucleotide/s of the present invention is/or functions as a messenger RNA (mRNA). As used herein, the term “messenger RNA” (mRNA) refers to any polynucleotide which encodes at least one any of the reconstituted epitope polypeptides or any DIII, DII, DI domains and E proteins of the invention or any derivative, variant or fusion protein thereof, and which is capable of being translated to produce the encoded any of the reconstituted epitope polypeptides or any DIII, DII, DI domains and E proteins, or fusion protein thereof, of the invention or any derivative or variant thereof, in vitro, in vivo, in situ or ex vivo. Another example of an isolated nucleic acid molecule that may be useful in the present disclosure, is a chemically-synthesized nucleic acid molecule that is not integrated in the genome of an organism. A nucleic acid molecule that has been isolated from a particular species is smaller than the complete DNA molecule of a chromosome from that species. The invention further relates to recombinant DNA constructs comprising the polynucleotides of the invention that encodes any of the reconstituted epitope polypeptides or any DIII, DII, DI domains and E proteins of the invention, or any variants thereof or any derivative, enantiomer, fusion protein or conjugate thereof. The constructs of the invention may further comprise additional elements such as promoters, regulatory and control elements, translation, expression and other signals, operably linked to the nucleic acid sequence of the invention. As used herein, the term “recombinant DNA” or “recombinant gene” refers to a nucleic acid comprising an open reading frame encoding one of the proteins of the invention.

Expression vectors are typically self-replicating DNA or RNA constructs containing the desired gene or its fragments, and operably linked genetic control elements that are recognized in a suitable host cell and effect expression of the desired genes. These control elements are capable of effecting expression within a suitable host. Generally, the genetic control elements can include a prokaryotic promoter system or a eukaryotic promoter expression control system. This typically includes a transcriptional promoter, an optional operator to control the onset of transcription, transcription enhancers to elevate the level of RNA expression, a sequence that encodes a suitable ribosome binding site, RNA splice junctions, sequences that terminate transcription and translation and so forth. Expression vectors usually contain an origin of replication that allows the vector to replicate independently of the host cell.

Accordingly, the term control and regulatory elements includes promoters, terminators and other expression control elements. For instance, any of a wide variety of expression control sequences that control the expression of a DNA sequence when operatively linked to it may be used in these vectors to express DNA sequences encoding any desired protein using the method of this invention. A vector may additionally include appropriate restriction sites, antibiotic resistance or other markers for selection of vector-containing cells. Plasmids are the most commonly used form of vector but other forms of vectors which serve an equivalent function and which are, or become, known in the art are suitable for use herein. As noted above, the nucleic acid molecule of the invention is used in accordance with some non-limiting embodiments as a nucleic acid vaccine, and thus, can be formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with the nucleic acid vaccines (e.g., for transplantation into a subject), hyaluronidase, nanoparticle mimics and combinations thereof. Still further, complexes, micelles, liposomes or particles can be prepared containing these lipidoids and therefore, can result in an effective delivery of the polynucleotide, as judged by the production of an encoded protein, following the injection of a lipidoid formulation via localized and/or systemic routes of administration. Lipidoid complexes of polynucleotides in accordance with the invention can be administered by various means including, but not limited to, intravenous, intramuscular, or subcutaneous routes. Complexes, micelles, liposomes or particles can be prepared containing these lipidoids and/or lipid nano-particles (LNP), and therefore, can result in an effective delivery of the polynucleotide, as judged by the production of an encoded protein, following the injection of a lipidoid formulation via localized and/or systemic routes of administration. Lipidoid complexes of polynucleotides can be administered by various means including, but not limited to, intravenous, intramuscular, or subcutaneous routes.

In vivo delivery of nucleic acids may be affected by many parameters, including, but not limited to, the formulation composition, nature of particle PEGylation, degree of loading, polynucleotide to lipid ratio, and biophysical parameters such as, but not limited to, particle size. As an example, small changes in the anchor chain length of poly(ethylene glycol) (PEG) lipids may result in significant effects on in vivo efficacy. Formulations with the different lipidoids, including, but not limited to penta [3-(1-laurylaminopropionyl)]-triethylenetetramine hydrochloride, CI 2-200 (including derivatives and variants), and MD1, are also encompassed by the present disclosure.

It should be understood that a nucleic acid vaccine as referred to herein, further encompasses any mixture of nucleic acid molecules that encode various polypeptides, specifically, at least 1, 2, 3, 4, 5, 6, 7, 8, 9, 10, 20, 30, 40, 50, 60, 70, 80, 90, 100 or more of the various reconstituted epitope polypeptides or any DIII, DII, DI domains and E proteins or any of the linkers of the invention, or ay fusion proteins thereof. Still further, the invention also provides any natural or artificial host cell and any components thereof (e.g., membrane fragments) comprising any of the nucleic acid sequence of the invention or expressing any polypeptide encoded thereby, or any combinations thereof.

A further aspect provided by the present disclosure relates to a composition comprising an effective amount of at least one of, at least one polypeptide comprising an amino acid sequence of at least one reconstituted epitope of a viral envelope protein, and/or any polypeptide, domain or viral envelope protein comprising the reconstituted epitope, and/or any domain or viral envelope protein comprising at least one linker that replaces at least one loop or any part thereof or amino acid residue/s thereof in the viral protein, and/or any multimeric and/or multivalent antigen displaying platform thereof, and any combinations thereof, any derivative, enantiomer, fusion protein, conjugate, polyvalent dendrimer thereof, and/or any nucleic acid sequence encoding the same or any matrix, nano- or micro-particle thereof. In some embodiments, the viral envelope protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII. Still further, the reconstituted epitope as disclosed herein comprises at least one linker and at least one fragment of the native envelope protein. In some embodiments, the composition of the present disclosure may optionally further comprise at least one pharmaceutically acceptable carrier/s, excipient/s, adjuvant/s, auxiliaries, and/or diluent/s.

In more specific embodiments of the composition/s disclosed herein, the reconstituted epitope is of any of the polypeptide as defined by the present disclosure. Still further, the composition disclosed herein may comprise any of the domains or viral envelope protein/s comprising at least one linker, as defined herein before. In yet some further embodiments, the composition of the present disclosure may comprise any of the multimeric and/or multivalent antigen displaying platform as defined by the present disclosure. Still further embodiments provide compositions comprising any nucleic acid sequence as defined by any one of the embodiments of the present disclosure. The compositions disclosed by the present disclosure comprise any one of the reconstituted epitope polypeptides or any DIII, DII, DI domains and E proteins of the invention, or any variants thereof or any derivative, enantiomer, fusion protein or conjugate thereof. The term “pharmaceutical composition” in the context of the invention means that the composition is of a grade and purity suitable for prophylactic or therapeutic administration to human subjects and is present together with at least one of carrier/s, diluent/s, excipient/s, adjuvant/s and/or additive/s that are pharmaceutically acceptable. The pharmaceutical composition may be suitable for any mode of administration whether oral or parenteral, by injection or by topical administration by inhalation, intranasal spray or intraocular drops. More specifically, pulmonary, oral, transmucosal, intestinal or parenteral delivery, including intramuscular, subcutaneous and intramedullary injections as well as rectal, intrathecal, direct intraventricular, intravenous, intraocular injections or any other medically acceptable methods of administration may be considered as appropriate administration mode for the compositions of the invention.

Pharmaceutical compositions according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations.

It should be understood that in addition to the ingredients particularly mentioned above, the formulations may also include other agents conventional in the art having regard to the type of formulation in question, for example those suitable for oral administration may include flavoring agents. As noted above, any of the compositions of the invention may comprise pharmaceutically acceptable carriers, vehicles, adjuvants, excipients, or diluents. As used herein pharmaceutically acceptable carriers, vehicles, adjuvants, excipients, or diluents, are well-known to those skilled in the art and are readily available to the public. It is preferred that the pharmaceutically acceptable carrier be one which is chemically inert to the active compounds and one which has no detrimental side effects or toxicity under the conditions of use. The choice of a carrier will be determined in part by the particular active agent, as well as by the particular method used to administer the composition. The carrier can be a solvent or a dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

Each carrier should be both pharmaceutically and physiologically acceptable in the sense of being compatible with the other ingredients and not injurious to the subject. Formulations include those suitable for immersion, oral, parenteral (including subcutaneous, intramuscular, intravenous, intraperitoneal, implantation for slow release and intradermal) administration. The formulations may conveniently be presented in unit dosage form and may be prepared by any methods well known in the art of pharmacy. The nature, availability and sources, and the administration of all such compounds including the effective amounts necessary to produce desirable effects in a subject are well known in the art and need not be further described herein.

A further aspect of the present disclosure relates to an anti-viral vaccine comprising at least one polypeptide comprising an amino acid sequence of at least one reconstituted epitope of a viral envelope protein, and/or any polypeptide, domain or viral envelope protein comprising the reconstituted epitope, any domain or viral envelope protein comprising at least one linker that replaces at least one loop or any part thereof, or amino acid residue/s thereof in said viral protein, and/or any multimeric and/or multivalent antigen displaying platform thereof, and any combinations thereof, any derivative, enantiomer, fusion protein, conjugate, polyvalent dendrimer thereof, and any nucleic acid sequence encoding the same, or any matrix, nano- or micro-particle thereof. The viral envelope protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII. Still further, in some embodiments, the reconstituted epitope comprises at least one linker and at least one fragment of the native envelope protein, the vaccine optionally further comprises at least one pharmaceutically acceptable carrier/s, excipient/s, adjuvant/s, auxiliaries, and/or diluent/s. In some embodiments, the anti-viral vaccine as disclosed herein, is capable of eliciting an immune response specific for this virus in a subject. Thus, in some embodiments, the immunogenic composition of the invention may induce an immune response in a subject. “Immune response” as used herein means the activation of a host's immune system, e.g., that of any eukaryotic multicellular organism, in response to the introduction of antigen. The immune response can be in the form of a cellular or humoral response, or both. It should be understood that the immune response as referred to herein, encompasses the immune response of any vertebrate or invertebrate organism. More specifically, a vertebrate organism as used herein encompasses any organism derived from any of the vertebrates groups that include Mammals (e.g., Marsupials, Primates, Rodents and Cetaceans), Birds, Fish, Amphibians and Reptiles. Subject applicable in the present disclosure are discussed in more detail herein after.

In some embodiments of the anti-viral vaccine disclosed herein, any of the polypeptide/s that comprise at least one reconstituted epitope as defined by the present disclosure. In yet some further embodiments, the anti-viral vaccine of the present disclosure, specifically, the anti viral vaccine directed against at least one virus of the Flaviviridae, specifically of the genus Flaviviruses, may encode any of the multimeric and/or multivalent antigen displaying platform as defined herein before. Still further embodiments provide anti-viral vaccine comprise any domain or viral envelope protein comprising at least one linker as defined by any one of the embodiments of the present disclosure. In yet some further embodiments, the anti-viral vaccine disclosed herein may comprise any of the nucleic acid sequences as disclosed in connection with other aspect of the invention. In some embodiments, the vaccine provided by the present disclosure may induce a humoral immune response in the subject administered the vaccine or the immunogenic composition. In some embodiments, the induced humoral immune response may be specific for the Flavivirus, specifically, the Dengue virus, Zika virus, WNV, YFV, or any of the viruses disclosed herein. The humoral immune response may be induced in the subject administered the vaccine by about 1.5-fold to about 100-fold, about 2-fold to about 90-fold, or about 3-fold to about 80-fold, as compared with subject not administered with the disclosed vaccine, specifically, an unvaccinated subject/s. The humoral immune response can be induced in the subject administered the vaccine by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 20-fold, at least about 30-fold, at least about 40-fold, at least about 50-fold, at least about 60-fold, at least about 70-fold, at least about 80-fold, at least about 90-fold, at least about 100-fold, or more, as compared with subject not administered with the disclosed vaccine, specifically, an unvaccinated subject/s. The humoral immune response induced by the vaccine may include an increased level of neutralizing antibodies associated with the subject administered the vaccine as compared to a subject not administered the vaccine. The neutralizing antibodies may be specific for the Dengue virus, Zika virus, WNV, YFV, any serotype, variant or mutant thereof, or any of the viruses disclosed herein. In some embodiments, neutralizing antibodies raised in the vaccinated subjects may be specific for the reconstituted epitopes of the invention. The neutralizing antibodies can provide protection against and/or treatment of infection by Dengue virus, Zika virus, WNV, YFV, or any of the viruses disclosed herein and against any associated pathologies in the subject administered the vaccine.

The humoral immune response induced by the vaccine may include an increased level of IgG antibodies associated with the subject administered the vaccine as compared with a subject that was not administered the vaccine. The level of IgG antibody associated with the subject administered the vaccine may be increased by about 1.5-fold to about 100-fold, about 2-fold to about 50-fold, or about 3-fold to about 25-fold in vaccinated subjects, as compared with subject/s that were not administered the vaccine. The level of IgG antibody in the subjects administered the vaccine can be increased by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 20-fold, at least about 30-fold, at least about 40-fold, at least about 50-fold, at least about 60-fold, at least about 70-fold, at least about 80-fold, at least about 90-fold, at least about 100-fold, or more, as compared with subject not administered with the disclosed vaccine, specifically, an unvaccinated subject/s.

In yet some further embodiments, the vaccine can induce a cellular immune response in the vaccinated subject, such response may be specific for at least one of the reconstituted epitopes, DIII, DII, DI domains and/or E proteins of the invention, or any combinations thereof. The phrase an immune response “specific for”, as used herein, is meant that the response is achieved only in the presence of the specific polypeptides or any nucleic acid sequences encoding the polypeptides. The induced cellular immune response may include eliciting a CD8⁺ T cell response, that may according to certain embodiments, include the production of cytokines such as interferon-gamma (IFN-γ), tumor necrosis factor alpha (TNF-alpha), interleukin-2 (IL-2), or any combinations thereof. In yet some further embodiments, the cellular immune response induced by the vaccine can include eliciting a CD4⁺ T cell response. In some embodiments, the CD4⁺ T cells may produce IFN-γ, TNF-α, IL-2, or a combination of IFN-γ and TNF-α. The vaccine may further induce an immune response when administered to different tissues such as the lungs or any airways, mucosal tissues, muscle or skin. The vaccine can further induce an immune response when administered via electroporation, or inhalation, or injection, or subcutaneously, or intramuscularly.

The vaccine may be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration. The subject can be a mammal, specifically a primate such as a human, as well as other mammals, specifically domestic animals for example, a camel or any other camelids, such as the llama, alpaca, guanaco, and vicuña of South America, a horse, a cow, a pig, a sheep, goat, a cat, a dog, or any laboratory animal, for example any rodent such as a rat, a mouse, a rabbit and the like.

It should be noted that the vaccine can be administered prophylactically or therapeutically. In prophylactic administration, the vaccines may be administered in an amount sufficient to induce an immune response. In therapeutic applications, the vaccines are administered to a subject in need thereof in an amount sufficient to elicit a therapeutic effect. An amount adequate to accomplish this is defined as “therapeutically effective dose.” Amounts effective for this use will depend on, e.g., the particular composition of the vaccine regimen administered, the manner of administration, the stage and severity of the disease, the general state of health of the patient, and the judgment of the prescribing physician.

The reconstituted epitope polypeptides or any DIII, DII, DI domains and E proteins of the invention, or any variants thereof or any derivative, enantiomer, fusion protein or conjugate thereof, of the disclosed vaccine/s can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the reconstituted epitope polypeptide. The vaccine can be delivered via a variety of routes. Typical delivery routes include parenteral administration, e.g., intradermal, intramuscular or subcutaneous delivery. Other routes include oral administration, intranasal, intravaginal and mucosal administration (such as intranasal, oral, intratracheal, and ocular). The vaccine can also be administered to muscle, or can be administered via intradermal or subcutaneous injections, or transdermally, such as by iontophoresis. Epidermal administration of the vaccine can also be employed. Epidermal administration can involve mechanically or chemically irritating the outermost layer of epidermis to stimulate an immune response to the irritant.

The vaccine can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine. The vaccine can be a liquid preparation such as a suspension, syrup or elixir. The vaccine can also be a preparation for parenteral, subcutaneous, intradermal, intramuscular or intravenous administration (e.g., injectable administration), such as a sterile suspension or emulsion. The vaccine can be incorporated into liposomes, microspheres or other polymer matrices. Liposomes can consist of phospholipids or other lipids, and can be nontoxic, physiologically acceptable and metabolizable carriers that are relatively simple to make and administer. Vaccine in a form suitable for direct or indirect electrotransport may be introduced (e.g., injected) using a needle-free injector into the tissue to be treated, usually by contacting the tissue surface with the injector so as to actuate delivery of a jet of the agent, with sufficient force to cause penetration of the vaccine into the tissue. For example, if the tissue to be treated is mucosa, skin or muscle, the agent is projected towards the mucosal or skin surface with sufficient force to cause the agent to penetrate through the stratum corneum and into dermal layers, or into underlying tissue and muscle, respectively. Needle-free injectors are well suited to deliver vaccines to all types of tissues, particularly to skin and mucosa. In some embodiments, a needle-free injector may be used to propel a liquid that contains the vaccine to the surface and into the subject's skin or mucosa. Representative examples of the various types of tissues that can be treated using the invention methods include pancreas, larynx, nasopharynx, hypopharynx, oropharynx, lip, throat, lung, heart, kidney, muscle, breast, colon, prostate, thymus, testis, skin, mucosal tissue, ovary, blood vessels, or any combination thereof. Mucosal vaccines may be, for example, liquid dosage forms, such as pharmaceutically acceptable emulsions, solutions, suspensions, syrups, and elixirs. Excipients suitable for such vaccines include, for example, inert diluents commonly used in the art, such as, water, saline, dextrose, glycerol, lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol. Excipients also can comprise various wetting, emulsifying, suspending, flavoring (e.g., sweetening), and/or perfuming agents. Oral mucosal vaccines also may, for example, be tableted or encapsulated for convenient administration. Such capsules or tablets can contain a controlled-release formulation. In the case of capsules, tablets, and pills, the dosage forms also can comprise buffering agents, such as sodium citrate, or magnesium or calcium carbonate or bicarbonate. Tablets and pills additionally can be prepared with enteric coatings. It is contemplated that the vaccine may be administered via the human, camel or avian patient's drinking water and/or food. “Parenteral administration” that is also contemplated by the invention includes subcutaneous injections, submucosal injections, intravenous injections, intramuscular injections, intrasternal injections, transcutaneous injections, and infusion. Injectable preparations (e.g., sterile injectable aqueous or oleaginous suspensions) can be formulated according to the known art using suitable excipients, such as vehicles, solvents, dispersing, wetting agents, emulsifying agents, and/or suspending agents. These typically include, for example, water, saline, dextrose, glycerol, ethanol, corn oil, cottonseed oil, peanut oil, sesame oil, benzyl alcohol, benzyl alcohol, 1,3-butanediol, Ringer's solution, isotonic sodium chloride solution, bland fixed oils (e.g., synthetic mono- or diglycerides), fatty acids (e.g., oleic acid), dimethyl acetamide, surfactants (e.g., ionic and non-ionic detergents), propylene glycol, and/or polyethylene glycols. Excipients also may include small amounts of other auxiliary substances, such as pH buffering agents. The vaccine may include one or more excipients that enhance the vaccinated patient's immune response (which may include an antibody response, cellular response, or both), thereby increasing the effectiveness of the vaccine. The adjuvant(s) may be a substance that has a direct (e.g., cytokine or Bacille Calmette-Guerin (“BCG”)) or indirect effect (liposomes) on cells of the patient's immune system. Examples of often suitable adjuvants include oils (e.g., mineral oils), metallic salts (e.g., aluminum hydroxide or aluminum phosphate), bacterial components (e.g., bacterial liposaccharides, Freund's adjuvants, and/or MDP), plant components (e.g., Quil A), and/or one or more substances that have a carrier effect (e.g., bentonite, latex particles, liposomes, and/or Quil A, ISCOM). As noted above, adjuvants also include, for example, CARBIGEN™ and carbopol. It should be recognized that this invention encompasses both vaccines that comprise an adjuvant(s), as well as vaccines that do not comprise any adjuvant. It is contemplated that the vaccine may be freeze-dried (or otherwise reduced in liquid volume) for storage, and then reconstituted in a liquid before or at the time of administration. Such reconstitution may be achieved using, for example, vaccine-grade water, or any appropriate solution. In brief, in manufacturing vaccine products it is important to have a good understanding of what factors can impact the safety, efficacy, and stability of the formulation all along the development path. The main phases in this process are: biophysical characterization of the antigen, evaluation of stabilizers, investigation of antigen interactions with adjuvants, evaluation of product contact materials such as sterile filter membranes, and monitoring stability both in real time and under accelerated conditions. Biophysical Characterization Phase refers to evaluation of the physical characteristics of the antigen, i.e., understanding how parameters such as pH, buffer species, and ionic strength impact the folded state of the antigen as well as the propensity of the antigen to aggregate. Knowing how characteristics of the formulation will impact physical stability of the antigen will aid selection of appropriate excipients during the development process. Empirical Phase refers to initiation of preformulation studies for the systematic development of a vaccine formulation. A logical place to start, is understanding how the physical stability of the antigen is impacted by changes in pH and temperature. The pH of the formulation can impact both the physical stability of the antigen, such as whether the antigen maintains the appropriate folding and if the antigen will aggregate, as well as the chemical stability of the antigen. The pH can impact the chemical degradation rate of many mechanisms of degradation such as hydrolysis, oxidation, and deamidation. The Empirical Phase Diagram offers a convenient way to display how the physical stability of an antigen is impacted with changes in pH and temperature. Generally, in this approach, characterization data are taken from various spectroscopic techniques such as second derivative UV/Vis, intrinsic fluorescence, extrinsic fluorescence, and circular dichroism are combined and transformed into data vectors to construct the empirical phase diagram. In addition to pH evaluation, the Empirical Phase Diagram approach can be utilized to determine the impact of other variables on antigen stability like buffer type and concentration, ionic strength, and impact of product contact material. Evaluation of Stabilizers refers to optimization of formulation parameters such as pH, ionic strength, and buffer species may not prove to be enough to stabilize an antigen for the typically desired 3-year shelf life of vaccine products. In this case stabilizing excipients need to be investigated for incorporation into the vaccine formulation. Evaluation of antigen stabilizers typically begins with investigation of generally regarded as safe (GRAS) excipients. By utilizing GRAS excipients, development may proceed more rapidly as regulatory concerns regarding safety of the formulation excipients will be lower. Since at the early stage of development the primary mechanism of antigen degradation may not be known it is important to evaluate excipients from various classes of stabilizers. Excipient screening such as monitoring of optical density or extrinsic fluorescence can be performed in a 96 well or more format to allow high-throughput screening of many excipients and excipient combinations at one time.

Correlation of Real-time and Accelerated Stability, refer to analysis of the stability of a formulation under extreme environmental conditions such as high temperatures. Correlation of accelerated stability with real-time data is valuable to support activities such as expiration dating and assessment of the impact of temperature excursions during shipment and storage of the vaccine for clinical trials. When initiating stability studies, it is important to understand potential mechanisms of antigen can degradation. In general, physical instability is associated with loss of protein structure and aggregation while common forms of chemical degradation are oxidation and deamidation. In addition to high temperature excursions, it is useful to determine the impact of other factors on formulation stability, such as exposure to environmental stresses such as cycles of freezing and thawing, extended exposure to light, and contact with various storage container materials. Adjuvants or Adjuvantation refers to enhancement of antigen immunogenicity. A side effect of vaccine antigens becoming more pure as purification technology has advanced is a reduction in the immunogenicity of the antigen. To retain antigen immunogenicity with more highly purified antigens, adjuvants can be incorporated into the vaccine formulation. Adjuvants interact with the immune system through various mechanisms thereby enhancing the immune response. Currently, the most utilized adjuvants in licensed products are aluminum salts and squalene-based oil-in-water emulsions. Aluminum-containing adjuvants, including aluminum hydroxide (AlO(OH) and aluminum phosphate (Al(OH)ₓ(PO₄)_{y} adjuvants, have a long history of use and an excellent safety profile. Sterile Filtration refers to prevention of microbial contamination of vaccines is an important part of producing a safe vaccine formulation. As vaccines are administered to infants, children, and adults who are generally healthy at the time of injection there is a high level of safety that must be ensured when manufacturing the vaccine product. Typically, this can be achieved through aseptic processing and sterile filtration of the vaccine formulation. However, formulations with aluminum-containing adjuvants cannot be sterilized by filtration due to the particle size of the adjuvant being greater than 0.2 μm. Materials used to prepare vaccines with aluminum-containing adjuvants must be sterilized prior to formulation and handled aseptically during the formulation and filling process. Sterile filter membranes are produced with various materials, typical membranes used in vaccine production are cellulose acetate, polyethersulfone and polyvinylidene fluoride.

A further aspect of the present disclosure relates to a method for treating, preventing, inhibiting, reducing, eliminating, protecting or delaying the onset of an infection or an infectious clinical condition caused by a virus in a subject in need thereof. In some embodiments, the therapeutic methods disclosed herein are applicable for any infection caused by any of the viruses of the Flaviviridae viruses, and specifically, of the Flavivirus genus, specifically, the Dengue virus, Zika virus, WNV, YFV, and any other virus discussed herein.

Thus, in some embodiments, at least for the case of infection with Dengue virus, the present disclosure provide therapy for any of the disclosed symptoms and associated pathologies. More specifically, common names for dengue fever include breakbone fever, vomiting and dandy fever; dengue hemorrhagic fever and dengue shock syndrome are the severe forms. The incubation period is 3 to 14 days, while the period of the illness is 3-7 days. Signs and symptoms may include severe headache; retro-orbital pain; muscle, joint, and bone pain; macular or maculopapular rash; and minor hemorrhagic manifestations, including petechiae, ecchymosis, purpura, epistaxis, bleeding gums, hematuria, or a positive tourniquet test result. In some embodiments, the method comprising the step of administering to the subject an effective amount of at least one polypeptide comprising an amino acid sequence of at least one reconstituted epitope of a viral envelope protein, and/or any polypeptide, domain or viral envelope protein comprising the reconstituted epitope, and/or any domain or viral envelope protein comprising at least one linker that replaces at least one loop or any part thereof or amino acid residue/s thereof in said viral protein. In some embodiments the viral envelope protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII. The reconstituted epitope comprises at least one linker and at least one fragment of the native envelope protein. Still further, in some alternative or additional embodiments the methods disclosed herein may involve the administration of any multimeric and/or multivalent antigen displaying platform of the polypeptides disclosed herein, any derivative, enantiomer, fusion protein, conjugate, polyvalent dendrimer thereof, any nucleic acid sequence encoding the same, and any combinations thereof, any compositions thereof and any vaccine thereof.

In some embodiments, the reconstituted epitope used by the methods disclosed herein may be any of the polypeptides defined by the present disclosure. Still further, in some alternative or additional embodiments, the therapeutic method disclosed herein may use any of the domain/s or viral envelope protein/s comprising at least one linker, as disclosed by the invention. In yet some further embodiments, the method of the present disclosure may use additionally or alternatively, any of the multimeric and/or multivalent antigen displaying platform as defined herein. Still further embodiments provide therapeutic methods that use any of the nucleic acid sequence/s defined by the present disclosure, and/or any of the composition/s, and/or vaccine/s is as defined by the present disclosure.

A further aspect of the present disclosure provides least one of, at least one polypeptide as defined by the present disclosure, at least one domain or viral envelope protein comprising at least one linker, as defined by the present disclosure, at least one multimeric and/or multivalent antigen displaying platform as defined by the present disclosure, at least one nucleic acid sequence as defined by the present disclosure, at least one composition as defined by the present disclosure, and at least one vaccine as defined by the present disclosure, for use in a method for treating, preventing, inhibiting, reducing, eliminating, protecting or delaying the onset of an infection or an infectious clinical condition caused by a virus in a subject in need thereof.

A further aspect of the present disclosure relates to a method of inducing an immune response against a virus of the Flaviviridae family in a subject in need thereof. In some embodiments, the method comprises administering to the subject an immunogenic effective amount of at least one polypeptide comprising an amino acid sequence of at least one of, at least one viral envelope protein, any polypeptide, domain or viral envelope protein comprising said reconstituted epitope, any domain or viral envelope protein comprising at least one linker that replaces at least one loop or any part thereof or amino acid residue/s thereof in said viral protein, any multimeric and/or multivalent antigen displaying platform thereof, and any combinations thereof, any derivative, enantiomer, fusion protein, conjugate, polyvalent dendrimer thereof, and any nucleic acid sequence encoding the same or any matrix, nano- or micro-particle thereof, or any composition or vaccine thereof. More specifically, the viral envelope protein, specifically, the native E protein as discussed in this aspect is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII. Still further, in some embodiments of the disclosed methods, the reconstituted epitope comprises at least one linker and at least one fragment of the native envelope protein. In some specific embodiments, the methods disclosed herein are particularly effective for eliciting a neutralizing antibody response to the virus in the treated subject, and/or for preventing Antibody Dependent Enhancement (ADE) in the subject. Antibody-dependent enhancement (ADE), also named immune enhancement or disease enhancement, is a phenomenon in which binding of a virus to suboptimal antibodies enhances its entry into host cells, followed by its replication. Antiviral antibodies promote viral infection of target immune cells by exploiting the phagocytic FcγR or complement pathway. After interaction with the virus the antibody binds Fc receptors (FcR) expressed on certain immune cells or some of the complement proteins. FcγR binds antibody via its fragment crystallizable region (Fc). Usually the process of phagocytosis is accompanied by the virus degradation, however, if the virus is not neutralized (either due to low affinity binding or targeting to a non-neutralizing epitope), antibody binding might result in a virus escape and therefore, enhanced infection. Thus, phagocytosis can cause viral replication, with the subsequent death of immune cells. The virus “deceives” the process of phagocytosis of immune cells and uses the host's antibodies as a Trojan horse. ADE may occur due to the non-neutralizing characteristic of the antibody, which bind viral epitopes other than those involved in a host cell attachment and entry. ADE may also happen due to the presence of sub-neutralizing concentrations of antibodies (binding to viral epitopes below the threshold for neutralization). In addition, ADE can be induced when the strength of antibody-antigen interaction is below the certain threshold. This phenomenon might lead to both increased virus infectivity and virulence. The viruses that can cause ADE frequently share some common features such as antigenic diversity, abilities to replicate and establish persistence in immune cells. ADE can occur during the development of a primary or secondary viral infection, as well as after vaccination with a subsequent virus challenge. It has been observed mainly with positive-strand RNA viruses. Among them are Flaviviruses such as Dengue virus, Yellow fever virus, Zika virus, Coronaviruses, including alpha- and betacoronaviruses, Orthomyxoviruses such as influenza, Retroviruses such as HIV, and Orthopneumoviruses such as RSV. The mechanism that involves phagocytosis of immune complexes via FcγRII/CD32 receptor is better understood compared to the complement receptor pathway. Cells that express this receptor are represented by monocytes, macrophages, some categories of dendritic cells and B-cells. ADE is mainly mediated by IgG antibodies, however, IgM along with complement, and IgA antibodies have also been shown to be trigger ADE. ADE may cause enhanced respiratory disease (ERD) and acute lung injury after respiratory virus infection with symptoms of monocytic infiltration and an excess of eosinophils in respiratory tract. ADE along with type 2 T helper cell-dependent mechanisms may contribute to a development of the vaccine associated disease enhancement (VADE), which is not limited to respiratory disease. Some vaccine candidates that targeted coronaviruses, RSV virus and Dengue virus elicited VADE, and were terminated from further development or became approved for use only for patients who have had those viruses before.

In more specific embodiments, the reconstituted epitope used by the methods disclosed herein may be any of the polypeptides defined by the present disclosure. Still further, in some alternative or additional embodiments, the therapeutic method disclosed herein may use any of the domain/s or viral envelope protein/s comprising at least one linker, as disclosed by the invention. In yet some further embodiments, the method of the present disclosure may use additionally or alternatively, any of the multimeric and/or multivalent antigen displaying platform as defined herein. Still further embodiments provide therapeutic methods that use any of the nucleic acid sequence/s defined by the present disclosure, and/or any of the composition/s, and/or vaccine/s is as defined by the present disclosure. Still further aspect provided herein relates to least one of, at least one polypeptide as defined by the present disclosure, at least one domain or viral envelope protein comprising at least one linker, as defined by the present disclosure, at least one multimeric and/or multivalent antigen displaying platform as defined by the present disclosure, at least one nucleic acid sequence as defined by the present disclosure, at least one composition as defined by the present disclosure, and at least one vaccine as defined by the present disclosure, for use in a method of inducing an immune response against a virus in a subject in need thereof. It should be noted that the term “induction of an immune response” is as defined herein before in connection with previous aspects of the present disclosure.

As discussed herein, the present disclosure provides reconstituted epitope that elicit the production of neutralizing antibodies. Binding of the neutralizing antibodies to their epitope in the viral E protein, perturbs the infection of the virus and thereby inhibits, reduces and attenuates infectivity of the virus. The reconstituted epitopes of the present disclosure are therefore referred to, in some embodiments of the invention, and functionally characterized as neutralizing reconstituted epitopes. It should be understood that the neutralizing antibodies, and therefore the neutralizing reconstituted epitopes of the present disclosure that elicit the production of such neutralizing antibodies, lead to inhibition, reduction, elimination, attenuation, retardation, decline, prevention or decrease of at least about 5%-99.9999%, specifically, at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 99.99%, 99.999%, 99.9999% or about 100%, of the infectivity of the virus and thereby prevents and reduces viral entry to the target host cell, reduces viral load and may further prevent or reduce at least one of morbidity, and any symptoms or conditions associated with the viral infection.

A further aspect of the present disclosure relates to a method for the preparation of a functional reconstituted epitope of a viral envelope protein. In some embodiments, the viral envelope protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII. More specifically, the method comprises the step of: first (a), screening a conformer library of epitopes of the viral envelope protein with at least one binding molecule. More specifically, the library comprising plurality of combinatorial display platforms or any display vehicles, each expressing a reconstituted epitope comprising at least one linker and at least one fragment of the native envelope protein. The next step (b), involves identifying and producing reconstituted epitope peptides which bind at least one of said binding molecules.

In some embodiments, of the methods disclosed herein, the binding molecule/s used for screening may be at least one of: (a) antibodies that neutralize the virus; (b) neutralizing antibodies of convalescent serum of at least one patient recovered from the virus infection; (c) the receptor for the virus or any fragments thereof; and (d) and any combinations of (a), (b) and (c). In some embodiments, neutralizing antibodies that may be applicable in the present method may be antibodies that compete directly or indirectly with receptor binding. In yet some further embodiments, the neutralizing antibodies used herein may be antibodies that lead to reduction in viral load. In some embodiments, the neutralizing antibody reduces and/or prevents at least one of morbidity, shock syndrome, hemorrhagic fever and any symptoms or conditions associated with the viral infection. Still further, in some embodiments the methods disclosed herein may be applicable for preparing at least one reconstituted epitope, specifically, neutralizing epitope for any virus that belongs to the Flaviviridae family. In more specific embodiments, the methods disclosed herein may be applicable for any virus that belongs to the Flavivirus genus. Still further, in some embodiments, the methods of the present disclosure may be applicable for a virus that may be any one of Dengue virus, Zika virus, yellow fever virus, West Nile virus, Tick-borne encephalitis virus, Japanese encephalitis virus or Tembusu virus.

In some specific embodiments, the method disclosed herein may be applicable for preparing a reconstituted epitope, specifically a reconstituted neutralizing epitope of a Dengue virus. In some embodiments, the native epitope comprises at least in part, at least one amino acid sequence of the DIII domain of the native E protein of the Dengue virus, and any fragments thereof. In more particular embodiments of this aspect, the native envelope protein comprises an amino acid sequence as denoted by SEQ ID NO: 96, 97, 98, 99, and any variants, mutants and homologs thereof. In some specific embodiments, the reconstituted epitope prepared by the methods discussed herein may comprise at least one fragment of the native E protein. In more specific embodiments, such reconstituted epitope comprises at least one of: (a), at least one amino acid sequence starting at any one of the amino acid residues 301, 296, 297, 298, 299, 300, 302, 303, 304, 305 and 306, and ending at any one of the amino acid residues 370, 365, 366, 367, 368, 369, 371, 372, 373, 374, 375. In some further additional or alternative embodiments, such reconstituted epitope may comprise (b), at least one amino acid sequence starting at any one of the amino acid residues 301, 296, 297, 298, 299, 300, 302, 303, 304, 305 and 306 and ending at any one of the amino acid residues 335, 329, 330, 331, 332, 333, 334, 336, 337, 338, 339, 340. Still further, in some further additional or alternative embodiments, such reconstituted epitope may comprise (c), at least one amino acid sequence starting at any one of the amino acid residues 356, 351, 352, 353, 354, 355, 357, 358, 359, 360, 361, and ending at any one of the amino acid residues 370, 365, 366, 367, 368, 369, 371, 372, 373, 374, 375.

In yet some further embodiments, the reconstituted epitope prepared by the methods disclosed herein may comprise an amino acid sequence of the native DIII domain of said E protein starting at any one of the amino acid residues 301, 296, 297, 298, 299, 300, 302, 303, 304, 305 and 306 and ending at any one of the amino acid residues 370, 365, 366, 367, 368, 369, 371, 372, 373, 374, 375. Still further, the native DIII domain comprises a loop comprising an amino acid sequence starting at any one of the amino acid residues 336, 333, 334, 335, 337, 338, 339, and ending at any one of the amino acid residues 355, 352, 353, 354, 356, 357, 358. It should be noted that in some embodiments, the at least one linker/s of the reconstituted epitope disclosed herein replaces such loop or any part thereof or amino acid residue/s thereof and any DIII domain fragment or amino acid residue/s thereof. In some embodiments, the reconstituted epitope prepared by the methods disclosed herein may comprise at least one linker and at least two fragments of the native E protein. In more specific embodiments, the at least two fragments of the reconstituted epitope prepared by the methods disclosed herein comprise:

As one fragment (a), the amino acid sequence of any one of: (i) residues M301 to I335 of the envelope protein; (ii) residues M301 to I335 of the envelope protein with at least one or two flanking amino acid residue/s; or (iii) any variant, mutant, parts or fragments of the amino acid sequence of residues M301 to I335 of the envelope protein. The second fragment (b), may comprise the amino acid sequence of any one of: (i) residues P356 to E370 of the envelope protein; (ii) residues P356 to E370 of the envelope protein with at least one or two flanking amino acid residue/s; or (iii) any variant, mutant, parts or fragments of the amino acid sequence of residues P356 to E370 of the envelope protein.

In more particular embodiments of the methods disclosed herein, the at least one linker of the reconstituted epitope is at least one of: (a), a bridging linker that bridges residue 335 with residue 356 of the of the envelope protein; (b), a linker attached to the N′ terminus of said at least one fragment; and (c), a linker attached to the C′ terminus of said at least one fragment.

In some embodiments, the methods disclosed herein may be applicable for preparing a reconstituted epitope, specifically, neutralizing epitope for a Zika virus.

In yet some further embodiments, the envelope protein of Zika virus comprises an amino acid sequence as denoted by SEQ ID NO: 100, or any variants, mutants and homologs thereof.

Still further, in some embodiments of the methods disclosed herein, the epitope comprises at least in part, at least one amino acid sequence of the DIII domain of the native E protein of said Zika virus, and any fragments thereof. In some embodiments, the DIII domain of the Zika virus E protein comprises residues 302 to 405.

In some embodiments, the reconstituted epitope prepared by the methods disclosed herein comprises at least one fragment of the native E protein. More specifically, such reconstituted epitope may comprise comprises at least one of:

(a), at least one amino acid sequence starting at any one of the amino acid residues 307, 302, 303, 304, 305, 306, 308, 309, 310, 311, and ending at any one of the amino acid residues 380, 375, 376, 377, 378, 379, 381, 382, 383, 384, 385; (b), at least one amino acid sequence starting at any one of the amino acid residues 307, 302, 303, 304, 305, 306, 308, 309, 310, 311, and ending at least one of the amino acid residues 340, 335, 336, 337, 338, 339, 341, 342, 343, 344, 345; (c), at least one amino acid sequence starting at any one of the amino acid residues 362, 357, 358, 359, 360, 361 and ending at least one of the amino acid residues 380, 375, 376, 377, 378, 379, 381, 382, 383, 384, 385. Still further, in some embodiments, the reconstituted epitope prepared by the methods disclosed herein comprises at least one linker and at least two fragments of the native E protein. More specifically, these at least two fragments comprise:

As one fragment (a), the amino acid sequence of any one of: (i) residues L307 to K340 of the envelope protein; (ii) residues L307 to K340 of the envelope protein with at least one or two flanking amino acid residue/s; or (iii) any variant, mutant, parts or fragments of the amino acid sequence of residues L307 to K340 of the envelope protein. As a second aspect (b), the amino acid sequence of any one of: (i) residues N362 to P380 of the envelope protein; (ii) residues N362 to P380 of the envelope protein with at least one or two flanking amino acid residue/s; or (iii) any variant, mutant, parts or fragments of the amino acid sequence of residues N362 to P380 of the envelope protein. In some embodiments, at least one linker of the reconstituted epitope prepared by the methods disclosed herein is a bridging linker that bridges residue 340 with residue 362 of the of the envelope protein.

Still further embodiments of the present disclosure provide methods for preparing a reconstituted epitope, specifically, neutralizing epitope of a Yellow Fever virus.

In more specific embodiments, the reconstituted epitope prepared by the methods disclosed herein is of an envelope protein of the YFV, that may comprise an amino acid sequence as denoted by SEQ ID NO: 101, and any variants, mutants and homologs thereof. Still further, in some embodiments, the reconstituted epitope prepared by the methods disclosed herein comprises at least in part, at least one amino acid sequence of the DIII domain of the native E protein of said Yellow Fever virus, and any fragments thereof. In more particular embodiments, the DIII domain of the Yellow Fever virus E protein comprises residues 292 to 392.

In yet some further embodiments, the reconstituted epitope prepared by the methods disclosed herein may comprise at least one fragment of the native E protein. Specifically, such fragment may comprise at least one of: (a), at least one amino acid sequence starting at any one of the amino acid residues 299, 294, 295, 296, 297, 298, 300, 301, 302, 303 or 304, and ending at any one of the amino acid residues 369, 364, 365, 366, 367, 368, 370, 371, 372, 373 or 374; (b), at least one amino acid sequence starting at any one of the amino acid residues 299, 294, 295, 296, 297, 298, 300, 301, 302, 303 or 304, and ending at any one of the amino acid residues 332, 327, 328, 329, 330 or 331; and (c), at least one amino acid sequence starting at any one of the amino acid residues 354, 349, 350, 351, 352, 353, 355, 356, 357, 358 or 359, and ending at any one of the amino acid residues 369, 364, 365, 366, 367, 368, 370, 371, 372, 373 or 374. In some embodiments, the reconstituted epitope prepared by the methods disclosed herein comprises an amino acid sequence of the native DIII domain of said Yellow Fever virus E protein starting at any one of the amino acid residues 299, 294, 295, 296, 297, 298, 300, 301, 302, 303 or 304, and ending at any one of the amino acid residues 369, 364, 365, 366, 367, 368, 370, 371, 372, 373 or 374. It should be noted that the native DIII domain comprises a loop comprising an amino acid sequence starting at any one of the amino acid residues 333, 328, 329, 330, 331, 332, 334, 335, 336, 337, or 338 and ending at any one of the amino acid residues 353, 348, 349, 350, 351, 352, 354, 355, 356, 357, or 358. Still further, the at least one linker/s of the reconstituted epitope prepared by the methods disclosed herein replaces this loop or any part thereof or amino acid residue/s thereof and any DIII domain fragment or amino acid residue/s thereof.

In more specific embodiments, the reconstituted epitope prepared by the methods disclosed herein may comprise at least one linker and at least two fragments of the native E protein. In more specific embodiments, such at least two fragments may comprise: as one fragment (a), the amino acid sequence of any one of: (i) residues 1299 to I332 of the envelope protein; (ii) residues I299 to I332 of the envelope protein with at least one or two flanking amino acid residue/s; or (iii) any variant, mutant, parts or fragments of the amino acid sequence of residues 1299 to I332 of the envelope protein; and as a second fragment (b), the amino acid sequence of any one of: (i) residues P354 to P369 of the envelope protein; (ii) residues P354 to P369 of the envelope protein with at least one or two flanking amino acid residue/s; or (iii) any variant, mutant, parts or fragments of the amino acid sequence of residues P354 to P369 of the envelope protein.

Still further, in some embodiments the at least one linker of the reconstituted epitope prepared by the methods disclosed herein is at least one of: (a), a bridging linker that bridges residue 332 with residue 354 of the of the Yellow Fever virus envelope protein; (b), a linker attached to the N′ terminus of said at least one fragment; and (c), a linker attached to the C′ terminus of said at least one fragment. In yet some further embodiments, the methods disclosed herein may be applicable for preparing at least one reconstituted epitope, specifically, neutralizing epitope for the West Nile virus (WNV). In some embodiments, the envelope protein of the WNV comprises an amino acid sequence as denoted by SEQ ID NO: 102, and any mutants, variants and homologs thereof. In yet some further embodiments, the reconstituted epitope prepared by the methods disclosed herein comprises at least in part, at least one amino acid sequence of the DIII domain of the native E protein of the West Nile virus, and any fragments thereof. In some embodiments, the DIII domain comprises residues 297 to 400 of the native E protein of West Nile virus.

Still further, in some embodiments, at least one fragment of the native E protein comprised within the reconstituted epitope prepared by the methods disclosed herein is at least one of: (a), at least one amino acid sequence starting at any one of residues 304, 299, 300, 301, 302, 303, 305, 306, 307, 308 or 309 and ending at any one of the amino acid residues 377, 372, 373, 374, 375, 376, 378, 379, 380, 381 or 382; (b), at least one amino acid sequence starting at any one of residues 304, 299, 300, 301, 302, 303, 305, 306, 307, 308 or 309 and ending at any one of the amino acid residues 338, 333, 334, 335, 336, 337, 339, 340, 341, 342, or 343; and (c), at least one amino acid sequence starting at any one of residues 360, 35, 356, 357, 358, 359, 361, 362, 363, 364 or 365 and ending at any one of the amino acid residues 377, 372, 373, 374, 375, 376, 378, 379, 380, 381 or 382. In yet some further embodiments, the reconstituted epitope prepared by the methods disclosed herein comprises an amino acid sequence of the native DIII domain of said West Nile virus E protein starting at any one of residues 304, 299, 300, 301, 302, 303, 305, 306, 307, 308 or 309 and ending at any one of the amino acid residues 377, 372, 373, 374, 375, 376, 378, 379, 380, 381 or 382. The native DIII domain comprises a loop comprising an amino acid sequence starting at any one of the amino acid residues 339, 334, 335, 336, 337, 338, 340, 341, 342, 343, or 344 and ending at any one of the amino acid residues 359, 354, 355, 356, 357, 358, 360, 361, 362, 363 or 364, and at least one of the linker/s of the reconstituted epitope prepared by the methods disclosed herein replaces this loop or any part thereof or amino acid residue/s thereof and any DIII domain fragment or amino acid residue/s thereof.

In some specific embodiments, the reconstituted epitope prepared by the methods disclosed herein reconstituted epitope comprises at least one linker and at least two fragments of the native E protein. More specifically, the at least two fragments comprise: as one fragment (a), the amino acid sequence of any one of: (i) residues V304 to V338 of the envelope protein; (ii) residues V304 to V338 of the envelope protein with at least one or two flanking amino acid residue/s; or (iii) any variant, mutant, parts or fragments of the amino acid sequence of residues V304 to V338 of the envelope protein; and as the second fragment (b), the amino acid sequence of any one of: (i) residues P360 to P377 of the envelope protein; (ii) residues P360 to P377 of the envelope protein with at least one or two flanking amino acid residue/s; or (iii) any variant, mutant, parts or fragments of the amino acid sequence of residues P360 to P377 of the envelope protein.

In some embodiment, the at least one linker of the reconstituted epitope prepared by the methods disclosed herein is at least one of: (a), a bridging linker that bridges residue 338 with residue 360 of the of the envelope protein; (b), a linker attached to the N′ terminus of said at least one fragment; and (c), a linker attached to the C′ terminus of said at least one fragment. In some specific embodiments, the at least one linker of the reconstituted epitope prepared by the methods disclosed herein may be an amino acid linker comprising 1 to 10 amino acid residues.

As indicated above, the reconstituted epitopes are in some embodiments used to constructed in conformer libraries containing combinatorial linkers. A “peptide library” is a collection of peptides, that may range from 2 to 100, specifically, from 3 to 90, 4 to 80, 5 to 70, 6 to 60, 7 to 50, 8 to 40, 9 to 30, 10 to 20 or 5 to 25 amino acid residues in length. The collection of peptides may be a random collection or it may be rationally designed based on the composition of the proteinaceous material of which the binding surface is a part.

The greater the number of different peptides in the library, the better. Preferably, in the case of a random peptide library, it may contain more than 106, 107, 108, 109 or more different peptides. In the peptide library, the peptides may be displayed by any means, such as, for example, peptides displayed on phage, a combinatorial library of synthetic peptides on any solid support, such as beads, plates etc. Phage display libraries of random peptides are well known in the art.

More specifically, as shown by the examples, the functional reconstituted epitopes of the invention were isolated by screening of a conformer peptide library, that enables the screening of library expressing genuine fragments of the native E protein inter-connected with a vast collection (millions) of combinatorial linkers, thus generating a “Conformer Library” where each linker enables the epitopes from the E protein fragments to assume a unique three-dimensional conformation. One of the factors contributing to the diversity of a phage-displayed peptide library is the method of library construction. When designing peptide-encoding oligonucleotide DNA to be inserted into the phage genome, the type of genetic code employed is of utmost importance because the observed frequency of amino acids displayed in the library is directly correlated to the number of codons encoding each amino acid. For instance, the standard 64-codon genetic code encodes each of the twenty amino acids and three stop codons with the number of codons per amino acid ranging from one (methionine, tryptophan) to six (leucine, serine, arginine). Amino acids encoded by higher numbers of codons have a greater chance of being incorporated into the library, while amino acids with fewer codons have a lesser chance. This discrepancy results in non-uniform amino acid frequencies and thus, limited peptide diversity. Ideally, a library would contain peptides with even 5% amino acid frequencies (one amino acid out of twenty) per each amino acid position within the peptides. To produce libraries with more even amino acid frequencies, phage-displayed peptide libraries are typically created using a reduced genetic code when designing the insert oligonucleotide DNA encoding the displayed peptides. The oligonucleotides are designed in the ‘NNK’ format, where N represents equal proportions of guanine, cytosine, thymine, and adenine nucleotides, and K represents equal proportions of thymine and guanine nucleotides. This method encodes all twenty amino acids and one stop codon, while smoothing the number of codons per amino acid to one, two, or three. Although the NNK method of library construction provides each amino acid a more equal opportunity of incorporation into the peptide library, the method still imparts some amino acid sequence bias because the amino acids are encoded by varying number codons. For example, in case of SupE suppression of the UAG stop codon Glutamine is over-represented.

A further aspect of the present disclosure relates to a method for producing an anti-viral vaccine comprising at least one reconstituted epitope of a viral envelope protein. More specifically, the viral envelop protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII. In some embodiments, the reconstituted epitope comprises at least one linker and at least one fragment of the native envelope protein. More specifically, the method may comprise the steps of: First step (a), involves preparing reconstituted functional epitope of an envelope protein of the virus by a method as defined by the preset disclosure. The second step (b), involves admixing at least one of the reconstituted functional epitope of an envelope protein of said virus or any derivative or enantiomer thereof, or any fusion protein, conjugate, or polyvalent dendrimer comprising the same with at least one adjuvant/s, carrier/s, excipient/s, auxiliaries, and/or diluent/s. A further aspect of the present disclosure relates to a method for the preparation, affinity selection and/or isolation of neutralizing antibodies that neutralize a virus. In some embodiments, the method comprising the steps of: First (a), contacting a serum or lymphocytes of at least one donor with an effective amount of reconstituted epitope of a viral envelope protein, any polypeptide, domain or viral envelope protein comprising the reconstituted epitope, any domain or viral envelope protein comprising at least one linker that replaces at least one loop or any part thereof or amino acid residue/s thereof in the viral protein. The viral envelope protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII. Still further, the reconstituted epitope comprises at least one linker and at least one fragment of the native envelope protein, or with any multimeric and/or multivalent antigen displaying platform thereof, any derivative, enantiomer, fusion protein, conjugate, polyvalent dendrimer thereof, and any combinations thereof. The second step (b), involves recovering the antibodies or at least one lymphocyte bound to the reconstituted epitope. In some embodiments, the antibody is a neutralizing antibody that reduces infectivity, and inhibits penetration of the virus to the target cell. In yet some further embodiments, such neutralizing antibody inhibits binding of at least one virus envelope protein to the cognate receptor in a target cell. Still further, in some embodiments, the neutralizing antibody may lead to reduction in viral load. In some embodiments, the neutralizing antibody reduces and/or prevents at least one of morbidity, shock syndrome, hemorrhagic fever and any symptoms or conditions associated with the viral infection.

More specifically, the neutralizing antibody as used herein, leads to an inhibition, reduction, elimination, attenuation, retardation, decline, prevention or decrease of at least about 5%-99.9999%, specifically, at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 99.99%, 99.999%, 99.9999% or about 100%, of the infectivity of the virus and thereby prevents and reduces viral entry to the target cell, reduces viral load and may further prevent or reduce at least one of morbidity, shock syndrome, hemorrhagic fever and any symptoms or conditions associated with the viral infection. In some particular embodiments, the method disclosed herein is applicable for any enveloped virus, for example, and virus of the Flaviviridae family. In yet some further embodiments, the method disclosed herein is configured for the production of monoclonal neutralizing antibodies that neutralize this virus. According to such embodiments, the method comprising the steps of: First (a), contacting lymphocytes of at least one donor with an effective amount of the reconstituted epitope, any multimeric and/or multivalent antigen displaying platform thereof, and any combinations thereof, any derivative, enantiomer, fusion protein, conjugate, polyvalent dendrimer thereof, associated directly or indirectly to a detectable moiety and/or any solid support. The second step (b), involves selection and single cell cloning of antibody producing lymphocyte bound to said reconstituted epitope.

In some alternative embodiments, the method disclosed herein may further comprise a step of cloning the nucleic acid sequence encoding the variable regions or any segments thereof of at least one of the heavy and the light chains of an antibody produced by these cells.

In yet some further alternative embodiments, the method disclosed herein is applicable for any enveloped virus, for example, and virus of the Flaviviridae family. In yet some further embodiments, the method disclosed herein is configured for the production of polyclonal neutralizing antibodies that neutralize the virus. According to these embodiments, the method comprising the steps of: (a), contacting serum of at least one donor or any immunoglobulin fraction thereof, with an effective amount of the reconstituted epitope of a viral envelope protein, any polypeptide, domain or viral envelope protein comprising the reconstituted epitope, any domain or viral envelope protein comprising at least one linker that replaces at least one loop or any part thereof or amino acid residue/s thereof in said viral protein, or with any multimeric and/or multivalent antigen displaying platform thereof, and any combinations thereof, any derivative, enantiomer, fusion protein, conjugate, polyvalent dendrimer thereof, associated directly or indirectly to a solid support and/or a detectable moiety. It should be noted that in some embodiments, the viral envelope protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII. Moreover, the reconstituted epitope comprises at least one linker and at least one fragment of the native envelope protein. The next step (b), involves recovering the antibodies bound to the reconstituted epitope immobilized to the solid support. The preset disclosure further provides a therapeutic passive vaccine comprising neutralizing antibodies that neutralize a virus. In some embodiments, such neutralizing antibodies were prepared by the method disclosed herein above.

A further aspect of the preset disclosure provides a method of screening for a compound that inhibits the penetration of a virus to at least one target cell, or the binding of at least one virus envelope protein to the cognate receptor in a target cell. The method comprising the step of: (a), contacting at least one candidate compound or a plurality of candidate compounds with an effective amount of at least one reconstituted epitope of a viral envelope protein, any polypeptide, domain or viral envelope protein comprising the reconstituted epitope, any domain or viral envelope protein comprising at least one linker that replaces at least one loop or any part thereof or amino acid residue/s thereof in said viral protein. The viral envelope protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII, wherein said reconstituted virus envelope protein comprises at least one linker and at least one fragment of the native envelope protein, any multimeric and/or multivalent antigen displaying platform thereof, and any combinations thereof, any derivative, enantiomer, fusion protein, conjugate, polyvalent dendrimer thereof, associated directly or indirectly to a solid support and/or a detectable moiety. The next step (b), involves recovering the candidate compound bound to the reconstituted epitope immobilized to said solid support and/or detectable moiety, thereby obtaining a compound that binds the virus envelope protein and inhibits or reduces viral infectivity. In some embodiments, the reconstituted epitope comprises at least one linker and at least one fragment of the native virus envelope protein. In some embodiments, the candidate compound is at least one of an antibody, an aptamer, a small molecule, a peptide and a nucleic acid molecule and any combinations thereof. In yet some further embodiments, the compound that inhibits viral entrance or penetration to a target cell, or binding of at least one virus envelope protein to the cognate receptor is an antibody. In yet some further embodiments, the plurality of the candidate antibody compounds is comprised in a phage display antibody library.

A further aspect of the present disclosure relates to a compound that inhibits or reduces viral infectivity, and/or inhibits binding of at least one virus envelope protein to the cognate receptor in a target cell. In some embodiments, the compound is prepared by the method as defined by the present disclosure. As indicated above, the present disclosure provides antibodies and methods for preparation of antibodies, as well as reconstituted epitopes that provide a powerful platform for eliciting the production of neutralizing antibodies.

The term ‘antibody’ as meant herein encompasses the whole antibodies as well as any antigen binding fragment (i.e., ‘antigen-binding portion) or single chain thereof. An antibody’ refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region (abbreviated herein as CH). Each light chain is comprised of a light chain variable region (abbreviated herein as VL) and a light chain constant region (abbreviated herein as CL). The VH and VL regions can be further subdivided into regions of hypervariability, termed ‘complementarity determining regions’ (CDRs), interspersed with regions that are more conserved, termed “framework regions” (FRs). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. The term ‘antigen-binding portion of an antibody, as used herein, refers to one or more fragments of an antibody that retain the ability to specifically bind to an antigen. It has been shown that the antigen-binding function of an antibody can be performed by fragments of a full-length antibody. Examples of binding fragments encompassed within the term ‘antigen-binding portion of an antibody include (i) a Fab fragment, a monovalent fragment consisting of the VL, VH, CL and Cm domains; (ii) a F(ab’)2 fragment, a bivalent fragment comprising two Fab fragments linked by a disulfide bridge at the hinge region; (iii) a Fd fragment consisting of the VH and Cm domains; (iv) a Fv fragment consisting of the VL and VH domains of a single arm of an antibody, (v) a dAb fragment, which consists of a VH domain; (vi) an isolated complementarity determining region (CDR), and (vii) a combination of two or more isolated CDRs which may optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules. Such single chain antibodies are also intended to be encompassed within the term “antigen-binding portion” of an antibody. A further example is binding-domain immunoglobulin fusion proteins comprising (i) a binding domain polypeptide that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region. The binding domain polypeptide can be a heavy chain variable region or a light chain variable region. These antibody fragments are obtained using conventional techniques known to those with skill in the art, and the fragments are screened for utility in the same manner as are intact antibodies.

More specifically, an ‘antibody fragment’ is a portion of an antibody such as F(ab′)2, F(ab)2, Fab′, Fab, and the like. Regardless of structure, an antibody fragment binds with the same antigen that is recognized by the intact antibody. For example, an anti-(polypeptide according to the present invention) monoclonal antibody fragment binds an epitope of a polypeptide according to the present invention. The term ‘antibody fragment’ also includes a synthetic or a genetically engineered polypeptide that binds to a specific antigen, such as polypeptides consisting of the light chain variable region, ‘Fv fragments consisting of the variable regions of the heavy and light chains, recombinant single chain polypeptide molecules in which light and heavy variable regions are connected by a peptide linker (scFv proteins), and minimal recognition units consisting of the amino acid residues that mimic the hypervariable region. Still further, an antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain. The antibody suitable for the invention may also be a bi-specific antibody (such as Bi-specific T-cell engagers-BiTEs) or a tri-specific antibody. The antibody suitable for the invention may also be a variable new antigen receptor antibody (V-NAR). VNARs are a class of small, immunoglobulin-like molecules from the shark immune system. Humanized versions of VNARs could be used. Still further, in some embodiments, the antibodies disclosed by the invention may be camelid single-domain antibodies (sdAbs), also known as heavy chain-only antibodies (HCAbs) or V_{H}Hs, and widely known as nanobodies. The term ‘epitope’ means a protein determinant capable of specific binding to an antibody. Epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents. Methods for preparing antibodies are known to the art. See, for example, Harlow & Lane (1988) Antibodies: a Laboratory Manual, Cold Spring Harbor Lab., Cold Spring Harbor, NY). Monoclonal antibodies may be prepared from a single B cell line taken from the spleen or lymph nodes of immunized animals, in particular rats or mice, by fusion with immortalized B cells under conditions which favor the growth of hybrid cells. The technique of generating monoclonal antibodies is described in many articles and textbooks, such as the above-noted Chapter 2 of Current Protocols in Immunology. Spleen or lymph node cells of these animals may be used in the same way as spleen or lymph node cells of protein-immunized animals, for the generation of monoclonal antibodies as described in Chapter 2 therein. The techniques used in generating monoclonal antibodies are further described in by Kohler and Milstein, Nature 256; 495-497, (1975), and in U.S. Pat. No. 4,376,110. Antibodies that are isolated from organisms other than humans, such as mice, rats, rabbits, cows, goats, horses, can be made more human-like through chimerization or humanization. It should be understood that the various antibodies and antigen-fragments thereof as defined herein, are applicable for any antibody (e.g., neutralizing antibodies used in the screening methods, diagnostic methods and kits or in the therapeutic vaccines) in any of the aspects of the invention.

In a further aspect thereof, the present disclosure provides a method for treating, inhibiting, reducing, eliminating, protecting or delaying the onset of a viral infection in a subject in need thereof. The method comprising the step of administering to the subject an effective amount of a therapeutic passive vaccine comprising neutralizing antibodies that neutralize the virus.

Still further aspect of the present disclosure provides an effective amount of the therapeutic passive vaccine comprising neutralizing antibodies that neutralize a virus or of a compound that inhibits or reduces viral infectivity and/or binding of at least one virus envelope protein to the cognate receptor in a target cell, for use in a method for treating, preventing, inhibiting, reducing, eliminating, protecting or delaying the onset of a viral infection in a subject in need thereof. In more specific embodiments, the passive vaccine is as defined by the present disclosure. A further aspect of the present invention relates to an antibody that specifically recognizes and binds at least one polypeptide comprising an amino acid sequence of at least one reconstituted epitope of a viral envelope protein, any polypeptide, domain or viral envelope protein comprising said reconstituted epitope, any domain or viral envelope protein comprising at least one linker that replaces at least one loop or any part thereof or amino acid residue/s thereof in the viral protein. Specifically, the viral envelope protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII. Still further, the reconstituted epitope comprises at least one linker and at least one fragment of the native envelope protein, any multimeric and/or multivalent antigen displaying platform thereof, and any combinations thereof, any derivative, enantiomer, fusion protein, conjugate, polyvalent dendrimer thereof.

In some embodiments, the antibody is a neutralizing antibody reduce infectivity that inhibits penetration of the virus to the target cell. In yet some further embodiments, such neutralizing antibody inhibits binding of at least one virus envelope protein to the cognate receptor in a target cell. Still further, in some embodiments, the neutralizing antibody may lead to reduction in viral load. In some embodiments, the neutralizing antibody reduces and/or prevents at least one of morbidity, shock syndrome, hemorrhagic fever and any symptoms or conditions associated with viral infection. In some embodiments, the antibody is prepared by the methods as described by the present disclosure. In some embodiments, the present disclosure encompasses any composition or passive vaccine comprising the disclosed neutralizing antibodies for therapeutic purposes.

Thus, the present disclosure provides in various aspects thereof therapeutic as well as prophylactic methods for subjects infected with viruses of the Flaviviridae, specifically, of the Flavivirus genus. The term “treatment” in accordance with disorders associated with infectious conditions may refer to one or more of the following: elimination, reducing or decreasing the intensity or frequency of disorders associated with said infectious condition. The treatment may be undertaken when disorders associated with said infection, incidence is beginning or may be a continuous administration, for example by administration every 1 to 14 days, to prevent or decrease occurrence of infectious condition in an individual prone to said condition. Such individual may be for example a subject having a compromised immune-system, in case of cancer patients undergoing chemotherapy or HIV infected subjects. Thus, the term “treatment” is also meant as prophylactic or ameliorating treatment.

The term “prophylaxis” refers to prevention or reduction the risk of occurrence of the biological or medical event, specifically, the occurrence or re occurrence of disorders associated with infectious disease, that is sought to be prevented in a tissue, a system, animal or human by a researcher, veterinarian, medical doctor or other clinician, and the term “prophylactically effective amount” is intended to mean that amount of a pharmaceutical composition that will achieve this goal. Thus, in particular embodiments, the methods of the invention are particularly effective in the prophylaxis, i.e., prevention of conditions associated with infectious viral disease. Thus, subjects administered with the compositions or vaccines of the invention are less likely to experience symptoms associated with said infectious condition that are also less likely to re-occur in a subject who has already experienced them in the past. The term “amelioration” as referred to herein, relates to a decrease in the symptoms, and improvement in a subject's condition brought about by the compositions and methods according to the invention, wherein said improvement may be manifested in the forms of inhibition of pathologic processes associated with any infectious viral disease, a significant reduction in their magnitude, or an improvement in a diseased subject physiological state. The term “inhibit” and all variations of this term is intended to encompass the restriction or prohibition of the progress and exacerbation of pathologic symptoms or a pathologic process progress, said pathologic process symptoms or process are associated with. The term “eliminate” relates to the substantial eradication or removal of the pathologic symptoms and possibly pathologic etiology, optionally, according to the methods of the invention described herein. The terms “delay”, “delaying the onset”, “retard” and all variations thereof are intended to encompass the slowing of the progress and/or exacerbation of an infectious disease, specifically, of viral infection and their symptoms slowing their progress, further exacerbation or development, so as to appear later than in the absence of the treatment according to the invention. As used herein, “disease”, “disorder”, “condition” and the like, as they relate to a subject's health, are used interchangeably and have meanings ascribed to each and all of such terms. The present invention relates to the treatment of subjects, or patients, in need thereof. By “patient” or “subject in need” it is meant any organism who may be affected by the above-mentioned conditions, and to whom the vaccinating and treatment methods herein described is desired, including humans, domestic and non-domestic mammals such as canine and feline subjects, bovine, simian, equine and murine subjects, rodents, domestic or wild birds, aquaculture, fish and exotic aquarium fish. It should be appreciated that the treated subject may be also any reptile or zoo animal and laboratory animals. More specifically, the composition/s and method/s of the invention are intended for mammals or avian subjects. By “mammalian subject” is meant any mammal for which the proposed therapy is desired, including any primate, specifically, human, camelids, bats, equine, canine, and feline subjects, most specifically humans. It should be noted that specifically in cases of non-human subjects, the method of the invention may be performed using administration via injection, drinking water, feed, spraying, oral gavage and directly into the digestive tract of subjects in need thereof. Single or multiple administrations of the compositions or vaccines of the invention are administered depending on the dosage and frequency as required and tolerated by the patient. In any event, the composition and/or vaccine should provide a sufficient quantity of the reconstituted epitope polypeptides of the invention to effectively vaccinate and thereby treat the patient. Preferably, the dosage is administered once but may be applied periodically until either a therapeutic result is achieved or until side effects warrant discontinuation of therapy. Generally, the dose is sufficient to treat or ameliorate symptoms or signs of disease without producing unacceptable toxicity to the patient. Still further, the reconstituted epitopes of the invention or any DIII, DII, DI domains and E proteins of the invention, or any variants thereof or any derivative, enantiomer, fusion protein or conjugate thereof or any compositions or kits thereof may be applied as a single daily dose or multiple daily doses, preferably, every 1 to 7 days. It is specifically contemplated that such application may be carried out once, twice, thrice, four times, five times or six times daily, or may be performed once daily, once every 2 days, once every 3 days, once every 4 days, once every 5 days, once every 6 days, once every week, two weeks, three weeks, four weeks or even a month. The application of the reconstituted epitope polypeptides of the invention, or any DIII, DII, DI domains and E proteins of the invention, or any variants thereof or any derivative, enantiomer, fusion protein or conjugate thereof or any compositions or kits thereof may last up to a day, two days, three days, four days, five days, six days, a week, two weeks, three weeks, four weeks, a month, two months three months or even more. Specifically, application may last from one day to one month. Most specifically, application may last from one day to 7 days.

The invention thus provides methods for inhibiting and preventing viral infection and treating an ameliorating any pathologic condition associated therewith, in a subject. It should be appreciated that such method may results in an inhibition, reduction, elimination, attenuation, retardation, decline, prevention or decrease of at least about 5%-99.9999%, about 10%-90%, about 15%-85%, about 20%-80%, about 25%-75%, about 30%-70%, about 35%-65%, about 40%-60% or about 45%-55%, and more specifically may be by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 99.99%, 99.999%, 99.9999% or about 100%, of the viral infections, or infectious condition associated therewith as discussed above. It should be appreciated that the therapeutic methods and administration modes discussed herein are applicable for any of the therapeutic methods provided by the invention. Specifically, herein after in connection with other aspects of the invention. In more specific embodiments, any of the therapeutic definitions defined herein are applicable for any therapeutic aspects of the invention that use any passive vaccines provided by the invention, and any compounds that prevent or inhibit binding of the virus to its cognate receptor, and any antibody produced and/or isolated by the invention and any composition thereof.

A further aspect of the present disclosure relates to a diagnostic method for the detection of a viral infection of at least one virus of the Flaviviridae family, in a mammalian subject. The diagnostic method discussed herein may comprise the steps of: (a) contacting at least one biological sample of the subject or any preparation thereof, with at least one of (i) at least one reconstituted epitope of a viral envelope protein, any polypeptide, domain or viral envelope protein comprising said reconstituted epitope, any domain or viral envelope protein comprising at least one linker that replaces at least one loop or any part thereof or amino acid residue/s thereof in said viral protein. The reconstituted epitope is associated directly or indirectly to a solid support and/or a detectable moiety. Alternatively, or additionally, the sample may be contacted with (ii), antibodies specific for the reconstituted virus envelope protein associated directly or indirectly to a solid support and/or a detectable moiety. Alternatively, or additionally, the sample may be contacted with (iii) any virus envelope protein binding molecule associated directly or indirectly to a solid support and/or to a detectable moiety. The next step (b), involves determining that the subject is infected with the virus of the Flaviviridae family, if said detectable moiety is detected in said sample.

In some embodiments, the envelope protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII, and wherein the reconstituted epitope comprises at least one linker and at least one fragment of the native envelope protein. In some embodiments, the reconstituted epitope of a virus envelope protein is as defined by the invention.

In yet some further embodiments, the antibody specific for the reconstituted epitope is any one of: (a), antibodies prepared by the method as disclosed herein; and (c) antibodies or envelope protein binding compounds prepared by screening a phage display antibody library with the reconstituted virus envelope, as defined by the present disclosure.

A further aspect of the present disclosure relates to a diagnostic kit comprising at least one of: (a) at least one reconstituted epitope of a viral envelope protein, any polypeptide, domain or viral envelope protein comprising the reconstituted epitope, any domain or viral envelope protein comprising at least one linker that replaces at least one loop or any part thereof or amino acid residue/s thereof in said viral protein, wherein said viral envelope protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII. The reconstituted epitope comprises at least one linker and at least one fragment of the native envelope protein, any multimeric and/or multivalent antigen displaying platform thereof, and any combinations thereof, associated directly or indirectly to a solid support and/or a detectable moiety; (b) antibodies specific for the reconstituted epitope associated directly or indirectly to a solid support and/or a detectable moiety; and (c) at least one reconstituted epitope binding molecule associated directly or indirectly to a solid support and/or a detectable moiety.

In some embodiments, the polypeptide is as defined by the invention, the multimeric and/or multivalent antigen displaying platform is as defined by the present disclosure. In some further embodiments, the detectable moiety, affinity moiety or tag associated directly or indirectly with the reconstituted functional epitope of the invention, may refer to any chemical moiety that can be used to provide a detectable signal, and or attachment or affinity to a solid support, and that can be attached to an encoding nucleic acid sequence or protein via a covalent bond or noncovalent interaction (e.g., through ionic or hydrogen bonding, or via immobilization, adsorption, or the like). Labels generally provide signals detectable by at least one of fluorescence, chemiluminescence, radioactivity, colorimetry, mass spectrometry, X-ray diffraction or absorption, magnetism, enzymatic activity, electrochemical active compounds, or the like. In some specific embodiments, the detectable moiety, affinity moiety or tag useful by the invention may be at least one of conductive, electrochemical, fluorescent, chemiluminescent, enzymatic, radioactive, magnetic, metal, and colorimetric label, or any combinations thereof. Examples of labels useful in connection with the invention, include, but are not limited to at least one of haptens, enzymes, enzyme substrates, coenzymes, enzyme inhibitors, fluorophores, quenchers, chromophores, magnetic particles or beads, redox sensitive moieties (e.g., electrochemically active moieties), luminescent markers, radioisotopes (including radionucleotides), conductive materials, or electrochemical materials that in some embodiments may be suitable for electrochemical detection, specifically, nano- and micro-sized materials, such as gold nanoparticles (GNPs), latex, carbon nanotubes (CNTs), graphene (GR), magnetic particles (MBs), quantum dots (QDs) and conductive polymers, biobarcodes and members of binding pairs. More specific examples include at least one of fluorescein, phycobiliprotein, tetraethyl rhodamine, and beta-galactosidase. Binding pairs may include biotin/Strepavidin, biotin/avidin, biotin/neutravidin, biotin/captavidin, GST/glutathione, maltose binding protein/maltose, calmodulin binding protein/calmodulin, enzyme-enzyme substrate, receptor-ligand binding pairs, and analogs and mutants of the binding pairs. It should be appreciated that the use of tags for labeling directly or indirectly the reconstituted functional epitope of the invention, or any scaffold displaying such reconstituted functional epitope is also encompassed by the invention. Non-limiting examples for such tag may include His-tag, Flag, HA, myc and the like. It should be further appreciated that the detectable moieties disclosed herein are applicable for any aspect of the invention. The invention further encompasses the use of any of the labeled or tagged reconstituted functional epitope, DIII, DII, DI domains or E proteins comprising a linker that replaces the loop thereof, in any of the aspects of the invention, specifically, in any of the methods for selecting antibodies or compounds that bind the neutralizing epitope and prevents and/or reduce viral penetration and infectivity. Particularly for methods for identifying neutralizing antibodies, as well as for the diagnostic methods and kits disclosed by the invention herein after.

All definitions, as defined and used herein, should be understood to control over dictionary definitions, definitions in documents incorporated by reference, and/or ordinary meanings of the defined terms. The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to ±10%. The indefinite articles “a” and “an,” as used herein in the specification and in the claims, unless clearly indicated to the contrary, should be understood to mean “at least one.” It must be noted that, as used in this specification and the appended claims, the singular forms “a”, “an” and “the” include plural referents unless the content clearly dictates otherwise. The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. It should also be understood that, unless clearly indicated to the contrary, in any methods claimed herein that include more than one step or act, the order of the steps or acts of the method is not necessarily limited to the order in which the steps or acts of the method are recited.

Throughout this specification and the Examples and claims which follow, all transitional phrases such as “comprising,” “including,” “carrying,” “having,” “containing,” “involving,” “holding,” “composed of,” and the like are to be understood to be open-ended, i.e., to mean including but not limited to. Specifically, it should understood to imply the inclusion of a stated integer or step or group of integers or steps but not the exclusion of any other integer or step or group of integers or steps. Only the transitional phrases “consisting of” and “consisting essentially of” shall be closed or semi-closed transitional phrases, respectively, as set forth in the United States Patent Office Manual of Patent Examining Procedures. More specifically, the terms “comprises”, “comprising”, “includes”, “including”, “having” and their conjugates mean “including but not limited to”. The term “consisting of means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements. Various embodiments and aspects of the present invention as delineated herein above and as claimed in the claims section below find experimental support in the following examples. Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof. The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

### Experimental Procedures

### Materials

### Bacterial Strains

TABLE 1
Bacterial strains used in the present disclosure.

| Strain | Genotype |
|---|---|
| DH5αF′ | F′ endA1 hsdR17 (rK⁻ mK⁺) supE44 thi-1 recA1 gyrA |
| | (Nal^{R}) relA1 Δ(lacIZYA-argF) U169 deoR |
| | (φ80dlacΔ(lacZ)M15) |
| DH5αF⁻ | F− endA1 hsdR17 (rK⁻ mK⁺) supE44 thi-1 recA1 gyrA |
| | (Nal^{R}) relA1 Δ(lacIZYA-argF) U169 deoR |
| | (φ80dlacΔ(lacZ)M15) |
| ER2738 | [F′proA+B+ lacIq Δ(lacZ)M15 zzf::Tn10 (tetr)] fhuA2 |
| | glnVΔ(lac-proAB) thi-1Δ(hsdS-mcrB)5 |
| TG1 | F′ traD36 proAB lacIqZ ΔM15] supE thi-1 Δ(lac-proAB) |
| | Δ(mcrB-hsdSM)5(rK− mK−) |
| Rosetta | F− dcm ompT hsdSn (rB− mB−) gal λ(DE3) |
| (DE3) | |

### Vectors

The fth1 vector was designed and constructed in our laboratory by David Enshell-Seijffers as described in detail in Enshell-Seijffers, et al., (Nucleic acids research Nucleic acids research 29.10 (2001). This vector enables the display of peptides at the N-terminus of the major coat protein, pVIII of the phage. This vector was further modified by Larisa Smelyanski to enable the display of protein fragments at the N-terminus of the pIII protein (Smelyanski and Gershoni, Virol J. (2011) 8:495).
pMALc vector is a commercial expression system (Maina et al., Gene 74, no. 2 (1988): 365-373) and was modified by the insertion of Aval restriction sites in 131-loop or in the carboxy-terminus of MBP.
pET30a-N6xHis-GST vector for cloning.
I53-50A vector is a computationally designed component that displays 20 copies of a trimeric viral protein which forms a nanoparticle through the self-assembly of 20 trimeric I53-50A and 12 pentameric I53-50B building blocks, and induces potent neutralizing antibody responses (King et al., Nature. 510 (7503): 103-108 (2014); Bale et al., Science 353.6297 (2016): 389-394.; Marcandalli et al., Cell 176.6 (2019): 1420-1431). The system was developed and kindly provided by Prof. Neil King from University of Washington.

### Restriction Enzymes

All restriction enzymes and buffers were purchased from New England Biolabs (NEB, Beverly, MA) and were used according to the manufacturer's instructions.

### Antibodies

TABLE 2
Antibodies used in this study.

| Antibody | Source | Description |
|---|---|---|
| Anti-M13 | Produced in-house | Anti-phage. Polyclonal. |
| mAb | | Raised in rabbits against the |
| | | fd phage. |
| | | Working dilution: |
| | | Dot-blot: 1:10,000 |
| | | ELISA: 1:5,000 |
| Anti-MBP | Produced in-house | Anti-MBP. Polyclonal. |
| | | Raised in rabbits against |
| | | MBP constructs. |
| | | Working dilution: |
| | | Dot-blot: 1:5,000 |
| | | ELISA: 1:2,500 |
| 4E5A | Kindly provided by the Toronto | Murine-derived DENV |
| 1A1D | Recombinant Antibody Centre | neutralizing antibody, |
| | (TRAC), University of Toronto | recognition of DIII |
| 4G2 | The Native Antigen Company | Rabbit anti Flavivirus |
| | | Envelope protein |

### Oligonucleotides

The linkers used in the present study are disclosed in Table 3.

TABLE 3
The oligonucleotides used in this study. All the oligonucleotides which
were used in this research were purchased from Integrated DNA
Technologies (IDT). “F” stands for forward primer (sense strand),
“R” stands for reverse primer (antisense strand). N = A/T/C/G,
K = T/G and M = A/C.

| No./ | | |
|---|---|---|
| SEQ ID NO: | Name | Sequence (5′-3′) |
| 1 | Native | ATGTGCACCGGTTCTTTCAAACTGGAAAAAGAAGTTGCG |
| SEQ ID | DIII- | GAAACCCAGCACGGTACCGTTCTGGTTCAGGTTAAATAC |
| NO: 103 | with | GAAGGTACCGACGCGCCGTGCAAAATCCCGTTCTCTACC |
| | loop | CAGGACGAAAAAGGTGTTACCCAGAACGGTCGTCTGAT |
| | | CACCGCGAACCCGATCGTTACCGACAAAGAAAAACCGG |
| | | TTAACATCGAAGCGGAA |
| 2 | Loop- | ATGTGCACCGGTTCTTTCAAACTGGAAAAAGAAGTTGCG |
| SEQ ID | less | GAAACCCAGCACGGTACCGTTCTGGTTCAGGTTAAATAC |
| NO: 104 | DIII | GAAGGTACCGACGCGCCGTGCAAAATCCCGATCGTTACC |
| | | GACAAAGAAAAACCGGTTAACATCGAAGCGGAA |
| 3 | DIII- | ATGTGCACCGGTTCTTTCAAACTGGAAAAAGAAGTTGCG |
| SEQ ID | 1NNK | GAAACCCAGCACGGTACCGTTCTGGTTCAGGTTAAATAC |
| NO: 105 | | GAAGGTACCGACGCGCCGTGCAAAATCNNKCCGATCGT |
| | | TACCGACAAAGAAAAACCGGTTAACATCGAAGCGGAA |
| 4 | DIII- | ATGTGCACCGGTTCTTTCAAACTGGAAAAAGAAGTTGCG |
| SEQ ID | 2NNK | GAAACCCAGCACGGTACCGTTCTGGTTCAGGTTAAATAC |
| NO: 106 | | GAAGGTACCGACGCGCCGTGCAAAATCNNKNNKCCGAT |
| | | CGTTACCGACAAAGAAAAACCGGTTAACATCGAAGCGG |
| | | AA |
| 5 | DIII- | ATGTGCACCGGTTCTTTCAAACTGGAAAAAGAAGTTGCG |
| SEQ ID | 3NNK | GAAACCCAGCACGGTACCGTTCTGGTTCAGGTTAAATAC |
| NO: 107 | | GAAGGTACCGACGCGCCGTGCAAAATCNNKNNKNNKCC |
| | | GATCGTTACCGACAAAGAAAAACCGGTTAACATCGAAG |
| | | CGGAA |
| 6 | DIII- | ATGTGCACCGGTTCTTTCAAACTGGAAAAAGAAGTTGCG |
| SEQ ID | 4NNK | GAAACCCAGCACGGTACCGTTCTGGTTCAGGTTAAATAC |
| NO: 108 | | GAAGGTACCGACGCGCCGTGCAAAATCNNKNNKNNKNN |
| | | KCCGATCGTTACCGACAAAGAAAAACCGGTTAACATCG |
| | | AAGCGGAA |
| 7 | DIII- | ATGTGCACCGGTTCTTTCAAACTGGAAAAAGAAGTTGCG |
| SEQ ID | 5NNK | GAAACCCAGCACGGTACCGTTCTGGTTCAGGTTAAATAC |
| NO: 109 | | GAAGGTACCGACGCGCCGTGCAAAATCNNKNNKNNKNN |
| | | KNNKCCGATCGTTACCGACAAAGAAAAACCGGTTAACA |
| | | TCGAAGCGGAA |
| 8 | DIII_ | TTCCGCTTCGATGTTAACCG |
| SEQ ID | complementary | |
| NO: 110 | | |
| 9 | 0NNK_F | ACTCCGCTCCAGAGCTCTGGATGTGCACCGGTTCTTTC |
| SEQ ID | | |
| NO: 111 | | |
| 10 | 1NNK_F | ACTCCGCTCCAGAGCTCTGGNNKATGTGCACCGGTTCTT |
| SEQ ID | | TC |
| NO: 112 | | |
| 11 | 2NNK_F | ACTCCGCTCCAGAGCTCTGGNNKNNKATGTGCACCGGTT |
| SEQ ID | | CTTTC |
| NO: 113 | | |
| 12 | 3NNK_F | ACTCCGCTCCAGAGCTCTGGNNKNNKNNKATGTGCACC |
| SEQ ID | | GGTTCTTTC |
| NO: 114 | | |
| 13 | 0NNK_R | ACAGTTTCCCAGACGTCTGGTTCCGCTTCGATGTTAACC |
| SEQ ID | | G |
| NO: 115 | | |
| 14 | 1NNK_R | ACAGTTTCCCAGACGTCTGGMNNTTCCGCTTCGATGTTA |
| SEQ ID | | ACCG |
| NO: 116 | | |
| 15 | 2NNK_R | ACAGTTTCCCAGACGTCTGGMNNMNNTTCCGCTTCGAT |
| SEQ ID | | GTTAACCG |
| NO: 117 | | |
| 16 | 3NNK_R | ACAGTTTCCCAGACGTCTGGMNNMNNMNNTTCCGCTTC |
| SEQ ID | | GATGTTAACCG |
| NO: 118 | | |
| 17 | MBP_ | TTCCAGGGCATTTCACATATGGGTATGTGCACCGGTTCTT |
| SEQ ID | A11_F | TC |
| NO: 119 | | |
| 18 | MBP_ | GTTTTATTTGAAGCTTATTTATTCCGCTTCGATGTTAACCG |
| SEQ ID | A11_R | G |
| NO: 120 | | |
| 19 | MBP_ | TTCCAGGGCATTTCACATATGACTGGTTTGATGTGCACCG |
| SEQ ID | C6_F | GT |
| NO: 121 | | |
| 20 | MBP_ | GTTTTATTTGAAGCTTATTTACTGAGTAGTTTCCGCTTCG |
| SEQ ID | C6_R | AT |
| NO: 122 | | |
| 21 | MBP_ | TTCCAGGGCATTTCACATATGACGAGTCGTATGTGCACCG |
| SEQ ID | F8_F | GT |
| NO: 123 | | |
| 22 | MBP_ | GTTTTATTTGAAGCTTATTTAAAGAGTTTCCGCTTCGATG |
| SEQ ID | F8_R | TT |
| NO: 124 | | |
| 23 | MBP_ | TTCCAGGGCATTTCACATATGCCTGCTATGTGCACCGGTT |
| SEQ ID | H1_F | CT |
| NO: 125 | | |
| 24 | MBP_ | GTTTTATTTGAAGCTTATTTACAGCAGTTCCGCTTCGATG |
| SEQ ID | H1_R | TT |
| NO: 126 | | |
| 25 | 20mer | CCCTCATAGTTAGCGTAACGA |
| SEQ ID | | |
| NO: 127 | | |
| 26 | 21mer | AATGTGTGGAATTGTGAGCGG |
| SEQ ID | | |
| NO: 54 | | |
| SEQ ID | Primer | TAAGAAGGAGATATACATATGGGTATGTGCACCGGTTC |
| NO: 128 | I53_A11_F | T |
| SEQ ID | Primer | GAATAGCTCCTCCATCTTCATTTCCGCTTCGATGTTAAC |
| NO: 129 | I53_A11_R | |
| SEQ ID | Primer | GAAGGAGATATACATATGACGAGTCGTATGTGCACC |
| NO: 130 | I53_F8_F | |
| SEQ ID | Primer | GAATAGCTCCTCCATCTTCATAAGAGTTTCCGCTTCGAT |
| NO: 131 | I53_F8_R | |
| SEQ ID | Primer | GAAGGAGATATACATATGCCTGCTATGTGCACCGGT |
| NO: 53 | I53_H1_F | |
| SEQ ID | Primer | TAGCTCCTCCATCTTCATCAGCAGTTCCGCTTC |
| NO: 69 | I53_H1_R | |

### Solutions and Media

All solutions and media were dissolved in double distilled water (DDW) unless indicated otherwise. Percent denotes weight/volume (w/v) unless indicated otherwise.

### Growth Media

LB (Lysogeny Broth): 1% Bacto tryptone, 0.5% Bacto yeast extract, 1% NaCl.

2×YT (Tryptone Yeast): 1.6% Bacto tryptone, 1% Bacto yeast extract, 0.5% NaCl.

TB (Terrific Broth): 1.2% Bacto tryptone, 2.4% Bacto yeast extract, 1:250 glycerol 1:10, potassium phosphate buffer pH=7.

SOB: 20 g Bacto tryptone, 5 gr Bacto yeast extract, 0.58 gr NaCl, 0.19 gr KCl.

SOC: per 100 ml SOB: 1 ml 2M glucose, 0.5 ml 2M MgSO₄, 0.5 ml 2M MgCl₂.

### Antibiotics

Tetracycline: 20 μg/ml (working concentration), Ampicillin 100 μg/ml (working concentration),

Kanamycin 50 μg/ml (working concentration), Carbenicillin 50 μg/ml (working concentration).

### Buffers

TBS (Tris-buffered saline) ×10: 0.5M Trizma base, 1.5M NaCl, pH adjusted to pH-7.5 with fuming HCl.

TBST: 0.01-0.5% Tween20, dissolved in TBS×1.

TBS-BSA (Bovine serum albumin): 3% BSA dissolved in TBS×1.

PBS (Potassium phosphate buffer) 0.166M KH₂PO₄, 0.716M K₂HPO₄, pH=7.

PBST: 0.01-0.5% Tween20, dissolved in PBS×1.

Elution buffer: 0.1M HCl, 1 mg/ml BSA, pH adjusted to pH=2.2 with solid glycine.

Neutralizing buffer: 1M Trizma base, pH adjusted to pH=9.1 with concentrated HCl.

PEG/NaCl: 3.3M NaCl, 33% polyethylene glycol 6,000.

Blocking solution: 5% skim milk, 20% horse serum dissolved in TBS×1.

ECL solution: 0.1M Tris-HCl (pH=8.9), 0.5% Luminol, 0.22% p-Coumaric acid.

### Solutions and Buffers for Agarose Gels

TAE ×50: 40 mM Tris-acetate, 1 mM EDTA (pH=8).

Loading buffer ×6: 0.25% bromophenol blue, 40% glycerol.

### Solutions and Buffers for Protein Electrophoresis

Tris-Glycine SDS (TGS): 25 mM Tris base.

Running Buffer ×10: 192 Mm Glycine, 0.1% SDS.

Sample buffer: 25 mM Tris-HCl (pH=6.4), 0.1% bromophenol blue, 10% glycerol, 2% SDS, 3% β-mercaptoethanol (optional).

### Experimental Procedures

### DNA Preparation and Sanger Sequencing

DNA preparation-Double-stranded DNA (dsDNA) was extracted using the Miniprep® Spin Miniprep Kit (Qiagen GmbH, Germany) for small volumes of bacterial culture (<10 ml). For extraction from larger volumes (>100 ml) of bacterial culture the NucleoBond® Xtra Midi/Maxi (MN, Düren, Germany) was used.

DNA Primers used for Sanger sequencing of constructs presented on fth1 vector, are 20mer and 21mer (detailed in Table 3).

### DNA Gel Electrophoresis

Agarose gel—Gel electrophoresis for DNA fragments analysis was carried out using 1-2% (depending on the size of the analyzed DNA fragments) agarose gels prepared in TAE×1 buffer.

MetaPhor agarose gel—1750 mg of metaphor agarose was dissolved in 50 ml TAE×1 buffer in a beaker. After boiling, the solution was cooled to ˜55° C., and 2.5 μl of ethidium bromide (10 mg/ml) were added. The gel was poured and allowed to cool to room temperature before use.

### Gel Extraction and Cleanup of PCR Products

HiYield™ Gel/PCR DNA Fragments Extraction Kit (RBC Bioscience, New Taipei City, Taiwan) was used to extract and clean up PCR samples, enzymatic reactions and DNA fragments that were cut out from agarose gel. Agencourt AMPure XP PCR Purification kit (Beckman Coulter Life Sciences, Indianapolis, United States) was used for purification and clean-up of PCR products.

### Construction of Conformer Library EFI (Residues M301-E370 of DIII)

Preparation of fth1-p3 for cloning: fth1-p3 vector was transformed to DH5a (F+) E. coli bacteria for growth in LB solution for 16 h. After growth, plasmid DNA purification was conducted using NucleoBond® Xtra Maxi kit.

The vector was then digested with BstXI in order to cut it in both BstXI sites of pIII, to allow insert incorporation. The cut vector was run on agarose gel 1% and extracted using HiYield™ Gel/PCR DNA Fragments Extraction Kit.

Preparation of inserts: oligos #1 to #2 (native loop and loop-less) were ordered as double stranded gBlocks, whereas oligos #3 to #7 are single stranded (Table 3).

The antisense 5′-oligonucleotide #8 was complementary to 20 bases at the 3′ of the sense 5′-oligonucleotides (oligonucleotides #3 to #7, Table 3). The two oligonucleotides were annealed to each other (200 pmol of each oligonucleotide were mixed and annealed by incubating of the mix for 20 min at 80° C., followed by slow cooling down at room temperature). The overhangs were filled in by a Klenow (NEB) reaction for 1 hr at 37° C. which was then inactivated at 75° C. for 20 min. Ethanol precipitation was conducted overnight at −80° C. and the resulting dsDNA was resuspended in 100 μl of ultra pure H2O.

For the PCR reaction, 5′-sense and 3′-antisense combinatorial PCR primers which contained 0, 1, 2 or 3 NNKs (#9 to #16, Table 3), were mixed and used for PCR of 7 different templates of the dsDNA described above. The primers contain 20 bases corresponding to the fth1 sequence, 0-3 NNK in the middle and 18-20 bases corresponding to the insert, were added to each side of the dsDNA fragment. The resulting 150-216 bp PCR fragments were separated using 2% agarose gel and purified using Agencourt AMPure XP PCR Purification kit.

Library construction (Gibson Assembly): The purified dsDNA fragments were cloned via the Gibson Assembly reaction into the BstXI (pIII)-digested fth1 linearized vector (Gibson et al., Nature methods 6, no. 5 (2009): 343-345; Thomas, Maynard and Gill, Nature Methods 12.11 (2015): i-ii.). The Gibson Assembly reaction mix consisted of the following: 80 ng digested linearized fth1 vector, 20-40 ng DNA insert and 10 μl Gibson Assembly Master Mix (NEB). Volumes were adjusted to 20 μl with DDW. The vials were incubated for 20 min at 50° C. followed by transformation via heat shock into DH5a ER2738 competent E. coli bacteria cells. The bacteria were then plated on LBlagar plates containing tetracycline and incubated overnight at 37° C. Colonies from the plate were picked and grown in 2 ml of LB containing tetracycline for DNA extracted via Miniprep® kit and sent for sequencing for validation. Following validation of proper cloning of each separate labrary, the Gibson Assembly reaction products were applied for ethanol precipitation overnight at −80° C. and dissolved in 10 μl ultra pure water. After validation and ethanol precipitation, transformation was done using TG1 bacteria cells by electroporation. The transformed cells were cultured in 100 ml YT medium with tetracycline and grown at 37° C. overnight with shaking at 225 rpm. Then, the culture was centrifuged at 8000 rpm, for 20 min. The supernatant was transferred into a new set of bottels and 40 ml of PEG/NaCl solution (33% PEG, 3.3M NaCl) was added and kept overnight at 4° C. for phage precipitation. The following day, extraction of phages from PEG-NaCl mix was conducted by centrifuging the precipitated phages for 45 min, 8000 rpm at 4° C., discarding the supernatant and dissolving the pellet in 1×TBS (for each 100 ml sup, 2 ml of sterile 1×TBS were added). The libraries were united and 1:500 10% sodium azide was added for preservation and stored at 4° C. Samples from the united library were transferred via heat shock into ER2738 competent E. coli bacteria cells. The infected bacteria were then plated on LBlagar plates containing tetracycline and incubated overnight at 37° C. Colonies from the plate were picked and grown in 2 ml of LB containing Tetracycline for DNA extracted via Miniprep® kit and sent for sequencing for validation.

### Determination of the Titer of the Phages

In order to determine the titer of the phages, an aliquot of 10 μl from the purified phages was serially diluted (10-fold dilutions) in TBS. Then, 2 μl drops of each dilution were spotted on a lawn of DH5αF′ bacteria with 0.5% agarose to produce plaques. The titers of plaque-forming units were calculated.

### Bio-Panning Protocol

Approximately 1×10¹⁰ of the combinatorial library (10 μl) were mixed with 10 μg of IgG in TBS-BSA solution (completed to 100 μl) in 0.5 ml vials (AXYGEN, PCR tubes, PCR-05-C) and incubated for 1 hr on a rotating mixer (ELMI, Intelli-Mixer™ RM-2L) at room temperature. Next, 50 μl of protein-G coated magnetic beads (Invitrogen, Dynabeads™ Protein G) were added, and the mix was incubated for an additional 30 min on a rotating mixer at room temperature. The vials were then placed on a magnetic stand (Promega, MagneSphere® Technology Magnetic Separation Stands) for 2 min to collect the beads and the supernatant was discarded. Subsequently, the beads were washed 3 times with 200 μl cold TBST, resuspended in 100 μl TBST, and transferred to a new set of vials. The vials were then placed on the magnetic stand and the supernatants were discarded. Bound phages were eluted with 105 μl of elution buffer for 10 min at room temperature. The eluate was collected and neutralized with 19 μl of neutralizing buffer. The elution procedure was repeated twice.

### Amplification of Combinatorial Library

Amplification of the combinatorial library was carried out as follows: 100 μl of an overnight E. coli DHFαF′ bacterial culture was added to 5 ml of 2×YT medium in a 50 ml tube. The bacterial suspension was incubated for about 5 h at 37° C., 225 rpm until OD_{600 nm}=1.5 was reached. Shaking speed was then reduced to 60 rpm for 30 min to allow the bacterial pili to regenerate. 150 μl of phages from the combinatorial library were added to the bacteria and incubation was continued for an additional 30 min. Every 10 min the tube was gently swirled to prevent aggregation. The 5 ml culture was then added to a 1 L flask containing 100 ml of 2×YT medium and 1 h later tetracycline was added (20 μg/ml final concentration). Incubation was continued overnight at 37° C., 225 rpm. The following day, the bacterial cultures were centrifuged at 8000 rpm for 20 min to pellet the bacteria. The supernatant was transferred into 250 ml centrifugation bottles, and phages were precipitated overnight at 4° C. with 0.4 volumes of PEG/NaCl. Next, the suspension was centrifuged at 8000 rpm for 45 min at 4° C. to pellet the phages, and the pellets were suspended in 2 ml of TBS. The suspended phages were centrifuged to pellet debris. A second and third Biopanning procedure was performed using the amplified phage suspensions from the previous rounds.

### Miniculture Preparations for Dot-Blot Analysis

For infection, 200 μl of an overnight E. coli DHFαF′ bacterial culture was added to 5 ml of 2×YT medium in a 50 ml tube. The bacterial suspension was incubated for about 5 h at 37° C., 225 rpm until OD_{600 nm}=1.5 was reached. Shaking speed was then reduced to 60 rpm for 30 min to allow the bacterial pili to regenerate. Then, diluted phages from the final elution were added to 30 ul grown bacteria and kept 15 min at room temperature to allow infection. LB medium was then added to the tubes to generate a final volume of 200 μl and incubated for additional 20 min at room temperature for phenotypic expression, then culture was placed on LB\agar plates containing Tetracycline to produce hundreds of tet-resistant colonies and incubated over night at 37° C.

On the following day, U-bottom sterile 96-well plates were filled with 200 μl of terrific broth (TB) containing 20 μg/ml tetracycline (1/1000 from stock) and 1/10 of buffer phosphate (PB).

Colonies were picked from the LBlagar plates described above by stabbing single colonies using sterile toothpicks and inoculating the wells of the plate by dipping the contaminated-tip of the toothpick into the media. The 96-well plates were sealed with parafilm and secured in a humidified box to reduce the amount of evaporation and shaken overnight (18 hours) at 160 rpm, 37° C., thus producing mini-cultures of phage clones.

### Identify Positive Phages-“Dot-Blot”

The following day the mini-cultures plates were centrifuged for 30 min at room temperature, to form a tight bacterial pellet. Using a multichannel pipettor, 125 μl/well of the supernatant (containing the phages) was transferred to a flat-bottom 96-well plate containing 50 μl/well of PEG/NaCl solution and mixed thoroughly. The phages were left for precipitation for 2 hours at 4° C. The original U-bottom plates containing the bacterial pellet (“master-plates”) were sealed with parafilm and stored at 4° C., for future confirmation of positive phages.

The flat-bottom plates were centrifuged at 4,000 rpm for 40 min at room temperature, to form phage pellets, the supernatants were removed and the pellets were thoroughly resuspended in a total of 110 μl/well TBS. The resuspended phages were analyzed by dot blotting as follows: 100 μl of phages were applied onto nitrocellulose (NC) membrane filters using a vacuum manifold (S&S Manifold). The membranes were quenched in blocking solution for 1 h at room temperature and incubated by gently rocking the membranes. Then, a primary Ab was added to generate a final concentration of 2 μg/ml and incubated 2 h at room temperature or overnight at 4° C., with gentle rocking. The membranes were then washed 6 times with TBST 0.05% and reacted with the proper secondary HRP-conjugated Ab at a concentration of 1:5, 000 in blocking solution for 45 min at room temperature and then washed 6 times with TBS. Signals were developed using the Enhanced Chemo-Luminescence (ECL) reaction.

### Confirmation of Positive Phages; Dot-Blot

To confirm the ability of the phages to bind the appropriate antibody, selected positive clones were grown again from the minicultures mentioned above as 2 ml overnight cultures in LB. The cultures were centrifuged to pellet the bacteria for 25 min at 14,000 rpm and 1.5 ml of each culture was transferred to a microcentrifuge tube containing 600 μl of PEG/NaCl for incubation of 1 h on ice to precipitate the phages. The suspension was centrifuged as above and the supernatant was discarded. The precipitated phages were resuspended in 200 μl of TBS by vortexing and 50 μl were applied onto a nitrocellulose membrane, to be reacted with the Ab of interest, by dot blotting using a vacuum manifold. Positive clones were grown as overnight cultures as above and maintained in 15% glycerol in −80° C. for further use.

### Large-Scale Production and Purification of Phages

The phage plasmids, displaying the peptides of interest, were transformed into DH5αF-bacteria. The infected bacteria were grown in 100 ml 2×YT/tet (20 μg/ml) medium overnight and then centrifuged at 8000 rpm for 20 min. The phage-containing supernatants were transferred to 40 ml PEG/NaCl solution (33% PEG, 3.3M NaCl), incubated at 4° C. overnight to precipitate the phages and then centrifuged (8000 rpm, 45 min). The precipitated phages were resuspended in a total volume of 10 ml TBS. The phages were precipitated again using 4 ml of PEG/NaCl (2 hr, 4° C. in ice), centrifuged (8000 rpm, 45 min) and resuspended in 2 ml TBS. The phages were centrifuged at 14,000 rpm for 10 min to discard debris, and supernatants were filtered through a 0.45 μm filter.

### Binding of Antibodies to Phage-Displayed Constructs Enzyme-Linked Immuno-Sorbent Assay (ELISA)

EIA/RIA 96-well flat bottom ELISA plates (Costar® Corning Incorporated, Corning, NY, USA) were coated overnight at 4° C. with 100 μl of 5-10 μg/ml 4E5A or 1A1D mAbs in PBS. The next day, the plates were washed with TBST 0.1%, blocked for 1 hour with milk 5% and incubated for 1.5 hours with 100 μl TBS containing 5×10¹⁰ phages/well. Wells were washed 5 times with TBST 0.1% and incubated with 100 μl milk 5% containing anti-M13 antibody for 1 hour. Wells were washed again and incubated with HRP-conjugated goat anti-rabbit (1:2,500) antibody (Jackson, West Grove, PA) in 5% milk for 45 min at room temperature. Following additional rounds of washing, plates were reacted with the TMB ELISA (TMB/E) substrate (Chemicon International, Temecula, CA). Absorbance was measured at 650 nm.

### Expression and Purification of Recombinant MBP Constructs

Four constructs of interest were subjected to PCR reaction with the complementary primers for MBP vector (#17-24, Table 3). The pMAL-p5x vector for cloning was double digested with NdeI and HindIII and via a Gibson Assembly reaction was cloned with the 4 constructs of interest.

The DNA of the pMAL-p5x vector carrying the desired insert was purified from the E. coli DH5αF-host and was used to transfect the E. coli Rosetta strain. Fresh transformants were used to prepare a 100 ml LB culture containing 100 μg/ml ampicillin and 0.4% glucose (to inhibit expression) at 37° C. until OD₆₀₀=0.6-0.7. A small sample of 2 ml was taken and referred to as “uninduced”. The remaining culture was treated with 0.5 mM IPTG for 4 hours at 30° C. and then centrifuged (5,000 rpm for 10 minutes). The pellets were kept in −20° C. The bacterial pellets were then resuspended in PBS 0.1% Triton X 100 and went through 5-6 rounds of sonication (30 seconds for each round) to extract the protein from the bacteria. Cells were harvested by centrifugation at 12,000 rpm for 20 min at 4° C. and supernatants were added to 200 μl nickel beads (Nickel ChroMatrix, Favorgen) and incubated at 4° C. overnight on a shaker. Immidozole (Fluka) was added to a final concentration of 5 mM to reduce any non-specific binding. On the next day beads with protein were centrifuged (1,000×g for 5 minutes) and washed with 50-75 ml PBS 20 mM immidozole. MBP constructs were eluted with high concentrations of immidozole (PBS+150 mM immidozole), concentration was determined using the Bradford assay (Kruger, 2009) and measuring absorbance at OD280. Proteins were dialyzed against PBS and analyzed for binding.

### Binding of Antibodies to MBP Constructs

Two types of ELISA tests were conducted:
Direct coating with MBP: EIA/RIA 96-well flat bottom ELISA plates (Costar® Corning Incorporated, Corning, NY, USA) were coated overnight with 10 μg/ml MBP constructs in PBS. The next day, the plates were washed with PBS, blocked for 1 hour with milk 5% and incubated for 1.5 hours with 100 μl PBS containing 5 μg 4E5A or 1A1D mAbs. Wells were washed 5 times with PBS and then reacted with 100 μl of milk 5% containing HRP-conjugated goat anti-mouse antibody (Jackson, West Grove, PA) 1:2,500 for 1 h. Wells were washed again and reacted with the TMB/E ELISA substrate (Chemicon International, Temecula, CA). Absorbance was measured at OD₆₅₀.

Direct coating with Ab: EIA/RIA 96-well flat bottom ELISA plates were coated overnight with 5-10 μg/ml antibody 4E5A or 1A1D in PBS. On the next day the plates were washed with PBS, blocked for 1 h with milk 5% and incubated for 1.5 hours with 100 μl PBS containing 1-5 μg MBP constructs. Wells were washed 5 times with PBS and then reacted with 100 μl of milk 5% containing anti-MBP antibody diluted 1:2,500 for 1 hour. Wells were washed again and incubated with HRP-conjugated goat anti-rabbit antibody (Jackson, West Grove, PA) in 5% milk, followed by reaction with TMB/E and absorbance reading at OD₆₅₀.

### Affinity Pull Down

Each mAb (5 μg) was separately incubated with the protein constructs (5 μg) in a TBS-BSA solution in 0.5 ml vials for 2 hours in room temperature, then 50 μl of protein-G coated magnetic beads (Invitrogen, Dynabeads™ Protein G) were added to the solutions for another 30 min of incubation. The vials were then placed on a magnetic stand (Promega, MagneSphere® Technology Magnetic Separation Stands) for 2 min to collect the beads and the supernatants were discarded. Subsequently, the beads were washed 3 times with 200 μl cold TBST, resuspended in 100 μl TBST, and transferred to a new set of vials. The vials were then placed on the magnetic stand and the supernatants were discarded. Bound proteins were eluted with 105 μl of elution buffer for 10 min at room temperature. The eluate was collected and neutralized with 19 μl of neutralizing buffer. The eluted constructs were applied to nitrocellulose membranes for dot-blot analyses. The membranes were incubated with primary Ab, washed, and then reacted with compatible secondary HRP-conjugated Ab for detection.

### Example 1

### Epitope-Based Vaccine of Dengue Virus

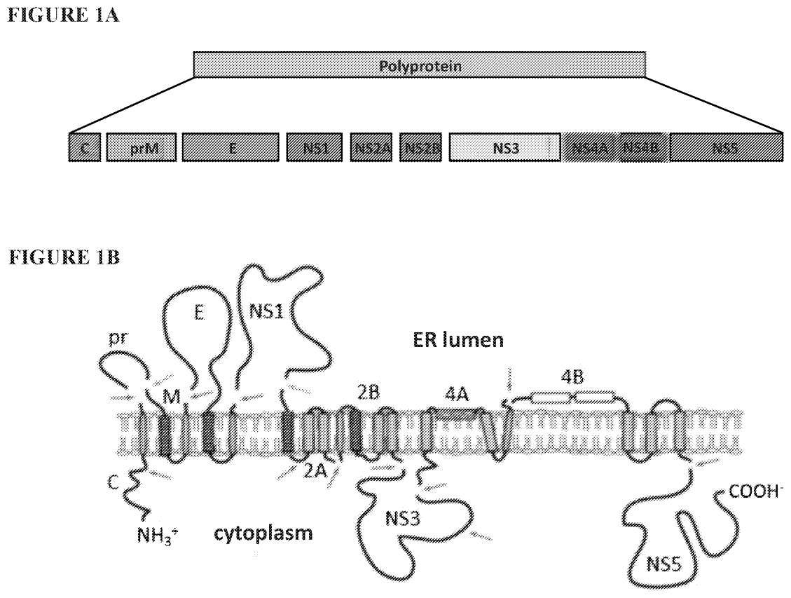

Dengue virus (DENV) positive RNA (11 kb) codes for one continuous polyprotein containing 3 structural proteins (Core-C, membrane protein—prM and Envelope—E) followed by 7 Non-Structural (NS) proteins as shown in FIG. 1. Here it was aimed to develop novel immunogens for vaccination against tropical pathogens, specifically the development of epitope-based immunogens specific for the DENV envelope protein. In view of the concern of ADE, the reconstitution of neutralizing epitopes was first addressed. Thus far, the main neutralizing epitopes of DENV have been associated with the DII and DIII domains of the envelope. Therefore, the non-limiting examples for reconstitution disclosed herein are focused on these two domains. However, it should be understood that the invention further encompasses the use of the DI domain for reconstitution as well.

Thus, as indicated above, two comprehensive conformer libraries E1 and E2, for epitopes of domains DII and DIII, respectively, have been constructed as fusions of the P3 protein of the filamentous bacteriophage fd. Both libraries have been screened against selected neutralizing antibodies. The E2 epitope (Domain III) of the DENV envelope protein target has proven to be extremely successful and multiple potential immunogens are being developed and characterized.

1a. Epitope-Based Vaccines for Dengue Fever Vaccination

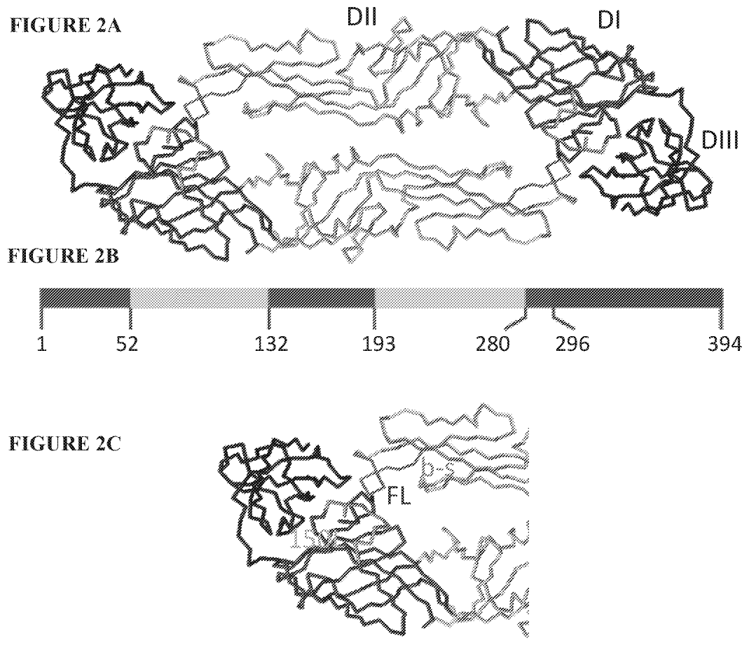

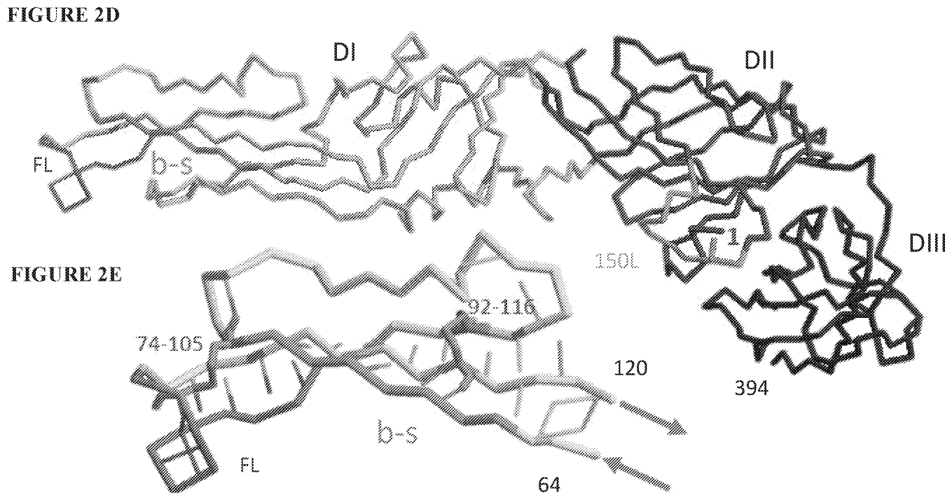

The DENV envelope protein consists of three structural domains (DI, DII and DIII) and is presented on the viral coat as a dimer of envelope proteins, oriented head to tail, with the DII domain of one subunit is juxtaposed across from the DIII domain of the opposing subunit and the DI domain bridging DII and DIII (see FIG. 2A-2D). Identifying specific neutralizing epitopes on the viral envelope and reconstituting them as potential vaccine immunogens is extremely important as DENV tends to elicit enhancing antibodies (Am. J. Trop. Med. Hyg. 40.4:444, 1989). Thus, focusing on discrete neutralizing epitopes should obviate the production of these enhancing antibodies. Here, two specific neutralizing epitopes, one derived from the DII and the other derived from the DIII domains were targeted. There is ample evidence that these can elicit the production of neutralizing antibodies, some of which have been shown to be broadly cross-reactive among the 4 DENV serotypes.

### 1B. Construction of Combinatorial Conformer Libraries

The technical approach was to produce comprehensive conformer libraries of selected epitopes using filamentous phage display. Because the two epitopes are greater than 60 amino acids in length, the libraries were constructed as N terminal fusions with the bacteriophage protein 3. Each of the epitopes was constructed with diverse linkers, and NNK codons were used to enable the incorporation of all 20 amino acids at each residue position.

### 1C. Epitope E1—Residues K64 to K120 of the DII Domain.

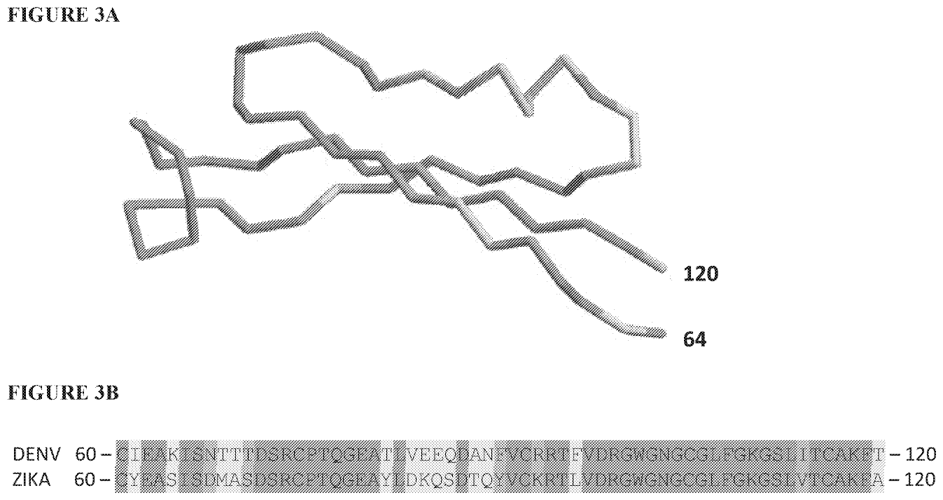

The first proposed epitope to be reconstituted is detailed in FIG. 2E. It should be noted that residues 64-120 (also denoted by the amino acid sequences as denoted by any one of SEQ ID NO: 26, 26, 27, 28 for serotypes 1, 2, 3 and 4, respectively), produce a compact and highly structured feature, supported by extensive hydrogen bonding and further locked into position by two disulfide bonds (74-105 and 92-116). Combinatorial linkers are to be introduced just preceding residue 64 and following residue 120. A homology between this epitope of Dengue virus and between the Zika virus is further shown in FIGS. 3A and 3B (see homology between SEQ ID NO: 14 and 15 in FIG. 3B). The construction of the conformer library was conducted using a modified fth1 expression vector in which a cloning cassette was introduced into the 5′ terminal end of the Protein 3 gene. The cassette consisted of two asymmetric BstXI sites, which ensure that the cut vector cannot close on itself and that the inserts are introduced only in the correct orientation. Inserts were introduced into the cut vector by Gibson assembly.

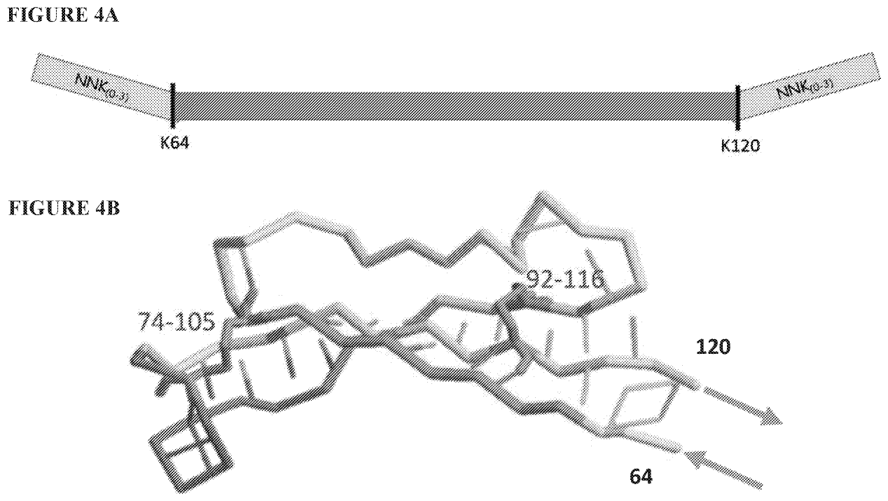

The design of the library consisted of 0-3 NNK codons at the 5′ end of the construct as well as 0-3 NNK codons at the 3′ end (FIG. 4A). The four cysteines included in this region were maintained to enable the formation of the two disulfide bridges (74-105 and 92-116, FIG. 4B) that are thought to stabilize the epitope structure.

In order to produce the combinatorial complexity of all possible linkers, a total of 16 libraries were produced by 16 PCR reactions. The libraries were then mixed to generate a master library that contained all possible linker lengths. The library was confirmed by cloning and sequencing 10 randomly selected clones. As can be seen in Table 4, good presentation of the various linkers was obtained.

The library was then screened with a total of three neutralizing antibodies (A11, B7 and C8). Each of the antibodies was used in 1-2 independent panning experiments and underwent as many as 5 capture/amplification rounds. Clonal phage picking was performed using aliquots of amplifications #3, #4 and #5. Clones were tested by dot-blot for binding to the antibody used during the selection. Thus far no candidate E1 based immunogens have been selected

TABLE 4
Sequencing results of randomly selected
clones from Epitope DII master library.

| Clone ID | 5′ NNK sequence | 3′ NNK sequence |
|---|---|---|
| EP1_1 | F R | TSR |
| SEC ID NO: 29 | | |
| EP1_2 | ALH | T |
| SEC ID NO: 30 | | |
| EP1_3 | S | H |
| SEC ID NO: 31 | | |
| EP1_4 | IKR | P |
| SEC ID NO: 32 | | |
| EP1_5 | R | ST |
| SEC ID NO: 33 | | |
| EP1_6 | HLL | TT |
| SEC ID NO: 34 | | |
| EP1_7 | NAP | — |
| SEC ID NO: 35 | | |
| EP1_8 | G | TRT |
| SEC ID NO: 36 | | |
| EP1_9 | SQI | — |
| SEC ID NO: 37 | | |
| EP1_10 | CAL | HT |
| SEC ID NO: 38 | | |

### 1D. Epitope E2—Residues M301 to E370 of the DIII Domain.

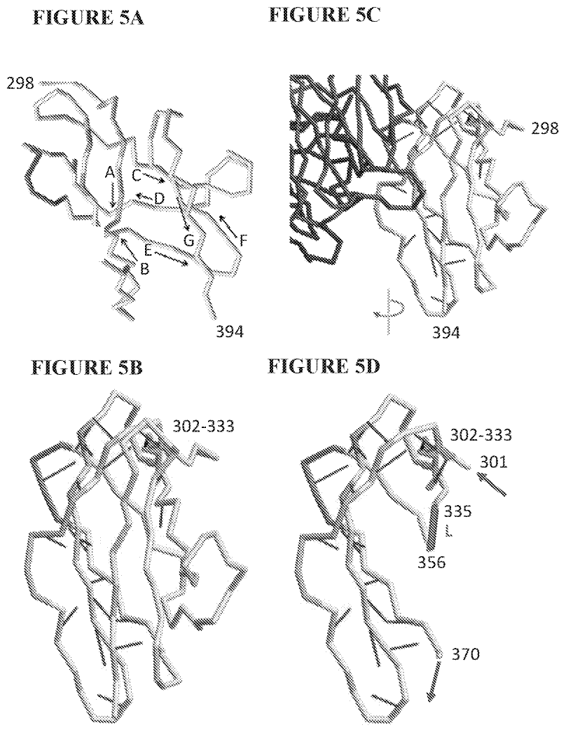

Next, the inventors addressed the construction and analysis of Epitope DIII. More specifically, Dengue virus has several neutralizing epitopes on the DIII domain that have been defined by crystallization (PDB ID: 2R69, 3UZQ, 3UZV, 3UZE, and 3UYP). A second epitope was thus constructed on the basis of the DIII domain and starts from Methionine 301 and ends with Glutamic Acid 370 as shown in FIG. 5. This construct differed from the epitope of Example 1C (epitope DII) in that it contained an internal linker that bridges the gap between residues 1335 and P356. Each construct could contain as many as three combinatorial linkers: 0-3 NNK at the 5′ end, 0-5 NNK internally and 0-3 NNK at the 3′ end. One dedicated construct contained 5′ and 3′ linkers and a native 20 amino acid internal loop instead of the bridging linker.

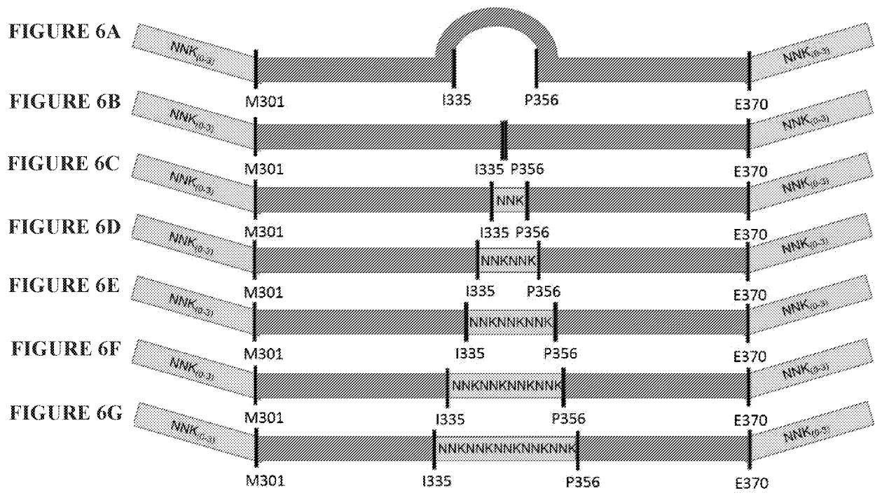

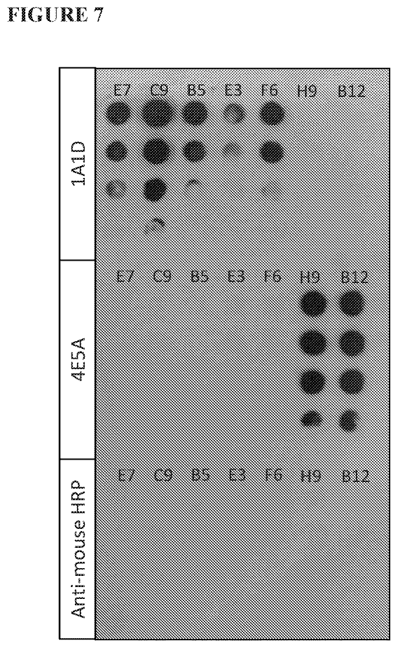

Construction of this library was conducted using 5′-sense and 3′-antisense combinatorial PCR primers that contained 0, 1, 2 or 3 NNKs. These primers were mixed and used for PCR of 7 different templates. One template contained the native complete sequence from 301 to 370 (as also denoted by the amino acid sequence of any one of SEQ ID NO: 39, 40, 41, 42, for serotypes 1, 2, 3 and 4, respectively)-including the loop (336-355, as also denoted by the amino acid sequence of any one of SEQ ID NO: 43, 44, 45, 46, for serotypes 1, 2, 3 and 4, respectively), (FIG. 6A). The second template started at 301 through 370, however the loop was omitted and residue 335 was linked directly to 356 (i.e., no internal linker) (FIG. 6B). Templates 3-7 contained 1-5 NNK codons, respectively, bridging residues 335 to 356 (FIG. 6C-6D). The PCR products were introduced into the BstXI cloning sites of the modified fth1 vector by Gibson assembly as was done for Epitope DII. The libraries were confirmed for expected complexity. Table 5 represents the specific results of the 40 selected clones.

TABLE 5
Sequencing results of randomly selected
clones from Epitope 2 library.

| Clone ID | 5′ NNK sequence | Internal linker | 3′ NNK sequence |
|---|---|---|---|
| EP2_1 | GE | — | — |
| EP2_2 | RS | Original loop | S |
| EP2_3 | ETC | — | RE |
| EP2_4 | WV | Original loop | TN |
| EP2_5 | — | EAG | — |
| EP2_6 | S | — | — |
| EP2_7 | IW | RF | M |
| EP2_8 | — | ANLVD | A |
| | | SEQ ID NO: 48 | |
| EP2_9 | VST | — | VK |
| EP2_10 | VV | Original loop | — |
| EP2_11 | V | RLNY | MT |
| | | SEQ ID NO: 49 | |
| EP2_12 | — | TKV | — |
| EP2_13 | — | EGLD | FYM |
| | | SEQ ID NO: 50 | |
| EP2_14 | D | Original loop | — |
| EP2_15 | — | — | — |
| EP2_16 | — | GGR | LN |
| EP2_17 | GRA | Original loop | LKA |
| EP2_18 | TF | — | — |
| EP2_19 | N | Original loop | DDS |
| EP2_20 | L | CLVN | YT |
| | | SEQ ID NO: 51 | |
| EP2_21 | D | ISVV | — |
| | | SEQ ID NO: 52 | |
| EP2_22 | AR | — | V |
| EP2_23 | PLW | V | D |
| EP2_24 | QSD | R | — |
| EP2_25 | IWE | GSGGS | K |
| | | SEQ ID NO: 1 | |
| EP2_26 | RS | A | — |
| EP2_27 | GG | — | PNV |
| EP2_28 | RRV | Original loop | — |
| EP2_29 | — | ELV | PT |
| EP2_30 | GE | — | — |
| EP2_31 | RS | Original loop | S |
| EP2_32 | — | EAG | — |
| EP2_33 | S | — | — |
| EP2_34 | IW | RF | M |
| EP2_35 | — | ANLVD | A |
| | | SEQ ID NO: 48 | |
| EP2_36 | D | — | P |
| EP2_37 | — | TKV | — |
| EP2_38 | — | EGLD | FYM |
| | | SEQ ID NO: 50 | |
| EP2_39 | VR | — | QKD |
| EP2_40 | V | RLNY | MT |
| | | SEQ ID NO: 55 | |

### 1E. Isolation of Functional Reconstituted Versions of Epitope DIII

The libraries were screened against two neutralizing antibodies (4E5A and 1A1D) multiple times and as many as 4 capture and amplification cycles. For each sample 400 clones were picked and tested by dot-blot for antibody recognition. Positive clones were validated and sent for sequencing. Table 6 shows the linker compositions for 7 positive clones isolated with the two antibodies tested. The question arose as to whether the positive constructs are cross-reactive for the two antibodies, i.e., do 4E5A specific clones bind 1A1D antibody and vice versa. As shown in FIG. 7, no cross-binding was detected.

Thus, specific experiments were conducted with the intent to isolate constructs that might be cross-reactive. The library was panned against one antibody and the eluted phages were panned against the other antibody. This was repeated for a number of cycles with a number of library aliquots. Moreover, this experiment was conducted once starting with 4E5A and once with 1A1D.

A total of 65 positive clones were isolated and validated. Sequence analyses showed that: (i) one clone was previously seen for antibody 4E5A; (ii) a total of 13 unique clones were found; (iii) 4 clones appeared to be cross-reactive (Table 7).

TABLE 6
Linker composition for 7 positive clones isolated with 1A1D or 4E5A.

| | | Clone | 5′ NNK | Internal | 3′ NNK | | |
|---|---|---|---|---|---|---|---|
| Clone # | Frequency | ID | sequence | linker | sequence | 4E5A | 1A1D |
| 1 | 9 | E7 | N | SKRG | T | | + |
| SEQ ID | | | | SEQ ID NO: | | | |
| NO: 56 | | | | 2 | | | |
| 2 | 1 | C9 | SR | WRLG | Y | | + |
| SEQ ID | | | | SEQ ID NO: | | | |
| NO: 57 | | | | 3 | | | |
| 3 | 1 | B5 | GR | QTGW | L | | + |
| SEQ ID | | | | SEQ ID NO: | | | |
| NO: 58 | | | | 4 | | | |
| 4 | 1 | E3 | K | GGWG | — | | + |
| SEQ ID | | | | SEQ ID NO: | | | |
| NO: 59 | | | | 5 | | | |
| 5 | 1 | F6 | P | RRL | RS | | + |
| SEQ ID | | | | | | | |
| NO: 60 | | | | | | | |
| 6 | 1 | H9 | RGA | NNG | YL | + | |
| SEQ ID | | | | | | | |
| NO: 61 | | | | | | | |
| 7 | 1 | B12 | S | GGG | RL | + | |
| SEQ ID | | | | | | | |
| NO: 62 | | | | | | | |

TABLE 7
Linker composition of positive clones isolated
from the cross-reactivity experiments.

| | | Clone | 5′ NNK | Internal | 3′ NNK | | |
|---|---|---|---|---|---|---|---|
| Clone # | Frequency | ID | sequence | linker | sequence | 4E5A | 1A1D |
| 1 | 16 | F8 | TSR | GLRG | TL | + | + |
| SEQ ID | | | | SEQ ID | | | |
| NO: 63 | | | | NO: 6 | | | |
| 2 | 14 | A11 | G | PFGSS | — | + | + |
| SEQ ID | | | | SEQ ID | | | |
| NO: 64 | | | | NO: 7 | | | |
| 3 | 16 | C7 | R | KGG | NLA | + | |
| SEQ ID | | | | | | | |
| NO: 65 | | | | | | | |
| 4 | 7 | D12 | GFP | PLGDH | RPV | + | |
| SEQ ID | | | | SEQ ID | | | |
| NO: 66 | | | | NO: 8 | | | |
| 5 | 2 | H1 | PA | GRGG | LL | + | + |
| SEQ ID | | | | SEQ ID | | | |
| NO: 67 | | | | NO: 9 | | | |
| 6 | 2 | E4 | P | AGIDH | — | + | |
| SEQ ID | | | | SEQ ID | | | |
| NO: 68 | | | | NO: 10 | | | |
| 7 | 2 | H9 | RGA | NNG | YL | + | |
| SEQ ID | | | | | | | |
| NO: 61 | | | | | | | |
| 8 | 1 | C1 | P | SPKG | — | + | |
| SEQ ID | | | | SEQ ID | | | |
| NO: 70 | | | | NO: 11 | | | |
| 9 | 1 | C2 | H | QGG | TW | + | |
| SEQ ID | | | | | | | |
| NO: 71 | | | | | | | |
| 10 | 1 | C6 | TGL | YSGQW | TTQ | + | + |
| SEQ ID | | | | SEQ ID | | | |
| NO: 72 | | | | NO: 12 | | | |
| 11 | 1 | D3 | P | RFG | YMR | + | |
| SEQ ID | | | | | | | |
| NO: 73 | | | | | | | |
| 12 | 1 | E2 | VP | EWN | T | + | |
| SEQ ID | | | | | | | |
| NO: 74 | | | | | | | |
| 13 | 1 | H10 | R | GDWG | NI | + | |
| SEQ ID | | | | SEQ ID | | | |
| NO: 75 | | | | NO: 13 | | | |

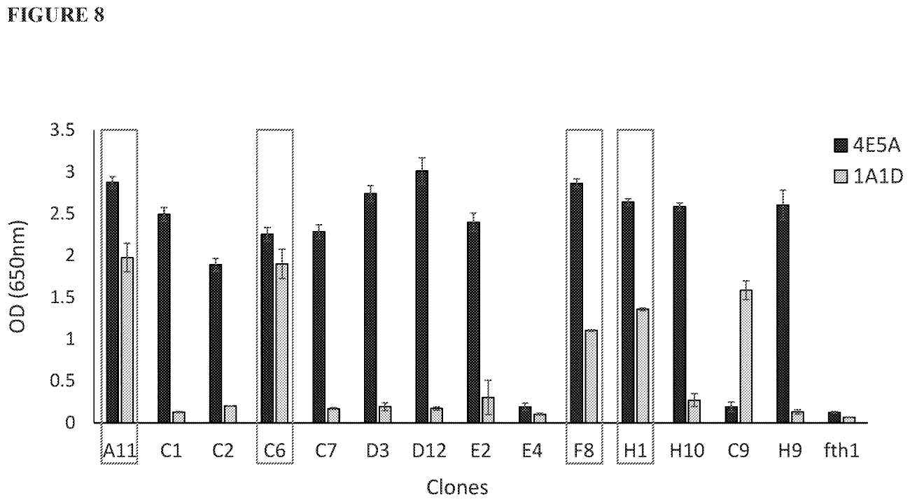

All 12 clones were further analyzed by ELISA test. “Sandwich ELISA” was performed with 1A1D and 4E5A as capture Abs, positive clones presented by the phages, rabbit anti-m13 Ab and Anti-rabbit-HRP Ab as detection Ab, TMBE substrate of HRP was submitted and read was recorded at OD 650 nm every 3 minutes, for 15 minutes (full saturation). ELISA test confirmed the results previously attained by dot-blot, ensuring the binding of each clone to the Ab it was isolated by and four clones were confirmed to be cross-reactive by both Abs (FIG. 8).

These results suggest that segments of the epitope, which represent part of the bona fide binding surface, can be reconstituted to resemble their native conformation and be sufficient for Ab recognition.

### Example 2

### Incorporation of Cross-Reactive Epitopes of Dengue Virus in Carrier Proteins for the Construction of Candidate Immunogens and ELISA Assay

Out of the 13 clones, 4 candidates were identified as being recognized by both neutralizing mAbs, and thus are good candidates for the production of epitope focused immunogen (EFI's), or epitope-based vaccine. The next step was to design the constructs as functional immunogens. For this, the phage displayed constructs needed to be incorporated into carrier proteins. The production of functional peptides presenting the physiological conformation is not necessarily promised. For the construction of the immunogens, three potential scaffolds were used: maltose binding protein (MBP) and glutathione S-transferase (GST), and I53-50A.

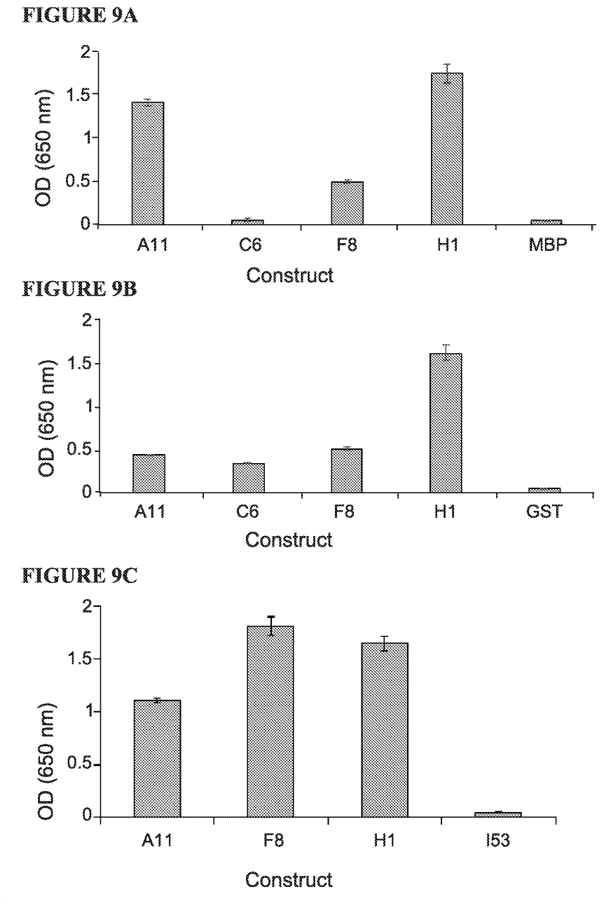

For the construction of the MBP-conjugates, pMAL-N6xHis-p5x vector was used, double digested by NdeI and HindIII. The candidate “positive” reconstituted constructs were amplified by PCR, and used as inserts that were introduced into the vector's cloning site by Gibson assembly. The annealed products were transformed into DH5a competent cells. Several bacterial clones of each of the 4 constructs (clones A11, C6, F8 and H1, also denoted by SEQ ID NO: 64, 72, 63, 67, respectively), were picked and their DNA sequences were confirmed. The verified-constructs were used to transform competent Rosetta cells that were induced for protein expression. The products were purified on Ni beads. All 4 candidate-reconstituted-MBP-conjugates were tested by ELISA for Ab recognition. The ELISA test results for 4E5A Ab showed high binding with constructs A11 and H1, relatively low binding to construct F8 and no binding to C6 and to the negative control of MBP vector (FIG. 9A) (also denoted by SEQ ID NOs: 132-135). None of the MBP-conjugates appeared to be recognized by 1A1D Ab.

A similar protocol was followed for the production of candidate reconstituted GST-conjugates, using pET30a-N6xHis-GST vector for cloning (double digested with BamH1 and HindIII). The sequenced and verified constructs were purified and tested by ELISA for Ab recognition. As was the case for the MBP-conjugates, none of the GST constructs SEQ ID NOs: 136-139) were recognized by 1A1D Ab. Construct H1 appeared to be highly recognized by 4E5A Ab, whereas constructs A11, C6 and F8 appeared to have relatively lower recognition (FIG. 9B).

A similar protocol was followed for the production of candidate reconstituted components, using I53-50A vector for cloning (double digested with NdeI). The sequenced and verified constructs were purified and tested by ELISA for Ab recognition. 4E5A Ab showed high binding with constructs A11, F8 and H1 and no binding to the negative control of 153 vector (also denoted by SEQ ID NOs: 140, 141, 142, respectively).

### Example 3

### Affinity Pull Down Assay of Dengue Reconstituted Epitopes

It seems that surface immobilization of the antigen as in ELISA tests might interfere with the ability of some of the mAbs to interact and bind their corresponding epitopes. The question was asked whether the antibodies can recognize and complex with the virus in solution.

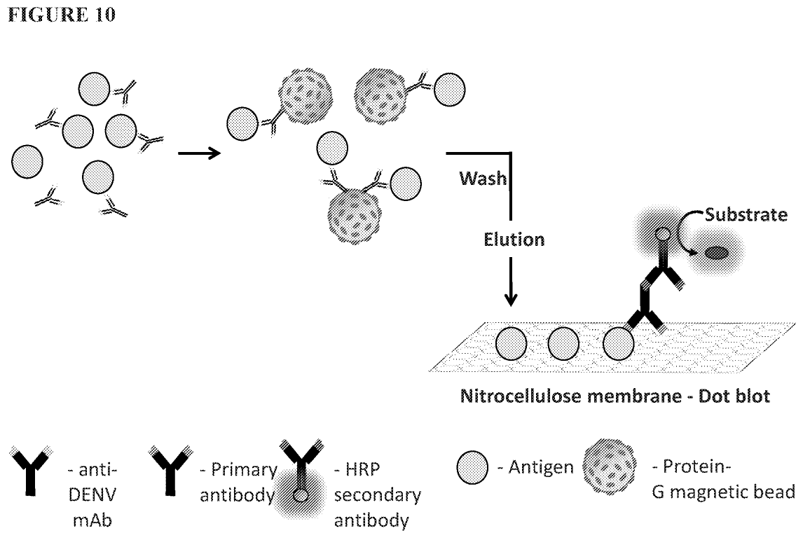

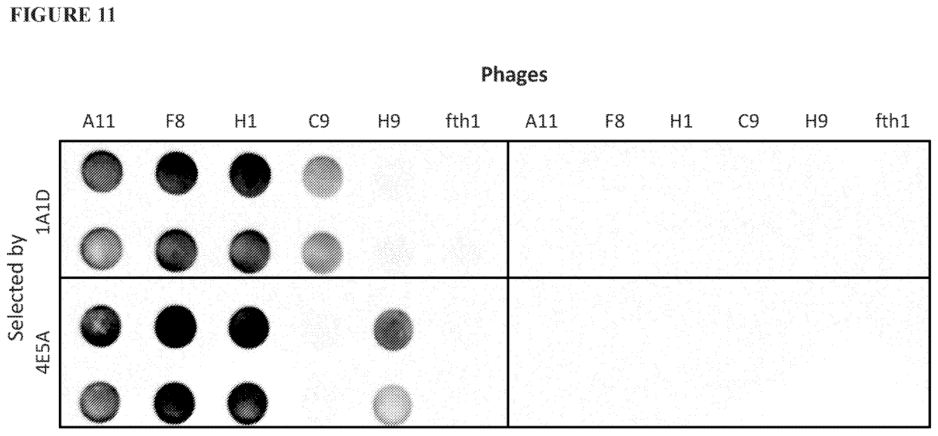

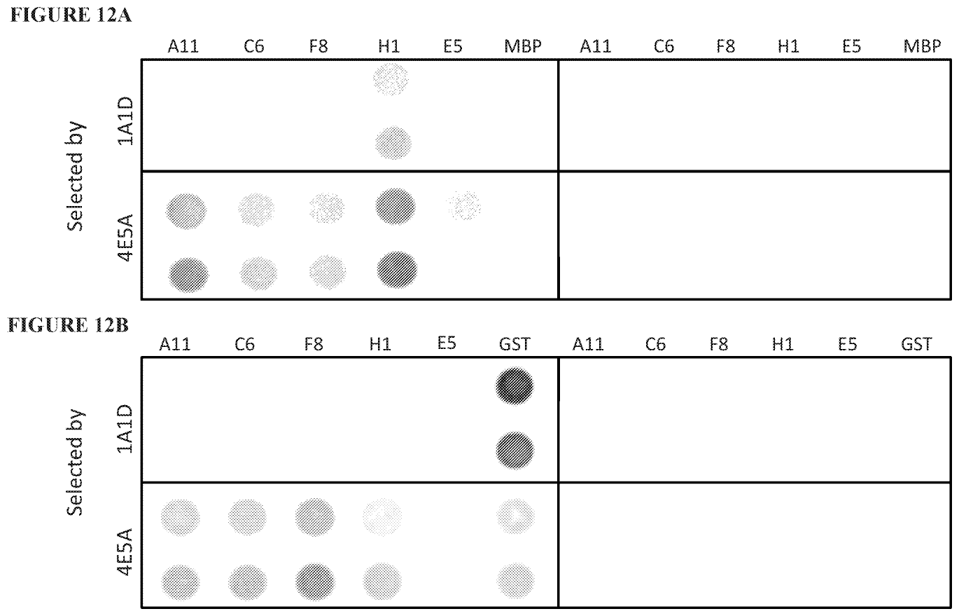

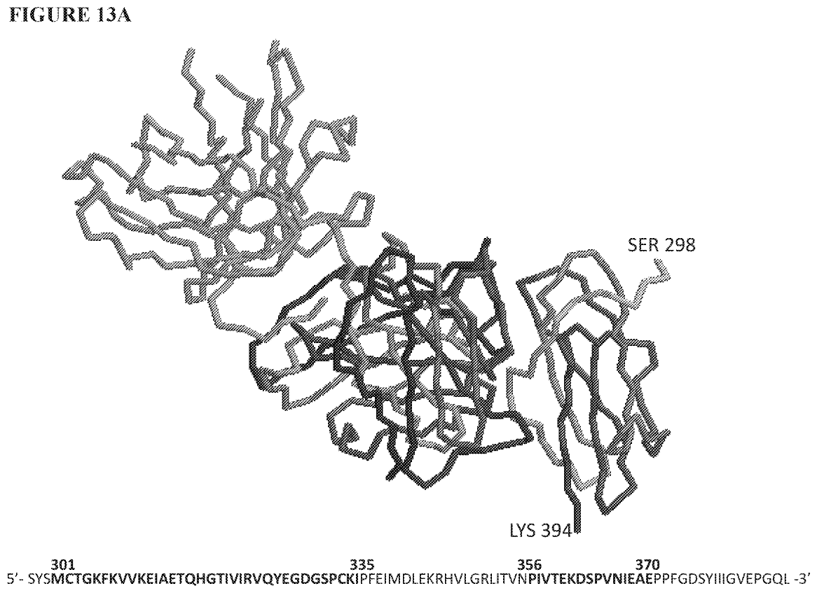

Therefore, an alternative procedure was developed which relies on the ability of the antibodies to interact in solution with their cognate antigen and then affinity pull down the antibody/antigen complex. Toward this aim, the mAbs were incubated with the protein constructs in a solution to which protein-G magnetic beads were later added. After incubation and washes, the constructs were eluted and applied to nitrocellulose membranes for dot-blot analyses (see scheme in FIG. 10). The validity of this protocol was first tested and confirmed on phage-displayed constructs. As illustrated in FIG. 11, this procedure works well and corresponds with the previously attained ELISA results; the cross binding of A11, F8 and H1 and the discriminatory binding of constructs C9 and H9, which are recognized by 1A1D and 4E5A, respectively. In view of these results the affinity pull down procedure was applied to evaluate the 4 MBP-conjugates (A11, C6, F8 and H1, also denoted by SEQ ID NO: 64, 72, 63, 67, respectively) and a non-related construct-E5 was used as a negative control (see FIG. 12A). The membrane was incubated with rabbit-anti-MBP Ab followed by a secondary anti-rabbit-HRP. By reacting the constructs with the Abs in solution, it was possible able to detect binding of construct H1 by 1A1D (FIG. 12A).

The results of testing the GST-conjugates with 1A1D and 4E5A Abs, were less clear-cut (FIG. 12B). The use of polyclonal sera derived from GST-immunized rabbits for detection, may be the reason for the non-specific binding, since it is not a “clean”, monoclonal reagent.

Overall, the ELISA and the affinity pull down test-results confirmed the binding of the reconstituted conjugates by at least one neutralizing Ab.

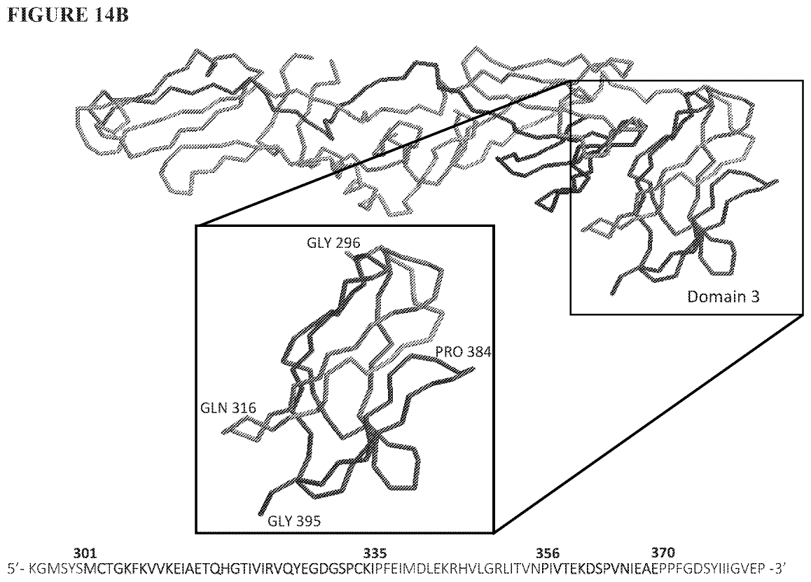

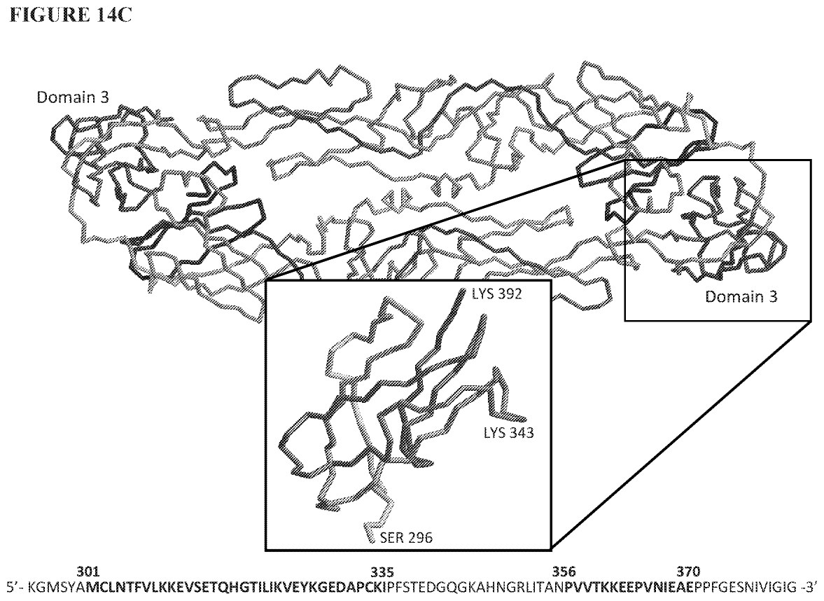

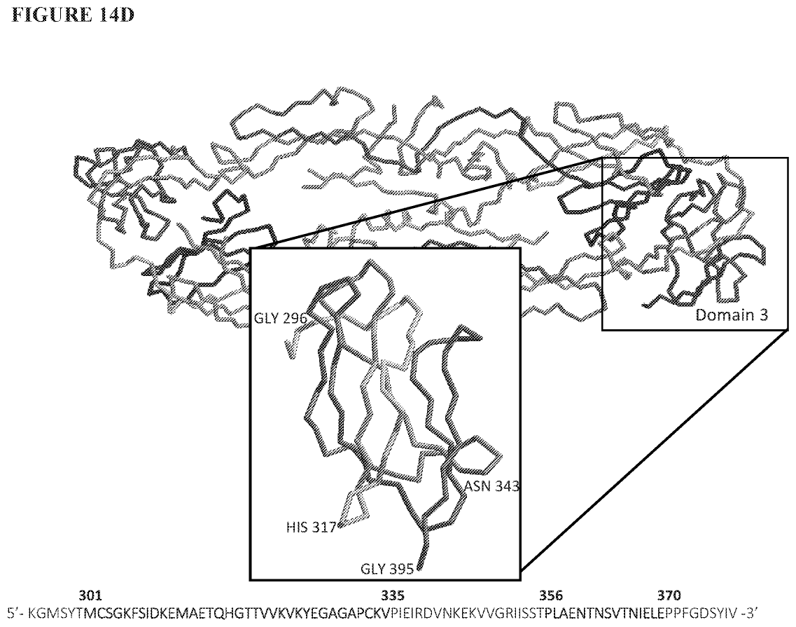

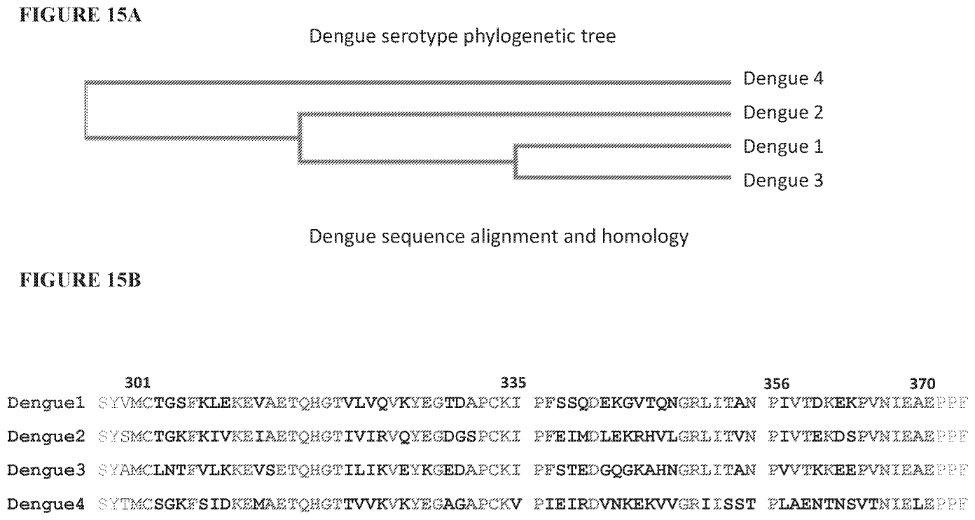

The structures of Domain III of the envelope protein of Dengue, in complexes with the relevant neutralizing mAbs that were used as detailed above are represented in FIG. 13A-13F, detailing also the PDB IDs and the serotypes. Furthermore, FIG. 14A-14B provides schematic presentations of the structure of different serotypes of the Dengue virus while detailing also the PDB IDs. FIG. 15 shows sequence alignments of DENV serotypes 1, 2, 3, and 4, as denoted by SEQ ID NOs: 76, 77, 78, 79, respectively.

### Example 4

### Optimization of a Broadly Cross-Reactive Immunogen

The inventors next aimed at producing an improved immunogen designed to elicit potent antibody responses that cross neutralize all 4 DENV serotypes, 1-4. For example, the lead constructs isolated using mAbs 1A1D and 4E5A, clones A11, C6, F8, H1 (also denoted by SEQ ID NO: 64, 72, 63, 67, respectively) as detailed in Table 7, are subjected to biased random mutagenesis of their linkers and screened under increased stringency against the Ab513 antibody [Robinson et al., Cell 162:493-504 (2015)].

The unique challenge in producing an effective prophylactic vaccine against DENV is that the virus exists as 4 distinct serotypes. Antibodies produced against one serotype can actually exacerbate the disease when one is infected with another serotype. A possible solution to this problem, designated Antibody Dependent Enhancement (ADE), is the production of antibodies that are highly cross neutralizing. The reconstitution and production of immunogens that are intended to elicit such highly cross neutralizing antibodies, are proposed herein.

Example 1D illustrates the reciprocal panning method to select and isolate reconstituted DIII domains that cross react with both antibody probes, 1A1D and 4E5A, (Clones A11, C6, F8, H1, Table 7). It is noteworthy to emphasize that mAb 4E5A is actually an improved version of the original murine mAb 4E11, which was modified to better bind the DIII domain of serotype 4. Further improvement of 4E5A by Ram Sasisekharan and colleagues has led to the production of Ab513. Ab513 has not only been demonstrated as markedly protective against all 4 serotypes, but also safer in the sense that this mAb is significantly reduced in its ability to mediate ADE.

Therefore, a general scheme to optimize the reconstituted immunogens of the present disclosure is to screen variant libraries of the constructs with a more potent cross neutralizing mAb such as the improved Ab513, and screen under conditions of ever-increasing stringency thus driving the selection towards variants that better complement the paratope of the improved Ab513.

In order to produce a comprehensive library of multiple variant immunogens, gBlock oligonucleotides are used in which the linker sequences are subjected to biased random mutagenesis (BRM) [Ophir R. and Gershoni J. M. Protein Engineering 8:143-146 (1995)]]. BRM is accomplished by using 4 tainted stock solutions of phosphoramidites in the synthesis of the segments of the gBlock that correspond to the linker segments of the constructs. The tainted stock solutions can contain 97% of one base phosphoramidite, e.g., G, contaminated with 1% of each of the remaining three phosphoramidites, i.e., A, C and T. Four tainted stock solutions are prepared to correspond to the 4 bases, where the major component is contaminated with the remaining 3 bases, respectively. In the production of the gBlock for each construct, the tainted stock solutions are used when generating the linker segments. This produces a massive library for each construct in which for every position of the linker-corresponding nucleic acid sequence, there exists a 97% chance of being correct with a 3% chance of base-variation. This creates a vast variety of linkers all heavily biased for the “correct” intended linker compositions. The variations afford the opportunity to select for variants that better conform to the improved Ab513 mAb.

This approach for immunogen-optimization is but one embodiment of the method of the present disclosure and should not be limiting in any way. Alternative variations and modifications of the methodology are anticipated. For example, the ratio of contamination of each phosphoramidite stock solution can be varied thus producing linkers of greater or lesser variation. Also, the present disclosure describes the use of Ab513, however any alternative mAb can be employed if it is expected to improve the cross reactivity of the construct and consequently to better elicit cross neutralizing antibodies. Thus for example, one can envision the use of 4 different mAbs to screen the BRM libraries in series and reciprocal fashion as described previously, where each of the 4 different mAbs are extremely discriminating and specific for each of the 4 serotypes respectively.

### Example 5

### Animal Experiment—Immunization and Analysis of Sera

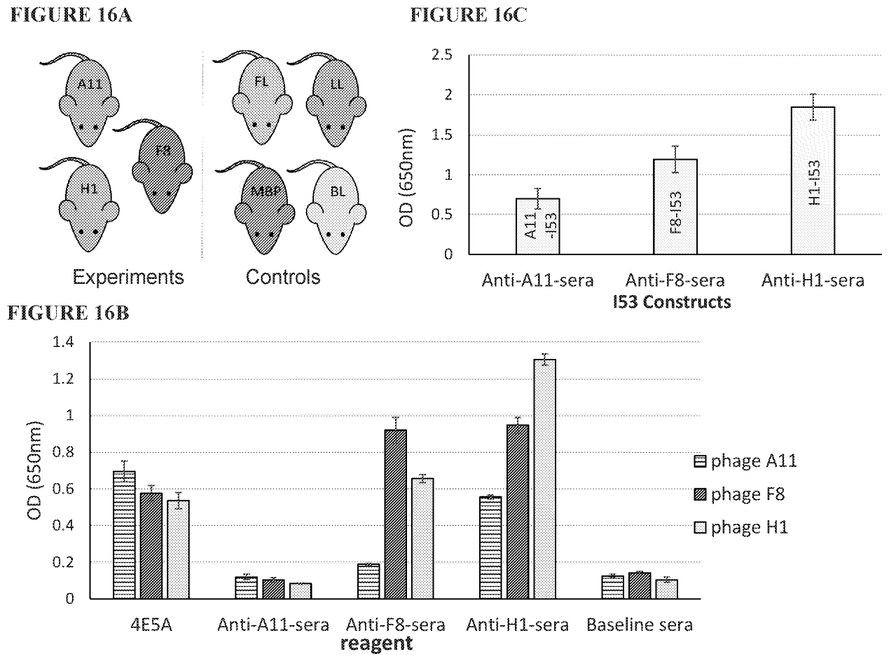

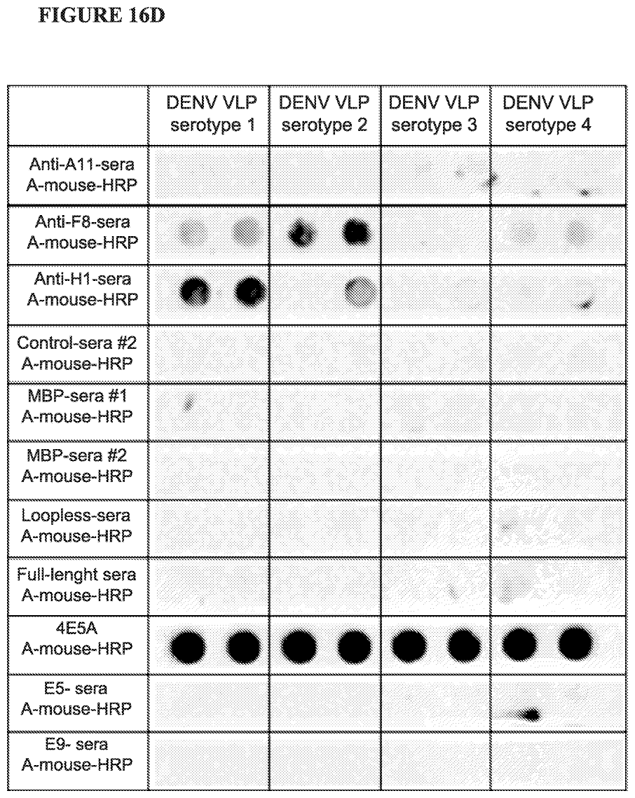

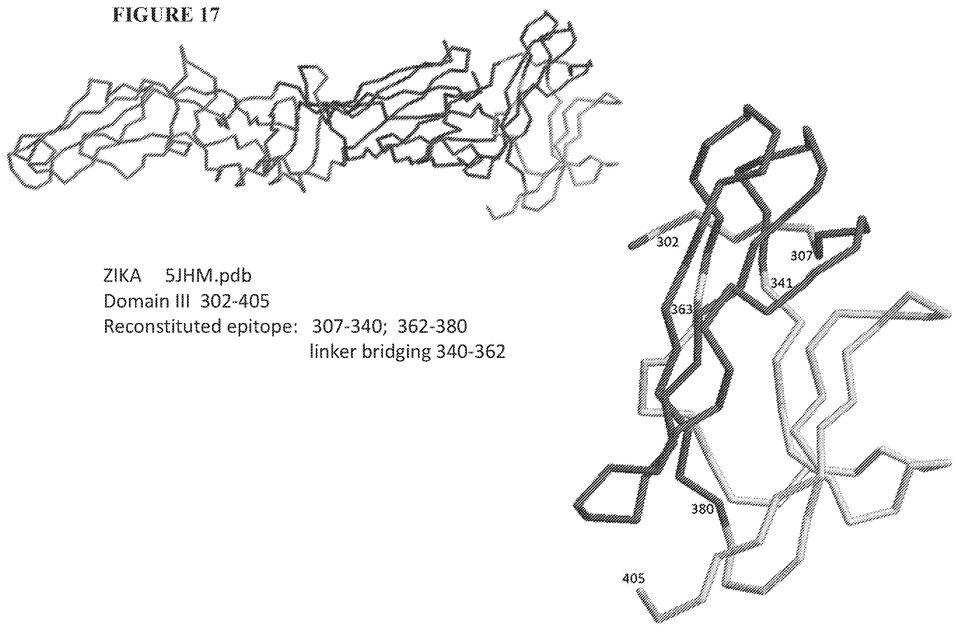

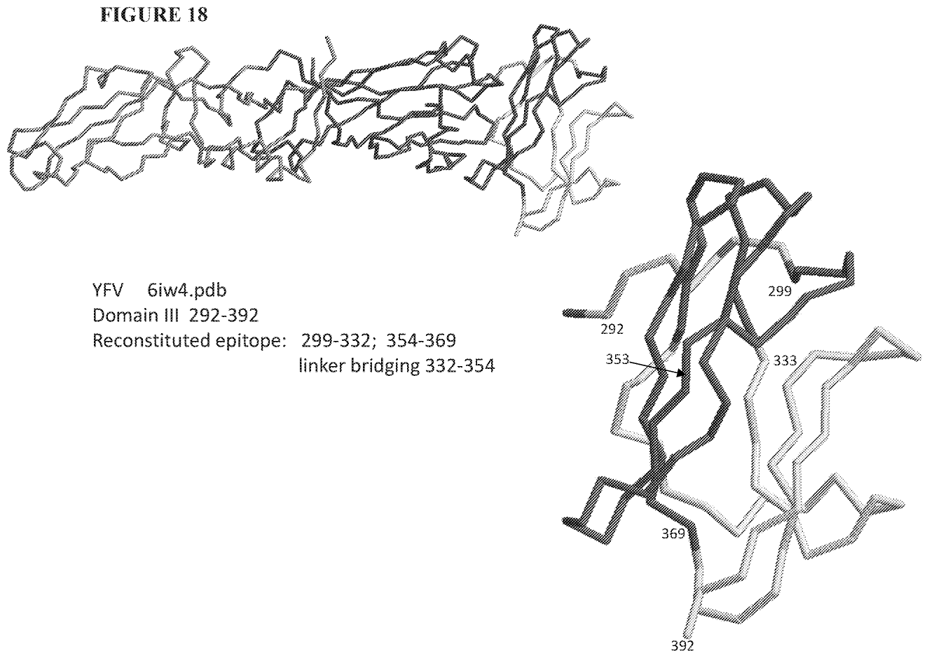

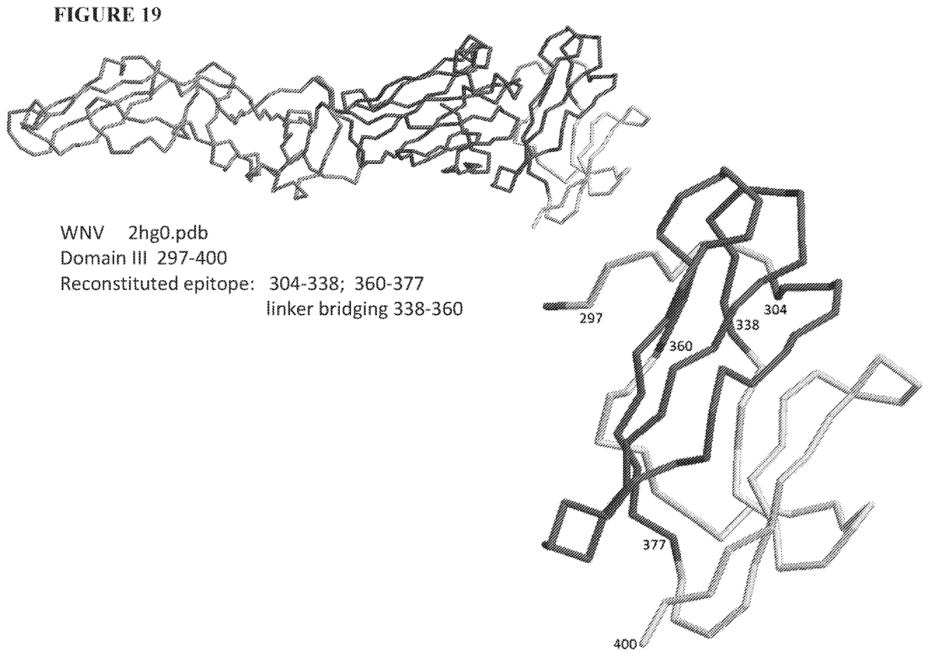

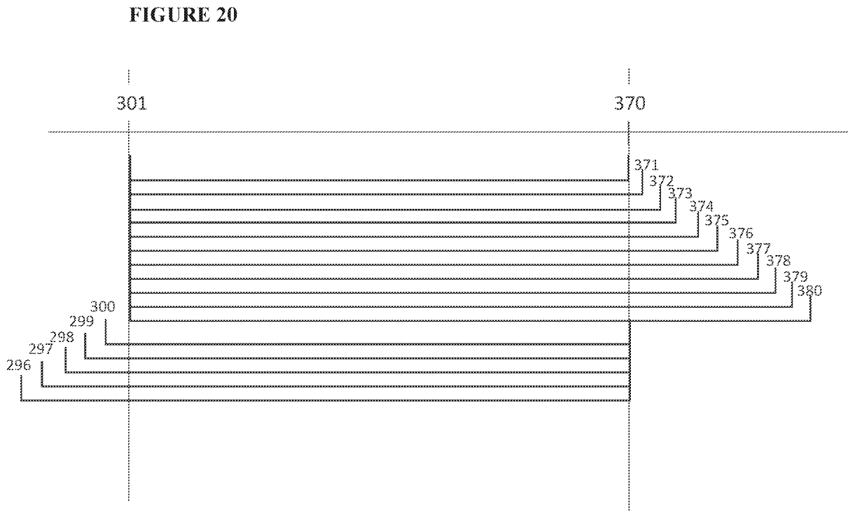

The 3 reconstituted epitopes that proved to be functional when incorporated with a carrier protein of various types (A11, F8 and H1), were selected to be used as immunogens for the inoculation of laboratory animals (mice). Seven arms of the experiment were as follows: MBP fused immunogens for A11, F8 and H1 reconstituted epitopes were prepared along with MBP control immunogen (no insert), MBP expressed full length DIII domain (FL) and loopless domain (LL) in which residue 335 is directly linked to 356 (i.e., no linker), and additional baseline (BL) arm that was not inoculated with any protein, and served as negative control for comparison for the immune response, hence 3 experimental groups compared to 4 control groups (FIG. 16A). Each experimental arm consisted of 5 female about 20-25 g C57BL/6 mice. The immunization performed using Imject™ Alum adjuvate. Each mouse was injected intraperitoneal with 50 μg antigen per injection. Three weeks after the initial immunization a boost was given followed three weeks later by a second boost (a total of three injections). Three weeks after the second boost the animals were sacrificed and the full volume of blood was collected from the heart, from which sera were separated and collected for examination. The collected sera were used for antigen recognition tested by ELISA using phage-displayed epitopes and I53-conjugated proteins as baits. Animals immunized with the F8 and H1 constructs were able to specifically bind both F8 and H1 constructs expressed as phages, and demonstrated cross-reactive features. The reaction of the A11 derived sera were similar to the baseline of pre-immune mice (FIG. 16B). Similarly, binding was demonstrated when the 153 antigens were tested (FIG. 16C). Next, sera were tested for Dengue VLP-binding of 4 serotypes. FIG. 16D demonstrates that the immunization with the recombinant F8 epitope elicits antibodies that recognize and cross react with Dengue VLPs of serotypes 1 and 2, whereas immunization with the recombinant H1 epitope elicits antibodies that recognize Dengue VLPs of serotype 1.

### Example 6

### Epitope-Based Vaccines of Zika Virus

Zika virus (ZV) contains a positive RNA that codes among other proteins an Envelope (E) Protein. Here, following the general scheme for the reconstitution of the Dengue Virus DIII epitope (see Figures and Tables cited in Example 1D above), a comprehensive conformer library for the reconstitution of a neutralizing epitope situated in the DIII domain of the E protein of ZV is constructed. The library is cloned as a fusion of the P3 protein of the filamentous bacteriophage fd. Once constructed the library is to be screened against neutralizing antibodies against ZV that bind the reconstituted DIII domain.

The ZV envelope protein consists of three structural domains (DI, DII and DIII, see FIG. 17) and is presented on the viral coat as a dimer of envelope proteins, oriented head to tail, with the DII domain of one subunit is juxtaposed across from the DIII domain of the opposing subunit and the DI domain bridging DII and DIII (see the general orientation as illustrated for Dengue virus in FIG. 2A-2D).

The technical approach for the reconstitution is to produce comprehensive conformer libraries of selected epitopes using filamentous phage display. Because the DIII epitope is greater than 60 amino acids in length, the library is constructed as N terminal fusions with the bacteriophage protein 3. The epitopes are constructed with diverse linkers, and NNK codons are used to enable the incorporation of all 20 amino acids at each residue position. For the reconstitution of the ZV DIII epitope described in FIG. 17, oligonucleotides and primers are produced as is described in Example 1 for Dengue Virus following Gibson Assembly into the BstX1 sites of the Protein 3 gene of the fth1 vector described above. The reconstituted construct contains a 5′ linker of 0-3 NNK codons preceding residue 307. An internal linker connecting residues 340 to 362 of 0-10 NNK codons is incorporated continuing to residue 380 where 0-3 NNK linkers are added. This library is cloned into the BstX1 sites of the vector and the phages are screened against neutralizing mAbs to affinity select those constructs whose linkers enable the segments of DIII (residues 307-340 and 362-380, of the amino acid sequence as denoted by SEQ ID NO: 100) to assume a physiological conformation. These affinity selected constructs are to be used as immunogens that elicit a protective neutralizing response in vaccines.

### Example 7

### Epitope-Based Vaccines of Yellow Fever Virus

Yellow Fever Virus (YFV) contains a positive RNA that codes among other proteins an Envelope (E) Protein. Here, following the general scheme for the reconstitution of the Dengue Virus DIII epitope (see Figures and Tables cited in Example 1D above), a comprehensive conformer library for the reconstitution of a neutralizing epitope situated in the DIII domain of the E protein of YFV is constructed. The library is cloned as a fusion of the P3 protein of the filamentous bacteriophage fd. Once constructed the library is to be screened against neutralizing antibodies against YFV that bind the reconstituted DIII domain.

The YFV envelope protein consists of three structural domains (DI, DII and DIII, see FIG. 18) and is presented on the viral coat as a dimer of envelope proteins, oriented head to tail, with the DII domain of one subunit is juxtaposed across from the DIII domain of the opposing subunit and the DI domain bridging DII and DIII (see the general orientation as illustrated for Dengue virus in FIG. 2A-2D).

The technical approach for the reconstitution is to produce comprehensive conformer libraries of selected epitopes using filamentous phage display. Because the DIII epitope is greater than 60 amino acids in length, the library is constructed as N terminal fusions with the bacteriophage protein 3. The epitopes are constructed with diverse linkers, and NNK codons are used to enable the incorporation of all 20 amino acids at each residue position. For the reconstitution of the YFV DIII epitope described in FIG. 18, oligonucleotides and primers are produced as is described in Example 1 for Dengue Virus following Gibson Assembly into the BstX1 sites of the Protein 3 gene of the fth1 vector described above. The reconstituted construct contains a 5′ linker of 0-3 NNK codons preceding residue 299. An internal linker connecting residues 332 to 352 of 0-10 NNK codons is incorporated continuing to residue 369 where 0-3 NNK linkers are added. This library is cloned into the BstX1 sites of the vector and the phages are screened against neutralizing mAbs to affinity select those constructs whose linkers enable the segments of DIII (residues 299-332 and 352-369 of SEQ ID NO: 101) to assume a physiological conformation. These affinity-selected constructs are to be used as immunogens that elicit a protective neutralizing response in vaccines.

### Example 8

### Epitope-Based Vaccines of West Nile Virus

West Nile virus (WNV) contains a positive RNA that codes among other proteins an Envelope (E) Protein. Here, following the general scheme for the reconstitution of the Dengue Virus DIII epitope (see Figures and Tables cited in Example 1D above), a comprehensive conformer library for the reconstitution of a neutralizing epitope situated in the DIII domain of the E protein of WNV is constructed. The library is cloned as a fusion of the P3 protein of the filamentous bacteriophage fd. Once constructed the library is to be screened against neutralizing antibodies against WNV that bind the reconstituted DIII domain.

The WNV envelope protein consists of three structural domains (DI, DII and DIII, see FIG. 19) and is presented on the viral coat as a dimer of envelope proteins, oriented head to tail, with the DII domain of one subunit is juxtaposed across from the DIII domain of the opposing subunit and the DI domain bridging DII and DIII (see the general orientation as illustrated for Dengue virus in FIG. 2A-2D).

The technical approach for the reconstitution is to produce comprehensive conformer libraries of selected epitopes using filamentous phage display. Because the DIII epitope is greater than 60 amino acids in length, the library is constructed as N terminal fusions with the bacteriophage protein 3. The epitopes are constructed with diverse linkers, and NNK codons are used to enable the incorporation of all 20 amino acids at each residue position. For the reconstitution of the WNV DIII epitope described in FIG. 19, oligonucleotides and primers are produced as is described in Example 1 for Dengue Virus following Gibson Assembly into the BstX1 sites of the Protein 3 gene of the fth1 vector described above. The reconstituted construct contains a 5′ linker of 0-3 NNK codons preceding residue 304. An internal linker connecting residues 338 to 360 of 0-10 NNK codons is incorporated continuing to residue 377 where 0-3 NNK linkers are added. This library is cloned into the BstX1 sites of the vector and the phages are screened against neutralizing mAbs to affinity select those constructs whose linkers enable the segments of DIII (residues 304-338 and 360-377 of SEQ ID NO: 102) to assume a physiological conformation. These affinity-selected constructs are to be used as immunogens that elicit a protective neutralizing response in vaccines.

1-55. (canceled)

56. A polypeptide comprising an amino acid sequence of at least one reconstituted epitope of a viral envelope protein, or any composition thereof, said viral envelop protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII, wherein said reconstituted epitope comprises at least one linker and at least one fragment of the native envelope protein.

57. The polypeptide according to claim 56, wherein said enveloped virus is of the Flaviviridae family, optionally, wherein at least one of:
(a) said virus of the Flaviviridae family is a virus of the Flavivirus genus;
(b) said virus is at least one of: Dengue virus, Zika virus, Yellow Fever virus, West Nile virus, Tick-borne encephalitis virus, Japanese encephalitis virus and Tembusu virus;

58. The polypeptide according to claim 56, wherein said virus is Dengue virus; optionally, at least one of:
(a) wherein said envelope protein comprises an amino acid sequence as denoted by any one of SEQ ID NO: 96, 97, 98 and 99, and any variants, mutants and homologs thereof; and
(b) wherein said epitope comprises at least in part, at least one amino acid sequence of the DIII domain of the native envelope protein (E protein) of said Dengue virus, and any fragments thereof.

59. The polypeptide according to claim 56, wherein at least one of:
(I) said at least one fragment of the native E protein comprises at least one of:
(a) at least one amino acid sequence starting at any one of the amino acid residues 301, 296, 297, 298, 299, 300, 302, 303, 304, 305 or 306, and ending at any one of the amino acid residues 370, 365, 366, 367, 368, 369, 371, 372, 373, 374, 375, 376, 377, 378, 379, 380, 381, 382, 383, 384, 385, 386, 387, 388, 389, 390, 391, 392, 393, 39, 395, 396, 397 or 398;
(b) at least one amino acid sequence starting at any one of the amino acid residues 301, 296, 297, 298, 299, 300, 302, 303, 304, 305 and 306 and ending at any one of the amino acid residues 335, 329, 330, 331, 332, 333, 334, 336, 337, 338, 339 or 340; and
(c) at least one amino acid sequence starting at any one of the amino acid residues 356, 351, 352, 353, 354, 355, 357, 358, 359, 360 or 361, and ending at any one of the amino acid residues 370, 365, 366, 367, 368, 369, 371, 372, 373, 374, 375, 376, 377, 378, 379, 380, 381, 382, 383, 384, 385, 386, 387, 388, 389, 390, 391, 392, 393, 39, 395, 396, 397 or 398; and/or
(II) said epitope comprises an amino acid sequence of the native DIII domain of said E protein starting at any one of the amino acid residues 301, 296, 297, 298, 299, 300, 302, 303, 304, 305 or 306 and ending at any one of the amino acid residues 370, 365, 366, 367, 368, 369, 371, 372, 373, 374, 375, 376, 377, 378, 379, 380, 381, 382, 383, 384, 385, 386, 387, 388, 389, 390, 391, 392, 393, 39, 395, 396, 397 or 398, wherein said native DIII domain comprises a loop comprising an amino acid sequence starting at any one of the amino acid residues 336, 333, 334, 335, 337, 338 or 339, and ending at any one of the amino acid residues 355, 352, 353, 354, 356, 357 or 358, and wherein at least one of said linker/s replaces said loop or any part thereof or amino acid residue/s thereof and any DIII domain fragment or amino acid residue/s thereof.

60. The polypeptide according to claim 56, wherein said reconstituted epitope comprises at least one linker and at least two fragments of the native E protein, wherein said at least two fragments comprise:
(A) the amino acid sequence of any one of:
(i) residues M301 to I335 of the envelope protein;
(ii) residues M301 to I335 of the envelope protein with at least one or two flanking amino acid residue/s; or
(iii) any variant, mutant, parts or fragments of the amino acid sequence of residues M301 to I335 of the envelope protein; and
(B) the amino acid sequence of any one of:
(i) residues P356 to E370 of the envelope protein;
(ii) residues P356 to E370 of the envelope protein with at least one or two flanking amino acid residue/s; or
(iii) any variant, mutant, parts or fragments of the amino acid sequence of residues P356 to E370 of the envelope protein; optionally, said at least one linker is at least one of:
(a) a bridging linker that bridges residue 335 with residue 356 of the of the envelope protein;
(b) a linker attached to the N′ terminus of said at least one fragment; and
(c) a linker attached to the C′ terminus of said at least one fragment.

61. The polypeptide according to claim 56, wherein said virus is a Zika virus, and wherein at least one of:
(I) said envelope protein comprises an amino acid sequence as denoted by SEQ ID NO: 100, and any variants, mutants and homologs thereof;
(II) said epitope comprises at least in part, at least one amino acid sequence of the DIII domain of the native E envelope protein of said Zika virus, and any fragments thereof;
(III) said at least one fragment of the native E protein comprises at least one of:
(a) at least one amino acid sequence starting at any one of the amino acid residues 307, 302, 303, 304, 305, 306, 308, 309, 310 or 311, and ending at any one of the amino acid residues 380, 375, 376, 377, 378, 379, 381, 382, 383, 384 or 385;
(b) at least one amino acid sequence starting at any one of the amino acid residues 307, 302, 303, 304, 305, 306, 308, 309, 310 or 311, and ending at least one of the amino acid residues 340, 335, 336, 337, 338, 339, 341, 342, 343, 344 or 345;
(c) at least one amino acid sequence starting at any one of the amino acid residues 362, 357, 358, 359, 360 or 361 and ending at least one of the amino acid residues 380, 375, 376, 377, 378, 379, 381, 382, 383, 384 or 385; and/or
(IV) said at least one linker is at least one of:
(a) a bridging linker that bridges residue 340 with residue 362 of the of the envelope protein;
(b) a linker attached to the N′ terminus of said at least one fragment; and
(c) a linker attached to the C′ terminus of said at least one fragment.

62. The polypeptide according to claim 56, wherein said virus is Yellow Fever virus, and wherein at least one of:
(I) said envelope protein comprises an amino acid sequence as denoted by SEQ ID NO: 101, and any variants, mutants and homologs thereof;
(II) said epitope comprises at least in part, at least one amino acid sequence of the DIII domain of the native E protein of said Yellow Fever virus, and any fragments thereof;
(III) said at least one fragment of the native E protein comprises at least one of:
(a) at least one amino acid sequence starting at any one of the amino acid residues 299, 294, 295, 296, 297, 298, 300, 301, 302, 303 or 304, and ending at any one of the amino acid residues 369, 364, 365, 366, 367, 368, 370, 371, 372, 373 or 374;
(b) at least one amino acid sequence starting at any one of the amino acid residues 299, 294, 295, 296, 297, 298, 300, 301, 302, 303 or 304, and ending at any one of the amino acid residues 332, 327, 328, 329, 330 or 331; and
(c) at least one amino acid sequence starting at any one of the amino acid residues 354, 349, 350, 351, 352, 353, 355, 356, 357, 358 or 359, and ending at any one of the amino acid residues 369, 364, 365, 366, 367, 368, 370, 371, 372, 373 or 374; and/or
(IV) said at least one linker is at least one of:
(a) a bridging linker that bridges residue 332 with residue 354 of the of the Yellow Fever virus envelope protein;
(b) a linker attached to the N′ terminus of said at least one fragment; and
(c) a linker attached to the C′ terminus of said at least one fragment.

63. The polypeptide according to claim 56, wherein said virus is West Nile virus, and wherein at least one of:
(I) said envelope protein comprises an amino acid sequence as denoted by SEQ ID NO:102, and any mutants, variants and homologs thereof;
(II) said epitope comprises at least in part, at least one amino acid sequence of the DIII domain of the native E protein of said West Nile virus, and any fragments thereof;
(III) said at least one fragment of the native E protein is at least one of:
(a) at least one amino acid sequence starting at any one of residues 304, 299, 300, 301, 302, 303, 305, 306, 307, 308 or 309 and ending at any one of the amino acid residues 377, 372, 373, 374, 375, 376, 378, 379, 380, 381 or 382;
(b) at least one amino acid sequence starting at any one of residues 304, 299, 300, 301, 302, 303, 305, 306, 307, 308 or 309 and ending at any one of the amino acid residues 338, 333, 334, 335, 336, 337, 339, 340, 341, 342, or 343; and
(c) at least one amino acid sequence starting at any one of residues 360, 35, 356, 357, 358, 359, 361, 362, 363, 364 or 365 and ending at any one of the amino acid residues 377, 372, 373, 374, 375, 376, 378, 379, 380, 381 or 382; and/or
(IV) said at least one linker is at least one of:
(a) a bridging linker that bridges residue 338 with residue 360 of the of the envelope protein;
(b) a linker attached to the N′ terminus of said at least one fragment; and
(c) a linker attached to the C′ terminus of said at least one fragment.

64. The polypeptide according to claim 56, wherein at least one of:
(a) said at least one linker is an amino acid linker comprising 1 to 10 amino acid residues;
(b) said at least one polypeptide is at least one DIII domain of a native E protein of a virus of the Flaviviridae family;
(c) said at least one polypeptide is at least one envelope protein (E protein) of a virus of the Flaviviridae family.

65. A DIII domain of an E protein of a virus of the Flaviviridae family, comprising the native DIII domain of an E protein of a virus of the Flaviviridae family or any fragments thereof and at least one linker, or any composition thereof, wherein at least one of said linker replaces a loop in said DIII domain, or any part thereof or amino acid residue/s thereof, wherein at least one of:
(a) said virus of the Flaviviridae family is a Dengue virus, and wherein said loop comprising an amino acid sequence starting at any one of the amino acid residues 336, 333, 334, 335, 337, 338 or 339, and ending at any one of the amino acid residues 355, 352, 353, 354, 356, 357 or 358;
(b) said virus of the Flaviviridae family is a Zika virus, and wherein said loop comprising an amino acid sequence starting at any one of the amino acid residues 341, 336, 337, 338, 339, 340, 342, 343, 344, 345 or 346 and ending at any one of the amino acid residues 361, 356, 357, 358, 359, 360, 362, 363, 364, 365 or 366;
(c) said virus of the Flaviviridae family is a Yellow Fever virus, and wherein said loop comprising an amino acid sequence starting at any one of the amino acid residues 333, 328, 329, 330, 331, 332, 334, 335, 336, 337, or 338 and ending at any one of the amino acid residues 353, 348, 349, 350, 351, 352, 354, 355, 356, 357, or 358;
(d) said virus of the Flaviviridae family is a West Nile virus, and wherein said loop comprising an amino acid sequence starting at any one of the amino acid residues 339, 334, 335, 336, 337, 338, 340, 341, 342, 343, or 344 and ending at any one of the amino acid residues 359, 354, 355, 356, 357, 358, 360, 361, 362, 363 or 364; and wherein at least one of said linker is a bridging linker, optionally, said DIII domain further comprises at least one linker flanking the N′ and/or C′ termini thereof.

66. An envelope protein (E protein) of a virus of the Flaviviridae family, comprising the native E protein of a virus of the Flaviviridae family or any fragments thereof and at least one linker, or any composition thereof, wherein at least one of said linker replaces a loop in the DIII domain of said envelope protein, or any part thereof or amino acid residue/s thereof, wherein at least one of:
(a) said virus of the Flaviviridae family is a Dengue virus, and wherein said loop comprising an amino acid sequence starting at any one of the amino acid residues 336, 333, 334, 335, 337, 338 or 339, and ending at any one of the amino acid residues 355, 352, 353, 354, 356, 357 or 358;
(b) said virus of the Flaviviridae family is a Zika virus, and wherein said loop comprising an amino acid sequence starting at any one of the amino acid residues 341, 336, 337, 338, 339, 340, 342, 343, 344, 345 or 346 and ending at any one of the amino acid residues 361, 356, 357, 358, 359, 360, 362, 363, 364, 365 or 366;
(c) said virus of the Flaviviridae family is a Yellow Fever virus, and wherein said loop comprising an amino acid sequence starting at any one of the amino acid residues 333, 328, 329, 330, 331, 332, 334, 335, 336, 337, or 338 and ending at any one of the amino acid residues 353, 348, 349, 350, 351, 352, 354, 355, 356, 357, or 358;
(d) said virus of the Flaviviridae family is a West Nile virus, and wherein said loop comprising an amino acid sequence starting at any one of the amino acid residues 339, 334, 335, 336, 337, 338, 340, 341, 342, 343, or 344 and ending at any one of the amino acid residues 359, 354, 355, 356, 357, 358, 360, 361, 362, 363 or 364; and wherein at least one of said linker is a bridging linker, optionally, said E protein further comprises at least one linker flanking the N′ and/or C′ termini of said DIII domain.

67. A multimeric and/or multivalent antigen displaying platform and/or nanoparticle scaffold comprising at least one reconstituted epitope of a viral envelope protein according to claim 56, any polypeptide, domain or viral envelope protein comprising said reconstituted epitope, any domain or viral envelope protein comprising at least one linker that replaces at least one loop or any part thereof or amino acid residue/s thereof in said viral protein, wherein said viral envelope protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII, wherein said reconstituted epitope comprises at least one linker and at least one fragment of the native envelope protein.

68. A nucleic acid sequence encoding at least one reconstituted epitope of a viral envelope protein according to claim 56, any polypeptide, domain or viral envelope protein comprising said reconstituted epitope, any domain or viral envelope protein comprising at least one linker that replaces at least one loop or any part thereof or amino acid residue/s thereof in said viral protein, any multimeric and/or multivalent antigen displaying platform thereof, and any combinations thereof, any fusion protein, conjugate, polyvalent dendrimer thereof, wherein said viral envelope protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII, wherein said reconstituted epitope comprises at least one linker and at least one fragment of the native envelope protein.

69. An anti-viral vaccine comprising at least one polypeptide comprising an amino acid sequence of at least one reconstituted epitope of a viral envelope protein, any polypeptide, domain or viral envelope protein comprising said reconstituted epitope, any domain or viral envelope protein comprising at least one linker that replaces at least one loop or any part thereof or amino acid residue/s thereof in said viral protein, any multimeric and/or multivalent antigen displaying platform thereof, and any combinations thereof, any derivative, enantiomer, fusion protein, conjugate, polyvalent dendrimer thereof, and any nucleic acid sequence encoding the same, or any matrix, nano- or micro-particle thereof, wherein said viral envelope protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII, and wherein said reconstituted epitope comprises at least one linker and at least one fragment of the native envelope protein, said vaccine optionally further comprises at least one pharmaceutically acceptable carrier/s, excipient/s, adjuvant/s, auxiliaries, and/or diluent/s, optionally, said vaccine is capable of eliciting an immune response specific for said virus in a subject.

70. A method for treating, preventing, inhibiting, reducing, eliminating, protecting or delaying the onset of an infection or an infectious clinical condition caused by a virus in a subject in need thereof, the method comprising the step of administering to said subject an effective amount of at least one polypeptide comprising an amino acid sequence of at least one reconstituted epitope of a viral envelope protein according to claim 56, any polypeptide, domain or viral envelope protein comprising said reconstituted epitope, any domain or viral envelope protein comprising at least one linker that replaces at least one loop or any part thereof or amino acid residue/s thereof in said viral protein, wherein said viral envelope protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII, wherein said reconstituted epitope comprises at least one linker and at least one fragment of the native envelope protein, or of any multimeric and/or multivalent antigen displaying platform thereof, any derivative, enantiomer, fusion protein, conjugate, polyvalent dendrimer thereof, any nucleic acid sequence encoding the same, and any combinations thereof, any compositions thereof and any vaccine thereof.

71. A method of inducing an immune response against a virus of the Flaviviridae family in a subject in need thereof, the method comprising administering to said subject an immunogenic effective amount of at least one polypeptide comprising an amino acid sequence of a viral envelope protein, any polypeptide, domain or viral envelope protein comprising said reconstituted epitope, any domain or viral envelope protein comprising at least one linker that replaces at least one loop or any part thereof or amino acid residue/s thereof in said viral protein, any multimeric and/or multivalent antigen displaying platform thereof, and any combinations thereof, any derivative, enantiomer, fusion protein, conjugate, polyvalent dendrimer thereof, and any nucleic acid sequence encoding the same or any matrix, nano- or micro-particle thereof, or any composition or vaccine thereof, wherein said viral envelope protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII, wherein said reconstituted epitope comprises at least one linker and at least one fragment of the native envelope protein, optionally, said method is for eliciting a neutralizing antibody response to said virus in said subject, and/or for preventing Antibody Dependent Enhancement (ADE) in said subject.

72. A method for the preparation of a functional reconstituted epitope of a viral envelope protein according to claim 56, wherein said viral envelope protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII, the method comprising the step of:
(a) screening a conformer library of epitopes of said viral envelope protein with at least one binding molecule, said library comprising plurality of combinatorial display platforms or any display vehicles, each expressing a reconstituted epitope comprising at least one linker and at least one fragment of the native envelope protein;
(b) identifying and producing reconstituted epitope peptides which bind at least one of said binding molecules; optionally, at least one of:
(I) said binding molecule is at least one of: (a) antibodies that neutralize the virus; (b) neutralizing antibodies of convalescent serum of at least one patient recovered from said virus infection; (c) the receptor for said virus or any fragments thereof; and (d) and any combinations of (a), (b) and (c); and/or
(II) said virus belongs to the Flaviviridae family.

73. A method for producing an anti-viral vaccine comprising at least one reconstituted epitope of a viral envelope protein according to claim 56, said viral envelop protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII, wherein said reconstituted epitope comprises at least one linker and at least one fragment of the native envelope protein, the method comprising the steps of:
(a) preparing reconstituted functional epitope of an envelope protein of said virus by a method comprising;
(i) screening a conformer library of epitopes of said viral envelope protein with at least one binding molecule, said library comprising plurality of combinatorial display platforms or any display vehicles, each expressing a reconstituted epitope comprising at least one linker and at least one fragment of the native envelope protein;
(ii) identifying and producing reconstituted epitope peptides which bind at least one of said binding molecules; and
(b) admixing at least one of said reconstituted functional epitope of an envelope protein of said virus or any derivative or enantiomer thereof, or any fusion protein, conjugate, or polyvalent dendrimer comprising the same with at least one adjuvant/s, carrier/s, excipient/s, auxiliaries, and/or diluent/s.

74. A method for the preparation, affinity selection and/or isolation of neutralizing antibodies that neutralize a virus, the method comprising the steps of:
(a) contacting a serum or lymphocytes of at least one donor with an effective amount of reconstituted epitope of a viral envelope protein according to claim 56, any polypeptide, domain or viral envelope protein comprising said reconstituted epitope, any domain or viral envelope protein comprising at least one linker that replaces at least one loop or any part thereof or amino acid residue/s thereof in said viral protein, wherein said viral envelope protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII, wherein said reconstituted epitope comprises at least one linker and at least one fragment of the native envelope protein, or with any multimeric and/or multivalent antigen displaying platform thereof, any derivative, enantiomer, fusion protein, conjugate, polyvalent dendrimer thereof, and any combinations thereof; and
(b) recovering the antibodies or at least one lymphocyte bound to said reconstituted epitope; optionally,
wherein said virus is of the Flaviviridae family, and wherein said method is for the production of monoclonal neutralizing antibodies that neutralize said virus, the method comprising the steps of:
(i) contacting lymphocytes of at least one donor with an effective amount of said reconstituted epitope, any multimeric and/or multivalent antigen displaying platform thereof, and any combinations thereof, any derivative, enantiomer, fusion protein, conjugate, polyvalent dendrimer thereof, associated directly or indirectly to a detectable moiety and/or any solid support; and
(ii) selection and single cell cloning of antibody producing lymphocyte bound to said reconstituted epitope.

75. The method according to claim 74, wherein said virus is of the Flaviviridae family, and wherein said method is for the production of polyclonal neutralizing antibodies that neutralize said virus, the method comprising the steps of:
(a) contacting serum of at least one donor or any immunoglobulin fraction thereof, with an effective amount of said reconstituted epitope of a viral envelope protein, any polypeptide, domain or viral envelope protein comprising said reconstituted epitope, any domain or viral envelope protein comprising at least one linker that replaces at least one loop or any part thereof or amino acid residue/s thereof in said viral protein, or with any multimeric and/or multivalent antigen displaying platform thereof, and any combinations thereof, any derivative, enantiomer, fusion protein, conjugate, polyvalent dendrimer thereof, associated directly or indirectly to a solid support and/or a detectable moiety, wherein said viral envelope protein is composed of three domains DI, DII and DIII and is presented on the viral coat of an enveloped virus as a dimer, oriented head to tail, with the DII domain of one subunit juxtaposed across from the DIII domain of the opposing subunit and the DI domain bridging DII and DIII, wherein said reconstituted epitope comprises at least one linker and at least one fragment of the native envelope protein; and
(b) recovering the antibodies bound to said reconstituted epitope immobilized to said solid support.
